(19) United States
(12) Patent Application Publication
(10) Pub. No.: US 2024/0216508 A1
(43) Pub. Date: Jul. 4, 2024

(54) ENGINEERED T CELLS CONDITIONALLY EXPRESSING A RECOMBINANT RECEPTOR, RELATED POLYNUCLEOTIDES AND METHODS

(71) Applicant: Juno Therapeutics GmbH, Munich (DE)

(72) Inventors: Mateusz Pawel POLTORAK, Munich (DE); Christian STEMBERGER, Munich (DE); Lothar GERMEROTH, Munich (DE)

(73) Assignee: Juno Therapeutics GmbH, Munich (DE)

(21) Appl. No.: 18/011,780

(22) PCT Filed: Jun. 25, 2021
(86) PCT No.: PCT/EP2021/067505

§ 371 Date: Dec. 20, 2022

Related U.S. Application Data
(60) Provisional application No. 63/044,984, filed on Jun. 26, 2020.

(51) Int. Cl. A61K 39/00; A61P 35/00; C07K 14/725; C12N 9/22; C12N 15/11; C12N 15/90

(52) U.S. Cl. CPC A61K 39/4611; A61K 39/4631; A61P 35/00; C07K 14/7051; C12N 9/22; C12N 15/11; C12N 15/907; A61K 2239/22; C07K 2319/02; C12N 2310/20

(57) ABSTRACT

Provided are engineered T cells that contain a modified T cell stimulation-associated locus encoding a recombinant receptor or a portion thereof. In some aspects, the nucleic acid sequence encoding the recombinant receptor or a portion thereof is operably linked to an endogenous transcriptional regulatory element of the T cell stimulation-associated locus, in some cases, engineered by targeted integration. In some aspects, the engineered cells conditionally express the recombinant receptor, such as upon stimulation or activation signal in the T cell. Also disclosed are cell compositions, nucleic acids for engineering cells, and methods and articles of manufacture for producing the engineered cells. In some embodiments, the engineered cells can be used in connection with cell therapy, including in connection with cancer immunotherapy comprising adoptive transfer of the engineered cells.

### CROSS-REFERENCE TO RELATED APPLICATIONS

This application is a national stage application under 35 U.S.C. § 371 of international application No. PCT/EP2021/067505, filed internationally on Jun. 25, 2021, which claims priority from U.S. provisional application No. 63/044,984, filed Jun. 26, 2020, entitled “ENGINEERED T CELLS CONDITIONALLY EXPRESSING A RECOMBINANT RECEPTOR, RELATED POLYNUCLEOTIDES AND METHODS,” the contents of each of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042013800SubSeqList.txt, created Jun. 28, 2023, which is 226,187 bytes in size. The information in the electronic format of the Sequence Listing is incorporated by reference in its entirety.

### FIELD

The present disclosure relates to engineered T cells that contain a modified T cell stimulation-associated locus encoding a recombinant receptor or a portion thereof. In some aspects, the nucleic acid sequence encoding the recombinant receptor or a portion thereof is operably linked to an endogenous transcriptional regulatory element of the T cell stimulation-associated locus, in some cases, engineered by targeted integration. In some aspects, the engineered cells conditionally express the recombinant receptor, such as upon stimulation or activation signal in the T cell. Also disclosed are cell compositions, nucleic acids for engineering cells, and methods and articles of manufacture for producing the engineered cells. In some embodiments, the engineered cells can be used in connection with cell therapy, including in connection with cancer immunotherapy comprising adoptive transfer of the engineered cells.

### BACKGROUND

Adoptive cell therapies that utilize recombinant receptors, such as chimeric antigen receptors (CARs) or recombinant T cell receptors (TCRs), to recognize antigens associated with a disease represent an attractive therapeutic modality for the treatment of cancers and other diseases. Improved strategies are needed for engineering T cells to express recombinant receptors, such as for use in adoptive immunotherapy, e.g., in treating cancer, infectious diseases and autoimmune diseases. Provided are methods, cells, compositions and kits for use in the methods that meet such needs.

### SUMMARY

Provided herein are engineered T cells including a modified T cell stimulation-associated locus containing a transgene encoding a recombinant receptor or a portion thereof, wherein the transgene is operably linked to an endogenous transcriptional regulatory element of the T cell stimulation-associated locus, in which the endogenous transcriptional regulatory element induces or upregulates expression of the operably linked transgene following a simulation or activation signal in the T cell.

In some of any embodiments, the endogenous transcriptional regulatory element is a promoter of an endogenous T cell stimulation-associated locus. In some of any embodiments, the transgene encoding the recombinant receptor or a portion thereof is present downstream of the promoter.

In some of any embodiments, the expression of the operably linked transgene is inducible, and is induced following the stimulation or activation signal in the cell. In some of any embodiments, the expression of the operably linked transgene is upregulated or induced within less than at or about 6 hours following the stimulation or activation signal in the T cells. In some of any embodiments, the expression of the operably linked transgene is upregulated or induced within less than at or about 12 hours following the stimulation or activation signal in the T cells. In some of any embodiments, the expression of the operably linked transgene is upregulated or induced within less than at or about 24 hours following the stimulation or activation signal in the T cells. In some of any embodiments, the expression of the operably linked transgene is upregulated or induced within less than at or about 36 hours following the stimulation or activation signal in the T cells. In some of any embodiments, the expression of the operably linked transgene is upregulated or induced within less than at or about 48 hours following the stimulation or activation signal in the T cells.

In some of any embodiments, the expression of the operably linked transgene is not permanent and/or may be reduced over time or in the absence of the stimulation or activation signal by the T cell. In some of any embodiments, following the upregulation or induction of expression or following a reduction or an absence of the simulation or activation signal in the T cells, the expression of the operably linked transgene is reduced or downregulated. In some of any embodiments, following the upregulation or induction of expression, or following the reduction or the absence of the simulation or activation signal in the T cells, the expression of the operably linked transgene is reduced or downregulated by greater than at or about 30%, 40%, 50%, 60%, 70%, 75%, 80%, 85%, 90% or 95% or more. In some of any embodiments, following the upregulation or induction of expression, the expression of the operably linked transgene is reduced or downregulated after at or about 1, 2, 3, 4, 5, 6, 7 or 8 days or more after the stimulation or activation signal in the T cells. In some of any embodiments, the expression of the operably linked transgene is reduced or downregulated within less than at or about 6, 12, 18, 24, 36 or 48 hours following the reduction or the absence of the simulation or activation signal in the T cell.

In some of any embodiments, the expression of the operably linked transgene is capable of being induced or upregulated again following a further simulation or activation signal in the T cells after the reduction or the absence of the simulation or activation signal. In some of any embodiments, the expression of the operably linked transgene is upregulated or induced within less than at or about 6, 12, 18, 24, 36 or 48 hours following the further simulation or activation signal in the T cells after the reduction or the absence of the simulation or activation signal. In some of any embodiments, the expression of the operably linked transgene is inducible, and is induced following the stimulation or activation signal in the cell. In some of any embodiments, the expression of the operably linked transgene is upregulated or induced within less than at or about 6 hours following the further simulation or activation signal in the T cells after the reduction or the absence of the simulation or activation signal. In some of any embodiments, the expression of the operably linked transgene is upregulated or induced within less than at or about 12 hours following the further simulation or activation signal in the T cells after the reduction or the absence of the simulation or activation signal. In some of any embodiments, the expression of the operably linked transgene is upregulated or induced within less than at or about 24 hours following the further simulation or activation signal in the T cells after the reduction or the absence of the simulation or activation signal. In some of any embodiments, the expression of the operably linked transgene is upregulated or induced within less than at or about 36 hours following the further simulation or activation signal in the T cells after the reduction or the absence of the simulation or activation signal. In some of any embodiments, the expression of the operably linked transgene is upregulated or induced within less than at or about 48 hours following the further simulation or activation signal in the T cells after the reduction or the absence of the simulation or activation signal.

In some of any embodiments, a translated product from an open reading frame of the endogenous T cell stimulation-associated locus is not expressed in the cell or a functional endogenous gene product of the endogenous T cell stimulation-associated locus is not expressed, following the simulation or activation signal in the T cell. In some of any embodiments, the modified T cell stimulation-associated locus includes a deletion, an insertion, a frameshift mutation or a nonsense mutation in the open reading frame of the endogenous T cell stimulation-associated locus.

In some of any embodiments, the endogenous T cell stimulation-associated locus is selected from among PDCD1, CD69, Nur77, FoxP3 and a HLA-DR locus. In some of any embodiments, the endogenous T cell stimulation-associated locus is a PDCD1 locus. In some of any embodiments, the endogenous T cell stimulation-associated locus is a CD69 locus. In some of any embodiments, the endogenous T cell stimulation-associated locus is a Nur77 locus. In some of any embodiments, the endogenous T cell stimulation-associated locus is a FoxP3 locus. In some of any embodiments, the endogenous T cell stimulation-associated locus is a HLA-DR locus. In some of any embodiments, the endogenous transcriptional regulatory element includes one or more response elements recognized by a transcription factor that is activated following the stimulation or activation signal.

In some of any embodiments, the recombinant receptor or portion thereof is capable of inducing or transmitting the stimulation or activation signal in the T cell.

In some of any embodiments, the recombinant receptor includes an intracellular region including an intracellular signaling domain of a component of the T cell receptor (TCR) complex, and the stimulation or activation signal in the T cells includes a signal through the intracellular signaling domain present in the recombinant receptor. In some of any embodiments, the recombinant receptor includes an intracellular region that includes an intracellular signaling domain including an immunoreceptor tyrosine-based activation motif (ITAM), and the stimulation or activation signal in the T cells includes a signal through the intracellular signaling domain present in the recombinant receptor. In some of any embodiments, the intracellular signaling domain of the recombinant receptor (e.g. CAR) may include a CD3zeta signaling domain, and the stimulation or activation signal in the T cells includes a signal through the intracellular signaling domain present in the recombinant receptor (e.g. the CAR).

In some of any embodiments, the recombinant receptor is a TCR and is able to recruit components of the T cell complex to induce or stimulate the activation signal in the T cell.

In some of any embodiments, the recombinant receptor includes an extracellular region including a binding domain that is capable of binding to or recognizing an agent (e.g. a target antigen). In some of any embodiments, a stimulation or activation signal is induced in the T cell upon binding or recognition of the agent by the recombinant receptor.

In some of any embodiments, the agent is a target antigen. In some of any embodiments, the target antigen is a recombinant protein or is an antigen expressed on the surface of a cell. In some of any embodiments, the target antigen is associated with, specific to, or expressed on a cell or tissue of a disease, disorder or condition. In some of any embodiments, the disease, disorder or condition is an infectious disease or disorder, an autoimmune disease, an inflammatory disease, or a tumor or a cancer. In some of any embodiments, the target antigen is a tumor antigen. In some of any embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some of any embodiments, the agent is an anti-idiotypic antibody that is specific to the extracellular domain of the recombinant receptor.

In some of any embodiments, the recombinant receptor is a chimeric antigen receptor (CAR). In some of any embodiments, the CAR includes an extracellular region that contains a binding domain, a transmembrane domain, and an intracellular region. In some of any embodiments, the extracellular region also includes a spacer. In some of any embodiments, the spacer is operably linked between the binding domain and the transmembrane domain. In some of any embodiments, the extracellular region includes a binding domain that is or includes an antibody or an antigen-binding fragment thereof, for example, a single-chain variable fragment (scFv).

In some of any embodiments, the intracellular region of the recombinant receptor (e.g. CAR) includes an intracellular signaling domain. In some of any embodiments, the intracellular signaling domain is or includes an intracellular signaling domain of a CD3 chain. In some of any embodiments, the intracellular signaling domain is or includes a CD3-zeta (CD3ζ) chain, or a signaling portion thereof. In some of any embodiments, the intracellular region includes one or more costimulatory signaling domain(s). In some of any embodiments, the intracellular region includes a CD3-zeta (CD3ζ) chain, or a signaling portion thereof, and one or more costimulatory signaling regions. In some of any embodiments, the one or more costimulatory signaling domain includes an intracellular signaling domain of a CD28, a 4-1BB or an ICOS or a signaling portion thereof. In some of any embodiments, the costimulatory signaling region includes an intracellular signaling domain of 4-1BB.

In some of any embodiments, the modified T cell stimulation-associated locus encodes a recombinant receptor that is a CAR, in which the CAR includes, from its N to C terminus in order: the extracellular binding domain, the spacer, the transmembrane domain and the intracellular region.

In some of any embodiments, the transgene includes in order a sequence of nucleotides encoding an extracellular binding domain, such as an scFv; a spacer, such as including a sequence from a human immunoglobulin hinge, such as from IgG1, IgG2 or IgG4 or a modified version thereof, such as further including a CH2 region and/or a CH3 region; and a transmembrane domain, such as from human CD28; a costimulatory signaling domain, such as from human 4-1BB; and an intracellular signaling domain, such as a CD3ζ chain or a portion thereof. In some of any embodiments, the modified T cell stimulation-associated locus includes in order: a sequence of nucleotides encoding an extracellular binding domain, such as an scFv; a spacer, such as including a sequence from a human immunoglobulin hinge, such as from IgG1, IgG2 or IgG4 or a modified version thereof, such as further including a CH2 region and/or a CH3 region; and a transmembrane domain, such as from human CD28; a costimulatory signaling domain, such as from human 4-1BB; and an intracellular signaling domain, such as a CD3ζ chain or a portion thereof.

In some of any embodiments, the transgene encodes the recombinant receptor, such as the full or complete sequence of the recombinant receptor. In some of any embodiments, the recombinant receptor is a single-chain polypeptide. For example, the recombinant receptor may be a chimeric antigen receptor (CAR) and the transgene encodes the chimeric antigen receptor (CAR), for example, the full or complete sequence of the CAR. In some of any embodiments, the recombinant receptor is a multiple-chain polypeptide, such as a two chain polypeptide. For example, the recombinant receptor may be a T cell receptor (TCR) that contains an alpha chain and a beta chain, and the transgene encodes both the alpha chain and beta chain of the TCR, such as the full or complete sequences of the alpha and beta chain of the TCR. In such an example, the separate chains of the TCR may be separated by a multicistronic element, such as a ribosome skip element (e.g. T2A or P2A) or an IRES.

In some of any embodiments, the transgene encodes a portion of the recombinant receptor. In embodiments, the portion of the recombinant receptor encoded by the transgene is capable of facilitating or allowing the same or similar (i.e. it retains) functional activity (e.g. antigen binding and receptor signaling activity), of the full-length recombinant receptor when the portion thereof is expressed from the T cell, such as following the simulation or activation signal in the T cell. In some of any embodiments, when expressed from the T cell the portion of the recombinant receptor can form a full recombinant receptor or a partial sequence thereof that retains functional activity (e.g. antigen binding and receptor signaling activity) of the full-length form of the recombinant receptor. In some of any embodiments, when expressed from the T cell, the portion of the recombinant receptor is able to facilitate or allow for at least 75%, 85%, 90%, or 100% of the full activity of the recombinant receptor. In some examples, when expressed from the T cell, the portion of the recombinant receptor encoded by the transgene is able to form a full functional receptor with another component of the recombinant receptor (e.g. another chain of the recombinant receptor) also expressed by the engineered T cell. In other examples, a recombinant receptor may be a single chain polypeptide (e.g. CAR), and the portion thereof may include a contiguous sequence of amino acids of the recombinant receptor that is necessary for the functional activity of the recombinant receptor, when it is expressed from the T cell. In such an embodiment, the portion thereof of the recombinant receptor may have a contiguous sequence of amino acids of a recombinant receptor that includes a sequence length that is at least about 85%, at least about 87%, at least about 90%, at least about 92%, at least about 95%, or at least about 97% of the length of a recombinant receptor, and that encodes a partial recombinant receptor that retains an activity of the recombinant receptor (e.g. antigen binding and receptor signaling activity).

In some of any embodiments, the portion thereof of the recombinant receptor is a polypeptide chain of the recombinant receptor, for example, in embodiments where the recombinant receptor is made up of multiple chains (e.g. a TCR containing an alpha chain and a beta chain, or a multi-chain CAR). For example, the other chain of the recombinant receptor is further expressed separately by the engineered T cell. In some of any embodiments, the recombinant receptor contains two separate polypeptide chains, in which the portion of the recombinant receptor encoded by the transgene is one chain of the recombinant receptor, and the engineered T cell further expresses the other chain of the recombinant receptor. In some of any embodiments, the other chain of the recombinant receptor is encoded by a second transgene that is separately contained in and able to be expressed by the T cell.

In some of any embodiments, the recombinant receptor is a CAR that is a multi-chain CAR. In some examples, the transgene encodes the multi-chain CAR, such as the full or entire sequence of the multi-chain CAR, for example a first chain and a second chain of a multi-chain CAR that has two chains. In such an example, the separate chains of the multi-chain CAR may be separated by a multicistronic element, such as a ribosome skip element (e.g. T2A or P2A) or an IRES. In other examples, the transgene encodes one chain of the multi-chain CAR, and the other chain of the multi-chain CAR is separately encoded by the engineered cell, for example by a second transgene. In some of any of the provided embodiments, the engineered T cell is capable of expressing a fully functional recombinant multi-chain CAR that retains or exhibits antigen-binding and receptor signaling activity, such as following a simulation or activation signal in the T cell.

In some of any embodiments, the recombinant receptor is a recombinant T cell receptor (TCR). In some of any embodiments, the recombinant TCR includes an alpha (TCRα) chain and a beta (TCRβ) chain and the transgene includes a nucleic acid sequence encoding the TCRα chain and/or a nucleic acid sequence encoding the TCRβ chain. In some examples, the transgene encodes the TCR, such as the full or entire sequence of the TCR including the TCRα and the TCRβ chain. In such an example, the separate chains of the TCR may be separated by a multicistronic element, such as a ribosome skip element (e.g. T2A or P2A) or an IRES. In some of any embodiments, the transgene encodes one of the TCRα chain or the TCRβ chain, and the other of the TCRα chain or the TCRβ chain is separately encoded by the engineered cell, for example by a second transgene. In some of any of the provided embodiments, the engineered T cell is capable of expressing a fully functional recombinant TCR that retains or exhibits antigen-binding and receptor signaling activity, such as following a simulation or activation signal in the T cell.

In some of any embodiments, the recombinant receptor is a TCR that contains a TCRα and a TCRβ chain. In some of any embodiments, the TCRα chain includes a constant (Cα) region including one or more introduced cysteine residues and/or the TCRβ chain includes a Cβ region including one or more introduced cysteine residues, in which the one or more introduced cysteine residues are capable of forming one or more non-native disulfide bridges between the alpha chain and beta chain. In some of any embodiments, the one or more introduced cysteine residues includes replacement of a non-cysteine residue with a cysteine residue. In some of any embodiments, the Cα region includes a cysteine at a position corresponding to position 48 with numbering as set forth in SEQ ID NO: 92; and/or the Cβ region includes a cysteine at a position corresponding to position 57 with numbering as set forth in SEQ ID NO: 96.

In some of any embodiments, the transgene includes a sequence of nucleotides encoding at least one further protein. In some of any embodiments, the at least one further protein is a surrogate marker, such as a marker to monitor or act as a surrogate for expression of the recombinant receptor by the engineered T cell. In some of any embodiments, the surrogate marker is a truncated receptor. In some of any embodiments, the truncated receptor lacks an intracellular signaling domain and/or is not capable of mediating intracellular signaling when bound by its ligand.

In some of any embodiments, the transgene further includes a multicistronic element(s). In some of any embodiments, the multicistronic element(s) includes a sequence encoding a ribosome skip element selected from among a T2A, a P2A, a E2A or a F2A, or an internal ribosome entry site (IRES).

In some of any embodiments, the multicistronic element is positioned between the sequence of nucleotides encoding the CAR and the sequence of nucleotides encoding the at least one further protein. In some of any embodiments, the recombinant receptor is a recombinant TCR, and the multicistronic element is positioned between a sequence of nucleotides encoding the TCRα and a sequence of nucleotides encoding the TCRβ. In some of any embodiments, the recombinant receptor is a multi-chain CAR, and the multicistronic element is positioned between a sequence of nucleotides encoding one chain of the multi-chain CAR and a sequence of nucleotides encoding another chain of the multi-chain CAR. In some of any embodiments, the multicistronic element(s) are upstream of the sequence of nucleotides encoding the recombinant receptor.

In some of any embodiments, the modified T cell stimulation-associated locus is produced by integration of the transgene encoding the recombinant receptor into the endogenous T cell stimulation-associated locus, such as by gene editing using homology-directed repair. In some of any embodiments, integration is by a) inducing a genetic disruption at one or more target site(s) at or near the endogenous T cell stimulation-associated locus; and b) introducing a polynucleotide for homology directed repair (HDR).

In some of any embodiments, the transgene encoding the recombinant receptor is integrated at or near the at least one target site in the T cell stimulation-associated locus. In some of any embodiments, the genetic disruption is effected by a zinc finger nuclease (ZFN), a TAL-effector nuclease (TALEN), or a CRISPR-Cas9 combination that specifically binds to, recognizes, or hybridizes to the target site. In some of any embodiments, the genetic disruption is effected by a CRISPR-Cas9 combination and the CRISPR-Cas9 combination includes a guide RNA (gRNA) having a targeting domain that is complementary to the at least one target site. In some of any embodiments, the CRISPR-Cas9 combination is a ribonucleoprotein (RNP) complex including the gRNA and a Cas9 protein. In some of any embodiments, the genetic disruption of is effected by the RNP introduced into a plurality of T cells via electroporation.

In some of any embodiments, the T cell stimulation-associated locus is PDCD1. In some of any embodiments, the genetic disruption is effected by a CRISPR-Cas9 combination including a gRNA and the gRNA has a targeting domain that is complementary to a target site in a PDCD1 gene. In some of any embodiments, the gRNA includes the sequence set forth in in any one of SEQ ID NOS: 75 and 104-109. In some of any embodiments, the gRNA includes the sequence set forth in SEQ ID NO:75.

In some of any embodiments, the T cell stimulation-associated locus is CD69. In some of any embodiments, the genetic disruption is effected by a CRISPR-Cas9 combination including a gRNA and the gRNA has a targeting domain that is complementary to a target site in a CD69 gene. In some of any embodiments, the gRNA includes the sequence set forth in any one of SEQ ID NOS: 116-121.

In some of any embodiments, the T cell stimulation-associated locus is Nur77. In some of any embodiments, the genetic disruption is effected by a CRISPR-Cas9 combination including a gRNA and the gRNA has a targeting domain that is complementary to a target site in a Nur77 gene. In some of any embodiments, the gRNA includes the sequence set forth in SEQ ID NO: 122-127 and 134-136.

In some of any embodiments, the T cell stimulation-associated locus is FoxP3. In some of any embodiments, the genetic disruption is effected by a CRISPR-Cas9 combination including a gRNA and the gRNA has a targeting domain that is complementary to a target site in a FoxP3 gene.

In some of any embodiments, the T cell stimulation-associated locus is a HLA-DR locus. 9. In some of any embodiments, the genetic disruption is effected by a CRISPR-Cas9 combination including a gRNA and the gRNA has a targeting domain that is complementary to a target site in a HLA-DR gene.

In some of any embodiments, the T cell further includes a genetic disruption at an endogenous T cell receptor alpha constant region (TRAC) gene and/or an endogenous T cell receptor beta constant region (TRBC) gene. In some of any embodiments, the T cell further includes a genetic disruption at an endogenous T cell receptor alpha constant region (TRAC) gene. In some of any embodiments, the T cell further includes a genetic disruption at an endogenous T cell receptor alpha constant region (TRBC) gene. In some of any embodiments, the T cell further includes a genetic disruption at an endogenous T cell receptor alpha constant region (TRAC) gene and an endogenous T cell receptor beta constant region (TRBC) gene. In some of any embodiments, the genetic disruption is effected by a zinc finger nuclease (ZFN), a TAL-effector nuclease (TALEN), or a CRISPR-Cas9 combination that specifically binds to, recognizes, or hybridizes to at least one target site, such as at least one target site within the TRAC gene or a TRBC gene, either TRBC1 or TRBC2 gene. In some of any embodiments, the genetic disruption is effected by a CRISPR-Cas9 combination and the CRISPR-Cas9 combination includes a guide RNA (gRNA) having a targeting domain that is complementary to the at least one target site. In some of any embodiments, the CRISPR-Cas9 combination is a ribonucleoprotein (RNP) complex including the gRNA and a Cas9 protein. In some of any embodiments, the genetic disruption is effected by the RNP introduced into a plurality of T cells via electroporation.

In some of any embodiments, the genetic disruption is effected by a CRISPR-Cas9 combination including a gRNA and the gRNA has a targeting domain that is complementary to at least one target site within the TRAC gene or a TRBC gene, either TRBC1 or TRBC2 gene. In some of any embodiments, the gRNA has a targeting domain that is complementary to a target site in a TRAC gene. In some of any embodiments, the gRNA includes the sequence set forth in any one of SEQ ID NOS: 77 and 188-218. In some of any embodiments, the gRNA includes the sequence set forth in SEQ ID NO:77. In some of any embodiments, the gRNA has a targeting domain that is complementary to a target site in a TRBC gene. In some of any embodiments, the gRNA includes the sequence set forth in any one of SEQ ID NOS: 219-276.

In some of any embodiments, signaling activity through the intracellular signaling domain of the encoded recombinant receptor in the absence of a simulation or activation signal in the T cells is reduced by greater than at or about 10%, 15%, 20%, 25%, 30%, 40%, 50% or more compared to an engineered T cell including a transgene encoding the same recombinant receptor present at a different location in the genome of the T cell or present at random locations in the genome of the T cell.

In some of any embodiments, the T cell is a CD8+ T cell or a CD4+ T cell or subtypes thereof. In some of any embodiments, the T cell is a primary T cell derived from a subject. In some of any embodiments, the subject is a human. In some embodiments, the T cell is a primary T cell derived from a human subject. In some embodiments, the T cell is a primary human T cell. In some of any embodiments, the T cell is derived from a multipotent or pluripotent cell. In some of any embodiments, the T cell is derived from an iPSC.

Also provided are compositions containing a plurality of any of the provided engineered cells. In some of any embodiments, the expression of the operably linked transgene is upregulated or induced in one or more cells in the composition within less than at or about 6, 12, 18, 24, 36 or 48 hours following the stimulation or activation signal in the T cells. In some of any embodiments, following a simulation or activation signal in the T cells, the frequency of cells expressing the operably linked transgene among the cells in the composition is greater than at or about 50%, 60%, 70%, 75%, 80%, 85%, 90% or 95% or more. In some of any embodiments, following the upregulation or induction of expression or following a reduction or an absence of the simulation or activation signal in the T cells, the expression of the operably linked transgene is reduced or downregulated in one or more cells in the composition.

In some of any embodiments, following the upregulation or induction of expression, or following the reduction or the absence of the simulation or activation signal in the T cells, the frequency of cells expressing the operably linked transgene among the cells in the composition is reduced by greater than at or about 30%, 40%, 50%, 60%, 70%, 75%, 80%, 85%, 90% or 95% or more. In some of any embodiments, following the upregulation or induction of expression, and the expression of the operably linked transgene is reduced or downregulated in one or more cells in the composition after at or about 1, 2, 3, 4, 5, 6, 7 or 8 days or more after the stimulation or activation signal in the T cells.

In some of any embodiments, the expression of the operably linked transgene in one or more of the cells in the composition is reduced or downregulated within less than at or about 6, 12, 18, 24, 36 or 48 hours following the reduction or an absence of the simulation or activation signal in the T cell. In some of any embodiments, following a reduction or an absence of the simulation or activation signal in the T cells, the frequency of cells expressing the recombinant receptor among the cells in the composition is less than at or about 50%, 40%, 30%, 25%, 20%, 15%, 10% or 5% or less.

In some of any embodiments, the composition includes CD4+ T cells and/or CD8+ T cells. In some of any embodiments, the composition includes CD4+ and CD8+ T cells and the ratio of CD4+ to CD8+ T cells is from or from about 1:3 to 3:1. In some of any embodiments, 1:1.

Also provided are polynucleotides that includes (a) a transgene encoding a recombinant receptor or a portion thereof, and (b) one or more homology arms linked to the transgene, in which the one or more homology arms comprise a sequence homologous to one or more region(s) of an endogenous T cell stimulation-associated locus in a T cell.

In some of any embodiments, the recombinant receptor or a portion thereof is encoded by a modified T cell stimulation-associated locus containing the transgene encoding the recombinant receptor or a portion thereof, when the recombinant receptor is expressed from a cell introduced with the polynucleotide. In some of any embodiments, the transgene is a sequence that is exogenous or heterologous to an open reading frame of the endogenous T cell stimulation-associated locus of a T cell.

In some of any embodiments, the T cell is a primary human T cell. In some of any embodiments, the T cell is a T cell derived from a subject. In some of any embodiments, the subject is a human. In some of any embodiments, the T cell is a human T cell.

In some of any embodiments, the one or more homology arm includes a 5′ homology arm and/or a 3′ homology arm. In some of any embodiments, the 5′ homology arm and 3′ homology arm includes nucleic acid sequences homologous to nucleic acid sequences surrounding a target site, in which the target site is within the T cell stimulation-associated locus. In some of any embodiments, the target site is downstream of an endogenous transcriptional regulatory element of the T cell stimulation-associated locus.

In some of any embodiments, the polynucleotide includes the structure [5′ homology arm]-[transgene]-[3′ homology arm]. In some of any embodiments, the 5′ homology arm and 3′ homology arm includes nucleic acid sequences homologous to nucleic acid sequences surrounding the at least one target site. In some of any embodiments, the 5′ homology arm and 3′ homology arm independently are between at or about 50 and at or about 750 nucleotides, at or about 50 and at or about 500 nucleotides, at or about 50 and at or about 250 nucleotides, at or about 50 and at or about 100 nucleotides, at or about 100 and at or about 750 nucleotides, at or about 100 and at or about 500 nucleotides, at or about 100 and at or about 250 nucleotides, at or about 250 and at or about 750 nucleotides, at or about 250 and at or about 500 nucleotides, in length. In some of any embodiments, the 5′ homology arm and 3′ homology arm independently are at or about 50, 60, 70, 80, 90, 100, 150, 200, 250, 300, 400 or 500 nucleotides, or any value between any of the foregoing, in length. In some of any embodiments, the 5′ homology arm and 3′ homology arm independently are less than at or about 100 nucleotides, in length. In some of any embodiments, the 5′ homology arm and 3′ homology arm independently are at or about 50, 60, 70, 80 or 90 nucleotides, or any value between any of the foregoing, in length.

In some of any embodiments, the T cell stimulation-associated locus is selected from among PDCD1, CD69, Nur77, FoxP3 and a HLA-DR locus.

In some of any embodiments, the T cell stimulation-associated locus is PDCD1. In some of any embodiments, the 5′ homology arm and 3′ homology arm comprise a sequence homologous to one or more region(s) of PDCD1. In some of any embodiments, the 5′ homology arm includes: a) a sequence including at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 66; b) a sequence including at or at least at or at least 150, 200, 250, 300, 350, 400, 450, 500, 550, or 600 contiguous nucleotides of the sequence set forth in SEQ ID NO:66; or c) the sequence set forth in SEQ ID NO: 66. In some of any embodiments, the 3′ homology arm includes: a) a sequence including at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 67; b) a sequence including at or at least at or at least 150, 200, 250, 300, 350, 400, 450, 500, 550, or 600 contiguous nucleotides of the sequence set forth in SEQ ID NO:67; or c) the sequence set forth in SEQ ID NO: 67.

In some of any embodiments, the T cell stimulation-associated locus is CD69. In some of any embodiments, the 5′ homology arm and 3′ homology arm comprise a sequence homologous to one or more region(s) of CD69.

In some of any embodiments, the T cell stimulation-associated locus is Nur77. In some of any embodiments, the 5′ homology arm and 3′ homology arm comprise a sequence homologous to one or more region(s) of Nur77.

In some of any embodiments, the T cell stimulation-associated locus is FoxP3. In some of any embodiments, the 5′ homology arm and 3′ homology arm comprise a sequence homologous to one or more region(s) of FoxP3.

In some of any embodiments, the T cell stimulation-associated locus is a HLA-DR locus. In some of any embodiments, the 5′ homology arm and 3′ homology arm comprise a sequence homologous to one or more region(s) of a HLA-DR locus.

In some of any embodiments, the recombinant receptor or portion thereof is capable of inducing or transmitting the stimulation or activation signal in the T cell.

In some of any embodiments, the recombinant receptor includes an intracellular region including an intracellular signaling domain of a component of the T cell receptor (TCR) complex and the stimulation or activation signal in the T cells includes a signal through the intracellular signaling domain present in the recombinant receptor, or the recombinant receptor includes an intracellular region including an intracellular signaling domain including an immunoreceptor tyrosine-based activation motif (ITAM) and the stimulation or activation signal in the T cells includes a signal through the intracellular signaling domain present in the recombinant receptor.

In some of any embodiments, the recombinant receptor includes an extracellular region including a binding domain that is capable of binding to or recognizing an agent. In some of any embodiments, a stimulation or activation signal is induced in the T cell upon binding of the agent.

In some of any embodiments, the agent is a target antigen. In some of any embodiments, the target antigen is a recombinant protein or is an antigen expressed on the surface of a cell. In some of any embodiments, the target antigen is associated with, specific to, or expressed on a cell or tissue of a disease, disorder or condition. In some of any embodiments, the disease, disorder or condition is an infectious disease or disorder, an autoimmune disease, an inflammatory disease, or a tumor or a cancer. In some of any embodiments, the target antigen is a tumor antigen. In some of any embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-11 and LAGE-12), carcinoembryonic antigen (CEA), a cyclin, cyclin A2, C—C Motif Chemokine Ligand 1 (CCL-11), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-12), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-122 receptor alpha (IL-122Rα), IL-113 receptor alpha 2 (IL-113Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-11), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-11), a pathogen-specific or pathogen-expressed antigen, or an antigen associated with a universal tag, and/or biotinylated molecules, and/or molecules expressed by HIV, HCV, HBV or other pathogens.

In some of any embodiments, the agent is an anti-idiotypic antibody.

In some of any embodiments, the recombinant receptor is a chimeric antigen receptor (CAR). In some of any embodiments, the CAR includes an extracellular region, a transmembrane domain, and an intracellular region. In some of any embodiments, the extracellular region includes a spacer. In some of any embodiments, the spacer is operably linked between the binding domain and the transmembrane domain. In some of any embodiments, the extracellular region includes a binding domain that is or includes an antibody or an antigen-binding fragment thereof.

In some of any embodiments, the intracellular region includes an intracellular signaling domain. In some of any embodiments, the intracellular signaling domain is or includes an intracellular signaling domain of a CD3 chain. In some of any embodiments, a CD3-zeta (CD3ζ) chain, or a signaling portion thereof. In some of any embodiments, the intracellular region includes one or more costimulatory signaling domain(s). In some of any embodiments, the one or more costimulatory signaling domain includes an intracellular signaling domain of a CD28, a 4-1BB or an ICOS or a signaling portion thereof. In some of any embodiments, the costimulatory signaling region includes an intracellular signaling domain of 4-1BB.

In some of any embodiments, the modified T cell stimulation-associated locus encodes a recombinant receptor that is a CAR, in which the CAR includes, from its N to C terminus in order: the extracellular binding domain, the spacer, the transmembrane domain and an intracellular region.

In some of any embodiments, the transgene comprises in order a sequence of nucleotides encoding an extracellular binding domain, such as an scFv; a spacer, such as comprising a sequence from a human immunoglobulin hinge, such as from IgG1, IgG2 or IgG4 or a modified version thereof, such as further comprising a CH2 region and/or a CH3 region; and a transmembrane domain, such as from human CD28; a costimulatory signaling domain, such as from human 4-1BB; and an intracellular signaling domain, such as a CD3ζ chain or a portion thereof; and/or the modified T cell stimulation-associated locus comprises in order: a sequence of nucleotides encoding an extracellular binding domain, such as an scFv; a spacer, such as comprising a sequence from a human immunoglobulin hinge, such as from IgG1, IgG2 or IgG4 or a modified version thereof, such as further comprising a CH2 region and/or a CH3 region; and a transmembrane domain, such as from human CD28; a costimulatory signaling domain, such as from human 4-1BB; and an intracellular signaling domain, such as a CD3ζ chain or a portion thereof.

In some of any embodiments, the transgene encodes the recombinant receptor.

In some of any embodiments, the transgene encodes a portion of the recombinant receptor. In some of any embodiments, the recombinant receptor contains two separate polypeptide chains, in which the portion of the recombinant receptor encoded by the transgene is one chain of the recombinant receptor. In some of any embodiments, the other chain of the recombinant receptor is encoded by a second transgene.

In some of any embodiments, the CAR is a multi-chain CAR. In some of any embodiments, transgene encodes one chain of the multi-chain CAR.

In some of any embodiments, the recombinant receptor is a recombinant T cell receptor (TCR). In some of any embodiments, recombinant TCR includes an alpha (TCRα) chain and a beta (TCRβ) chain and the transgene includes a nucleic acid sequence encoding the TCRα chain and/or a nucleic acid sequence encoding the TCRβ chain. In some of any embodiments, the transgene encodes one of the TCRα chain or the TCRβ chain.

In some of any embodiments, the TCRα chain includes a constant (Cα) region including one or more introduced cysteine residues and/or the TCRβ chain includes a Cβ region including one or more introduced cysteine residues, in which the one or more introduced cysteine residues are capable of forming one or more non-native disulfide bridges between the alpha chain and beta chain. In some of any embodiments, the one or more introduced cysteine residues includes replacement of a non-cysteine residue with a cysteine residue. In some of any embodiments, the Cα region includes a cysteine at a position corresponding to position 48 with numbering as set forth in SEQ ID NO: 92; and/or the CP region includes a cysteine at a position corresponding to position 57 with numbering as set forth in SEQ ID NO: 96.

In some of any embodiments, the transgene includes a sequence of nucleotides encoding at least one further protein. In some of any embodiments, the at least one further protein is a surrogate marker. In some of any embodiments, the surrogate marker is a truncated receptor. In some of any embodiments, the truncated receptor lacks an intracellular signaling domain and/or is not capable of mediating intracellular signaling when bound by its ligand.

In some of any embodiments, the transgene further includes a multicistronic element(s). In some of any embodiments, the multicistronic element(s) includes a sequence encoding a ribosome skip element selected from among a T2A, a P2A, a E2A or a F2A or an internal ribosome entry site (IRES).

In some of any embodiments, the multicistronic element is positioned between the sequence of nucleotides encoding the CAR and the sequence of nucleotides encoding the at least one further protein; the recombinant receptor is a recombinant TCR, and the multicistronic element is positioned between a sequence of nucleotides encoding the TCRα and a sequence of nucleotides encoding the TCRβ; the recombinant receptor is a multi-chain CAR, and the multicistronic element is positioned between a sequence of nucleotides encoding one chain of the multi-chain CAR and a sequence of nucleotides encoding another chain of the multi-chain CAR; and/or the multicistronic element(s) are upstream of the sequence of nucleotides encoding the recombinant receptor.

In some of any embodiments, the polynucleotide is a linear polynucleotide. In some of any embodiments, the polynucleotide is a double-stranded polynucleotide. In some of any embodiments, the polynucleotide is a single-stranded polynucleotide. In some of any embodiments, the polynucleotide is comprised in a viral vector. In some of any embodiments, the viral vector is an AAV vector. In some of any embodiments, the viral vector is a retroviral vector. In some of any embodiments, the viral vector is a lentiviral vector.

In some of any embodiments, the polynucleotide is at or about 1000, 1250, 1500, 1750, 2000, 2250, 2500, 2750, 3000, 3250, 3500, 3750 or 4000 nucleotides in length, or any value between any of the foregoing. In some of any embodiments, the polynucleotide is between at or about 1500 and at or about 2500 nucleotides or at or about 1750 and at or about 2250 nucleotides in length.

Also provided are methods of producing a genetically engineered T cell, the method including: (a) introducing, into a T cell, one or more agent(s) capable of inducing a genetic disruption at a target site within an endogenous T cell stimulation-associated locus of the T cell; and (b) introducing any of the provided polynucleotides into a T cell including a genetic disruption at a T cell stimulation-associated locus, in which the method produces a modified T cell stimulation-associated locus, said modified T cell stimulation-associated locus containing a transgene encoding the recombinant receptor or a portion thereof. In some of any embodiments, the transgene encoding a recombinant receptor or a portion thereof is integrated within the endogenous T cell stimulation-associated locus via homology directed repair (HDR).

Also provided are methods of producing a genetically engineered T cell, the method including introducing, into a T cell, a polynucleotide including a transgene encoding a recombinant receptor or a portion thereof, said T cell having a genetic disruption within a T cell stimulation-associated locus of the T cell, in which the transgene encoding the recombinant receptor or a portion thereof is integrated within the endogenous T cell stimulation-associated locus via homology directed repair (HDR).

In some of any embodiments, the genetic disruption is carried out by introducing, into a T cell, one or more agent(s) capable of inducing a genetic disruption at a target site within an endogenous T cell stimulation-associated locus of the T cell. In some of any embodiments, the method produces a modified T cell stimulation-associated locus, said modified T cell stimulation-associated locus containing a transgene encoding a recombinant receptor or a portion thereof.

In some of any embodiments, the polynucleotide further includes one or more homology arm(s) linked to the nucleic acid sequence, in which the one or more homology arm(s) comprise a sequence homologous to one or more region(s) of an endogenous T cell stimulation-associated locus in a T cell. In some of any embodiments, the one or more homology arm includes a 5′ homology arm and/or a 3′ homology arm. In some of any embodiments, the 5′ homology arm and 3′ homology arm includes nucleic acid sequences homologous to nucleic acid sequences surrounding a target site, in which the target site is within the T cell stimulation-associated locus.

In some of any embodiments, the target site is downstream of an endogenous transcriptional regulatory element of the T cell stimulation-associated locus.

In some of any embodiments, the polynucleotide includes the structure [5′ homology arm]-[transgene]-[3′ homology arm]. In some of any embodiments, the 5′ homology arm and 3′ homology arm includes nucleic acid sequences homologous to nucleic acid sequences surrounding the at least one target site. In some of any embodiments, the 5′ homology arm and 3′ homology arm independently are between at or about 50 and at or about 750 nucleotides, at or about 50 and at or about 500 nucleotides, at or about 50 and at or about 250 nucleotides, at or about 50 and at or about 100 nucleotides, at or about 100 and at or about 750 nucleotides, at or about 100 and at or about 500 nucleotides, at or about 100 and at or about 250 nucleotides, at or about 250 and at or about 750 nucleotides, at or about 250 and at or about 500 nucleotides, in length. In some of any embodiments, the 5′ homology arm and 3′ homology arm independently are at or about 50, 60, 70, 80, 90, 100, 150, 200, 250, 300, 400 or 500 nucleotides, or any value between any of the foregoing, in length. In some of any embodiments, the 5′ homology arm and 3′ homology arm independently are less than at or about 100 nucleotides, in length. In some of any embodiments, the 5′ homology arm and 3′ homology arm independently are at or about 50, 60, 70, 80 or 90 nucleotides, or any value between any of the foregoing, in length.

In some of any embodiments, the T cell stimulation-associated locus is selected from among PDCD1, CD69, Nur77, FoxP3 and a HLA-DR locus.

In some of any embodiments, the T cell stimulation-associated locus is PDCD1. In some of any embodiments, the 5′ homology arm and 3′ homology arm comprise a sequence homologous to one or more region(s) of PDCD1. In some of any embodiments, the 5′ homology arm includes: a) a sequence including at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 66; b) a sequence including at or at least at or at least 150, 200, 250, 300, 350, 400, 450, 500, 550, or 600 contiguous nucleotides of the sequence set forth in SEQ ID NO:66; or c) the sequence set forth in SEQ ID NO: 66. In some of any embodiments, the 3′ homology arm includes: a) a sequence including at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 67; b) a sequence including at or at least at or at least 150, 200, 250, 300, 350, 400, 450, 500, 550, or 600 contiguous nucleotides of the sequence set forth in SEQ ID NO:67; or c) the sequence set forth in SEQ ID NO: 67.

In some of any embodiments, the T cell stimulation-associated locus is CD69. In some of any embodiments, the 5′ homology arm and 3′ homology arm comprise a sequence homologous to one or more region(s) of CD69.

In some of any embodiments, the T cell stimulation-associated locus is Nur77. In some of any embodiments, the 5′ homology arm and 3′ homology arm comprise a sequence homologous to one or more region(s) of Nur77.

In some of any embodiments, the T cell stimulation-associated locus is FoxP3. In some of any embodiments, the 5′ homology arm and 3′ homology arm comprise a sequence homologous to one or more region(s) of FoxP3.

In some of any embodiments, the T cell stimulation-associated locus is a HLA-DR locus. In some of any embodiments, the 5′ homology arm and 3′ homology arm comprise a sequence homologous to one or more region(s) of a HLA-DR locus.

In some of any embodiments, the recombinant receptor or portion thereof is capable of inducing or transmitting the stimulation or activation signal in the T cell.

In some of any embodiments, the recombinant receptor includes an intracellular region including an intracellular signaling domain of a component of the T cell receptor (TCR) complex and the stimulation or activation signal in the T cells includes a signal through the intracellular signaling domain present in the recombinant receptor, or the recombinant receptor includes an intracellular region including an intracellular signaling domain including an immunoreceptor tyrosine-based activation motif (ITAM) and the stimulation or activation signal in the T cells includes a signal through the intracellular signaling domain present in the recombinant receptor.

In some of any embodiments, the recombinant receptor includes an extracellular region including a binding domain that is capable of binding to or recognizing an agent. In some of any embodiments, a stimulation or activation signal is induced in the T cell upon binding of the agent.

In some of any embodiments, the agent is a target antigen. In some of any embodiments, the target antigen is a recombinant protein or is an antigen expressed on the surface of a cell. In some of any embodiments, the target antigen is associated with, specific to, or expressed on a cell or tissue of a disease, disorder or condition. In some of any embodiments, the disease, disorder or condition is an infectious disease or disorder, an autoimmune disease, an inflammatory disease, or a tumor or a cancer.

In some of any embodiments, the target antigen is a tumor antigen, a pathogen-specific or pathogen-expressed antigen, an inflammatory antigen or an autoantigen. In some of any embodiments, the target antigen is a tumor antigen. In some of any embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-11 and LAGE-12), carcinoembryonic antigen (CEA), a cyclin, cyclin A2, C—C Motif Chemokine Ligand 1 (CCL-11), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-12), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-122 receptor alpha (IL-122Rα), IL-113 receptor alpha 2 (IL-113Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-11), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-11), a pathogen-specific or pathogen-expressed antigen, or an antigen associated with a universal tag, and/or biotinylated molecules, and/or molecules expressed by HIV, HCV, HBV or other pathogens.

In some of any embodiments, the genetic disruption is effected by a zinc finger nuclease (ZFN), a TAL-effector nuclease (TALEN), or a CRISPR-Cas9 combination that specifically binds to, recognizes, or hybridizes to the target site. In some of any embodiments, the genetic disruption is effected by a CRISPR-Cas9 combination and the CRISPR-Cas9 combination includes a guide RNA (gRNA) having a targeting domain that is complementary to the at least one target site. In some of any embodiments, the CRISPR-Cas9 combination is a ribonucleoprotein (RNP) complex including the gRNA and a Cas9 protein. In some of any embodiments, the genetic disruption of is effected by the RNP introduced into a plurality of T cells via electroporation.

In some of any embodiments, the T cell stimulation-associated locus is PDCD1. In some of any embodiments, the genetic disruption is effected by a CRISPR-Cas9 combination including a gRNA and the gRNA has a targeting domain that is complementary to a target site in a PDCD1 gene. In some of any embodiments, the gRNA includes the sequence set forth in in any one of SEQ ID NOS: 75 and 104-109. In some of any embodiments, the gRNA includes the sequence set forth in SEQ ID NO:75.

In some of any embodiments, the T cell stimulation-associated locus is CD69. In some of any embodiments, the genetic disruption is effected by a CRISPR-Cas9 combination including a gRNA and the gRNA has a targeting domain that is complementary to a target site in a CD69 gene. In some of any embodiments, the gRNA includes the sequence set forth in any one of SEQ ID NOS: 116-121.

In some of any embodiments, the T cell stimulation-associated locus is Nur77. In some of any embodiments, the genetic disruption is effected by a CRISPR-Cas9 combination including a gRNA and the gRNA has a targeting domain that is complementary to a target site in a Nur77 gene. In some of any embodiments, the gRNA includes the sequence set forth in SEQ ID NO: 122-127 and 134-136.

In some of any embodiments, the T cell stimulation-associated locus is FoxP3.

In some of any embodiments, the T cell stimulation-associated locus is a HLA-DR locus.

In some of any embodiments, the T cell further includes a genetic disruption at an endogenous T cell receptor alpha constant region (TRAC) gene and/or an endogenous T cell receptor beta constant region (TRBC) gene. In some of any embodiments, the genetic disruption is effected by a zinc finger nuclease (ZFN), a TAL-effector nuclease (TALEN), or a CRISPR-Cas9 combination that specifically binds to, recognizes, or hybridizes to at least one target site is within the TRAC, TRBC1 and/or TRBC2 gene. In some of any embodiments, the genetic disruption is effected by a CRISPR-Cas9 combination and the CRISPR-Cas9 combination includes a guide RNA (gRNA) having a targeting domain that is complementary to the at least one target site. In some of any embodiments, the CRISPR-Cas9 combination is a ribonucleoprotein (RNP) complex including the gRNA and a Cas9 protein. In some of any embodiments, the genetic disruption of is effected by the RNP introduced into a plurality of T cells via electroporation. In some of any embodiments, the genetic disruption is effected by a CRISPR-Cas9 combination including a gRNA and the gRNA has a targeting domain that is complementary to at least one target site is within the TRAC, TRBC1 and/or TRBC2 gene.

In some of any embodiments, the gRNA has a targeting domain that is complementary to a target site in a TRAC gene. In some of any embodiments, the gRNA includes the sequence set forth in any one of SEQ ID NOS: 77 and 188-218. In some of any embodiments, the gRNA includes the sequence set forth in SEQ ID NO:77. In some of any embodiments, the gRNA has a targeting domain that is complementary to a target site in a TRBC gene. In some of any embodiments, the gRNA includes the sequence set forth in any one of SEQ ID NOS: 219-276.

In some of any embodiments, the recombinant receptor is a chimeric antigen receptor (CAR). In some of any embodiments, the encoded recombinant receptor is or includes recombinant T cell receptor (TCR).

In some of any embodiments, the RNP is introduced via electroporation, particle gun, calcium phosphate transfection, cell compression or squeezing. In some of any embodiments, the RNP is introduced via electroporation. In some of any embodiments, the RNP introduced into a plurality of T cells via electroporation. In some of any embodiments, the concentration of the RNP is from at or about 1 μM to at or about 5 μM. In some of any embodiments, the concentration of the RNP is at or about 2 μM.

In some of any embodiments, T cells comprise CD8+ T cell and/or CD4+ T cells or subtypes thereof. In some of any embodiments, the T cells are autologous to the subject. In some of any embodiments, the T cell is a primary T cell derived from a subject. In some of any embodiments, the subject is a human. In some of any embodiments, the T cells are allogeneic to the subject. In some of any embodiments, the T cell is derived from a multipotent or pluripotent cell. In some of any embodiments, the T cell is derived from an iPSC.

In some of any embodiments, the polynucleotide is a linear polynucleotide. In some of any embodiments, the polynucleotide is a double-stranded polynucleotide. In some of any embodiments, the polynucleotide is a single-stranded polynucleotide. In some of any embodiments, the polynucleotide is comprised in a viral vector. In some of any embodiments, the viral vector is an AAV vector. In some of any embodiments, the viral vector is a retroviral vector. In some of any embodiments, the viral vector is a lentiviral vector.

In some of any embodiments, the polynucleotide is at or about 1000, 1250, 1500, 1750, 2000, 2250, 2500, 2750, 3000, 3250, 3500, 3750 or 4000 nucleotides in length, or any value between any of the foregoing. In some of any embodiments, the polynucleotide is between at or about 1500 and at or about 2500 nucleotides or at or about 1750 and at or about 2250 nucleotides in length.

In some of any embodiments, the one or more agent(s) and the polynucleotide are introduced simultaneously or sequentially, in any order. In some of any embodiments, the one or more agent(s) and the polynucleotide are introduced simultaneously. In some of any embodiments, the polynucleotide is introduced after the introduction of the one or more agent(s). In some of any embodiments, the polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the agent.

In some of any embodiments, prior to the introducing of the one or more agent(s) and/or the polynucleotide, the method includes incubating the cells, in vitro with a stimulatory agent(s) under conditions to stimulate or activate the one or more immune cells. In some of any embodiments, the stimulatory agent(s) includes and anti-CD3 and/or anti-CD28 antibodies. In some of any embodiments, the stimulatory agent(s) includes an oligomeric particle reagent including anti-CD3 and/or anti-CD28 antibodies. In some of any embodiments, the stimulatory agent(s) includes beads coated with anti-CD3 and/or anti-CD28 antibodies.

In some of any embodiments, the method further includes incubating the cells prior to, during or subsequent to the introducing of the one or more agents and/or the introducing of the polynucleotide with one or more recombinant cytokines. In some of any embodiments, the one or more recombinant cytokines are selected from the group consisting of IL-2, IL-7, and IL-15. In some of any embodiments, the one or more recombinant cytokine is added at a concentration selected from a concentration of IL-2 from at or about 10 U/mL to at or about 200 U/mL. In some of any embodiments, the one or more recombinant cytokine is added at a concentration of at or about 50 IU/mL to at or about 100 U/mL; IL-7 at a concentration of 0.5 ng/mL to 50 ng/mL. In some of any embodiments, the one or more recombinant cytokine is added at a concentration of at or about 5 ng/mL to at or about 10 ng/mL and/or IL-15 at a concentration of 0.1 ng/mL to 20 ng/mL. In some of any embodiments, the one or more recombinant cytokine is added at a concentration of at or about 0.5 ng/mL to at or about 5 ng/mL.

In some of any embodiments, the incubation is carried out subsequent to the introducing of the one or more agents and the introducing of the polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days. In some of any embodiments, the incubation is carried out for up to or about 7 days.

Also provided is an engineered T cell or a plurality of engineered T cells generated using any of the provided methods.

Also provided is a composition that includes any of the provided engineered cells or a plurality of any of the provided engineered cells.

Also provided is a composition containing a plurality of any of the provided engineered cells.

In some of any embodiments, the expression of the operably linked transgene is upregulated or induced in one or more cells in the composition within less than at or about 6, 12, 18, 24, 36 or 48 hours following the stimulation or activation signal in the T cells. In some of any embodiments, following a simulation or activation signal in the T cells, the frequency of cells expressing the operably linked transgene among the cells in the composition is greater than at or about 50%, 60%, 70%, 75%, 80%, 85%, 90% or 95% or more. In some of any embodiments, following the upregulation or induction of expression or following a reduction or an absence of the simulation or activation signal in the T cells, the expression of the operably linked transgene is reduced or downregulated in one or more cells in the composition.

In some of any embodiments, following the upregulation or induction of expression, or following the reduction or the absence of the simulation or activation signal in the T cells, the frequency of cells expressing the operably linked transgene among the cells in the composition is reduced by greater than at or about 30%, 40%, 50%, 60%, 70%, 75%, 80%, 85%, 90% or 95% or more. In some of any embodiments, following the upregulation or induction of expression, and the expression of the operably linked transgene is reduced or downregulated in one or more cells in the composition after at or about 1, 2, 3, 4, 5, 6, 7 or 8 days or more after the stimulation or activation signal in the T cells.

In some of any embodiments, the expression of the operably linked transgene in one or more of the cells in the composition is reduced or downregulated within less than at or about 6, 12, 18, 24, 36 or 48 hours following the reduction or an absence of the simulation or activation signal in the T cell. In some of any embodiments, following a reduction or an absence of the simulation or activation signal in the T cells, the frequency of cells expressing the recombinant receptor among the cells in the composition is less than at or about 50%, 40%, 30%, 25%, 20%, 15%, 10% or 5% or less.

In some of any embodiments, the composition includes CD4+ T cells and/or CD8+ T cells. In some of any embodiments, the composition includes CD4+ and CD8+ T cells and the ratio of CD4+ to CD8+ T cells is from or from about 1:3 to 3:1. In some of any embodiments, 1:1.

Also provided are methods of treatment including administering any of the provided engineered cells or any of the provided engineered compositions to a subject having a disease or disorder.

Also provided are uses of any of the provided engineered cells or any of the provided engineered compositions for the treatment of a disease or disorder.

Also provided are uses of any of the provided engineered cells or any of the provided engineered compositions in the manufacture of a medicament for treating a disease or disorder.

Also provided is any of the provided engineered cells or any of the provided engineered compositions for use in the treatment of a disease or disorder.

In some of any embodiments, the disease or disorder is a cancer or a tumor. In some of any embodiments, the cancer or the tumor is a hematologic malignancy. In some of any embodiments, the cancer or the tumor a lymphoma, a leukemia, or a plasma cell malignancy. In some of any embodiments, the cancer is a lymphoma and the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL). In some of any embodiments, the cancer is a leukemia and the leukemia is chronic lymphocytic leukemia (CLL), plasma cell leukemia or acute lymphocytic leukemia (ALL). In some of any embodiments, the cancer is a plasma cell malignancy and the plasma cell malignancy is multiple myeloma (MM).

In some of any embodiments, the tumor is a solid tumor. In some of any embodiments, the solid tumor is a non-small cell lung cancer (NSCLC) or a head and neck squamous cell carcinoma (HNSCC).

Also provided are kits that includes one or more agent(s) capable of inducing a genetic disruption at a target site within a T cell stimulation-associated locus; and any of the provided polynucleotides.

Also provided are kits that includes one or more agent(s) capable of inducing a genetic disruption at a target site within a T cell stimulation-associated locus; and a polynucleotide including a nucleic acid sequence encoding recombinant receptor or a portion thereof, in which the transgene encoding the recombinant receptor or antigen-binding fragment or chain thereof is targeted for integration at or near the target site via homology directed repair (HDR); and instructions for carrying out any of the provided methods.

### BRIEF DESCRIPTION OF THE DRAWINGS

FIG. 1 shows a schematic depicting engineered T cells conditionally expressing a recombinant receptor. In some cases, the expression of a recombinant receptor is under operable control of a T cell stimulation-associated locus. In an exemplary engineered T cell provided herein, containing a modified T cell stimulation-associated locus comprising a transgene encoding a recombinant receptor or a portion thereof operably linked to an endogenous transcriptional regulatory element of the T cell stimulation-associated locus, the encoded recombinant receptor can be expressed following a stimulation or activation signal in the T cell. In some cases, the endogenous TCR expression is reduced or suppressed by introducing a genetic disruption at a locus encoding a component of the endogenous TCR, for example, endogenous T cell receptor alpha constant region (TRAC) gene and/or an endogenous T cell receptor beta constant region (TRBC) gene. In some cases, if signal transduction through the recombinant receptor is reduced or eliminated due to the elimination of target cells expressing the target antigen, expression of the recombinant receptor is reduced or eliminated.

FIG. 2 shows the percent of cells expressing various markers, including gene products of various T cell stimulation-associated loci, over time after stimulation with a reagent containing an anti-CD3 and anti-CD28 antibodies at day 0. Expression of the markers were assessed by flow cytometry. Around day 7 after the initial stimulation, cells were re-stimulated using the same reagent and the percentage of cells expressing the markers were assessed over time.

FIGS. 3A-3B depict flow cytometry plots for expression of CD3, PD-1 and the chimeric antigen receptor (CAR), in cells introduced with ribonucleoprotein (RNP) complexes containing PDCD1-targeting gRNA (PD-1 KO), RNP complexes containing TRAC-targeting gRNA (TRAC KO), polynucleotides containing CAR-encoding sequences for HDR for targeting at the PDCD1 locus (PD-1 KI CAR), polynucleotides containing CAR-encoding sequences for HDR for targeting at the TRAC locus (TRAC KI CAR), or a combination thereof.

FIGS. 4A-4C show flow cytometry plots for expression of CD3, PD-1, CD69 and the chimeric antigen receptor (CAR), in engineered T cells comprising sequences encoding the CAR under operable control of the endogenous PDCD1 locus (PD-1 KI CAR) or TRAC transcriptional regulatory elements (TRAC KI CAR), after a time of rest after an initial stimulation, or after a re-stimulation after initial stimulation and rest.

FIGS. 5A-5B depict the percentage of CAR+ cells over time, with or without re-stimulation, in engineered T cells comprising sequences encoding the CAR under operable control of the endogenous PDCD1 locus (PD-1 KI CAR) or TRAC transcriptional regulatory elements (TRAC KI CAR), after a time of rest after an initial stimulation, or after re-stimulation after initial stimulation and rest.

FIG. 6A depict flow cytometry plots for expression of the exemplary anti-CD19 CAR (as detected using an anti-idiotypic antibody) under the control of the PDCD1 promoter (PD1 KI CAR) and CD8, at 7 days after electroporation, after a first round of stimulation by co-culturing with irradiated CD19-expressing lymphoblastoid cell lines (LCLs) for 7 days, and after two rounds of stimulation by co-culturing with irradiated CD19-expressing LCLs, in cells in which the exemplary anti-CD19 CAR was expressed under operable control of the endogenous PDCD1 locus (PD1 KI CAR). FIG. 6B depicts the fold expansion of the cells after first round and second round of cells in the PD1KI CAR cells, PDCD1 KO cells (electroporated with PDCD1 targeting RNP complexes only, without polynucleotide encoding the exemplary CAR; PD1 KO), mock treated cells (Neg. control), and/or cells expressing the same exemplary CAR engineered using a lentiviral vector (LV control).

FIG. 7A depict flow cytometry plots for expression of the exemplary anti-CD19 CAR (as detected using an anti-idiotypic antibody; aID) under the control of the PDCD1 promoter (PD1 KI CAR) and CD8, after a first round of stimulation by co-culturing with irradiated CD19-expressing LCLs (1^{st} stim), in cells that were rested without re-stimulation (rested) or subject to a re-stimulation (2^{nd} stim). FIG. 7B shows the mean fluorescence intensity (MFI) of CAR expression (as detected using an anti-idiotypic antibody; left), percentage of CAR-expressing cells (middle) and the percentage of CD25+ CD69+ cells (right), in PD1 KO cells, PD1 KI CAR cells that were rested without re-stimulation (PD1 KI CAR rested) and PD1 KI CAR cells that were subject to a re-stimulation (PD1 KI CAR restim). FIG. 7C shows the integrated mean fluorescence intensity (MFI) of CAR expression, in PD1 KO cells, PD1 KI CAR cells that were rested without re-stimulation (PD1 KI CAR rested) and PD1 KI CAR cells that were subject to a re-stimulation (PD1 KI CAR restim). FIG. 7D depicts cytolytic activity of PD1 KO cells, PD1 KI CAR cells that were rested without re-stimulation (PD1 KI CAR rested) and PD1 KI CAR cells that were subject to a re-stimulation (PD1 KI CAR restim), for target cells expressing CD19. Mock treated cells (Neg. control), LV control and human embryonic kidney (HEK) cells were used as controls.

FIG. 8A depicts a schematic of a timeline for assessing the in vivo anti-tumor activity in a mouse model, injected with Raji lymphoma tumor cells transfected with firefly luciferase, and with cells expressing an exemplary anti-CD19 CAR under operable control of the endogenous PDCD1 locus (PD-1 KI CAR) or by lentiviral delivery (LV control). FIG. 8B depicts tumor growth over time as indicated by measuring average radiance by bioluminescence. FIG. 8C depicts the survival of each group of mice over time. FIG. 8D depicts the results of bioluminescence imaging if the mice, indicating the presence of the tumor at day −1, day 7, day 14 and day 28.

### DETAILED DESCRIPTION

Provided herein are engineered cells, such as engineered T cells, that contain a modified genetic locus, such as a modified T cell stimulation-associated locus. In some aspects, the engineered cells contain a heterologous or exogenous nucleic acid sequence (e.g., a transgene) encoding a receptor (for example, a chimeric antigen receptor or a recombinant T cell receptor) or a portion thereof, operably linked to an endogenous transcriptional regulatory element of a T cell stimulation-associated locus. In some aspects, the endogenous transcriptional regulatory element induces or upregulates expression of the operably linked nucleic acid sequence following a simulation or activation signal in the T cell.

In some aspects, the expression from the transcriptional regulatory elements of a T cell stimulation-associated locus is responsive to a signal through the intracellular signaling region of the recombinant receptor, such as a primary activation signal in a T cell, a signaling domain of a T cell receptor (TCR) component, and/or a signaling domain comprising an immunoreceptor tyrosine-based activation motif (ITAM). In some embodiments, exemplary T cell stimulation-associated loci include, but are not limited to, PDCD1 (encoding PD-1), CD69, Nur77 (encoding NR4A1), FoxP3 or a HLA-DR locus.

Also provided are methods for producing genetically engineered cells containing a modified T cell stimulation-associated locus expressing a recombinant receptor or a portion thereof. The provided embodiments involve specifically targeting nucleic acid sequence encoding the recombinant receptor or a portion thereof to the endogenous T cell stimulation-associated locus. In some contexts, the provided embodiments involve inducing a targeted genetic disruption, e.g., generation of a DNA break, using gene editing methods, and homology-directed repair (HDR) for targeted integration of the recombinant receptor-encoding nucleic acid sequences at the endogenous T cell stimulation-associated locus. Also provided are related cell compositions, nucleic acids and kits for use in generation of the engineered cells provided herein and/or the methods provided herein.

T cell-based therapies, such as adoptive T cell therapies (including those involving the administration of engineered cells expressing recombinant, engineered or chimeric receptors specific for a disease or disorder of interest, such as a chimeric antigen receptor (CAR), a recombinant T cell receptor (TCR) or other recombinant, engineered or chimeric receptors) can be effective in the treatment of cancer and other diseases and disorders. In certain contexts, other approaches for designing and generating engineered cells for adoptive cell therapy may not always be entirely satisfactory. In some aspects, engineered cells comprising a recombinant receptor, may in some cases target healthy cells expressing the antigen recognized by the recombinant receptors. In some cases, the recombinant receptor expressing cells cannot distinguish between diseased cells, e.g., tumor cells, and normal cells expressing the antigen. In some aspects, after the engineered cells target and remove diseased cells, e.g., tumor cells expressing the target antigen, continued persistence of recombinant receptor expressing cells can target healthy cells that express the antigen, resulting in undesirable effect. For example, in some cases, continued persistence of anti-CD19 CAR-expressing cells after tumor clearance can result in B cell aplasia, due to the attack of CD19-expressing healthy B cells by the CAR-expressing cells.

In some aspects, the provided embodiments involve inducing a targeted genetic disruption and integration of transgenes encoding a recombinant receptor or a portion thereof, by HDR at an endogenous T cell stimulation-associated locus. In some aspects, the transgene encoding a recombinant receptor or a portion thereof is operably linked to the endogenous transcriptional regulatory element(s) of a T cell stimulation-associated locus. The expression of the operably linked transgene, e.g., encoding the recombinant receptor or a portion thereof, is controlled by the endogenous transcriptional regulatory elements, such as the promoter, of the T cell stimulation-associated locus. In some aspects, the endogenous transcriptional regulatory elements induce or upregulate expression of the operably linked transgene, e.g., encoding a recombinant receptor, following a simulation or activation signal in the T cell. In some aspects, the endogenous transcriptional regulatory elements reduce or downregulate expression of the operably linked transgene, e.g., encoding a recombinant receptor, following a reduction or an absence of the simulation or activation signal in the T cell. Accordingly, the expression of the recombinant receptor can be controlled based on the presence of a stimulation or activation-associated signal in the T cell.

In some aspects, the provided embodiments are based on observations that T cell comprising a recombinant receptor, e.g., CAR, expressed under the control of a T cell stimulation associated locus, e.g., PDCD1, is transiently induced or upregulated upon a stimulation or activation signal. The recombinant receptor was observed to be expressed in response to a re-stimulation signal after a period of resting. Expression of exemplary T cell stimulation-associated gene products were observed to be induced upon an initial stimulation signal, e.g., via an anti-CD3 and anti-CD28 antibody, which, in a period of time after the initial stimulation, is reduced. Following a re-stimulation after a certain period, the expression of the exemplary T cell stimulation-associated gene products increased after the reduction. The provided embodiments are also based on the observation that following re-stimulation, the recombinant receptor, e.g., CAR, expressed under the control of a T cell stimulation associated locus, e.g., PDCD1, is again induced or upregulated again, and the T cell expressing the recombinant receptor is capable of killing target cells efficiently.

In some aspects, gene expression under the operable control of the transcriptional regulatory elements of the T cell stimulation-associated locus is responsive to the T cell stimulation or activation signal. Such responsiveness permits regulation of the expression of recombinant receptor and helps minimize undesired effects on healthy cells expressing the target antigen or undesired function of the recombinant receptor expressing cells, without further drawbacks to the function of the recombinant receptor-expressing cells.

In some cases, a fully functional or an unmodified endogenous gene product of the T cell stimulation-associated locus is expressed in the cell, such that the endogenous gene product and the recombinant receptor is co-expressed. In some cases, the endogenous gene product of the T cell stimulation-associated locus is not expressed, or knocked out.

In some aspects, the engineered cell further comprises a genetic disruption of endogenous T cell receptor-encoding gene(s) in the cell. For example, an endogenous T cell receptor alpha constant region (TRAC) gene and/or an endogenous T cell receptor beta constant region (TRBC) gene is disrupted in the T cell. In some aspects, such disruption also prevents antigen-independent, tonic signaling within the recombinant receptor expressing cells, and minimize unregulated expression of an endogenous T cell receptor in the T cell. In some embodiments, antigen-independent signaling of the encoded recombinant receptor is reduced by greater than at or about 10%, 15%, 20%, 25%, 30%, 40%, 50% or more compared to an engineered cell comprising a transgene encoding the same recombinant receptor that is randomly integrated.

In some aspects, the provided embodiments offer an advantage that, the disruption of the endogenous T cell receptor-encoding gene(s) in the cell, together with the expression of the recombinant receptor under the control of the transcriptional regulatory elements of a T cell stimulation-associated locus, generates a feedback loop for expression of the recombinant receptor. In such cases, the stimulation or activation of the T cell is controlled by or dependent on stimulation signal transmitted or transduced through the signaling region of the recombinant receptor, for example, by virtue of the binding or recognition of the target antigen by the recombinant receptor, and the stimulation or activation signal is reduced or eliminated in the absence of antigen-specific signal. In some contexts, the recombinant receptor and the cell expressing the recombinant receptor retains their cytotoxic function against target cells, until the target cells are eliminated or the target cells are no longer available. In some aspects, following removal of target cells, e.g., diseased cells expressing the target antigen, stimulation or activation signal in the recombinant receptor expressing cells is reduced, and the cells become less responsive or unresponsive, permitting healthy normal cell population to be unaffected by the recombinant receptor-expressing cells. In some cases, the lack of expression of the endogenous T cell receptor prevents undesired re-activation of the recombinant receptor expressing cells. In some embodiments, the provided cells, compositions and methods can result in improved cell therapies, particularly for cell therapies that target or are specific for an antigen in a tumor microenvironment. In some contexts, the provided cells, compositions and methods also offer advantages in controlling and regulating expression of the recombinant receptor, e.g. CAR, on cells of the cell therapy. In some aspects, the provided engineered cells and methods permit refined temporal regulation of the recombinant receptor-expressing cells, and minimizes undesired antigen-independent effect of the recombinant receptor-expressing cells. In some cases, the provided embodiments permit recovery of healthy cells after administration of recombinant receptor-expressing cells.

In some contexts, the recombinant receptors encoded from the modified T cell stimulation-associated locus in engineered cells provided herein can be encoded under the control of endogenous regulatory elements of the T cell stimulation-associated locus, e.g., cis regulatory elements, such as the promoter, or the 5′ and/or 3′ untranslated regions (UTRs) of the endogenous T cell stimulation-associated locus. In some aspects, such embodiments allow the recombinant receptor, e.g., CAR, or a portion thereof, to be expressed and/or the expression is regulated at a similar time, duration, level, and with similar expression kinetics as the endogenous T cell stimulation-associated locus.

In some aspects, the entire recombinant receptor or a full length recombinant receptor is encoded from the modified T cell stimulation-associated locus in the engineered cells. In some aspects, a portion of the recombinant receptor, such as a domain or a region of the recombinant receptor, or one or more chains of a recombinant receptor that comprises multiple chains (e.g., a multi-chain CAR or a recombinant a recombinant T cell receptor (TCR) comprising two or more chains) is encoded from the modified T cell stimulation-associated locus in the engineered cells. In some aspects, a remaining portion (for example, another chain or another domain of the recombinant receptor) is encoded by a second transgene present in the engineered cell.

In some contexts, optimal efficacy of engineered cells can depend on the ability of the administered cells to express the recombinant receptor, including with uniform, homogenous, consistent and/or regulated expression of the receptors among cells, such as a population of immune cells and/or cells in a therapeutic cell composition, and for the recombinant receptor to recognize and bind to a target, e.g., target antigen, within the subject, tumors, and environments thereof. In some cases, available methods for introducing a recombinant receptor, such as a CAR, into a cell is by random integration of sequences encoding the recombinant receptor. In certain respects, such methods are not entirely satisfactory. In some aspects, random integration can result in possible insertional mutagenesis and/or genetic disruption of one more genetic loci in the cell, including those that may be important for cell function and activity. In some cases, semi-random or random integration of a transgene encoding the receptor into the genome of the cell may, in some cases, result in adverse and/or unwanted effects due to integration of the nucleic acid sequence into an undesired location in the genome, e.g., into an essential gene or a gene critical in regulating the activity of the cell.

In some cases, random integration may result in variable integration of the sequences encoding the recombinant receptor, which can result in inconsistent or unregulated expression, variable copy number of the nucleic acids, and/or variability of receptor expression within cells of the cell composition, such as a therapeutic cell composition. In some cases, random integration of a nucleic acid sequence encoding the receptor can result in variegated, heterogeneous, non-uniform, unregulated and/or suboptimal expression or antigen binding, oncogenic transformation and transcriptional silencing of the nucleic acid sequence, depending on the site of integration and/or nucleic acid sequence copy number. In some aspects, heterogeneous and non-uniform expression in a cell population can lead to inconsistencies or instability of expression and/or antigen binding by the recombinant receptor, unpredictability of the function or reduction in function of the engineered cells and/or a non-uniform drug product, thereby reducing the efficacy of the engineered cells. In some aspects, use of particular random integration vectors, such as certain lentiviral vectors, requires confirmation that the engineered cells do not contain replication competent virus. Improved strategies are needed to achieve consistent expression levels and function of the recombinant receptors while minimizing random integration of nucleic acids and/or heterogeneous expression in a population.

In some contexts, the provided embodiments relate to engineering a cell to have nucleic acids encoding a recombinant receptor to be integrated into the endogenous T cell stimulation-associated locus of a cell, e.g. T cell, by homology-directed repair (HDR). In some aspects, HDR can mediate the site specific integration of transgenes (such as transgenes encoding a recombinant receptor or a portion thereof), at or near a target site, such as an endogenous T cell stimulation-associated locus. In some embodiments, the presence of a genetic disruption (for example, at the endogenous T cell stimulation-associated locus) and a template polynucleotide containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the homology arms included in the template polynucleotide, cellular DNA repair machinery can use the template polynucleotide to repair the DNA break and resynthesize genetic information at the site of the genetic disruption, thereby effectively inserting or integrating the sequences between the homology arms (such as transgenes encoding a recombinant receptor or a portion thereof) at or near the site of the genetic disruption. The provided embodiments can generate cells containing a modified T cell stimulation-associated locus encoding a recombinant receptor or a portion thereof, where transgenes encoding a recombinant receptor or a portion thereof is integrated into the endogenous T cell stimulation-associated locus by HDR.

In some aspects, the provided embodiments offer advantages in producing engineered cells with improved and/or more efficient targeting of the nucleic acids encoding the recombinant receptor into the cell and can result in improved activity and/or function of the engineered cell. In some cases, the provided embodiments minimize possible semi-random or random integration and/or heterogeneous, unregulated or variegated expression, and result in improved, uniform, homogeneous, consistent, regulated and/or stable expression of the recombinant receptor or having reduced, low or no possibility of insertional mutagenesis.

In some aspects, compared to other methods of producing genetically engineered immune cells expressing a recombinant receptor, e.g., TCR or CAR, the provided embodiments allow for a more stable, physiological, controllable, regulated, uniform, consistent and/or homogeneous expression of the recombinant receptor. In some cases, the methods result in the generation of more consistent and more predictable drug product, e.g. cell composition containing the engineered cells, which can result in a safer therapy for treated patients. In some aspects, the provided embodiments also allow predictable and consistent integration at a single gene locus or a multiple gene loci of interest. In some embodiments, the provided embodiments can also result in generating a cell population with consistent copy number (typically, 1 or 2) of the nucleic acids that are integrated in the cells of the population, which, in some aspects, provide consistency in recombinant receptor expression and expression of the endogenous receptor genes within a cell population. In some cases, the provided embodiments do not involve the use of a viral vector for integration and thus can reduce the need for confirmation that the engineered cells do not contain replication competent virus, thereby improving the safety of the cell composition.

Also provided are methods for engineering, preparing, and producing the engineered cells, and kits and devices for generating or producing the engineered cells. Also provided are cells and cell compositions generated by the methods. Also provided are polynucleotides, e.g., linear polynucleotides, that contain a nucleic acid sequence encoding a recombinant receptor or a portion thereof, and methods for introducing such polynucleotides into the cells, such as by transduction or by physical delivery, such as electroporation. Also provided are compositions containing the engineered cells, and methods, kits, and devices for administering the cells and compositions to subjects, such as for adoptive cell therapy. In some aspects, the cells are isolated from a subject, engineered, and administered to the same subject. In other aspects, they are isolated from one subject, engineered, and administered to another subject. In some embodiments, the provided polynucleotides, transgenes, and/or vectors, when delivered into immune cells, result in the expression of recombinant receptors, e.g., TCRs or CARs, that can modulate T cell activity, and, in some cases, can modulate T cell differentiation or homeostasis. The resulting genetically engineered cells or cell compositions can be used in adoptive cell therapy methods.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. CELLS CONDITIONALLY EXPRESSING A RECOMBINANT RECEPTOR

Provided herein are engineered immune cells, such as engineered T cells, that contains a transgene encoding a recombinant receptor or a portion thereof, present at a genomic locus, such as a T cell stimulation-associated locus. In some embodiments, the engineered cells contain a modified T cell stimulation-associated locus, e.g., PDCD1, CD69, Nur77, FoxP3 or a HLA-DR locus, that comprises a transgene (i.e., heterologous or exogenous nucleic acid sequence) that contains a nucleic acid sequence encoding a recombinant receptor (for example, a chimeric antigen receptor or a recombinant T cell receptor) or a portion thereof. In some aspects, the nucleic acid sequence encoding the recombinant receptor or a portion thereof is under operative control of endogenous regulatory element(s) of the T cell stimulation-associated locus. In some embodiments, the endogenous transcriptional regulatory element induces or upregulates, such as transiently induces or upregulates, expression of the operably linked transgene, e.g., encoding a recombinant receptor or a portion thereof, following a simulation or activation signal in the T cell. In some contexts, in the provided cells comprising the modified T cell stimulation-associated locus, expression of the encoded recombinant receptor or portion thereof is similar to or mimics the expression of the endogenous gene product of the T cell stimulation-associated locus in a cell that is not modified. In some aspects, the temporal regulation and level and kinetics of expression of the encoded recombinant receptor or a portion thereof is similar to those of the endogenous gene product of the T cell stimulation-associated locus. For example, in some aspects, the encoded recombinant receptor is transiently induced or upregulated following a stimulation or activation signal in the T cell, and is reduced or downregulated in the absence of such signal. In some aspects, provided embodiments permit a refined and conditional regulation of expression of the encoded recombinant receptor.

Provided herein are engineered T cells comprising a modified T cell stimulation-associated locus comprising a transgene encoding a recombinant receptor or a portion thereof operably linked to an endogenous transcriptional regulatory element of the T cell stimulation-associated locus, wherein the endogenous transcriptional regulatory element induces or upregulates expression of the operably linked transgene following a simulation or activation signal in the T cell. In some embodiments, the endogenous transcriptional regulatory element is a promoter of an endogenous T cell stimulation-associated locus. In some aspects, the transgene encoding the recombinant receptor or a portion thereof is present downstream of the promoter.

Also provided are engineered cells, such as engineered T cells, that comprise a nucleic acid sequence encoding a recombinant receptor or a portion thereof operably linked to an endogenous transcriptional regulatory element of a T cell stimulation-associated locus. In some embodiments, the endogenous transcriptional regulatory element of the T cell stimulation-associated locus induces or upregulates expression of the operably linked nucleic acid sequence following a simulation or activation signal in the T cells responsive to stimulation or activation signal in the T cell. In some aspects, the transcriptional regulatory element of the endogenous T cell stimulation-associated locus is responsive to stimulation or activation signal in the T cell.

Also provided are engineered T cells that comprise a nucleic acid encoding a recombinant receptor or a portion thereof operably linked to a transcriptional regulatory element of an endogenous T cell stimulation-associated locus, wherein the endogenous transcriptional regulatory element of the T cell stimulation-associated locus induces or upregulates expression of the operably linked nucleic acid sequence following a simulation or activation signal in the T cells responsive to stimulation or activation signal in the T cell. Also provided are engineered T cells that comprise a nucleic acid encoding a recombinant receptor or a portion thereof operably linked to a transcriptional regulatory element of an endogenous T cell stimulation-associated locus, wherein the transcriptional regulatory element of the endogenous T cell stimulation-associated locus is responsive to stimulation or activation signal in the T cell.

In some embodiments, the expression of the operably linked transgene is transient after the stimulation or activation signal in the T cell. In some aspects, the expression is reduced or downregulated following the upregulation or induction of expression in the absence of further stimulation or activation, or following a reduction or absence of the simulation or activation signal in the T cells. In some embodiments, the expression of the operably linked transgene is capable of being induced or upregulated again following a further simulation or activation signal in the T cells after a reduction or absence of the signal.

### A. Stimulation or Activation-Associated Loci and Gene Expression

In some aspects, provided are engineered cells that conditionally express a recombinant receptor. In some embodiments, the engineered cells contain nucleic acid sequences, such as transgene or heterologous sequences, encoding a recombinant receptor. In some aspects, the transgene encoding a recombinant receptor is expressed conditionally, for example, in the presence of a particular condition or signal in the engineered cell or the environment. In some aspects, the recombinant receptor is not expressed, or the expression is reduced, in the absence of the particular condition or signal in the engineered cell or the environment. In some aspects, the expression of the recombinant receptor is controlled by transcriptional regulatory element(s) of a T cell stimulation-associated locus, such as PDCD1 (encoding PD-1), CD69, Nur77 (encoding NR4A1), FoxP3 or a HLA-DR locus. In some aspects, the engineered cell contains a modified T cell stimulation-associated locus comprising a transgene encoding a recombinant receptor or a portion thereof. In some aspects, the transgene encoding the recombinant receptor or a portion thereof is operably linked to an endogenous transcriptional regulatory element of the T cell stimulation-associated locus, wherein the endogenous transcriptional regulatory element induces or upregulates expression of the operably linked transgene following a simulation or activation signal in the T cell. In some of any embodiments, the endogenous transcriptional regulatory element is a promoter of an endogenous T cell stimulation-associated locus.

In some aspects, the expression from the T cell stimulation-associated locus, e.g., under the control of the endogenous transcriptional regulatory element of the T cell stimulation-associated locus, is induced or upregulated following the presence of a stimulation or activation signal in the engineered cell, e.g., engineered T cell. In some aspects, the stimulation or activation signal includes a signal through a signaling domain of a T cell receptor (TCR) component, and/or signal through a signaling domain comprising an immunoreceptor tyrosine-based activation motif (ITAM). For example, in some aspects, signal from ligation of the encoded recombinant receptor, e.g., CAR or TCR, can provide the activation or stimulation signal that induces the expression from the T cell stimulation-associated locus. In some aspects, signal from ligation of receptors containing signaling domains that contain motifs such as the ITAM can provide the activation or stimulation signal that induces the expression from the T cell stimulation-associated locus.

In some embodiments, the presence of a T cell stimulation or activation signal in the engineered T cell can induce the expression of the encoded recombinant receptor, and can result in further induction, the expression or upregulation of the encoded recombinant receptor. In some aspects, such conditional regulation of expression of the encoded recombinant receptor can result in a positive feedback loop, or a feed-forward loop that can permit increased or amplified expression of the recombinant receptor following a stimulation or activation signal in the engineered T cell. In some embodiments, binding of an agent to an extracellular binding domain of the recombinant receptor results in the inducing or transmitting of the stimulation or activation signal in the cell. In some embodiments, the stimulation or activation signal in the T cell is transmitted through an intracellular signaling region of the recombinant receptor upon the binding of an agent to an extracellular binding domain of the recombinant receptor.

In some aspects, the expression of the operably linked transgene, e.g., the transgene encoding a recombinant receptor or a portion thereof operably linked to the endogenous transcriptional regulatory element of the T cell stimulation-associated locus, is upregulated or induced within less than at or about 6, 12, 18, 24, 36 or 48 hours following the stimulation or activation signal in the T cells. In some embodiments, the expression of the operably linked transgene is upregulated or induced within less than at or about 24 hours following the stimulation or activation signal in the T cells. In some embodiments, the expression of the operably linked transgene is upregulated or induced by greater than at or about 30%, 40%, 50%, 60%, 70%, 75%, 80%, 85%, 90% or 95% or more. In some aspects, the upregulation or induction is compared to the expression of the operably linked transgene, before, or in the absence of the stimulation or activation signal in the T cells, or the minimum expression of the operably linked transgene prior to the stimulation or activation signal in the T cells.

In some aspects, induction or upregulation of expression is repeatedly controllable in the presence of the stimulation or activation signal in the T cells. In some aspects, such controlled expression ensures that the recombinant receptor, e.g. CAR, is only expressed at high levels in environments where the T cells are being activated, such as in sites where antigen recognized by the CAR is present. In some aspects, once the activation or stimulation signal terminates, such as due to loss of antigen as may occur after cytolytic killing of a tumor, the expression of the CAR is downregulated or reduced. In some embodiments, the induction or upregulation of expression of the transgene is transient for the period of the stimulation or activation signal and then is reduced or downregulated.

In some embodiments, after the stimulation or activation signal in the T cells, for example, an initial stimulation or activation signal (for example, via the recombinant receptor) in the T cells, and the subsequent upregulation or induction of expression of the operably linked transgene (e.g., encoding the recombinant receptor or a portion thereof), the expression of the operably linked transgene is reduced or downregulated. In some embodiments, the expression of the operably linked transgene is reduced or downregulated following the upregulation or induction of expression, or following a reduction or an absence of the simulation or activation signal in the T cells.

In some embodiments, the expression of the operably linked transgene is reduced or downregulated after a certain period of upregulation or induction of expression following the stimulation or activation signal (e.g., the initial stimulation or activation signal) in the T cells. In some embodiments, the stimulation or activation signal in the T cells is an initial stimulation or activation signal in the T cells, and the expression of the operably linked transgene is reduced or downregulated after at or about 1, 2, 3, 4, 5, 6, 7 or 8 days or more after the initial stimulation or activation signal in the T cells. In some embodiments, following the upregulation or induction of expression that follows the stimulation or activation signal in the T cells, and the expression of the operably linked transgene is reduced or downregulated after at or about 1, 2, 3, 4, 5, 6, 7 or 8 days or more after the initial stimulation or activation signal in the T cells. In some embodiments, the expression of the operably linked transgene is reduced or downregulated after at or about 2, 3 or 4 days or more after the initial stimulation or activation signal in the T cells. In some embodiments, the expression of the operably linked transgene is reduced or downregulated after at or about 2 days after the initial stimulation or activation signal in the T cells.

In some embodiments, following a reduction or an absence of the simulation or activation signal in the T cells, the expression of the operably linked transgene, e.g., encoding the recombinant receptor or a portion thereof, is reduced or downregulated. In some embodiments, the expression of the operably linked transgene is reduced or downregulated within less than at or about 6, 12, 18, 24, 36 or 48 hours following the reduction or an absence of the simulation or activation signal in the T cell. In some embodiments, after the stimulation or activation signal in the T cells, or following a reduction or an absence of the simulation or activation signal in the T cells, the expression of the operably linked transgene, e.g., encoding a recombinant receptor or a portion thereof, is reduced by greater than at or about 30%, 40%, 50%, 60%, 70%, 75%, 80%, 85%, 90% or 95% or more. In some aspects, the reduction is compared to the maximum expression of the operably linked transgene, for example, following the initial stimulation or activation signal in the T cells. In some aspects, the reduction is compared to the maximum expression of the operably linked transgene, for example, following the initial stimulation or activation signal in the T cells.

In some aspects, such conditional regulation of expression of the encoded recombinant receptor can result in a feed-back loop that can limit or prevent expression of the encoded recombinant receptor in the absence of stimulation or activation signal, for example, in the absence of target antigens or target cells. Such conditional regulation can reduce non-specific activation of the engineered cells or activation of the engineered cells in the absence of target antigen. In some aspects, following removal of target cells, e.g., malignant or tumor cells expressing the target antigen, engineered T cells can express fewer recombinant receptors and become less responsive, allowing healthy cell populations to recover and reducing non-specific activation or stimulation of the engineered cells.

In some embodiments, the expression of the operably linked transgene is capable of being induced or upregulated again following a further simulation or activation signal in the T cells after a reduction or absence of the signal. In some embodiments, the expression of the operably linked transgene is upregulated or induced within less than at or about 6, 12, 18, 24, 36 or 48 hours following the further simulation or activation signal in the T cells after a reduction or absence of the signal. In some embodiments, the expression of the operably linked transgene is upregulated or induced within less than at or about 24 hours following the further simulation or activation signal in the T cells after a reduction or absence of the signal. In some embodiments, the expression of the operably linked transgene is upregulated or induced by greater than at or about 30%, 40%, 50%, 60%, 70%, 75%, 80%, 85%, 90% or 95% or more. In some aspects, the upregulation or induction is compared to the expression of the operably linked transgene, before, or in the absence of the further stimulation or activation signal in the T cells, the minimum expression of the operably linked transgene prior to the further stimulation or activation signal in the T cells. In some embodiments, following the further simulation or activation signal in the T cells the mean level of expression of the recombinant receptor is increased by greater than at or about 40%, 50%, 60%, 70%, 80%, 90%, 100% or more.

In some embodiments, the T cell stimulation-associated locus encodes molecule that is transiently upregulated or induced upon T cell. In some embodiments, exemplary T cell stimulation-associated locus includes PDCD1 (encoding PD-1), CD69, Nur77 (encoding NR4A1), FoxP3 or a HLA-DR locus. In some aspects, the transgene (e.g., encoding a recombinant receptor or a portion thereof), is operably linked to the transcriptional regulatory element, such as the promoter, of the endogenous PDCD1 (encoding PD-1), CD69, Nur77 (encoding NR4A1), FoxP3 or a HLA-DR locus. In some embodiments, the transgene (e.g., encoding a recombinant receptor or a portion thereof) is integrated at or near the endogenous PDCD1 (encoding PD-1), CD69, Nur77 (encoding NR4A1), FoxP3 or a HLA-DR locus. In some aspects, the transgene is operably linked to, and integrated downstream of, the promoter of the T cell stimulation-associated locus. In some aspects, the transgene is operably linked to, and integrated downstream of, the endogenous PDCD1 promoter. In some aspects, the transgene is operably linked to, and integrated downstream of, the endogenous CD69 promoter. In some aspects, the transgene is operably linked to, and integrated downstream of, the endogenous Nur77 promoter. In some aspects, the transgene is operably linked to, and integrated downstream of, the endogenous FoxP3 promoter. In some aspects, the transgene is operably linked to, and integrated downstream of, the endogenous HLA-DR promoter.

In some embodiments, the transgene, e.g., encoding a recombinant receptor or a portion thereof, is associated with, under operable control of and/or regulated by a T cell stimulation-associated locus. In some embodiments, the recombinant receptor is encoded by a nucleic acid sequence under the operable control of an endogenous T cell stimulation-associated locus. In some aspects, the expression of the transgene, e.g., encoding a recombinant receptor or a portion thereof, is regulated by regulatory element that is responsive to the quality and/or strength of the signal through the intracellular signaling region and/or binding and/or recognition of the recombinant receptor to a target antigen or epitope. In some embodiments, a “T cell stimulation-associated locus” is an endogenous gene locus that is responsive to a signal transduced through a components of the TCR complex of a T cell, or a recombinant receptor comprising intracellular signaling regions that comprise a component of the TCR complex or a portion thereof, and/or antigen or epitope binding by a receptor, e.g. T cell receptor (TCR) or a recombinant receptor, present or expressed on a T cell. In some embodiments, the T cell stimulation-associated locus can be regulated by a canonical factor that is part of the normal downstream signaling pathway of T cells. In some embodiments, antigen or epitope binding and/or signal or activity through the intracellular signaling region of the recombinant receptor, e.g., CAR or a TCR, induces signaling that induces the T cell stimulation-associated locus to express the transgene, e.g., encoding the recombinant receptor. Detectable expression of the endogenous gene product and/or the transgene can then be monitored as an indicator of T cell activation.

In some embodiments, the T cell stimulation-associated locus contains one or more regulatory elements, such as one or more transcriptional control elements, whose expression depends on or is associated with activation of components of the TCR complex, whereby the regulatory domain or element is recognized by a transcription factor to drive expression of such gene. In some embodiments, the T cell stimulation-associated locus contains a promoter, enhancer or other response element or portion thereof, recognized by a transcription factor to drive expression of a gene whose activity is normally turned on or activated by T cell stimulation or activation. In some embodiments, the T cell stimulation-associated locus can contain a regulatory domain or region (e.g. promoter, enhancer or other response element) of a transcription factor whose activity is turned on by T cell stimulation or activation. In some embodiments, the T cell stimulation-associated locus is responsive to one or more of the quality and/or strength of the signal through the intracellular signaling region and/or binding and/or recognition of the recombinant receptor to a target antigen, ligand or epitope. In some embodiments, the regulatory element is responsive to one or more of the state of the endogenous TCR binding to an antigen or epitope, T cell stimulation or activation, signal strength through the TCR and/or quality of the signaling through the intracellular signaling region of the endogenous TCR.

In some embodiments, the endogenous T cell stimulation-associated locus contain one or more regulatory elements, such as a transcriptional regulatory element, such as promoter, enhancer or response element, that contain one or more binding site for a T cell transcription factor, and that thereby is associated with the downstream activity of a T cell transcription factor. In some embodiments, the transcription factor is nuclear factor of activated T cells (NFAT), C/EBP, STAT1, STAT2, or NF-κB. In some embodiments, the T cell stimulation-associated locus contains one or more response elements recognized by a nuclear factor of activated T cells (NFAT), C/EBP, STAT1, STAT2, or NF-κB. In some embodiments, the T cell stimulation-associated locus can contain one or more regulatory elements recognized by or responsive to one or two, and in some cases three or more, unique transcription factors.

In some embodiments, the T cell stimulation-associated locus comprises one or more response elements recognized by a transcription factor that is activated upon stimulation of T cells through an endogenous TCR complex. In some embodiments, regulatory regions of genes contain multiple regulatory elements that can be responsive to more than one signaling pathway in a cell. In some embodiments, the T cell stimulation-associated locus contains one or more regulatory elements recognized by NFAT. In some embodiments, the T cell stimulation-associated locus contains one or more regulatory elements recognized by NF-κB.

In some embodiments, the T cell stimulation-associated locus is associated with NFAT activity and/or NFAT-regulated signal transduction. The NFAT family of transcription factors plays a role in the transcriptional regulation of cytokine genes and other genes involved in the immune response, including in response to T cell activation. In some embodiments, the T cell stimulation-associated locus contains a regulatory element, such as a promoter, enhancer or response element, that contains a binding site and/or is recognized by NFAT and that can drive the expression of a transgene, e.g., encoding a recombinant receptor, operably connected thereto.

In some embodiments, the T cell stimulation-associated locus is associated with the activity of NF-κB and/or NF-κB-mediated signal transduction. Activation of NF-κB is dependent on stimulation of the TCR (i.e. via CD3 signaling) and co-stimulation via CD28, and can be regulated by ligation of both CD3 and CD28. While CD28 or CD3 signaling can induce NF-κB transcription, co-ligation of CD28 with TCR signaling (i.e. CD3 signaling) can produce greater transcriptional activity (Thaker et al. (2015) Immunology Letters, 163:113-119). In some embodiments, the T cell stimulation-associated locus can be a transcriptional regulatory element, such as a promoter, enhancer or response element, that contains one or more binding sites and/or that is recognized by NF-κB and that can drive the expression of a transgene, e.g., encoding a recombinant receptor, operably connected thereto. In some cases, a T cell stimulation-associated locus that contains a regulatory element responsive to NF-κB signaling can be an indicator of the quality of T cell signaling and the presence of both TCR-mediated signaling and costimulatory signaling.

In some embodiments, expression regulated by regulatory elements of a T cell stimulation-associated locus depends on, is induced and/or is upregulated upon T cell signaling. For example, the regulatory domain or element can be a promoter or portion thereof of an endogenous T cell stimulation-associated locus. In some embodiments, the promoter or portion thereof can contain a binding site and/or be recognized by one or more transcription factors.

In some embodiments, the T cell stimulation-associated locus contain a transcriptional regulatory element, such as a promoter or enhancer or other response element, that is or is part of the endogenous gene loci regulating expression of a T cell transcription factor, which are genes whose expression can be induced by T cell signaling or activation. In some embodiments, the transcription factor is nuclear factor of activated T cells (NFAT), nerve growth factor IB (also known as Nur77, NR4A1), C/EBP, STAT1, STAT2, and NFκB.

In some embodiments, the recombinant receptor is encoded by a nucleic acid sequence under the operable control of a T cell stimulation-associated locus, such as a regulatory element that is responsive to the quality and/or strength of the signal through an antigen receptor such as a TCR complex. In some aspects the T cell stimulation-associated locus is responsive to the quality and/or strength of signal through the intracellular signaling region of, and/or in response to the binding to and/or recognition of a recombinant receptor (such as the receptor operably linked to the endogenous T cell stimulation-associated locus) a target antigen or epitope.

In some of any of the provided embodiments, the T cell stimulation-associated locus is PDCD1. In some embodiments, the provided engineered cells comprise a modified PDCD1 locus that comprises a transgene encoding a recombinant receptor or a portion thereof, operably linked to the endogenous transcriptional regulatory element of PDCD1. PDCD1 encodes the inhibitory receptor programmed cell death-1 (PD-1; also known as PDCD1; CD279; PD-1; PD1; SLEB2; hPD-1; hPD-1; or hSLE1), which is a mediator of central and peripheral immune tolerance and immune exhaustion. In some aspects, PD-1 expression on CD8 T cells correlate with the presence and/or strength of signal through the T cell receptor (TCR). In some aspects, TCR stimulation initiates a signaling cascade through the calcineurin pathway, resulting in activation and translocation of the transcription factor NFATc1 (also known as NFAT2). In some aspects, NFATc1 is involved in initial activation-induced expression of PD-1 in CD4 and CD8 T cells, and other regulatory mechanism is also involved in maintaining and augmenting expression chronic antigen exposure (see, e.g., Bally et al., J Immunol (2016) 196 (6) 2431-2437; Simon et al., Oncoimmunology. 2018; 7(1): e1364828; Arasanz et al., Oncotarget. 2017 Aug. 1; 8(31): 51936-51945).

In some of any of the provided embodiments, the T cell stimulation-associated locus is CD69. In some embodiments, the provided engineered cells comprise a modified CD69 locus that comprises a transgene encoding a recombinant receptor or a portion thereof, operably linked to the endogenous transcriptional regulatory element of CD69. CD69 encodes the transmembrane C-Type lectin protein Cluster of Differentiation 69 (CD69; also known as AIM; BL-AC/P26; CLEC2C; EA1; GP32/28; or MLR-3). CD69 is early activation marker that is expressed in T cells, hematopoietic stem cells, natural killer (NK) cells, dendritic cells (DC) and other immune cells. CD69 is an early inducible cell surface glycoprotein acquired during lymphoid activation, is involved in lymphocyte proliferation and functions as a signal-transmitting receptor in lymphocytes, including natural killer (NK) cells, and platelets. CD69 expression is rapidly induced, e.g., is detected early after activation (30-60 min), on the surface of T lymphocytes after TCR/CD3 engagement, activating cytokines and polyclonal, mitogenic stimulation. Transcriptional expression of the CD69 gene declines rapidly after 4 to 6 hours. CD69 protein expression can be detected as early as 2 to 3 hours after stimulation (see, e.g., Alari-Pahissa et al., PLoS One. 2012; 7(10): e48593; Cibrian et al., Eur J Immunol. 2017 June; 47(6): 946-953).

In some of any of the provided embodiments, the T cell stimulation-associated locus is Nur77. In some embodiments, the provided engineered cells comprise a modified Nur77 locus that comprises a transgene encoding a recombinant receptor or a portion thereof, operably linked to the endogenous transcriptional regulatory element of Nur77. Nur77 encodes the nerve growth factor IB (NGFIB; also known as Nr4a1, nerve growth factor IB (NGFIB), GFRP1; Gfrp; HMR; Hbr-1; Hbr1; Hmr; N10; NAK-1; NGFI-B; NGFIB; NP10; Ngfi-b; Orphan nuclear receptor HMR; ST-59; TIS1; TR3; TR3 orphan receptor; early response protein NAK1; growth factor-inducible nuclear protein N10; hormone receptor; immediate early gene transcription factor NGFI-B; nerve growth factor IB nuclear receptor variant 1; nerve growth factor induced protein I-B; nerve growth factor-induced protein I-B; neural orphan nuclear receptor NUR77; nhr-6; nr4a1; nuclear hormone receptor NUR/77; nuclear protein N10; nuclear receptor subfamily 4 group A member 1; orphan nuclear receptor NGFI-B; orphan nuclear receptor NR4A1; orphan nuclear receptor TR3; steroid receptor TR3; testicular receptor 3; or zgc:92434), member of the Nur nuclear receptor family of intracellular transcription factors. In some contexts, Nur77 expression is sensitive to a primary activation signal in a T cell, signals from a signaling domain of a T cell receptor (TCR) component, and/or a signaling domain comprising an immunoreceptor tyrosine-based activation motif (ITAM). In some contexts, the expression of Nur77 is dose-responsive to signals through the signaling regions.

Nur77 is an immediate-early response gene expressed in T cells within hours after TCR stimulation, and can be induced by phytohemagglutinin in human lymphocytes and by serum stimulation of arrested fibroblasts. Nur77 is induced in response to signaling through, or activation of signal from, the endogenous TCR complex, engagement of the endogenous TCR and/or via molecules containing immunoreceptor tyrosine-based activation motif (ITAM) that are involved in the signal from the TCR complex, e.g., CD3-zeta signaling regions. Nur77 gene product itself generally can bind to regulatory elements associated with the promoters of several genes to induce downstream expression of genes. The level or extent of expression of Nur77 can serve as an indicator for strength of T cell signals, e.g., TCR signals (Moran et al. (2011) JEM, 208:1279-1289). Thus, in some embodiments, expression of a reporter molecule operably connected to a transcriptional regulatory element or elements of the Nur77 gene locus, or portion thereof, can provide an indicator of the strength of T cells signaling. Further, Nur77 expression is generally not affected or influenced by other signaling pathways such as cytokine signaling or toll-like receptor (TLR) signaling (see, e.g., Ashouri et al., (2017) J. Immunol. 198:657-668), which may act in a cell extrinsic manner and may not depend on signaling through the recombinant receptor.

In some of any of the provided embodiments, the T cell stimulation-associated locus is FOXP3. In some embodiments, the provided engineered cells comprise a modified FOXP3 locus that comprises a transgene encoding a recombinant receptor or a portion thereof, operably linked to the endogenous transcriptional regulatory element of FOXP3. The FOXP3 gene encodes Forkhead box P3 (Foxp3; also known as scurfin, AIID, DIETER, IPEX, JM2, PIDX, XPID), a protein involved in immune system responses, in some cases, considered a regulator of the regulatory pathway in the development and function of regulatory T cells. The expression of FoxP3 is induced by T cells, including in activated non-suppressive T cells or suppressor T cells, following stimulation or activation. The expression is transient in CD8+CD25+ T cells but in some cases, such as in CD4+CD25+ regulatory T cells, the expression can be more stable (see, e.g., Kmieciak et al., J Transl Med. 2009; 7: 89; Wang et al., Eur J Immunol. 2007 January; 37(1):129-38; Yu et al., Oncol Lett. 2018 June; 15(6): 8187-8194).

In some of any of the provided embodiments, the T cell stimulation-associated locus is a HLA-DR locus. In some embodiments, the provided engineered cells comprise a modified HLA-DR gene locus that comprises a transgene encoding a recombinant receptor or a portion thereof, operably linked to the endogenous transcriptional regulatory element of an HLA-DR gene locus. HLA-DR is a human class II major histocompatibility complex (MHC) antigen which is constitutively expressed on the surface of B lymphocytes, monocytes, and macrophages and appears at the late stages of activation on T and NK cells. In some aspects, HLA-DR is a late stage activation marker (see, e.g., Bajnok et al., Mediators of Inflammation (2017) Article ID 8045161; Revenfeld et al., Int J Mol Sci. 2017 July; 18(7): 1603; Reddy et al., J. Immun. Methods. 2004, 293(1-2): 127-142). HLA-DR is an MHC class II cell surface receptor encoded by the human leukocyte antigen complex, on chromosome 6 region 6p21.31. HLA-DR is encoded by several loci and several genes of different function at each locus. The DR α-chain is encoded by the HLA-DRA locus. The DR β-chain is encoded by several different loci, including HLA-DRB1 to HLA-DRB9, in which only some of them are present in each individual. The HLA-DRB1 locus is ubiquitous and encodes a very large number of functionally variable gene products (HLA-DR1 to HLA-DR17) (see, e.g., Marsh et al., Tissue Antigens. 2010 April; 75(4): 291-455).

In some aspects, the level, amount, pattern and timing of expression of the T cell stimulation-associated locus, and/or the transgene, e.g., operably linked to the T cell stimulation-associated locus, can be determined by employing an assay to assess gene expression, in the presence of a T cell stimulation or activation signal. In some aspects, detecting the level, amount, pattern and timing of expression of the T cell stimulation-associated locus, and/or the transgene, e.g., operably linked to the T cell stimulation-associated locus, includes performing an in vitro assay or in vivo.

In some embodiments, the assay includes an assay that detects the level of the expressed gene product (e.g., polypeptide or protein encoded by the T cell stimulation-associated locus), for example, an immunoassay, an aptamer-based assay, a histological or cytological assay, or the level of the expressed ribonucleic acid (RNA), such as an mRNA expression level assay. In some embodiments, the level, amount, pattern and timing of expression of the T cell stimulation-associated locus, and/or the transgene, e.g., operably linked to the T cell stimulation-associated locus, are detected by assays such as immunocytochemistry or immunohistochemistry, an enzyme linked immunosorbent assay (ELISA; including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), immunoblotting, immunoprecipitation, radioimmunoassay (RIA), immunostaining, flow cytometry assay, surface plasmon resonance (SPR), chemiluminescence assay, lateral flow immunoassay, inhibition assay, avidity assay, nucleic acid-based or protein-based aptamer techniques, high performance liquid chromatography (HPLC), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays). In some embodiments, the level, amount, pattern and timing of expression of the T cell stimulation-associated locus, and/or the transgene, e.g., operably linked to the T cell stimulation-associated locus, is determined using a binding reagent that specifically binds to the gene product of the T cell stimulation-associated locus or the encoded recombinant receptor. In some cases, the binding reagent is an antibody or antigen-binding fragment thereof, an aptamer or a nucleic acid probe.

In some embodiments, the level, amount, pattern and timing of expression of the T cell stimulation-associated locus, and/or the transgene, e.g., operably linked to the T cell stimulation-associated locus, is determined using methods to detect the amount, level or expression of nucleic acids, such as the messenger RNA. In certain embodiments, the assay includes detecting, measuring, assessing, and/or quantifying the level of a polynucleotide, e.g., mRNA from the T cell stimulation-associated locus or the mRNA produced from the transgene. In some aspects, the amount or level of a polynucleotide can be assessed, measured, determined, and/or quantified by polymerase chain reaction (PCR), including reverse transcriptase (rt) PCR, droplet digital PCR, real-time and quantitative PCR methods (including, e.g., TAQMAN®, molecular beacon, LIGHTUP™, SCORPION™, SIMPLEPROBES®; see, e.g., U.S. Pat. Nos. 5,538,848; 5,925,517; 6,174,670; 6,329,144; 6,326,145 and 6,635,427); northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other methods known in the art, such as discussed in Shendure et al., Nat. Rev. Genet. 5:335-44 (2004) or Nowrousian, Eukaryotic Cell 9(9): 1300-1310 (2010), including such specific platforms as HELICOS®, ROCHE® 454, ILLUMINA®/SOLEXA®, ABI SOLiD®, and POLONATOR® sequencing. In some of any embodiments, the levels of a polynucleotide are measured by qRT-PCR. In some embodiments, the qRT-PCR uses three nucleic acid sets for each gene, where the three nucleic acids comprise a primer pair together with a probe that binds between the regions of a target nucleic acid where the primers bind.

In some embodiments, the level, amount, pattern and timing of expression of the T cell stimulation-associated locus, and/or the transgene, e.g., operably linked to the T cell stimulation-associated locus, is determined by sequencing the polynucleotides. In some embodiments, the sequencing is performed by a non-Sanger sequencing method and/or a next generation sequencing (NGS) technique. Examples of Next Generation Sequencing techniques include, but are not limited to Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing. In some embodiments, the NGS technique is RNA sequencing (RNA-Seq). RNA sequencing methods have been adapted for the most common DNA sequencing platforms, such as HiSeq systems (Illumina), 454 Genome Sequencer FLX System (Roche), Applied Biosystems SOLiD (Life Technologies), IonTorrent (Life Technologies). These platforms generally require initial reverse transcription of RNA into cDNA. Conversely, the single molecule sequencer HeliScope (Helicos BioSciences) is able to use RNA as a template for sequencing. A proof of principle for direct RNA sequencing on the PacBio RS platform has also been demonstrated (Pacific Bioscience). In some embodiments, the one or more RNA gene products are assessed, measured, determined, and/or quantified by RNAseq.

In some aspects, the level, amount, pattern and timing of expression of the T cell stimulation-associated locus, and/or the transgene, e.g., operably linked to the T cell stimulation-associated locus, can be determined in cells after exposure to a stimulation or activation signal. In some aspects, the expression of the T cell stimulation-associated locus and/or the transgene is determined by incubating the cells with an agent that provides a stimulation or activation signal. In some aspects, the level, amount and pattern of expression of the T cell stimulation-associated locus, and/or the transgene, e.g., operably linked to the T cell stimulation-associated locus, can be assessed at various times after exposure to the stimulation or activation signal. In some aspects, the level, amount and pattern of expression of the T cell stimulation-associated locus, and/or the transgene, e.g., operably linked to the T cell stimulation-associated locus, can be determined after a re-stimulation, repeated stimulation or serial stimulation. In some embodiments, the expression of the transgene can be assessed after incubation of T cells in the presence or absence of an agent that binds to the binding domain of the recombinant receptor and/or an agent that induces or is capable of inducing a signal through the intracellular signaling region of the recombinant receptor.

In some aspects, exemplary agents that can provide a stimulation or activation signal to assess the level, amount or pattern of expression includes those providing antigen-independent stimulation, e.g., agents containing anti-CD3 and/or anti-CD28 antibodies, such as a bead conjugated with an anti-CD3 and anti-CD28 antibodies, or soluble multimeric or oligomeric reagents loaded with anti-CD3/anti-CD28 antibodies or antibody fragments; or antigen-specific stimulation for the recombinant receptor, for example, purified or recombinant antigen that the recombinant receptor binds or recognizes, e.g., the target antigen or ligand of the antigen- or ligand-binding domain of the recombinant receptor. Other exemplary agents that can provide a stimulation or activation signal to the T cells for assessing the level, amount or pattern of expression include phorbol 12-myristate 13-acetate (PMA), also known as 12-O-tetradecanoylphorbol 13-acetate (TPA), ionomycin and/or Concanavalin A (Con A).

### II. METHODS FOR PRODUCING CELLS CONDITIONALLY EXPRESSING A RECOMBINANT RECEPTOR BY HOMOLOGY-DIRECTED REPAIR (HDR)

Provided herein are methods of generating or producing genetically engineered cells comprising a modified T cell stimulation-associated locus (for example, PDCD1 (encoding PD-1), CD69, Nur77 (encoding NR4A1), FoxP3 or a HLA-DR locus) in which the modified T cell stimulation-associated locus includes a transgene (e.g., heterologous or exogenous nucleic acid sequences) encoding a recombinant receptor, such as a chimeric antigen receptor (CAR) or a T cell receptor (TCR). In some aspects, the modified T cell stimulation-associated locus in the genetically engineered cell comprises a transgene encoding a recombinant receptor or a portion thereof, integrated into an endogenous T cell stimulation-associated locus. In some embodiments, provided are methods that involve inducing a targeted genetic disruption and homology-dependent repair (HDR), using template polynucleotides containing the transgene encoding a recombinant receptor or a portion thereof, thereby targeting integration of the transgene at the T cell stimulation-associated locus. Also provided are cells and cell compositions generated by the methods, and polynucleotides, e.g., template polynucleotides, and kits for use in the methods.

In some aspects, the provided embodiments employ HDR for targeted integration of the recombinant or heterologous sequences into the T cell stimulation-associated locus. In some cases, the methods involve introducing one or more targeted genetic disruption(s), e.g., DNA break, at the endogenous T cell stimulation-associated locus by gene editing techniques, combined with targeted integration of a transgene encoding a recombinant receptor or a portion thereof by HDR. In some aspects, the one or more targeted genetic disruption(s) is carried out by introduction of one or more agent(s) capable of introducing the genetic disruption(s). In some embodiments, the HDR step entails a disruption or a break, e.g., a double-stranded break, in the DNA at the target genomic location. In some embodiments, the DNA break is induced by employing gene editing methods, e.g., targeted nucleases. In some embodiments, the methods generate an engineered cell that is knocked-out for expression of T cell stimulation-associated locus. In some embodiments, the methods generate an engineered cell that retains the expression of T cell stimulation-associated locus. In some aspects, after carrying out the methods, the engineered T cell comprises a transgene encoding a recombinant receptor or a portion thereof operably linked to an endogenous transcriptional regulatory element of the T cell stimulation-associated locus. In some aspects, the endogenous transcriptional regulatory element induces or upregulates expression of the operably linked transgene following a simulation or activation signal in the T cell.

In some aspects, the provided methods involve introducing one or more agent(s) capable of inducing a genetic disruption of at a target site within a T cell stimulation-associated locus into a T cell; and introducing into the T cell a polynucleotide, e.g., a template polynucleotide, comprising a transgene and one or more homology arms. In some aspects, the transgene contains a sequence of nucleotides encoding a recombinant receptor or a portion thereof. In some embodiments, the nucleic acid sequence is targeted for integration within the T cell stimulation-associated locus via homology directed repair (HDR).

In some aspects, the provided methods involve introducing a polynucleotide comprising a transgene encoding a recombinant receptor or a portion thereof comprising into a T cell having a genetic disruption of within a T cell stimulation-associated locus, wherein the genetic disruption has been induced by one or more agents capable of inducing a genetic disruption of one or more target site within the T cell stimulation-associated locus, and wherein the nucleic acid sequence is targeted for integration within the T cell stimulation-associated locus via HDR. In some embodiments, also provided are compositions containing a population of cells that have been engineered to express a recombinant receptor, e.g., a CAR or a TCR, such that the cell population that exhibits more improved, uniform, homogeneous, regulated and/or stable expression and/or antigen binding by the recombinant receptor, including genetically engineered immune cells produced by any of the provided methods. In some aspects, the provided embodiments permit regulation of expression, such as conditional expression of the linked transgene, e.g., encoding a recombinant receptor, upon stimulation of the T cell, and reduced or downregulated expression following a reduction or an absence of the simulation or activation signal in the T cell.

In some aspects, the embodiments involve generating a targeted DNA break using gene editing methods and/or targeted nucleases, followed by HDR based on one or more template polynucleotide(s), e.g., template polynucleotide(s) that contains homology sequences that are homologous to sequences at the endogenous T cell stimulation-associated locus linked to a transgene encoding recombinant receptor or a portion thereof and in some cases nucleic acid sequence encoding other molecules, to specifically target and integrate the transgene at or near the DNA break. Thus, in some aspects, the methods involve a step of inducing a targeted genetic disruption (e.g., gene editing) and introducing a polynucleotide, e.g., a template polynucleotide comprising a transgene, into the cell (e.g., HDR).

In some embodiments, the targeted genetic disruption and targeted integration of the transgene by HDR occurs at one or more target site(s) at the endogenous T cell stimulation-associated locus. In some aspects, the targeted integration occurs within the open reading frame sequence of the endogenous T cell stimulation-associated locus. In some aspects, targeted integration of the transgene results in a knock-out of the endogenous T cell stimulation-associated locus gene, e.g., such that the expression of the endogenous gene is eliminated.

In some aspects, the transgene has been integrated into the T cell stimulation-associated locus, e.g., by homology-directed repair (HDR) within an exon of an open reading frame or a partial sequence thereof of the endogenous T cell stimulation-associated locus, such that the sequences encoding the chimeric receptor or a portion thereof is in-frame with the sequence of the exon. In some aspects, all or a portion of the endogenous T cell stimulation-associated locus, such as the portion upstream of the integrated transgene, and the recombinant receptor or portion thereof are expressed in the modified T cell stimulation-associated locus, in some cases separated by a multicistronic element.

In some embodiments, a template polynucleotide is introduced into the engineered cell, prior to, simultaneously with, or subsequent to introduction of one or more agent(s) capable of inducing one or more targeted genetic disruption. In the presence of one or more targeted genetic disruption, e.g., DNA break, the template polynucleotide can be used as a DNA repair template, to effectively integrate the transgene, at or near the site of the targeted genetic disruption by HDR, based on homology between the endogenous gene sequence surrounding the genetic disruption and the one or more homology arms, such as the 5′ and/or 3′ homology arms included in the template polynucleotide.

In some aspects, the template polynucleotide and the one or more agent(s) capable of inducing one or more targeted genetic disruption is introduced simultaneously. In some aspects, the template polynucleotide and the one or more agent(s) capable of inducing one or more targeted genetic disruption is introduced using any delivery method described herein, e.g., in Section II.A.3 and II.B.3. In some aspects, the template polynucleotide and the one or more agent(s) capable of inducing one or more targeted genetic disruption is introduced via a physical delivery method, such as via electroporation, particle gun, calcium phosphate transfection, cell compression or squeezing. In some aspects, the template polynucleotide and the one or more agent(s) capable of inducing one or more targeted genetic disruption is introduced simultaneously, via electroporation.

In some aspects, the two steps can be performed sequentially. In some embodiments, the gene editing and HDR steps are performed simultaneously and/or in one experimental reaction. In some embodiments, the gene editing and HDR steps are performed consecutively or sequentially, in one or consecutive experimental reaction(s). In some embodiments, the gene editing and HDR steps are performed in separate experimental reactions, simultaneously or at different times.

The immune cells can include a population of cells containing T cells. Such cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+ T cells, CD8+ T cells or CD4+ and CD8+ T cells. In some embodiments, the step of introducing the polynucleotide template and the step of introducing the agent (e.g. Cas9/gRNA RNP) can occur simultaneously or sequentially in any order. In some of any embodiments, the polynucleotide template is introduced into the immune cells after inducing the genetic disruption by the step of introducing the agent(s) (e.g. Cas9/gRNA RNP). In some embodiments, prior to, during and/or subsequent to introduction of the polynucleotide template and one or more agents (e.g. Cas9/gRNA RNP), the cells are cultured or incubated under conditions to stimulate expansion and/or proliferation of cells.

In some of any embodiments of the provided methods, the introduction of the template polynucleotide is performed simultaneously with the introduction of the one or more agent capable of inducing a genetic disruption. Any method for introducing the one or more agent(s) can be employed as described, depending on the particular agent(s) used for inducing the genetic disruption. In some aspects, the disruption is carried out by gene editing, such as using an RNA-guided nuclease such as a clustered regularly interspersed short palindromic nucleic acid (CRISPR)-Cas system, such as CRISPR-Cas9 system, specific for the T cell stimulation-associated locus being disrupted. In some embodiments, an agent containing a Cas9 and a guide RNA (gRNA) containing a targeting domain, which targets a region of the T cell stimulation-associated locus, is introduced into the cell. In some embodiments, the agent is or comprises a ribonucleoprotein (RNP) complex of Cas9 and gRNA containing the T cell stimulation-associated locus-targeted targeting domain (Cas9/gRNA RNP). In some embodiment, the introduction includes contacting the agent or portion thereof with the cells, in vitro, which can include cultivating or incubating the cell and agent for up to 24, 36 or 48 hours or 3, 4, 5, 6, 7, or 8 days. In some embodiments, the introduction further can include effecting delivery of the agent and/or a polynucleotide comprising the transgene, such as a template for HDR, into the cells. In various embodiments, the methods, compositions and cells according to the present disclosure utilize direct delivery of ribonucleoprotein (RNP) complexes of Cas9 and gRNA and/or the template polynucleotide to cells, for example by electroporation. In some cases, electroporation of the cells to be modified includes cold-shocking the cells, e.g. at 32° C. following electroporation of the cells and prior to plating.

Any method for introducing the template polynucleotide can be employed as described, depending on the particular methods used for delivery of the template polynucleotide to cells. In some embodiments, the template polynucleotide is a linear polynucleotide. In some embodiments, the template polynucleotide is a single-stranded linear polynucleotide. In some embodiments, the template polynucleotide is a double-stranded linear polynucleotide. In some aspects, the template polynucleotide is delivered into the cells by a physical delivery means, such as electroporation, separately or together with one or more agent(s) to induce a genetic disruption at one or more of the target sites in the genome.

Exemplary methods include those for transfer of nucleic acids encoding the receptors, including via viral, e.g., retroviral or lentiviral, transduction, transposons, and electroporation. In some of any embodiments, viral transduction methods are employed. In some embodiments, template polynucleotides can be transferred or introduced into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557. In some of any embodiments, the viral vector is an AAV such as an AAV2 or an AAV6.

In such aspects of the provided methods, a template polynucleotide is introduced into the cells after introduction with the one or more agent(s), such as Cas9/gRNA RNP, e.g. that has been introduced via electroporation. In some embodiments, the template polynucleotide is introduced immediately after the introduction of the one or more agents capable of inducing a genetic disruption. In some embodiments, the template polynucleotide is introduced into the cells within at or about 30 seconds, within at or about 1 minute, within at or about 2 minutes, within at or about 3 minutes, within at or about 4 minutes, within at or about 5 minutes, within at or about 6 minutes, within at or about 6 minutes, within at or about 8 minutes, within at or about 9 minutes, within at or about 10 minutes, within at or about 15 minutes, within at or about 20 minutes, within at or about 30 minutes, within at or about 40 minutes, within at or about 50 minutes, within at or about 60 minutes, within at or about 90 minutes, within at or about 2 hours, within at or about 3 hours or within at or about 4 hours after the introduction of one or more agents capable of inducing a genetic disruption. In some embodiments, the template polynucleotide is introduced into cells at time between at or about 15 minutes and at or about 4 hours after introducing the one or more agent(s), such as between at or about 15 minutes and at or about 3 hours, between at or about 15 minutes and at or about 2 hours, between at or about 15 minutes and at or about 1 hour, between at or about 15 minutes and at or about 30 minutes, between at or about 30 minutes and at or about 4 hours, between at or about 30 minutes and at or about 3 hours, between at or about 30 minutes and at or about 2 hours, between at or about 30 minutes and at or about 1 hour, between at or about 1 hour and at or about 4 hours, between at or about 1 hour and at or about 3 hours, between at or about 1 hour and at or about 2 hours, between at or about 2 hours and at or about 4 hours, between at or about 2 hours and at or about 3 hours or between at or about 3 hours and at or about 4 hours. In some embodiments, the template polynucleotide is introduced into cells at or about 2 hours after the introduction of the one or more agents, such as Cas9/gRNA RNP, e.g. that has been introduced via electroporation.

In some embodiments, prior to, during or subsequent to contacting the agent with the cells and/or prior to, during or subsequent to effecting delivery (e.g. electroporation), the provided methods include incubating the cells in the presence of a cytokine, a stimulating agent and/or an agent that is capable of inducing proliferation, stimulation or activation of the immune cells (e.g. T cells). In some embodiments, at least a portion of the incubation is in the presence of a stimulating agent that is or comprises an antibody specific for CD3, an antibody specific for CD28 and/or a cytokine, such as anti-CD3/anti-CD28 beads. In some embodiments, at least a portion of the incubation is in the presence of a cytokine, such as one or more of recombinant IL-2, recombinant IL-7 and/or recombinant IL-15. In some embodiments, the incubation is for up to 8 days before or after the introduction with the one or more agent(s), such as Cas9/gRNA RNP, e.g. via electroporation, and template polynucleotide, such as up to 24 hours, 36 hours or 48 hours or 3, 4, 5, 6, 7 or 8 days.

In some embodiments, the method includes activating or stimulating cells with a stimulating agent (e.g. anti-CD3/anti-CD28 antibodies) prior to introducing the agent, e.g. Cas9/gRNA RNP, and the polynucleotide template. In some embodiments, the incubation in the presence of a stimulating agent (e.g. anti-CD3/anti-CD28) is for 6 hours to 96 hours, such as 24-48 hours or 24-36 hours prior to the introduction with the one or more agent(s), such as Cas9/gRNA RNP, e.g. via electroporation. In some embodiments, the incubation with the stimulating agents can further include the presence of a cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). In some embodiments the stimulating agent(s) (e.g. anti-CD3/anti-CD28 antibodies) is washed or removed from the cells prior to introducing or delivering into the cells the agent(s) capable of inducing a genetic disruption Cas9/gRNA RNP and/or the polynucleotide template. In some embodiments, prior to the introducing of the agent(s), the cells are rested, e.g. by removal of any stimulating or activating agent. In some embodiments, prior to introducing the agent(s), the stimulating or activating agent and/or cytokines are not removed.

In some embodiments, subsequent to the introduction of the agent(s), e.g. Cas9/gRNA, and/or the polynucleotide template the cells are incubated, cultivated or cultured in the presence of a recombinant cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). The cells can be incubated or cultivated under conditions to induce proliferation or expansion of the cells. In some embodiments, the cells can be incubated or cultivated until a threshold number of cells is achieved for harvest, e.g. a therapeutically effective dose.

In some embodiments, the incubation during any portion of the process or all of the process can be at a temperature of 30° C.±2° C. to 39° C.±2° C., such as at least or about at least 30° C.±2° C., 32° C.±2° C., 34° C.±2° C. or 37° C.±2° C. In some embodiments, at least a portion of the incubation is at 30° C.±2° C. and at least a portion of the incubation is at 37° C.±2° C.

In some aspects, the provided embodiments allow the recombinant receptor to be expressed under the control of endogenous transcriptional regulatory element of the T cell stimulation-associated locus, e.g., the endogenous promoter of the T cell stimulation-associated locus. In some aspects, the provided embodiments allow the nucleic acids encoding the recombinant receptor to be operably linked to the endogenous regulatory or control elements, e.g., cis regulatory elements, such as the promoter, or the 5′ and/or 3′ untranslated regions (UTRs) of the endogenous T cell stimulation-associated locus. Thus, in some aspects, the provided embodiments allow the recombinant receptor, e.g., CAR, to be expressed and/or the expression is conditionally, temporally and/or quantitatively regulated similarly to the endogenous T cell stimulation-associated locus. In some aspects, the expression of the operably linked transgene is upregulated or induced following the stimulation or activation signal in the T cells. In some aspects, the expression of the operably linked transgene is reduced or downregulated following a reduction or an absence of the simulation or activation signal in the T cells. In some aspects, the expression of the operably linked transgene is capable of being induced or upregulated again following a further simulation or activation signal in the T cells after a reduction or absence of the signal.

### A. Genetic Disruption

In some embodiments, one or more targeted genetic disruption is induced at the one or more endogenous T cell stimulation-associated locus. In some embodiments, the targeted genetic disruption is induced in or near an exon of the endogenous T cell stimulation-associated locus. In some embodiments, the targeted genetic disruption is induced in or near an intron of the endogenous T cell stimulation-associated locus. In some embodiments, the targeted genetic disruption is induced in or near a promoter of an endogenous T cell stimulation-associated locus. In some aspects, the presence of the one or more targeted genetic disruption and a polynucleotide, e.g., a template polynucleotide that contains a transgene encoding a recombinant receptor or a portion thereof, can result in targeted integration of the transgene at or near the one or more genetic disruption at the endogenous T cell stimulation-associated locus.

In some embodiments, genetic disruption results in a DNA break, such as a double-strand break (DSB) or a cleavage, or a nick, such as a single-strand break (SSB), at one or more target site in the genome. In some embodiments, at the site of the genetic disruption, e.g., DNA break or nick, action of cellular DNA repair mechanisms can result in knock-out, insertion, missense or frameshift mutation, such as a biallelic frameshift mutation, deletion of all or part of the gene; or, in the presence of a repair template, e.g., a template polynucleotide, can alter the DNA sequence based on the repair template, such as integration or insertion of the nucleic acid sequences contained in the template polynucleotide (e.g., any described in Section II.B.2 herein). In some embodiments, the genetic disruption can be targeted to one or more exon of a gene or portion thereof. In some embodiments, the genetic disruption can be targeted near a desired site of targeted integration of exogenous sequences, e.g., exogenous sequences encoding a recombinant receptor. In some embodiments, the modified T cell stimulation-associated locus, after integration of the transgene encoding a recombinant receptor or a portion thereof, comprises a deletion, an insertion, a frameshift mutation or a nonsense mutation in the open reading frame of the endogenous T cell stimulation-associated locus. In some aspects, the endogenous gene product of the T cell stimulation-associated locus is not produced, or is truncated, or is non-functional in the cell. In some aspects, the endogenous gene product of the T cell stimulation-associated locus is produced in full length or is functional in the cell.

In some embodiments, a DNA binding protein or DNA-binding nucleic acid, which specifically binds to or hybridizes to the sequences at a region near one of the at least one target site(s), is used for targeted disruption. In some embodiments, template polynucleotides, e.g., template polynucleotides that include nucleic acid sequence encoding a recombinant receptor or a portion thereof, and homology sequences, can be introduced for targeted integration by HDR of the recombinant receptor-encoding sequences at or near the site of the genetic disruption, such as described herein, for example, in Section II.A.

In some embodiments, the genetic disruption is carried by introducing one or more agent(s) capable of inducing a genetic disruption. In some embodiments, such agents comprise a DNA binding protein or DNA-binding nucleic acid that specifically binds to or hybridizes to the gene. In some embodiments, the agent comprises various components, such as a fusion protein comprising a DNA-targeting protein and a nuclease or an RNA-guided nuclease. In some embodiments, the agents can target one or more target sites or target locations. In some aspects, a pair of single stranded breaks (e.g., nicks) on each side of the target site can be generated.

In provided embodiments, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors. Methods, such as electroporation, also can be used to introduce or deliver protein or ribonucleoprotein (RNP), e.g. containing the Cas9 protein in complex with a targeting gRNA, to cells of interest.

In some embodiments, the genetic disruption occurs at a target site (also known as “target position,” “target DNA sequence” or “target location”), for example, at the endogenous T cell stimulation-associated locus. In some embodiments, the target site includes a site on a target DNA (e.g., genomic DNA) that is modified by the one or more agent(s) capable of inducing a genetic disruption, e.g., a Cas9 molecule complexed with a gRNA that specifies the target site. For example, the target site can include locations in the DNA at the endogenous T cell stimulation-associated locus, where cleavage or DNA breaks occur. In some aspects, integration of nucleic acid sequences by HDR can occur at or near the target site or target sequence. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence.

### 1. Target Site at Exemplary Endogenous T Cell Stimulation-Associated Loci

In some embodiments, the genetic disruption and/or integration of the transgene encoding a recombinant receptor or a portion thereof, via homology-directed repair (HDR), are targeted at an endogenous or genomic T cell stimulation-associated locus described herein. In some aspects, by virtue of the genetic disruption at the target site described herein, and the presence of template polynucleotides for targeted integration of the transgene at the T cell stimulation-associated locus, the resulting engineered T cell contains a transgene encoding a recombinant receptor or a portion thereof operably linked to an endogenous transcriptional regulatory element of the T cell stimulation-associated locus. In some aspects, the endogenous transcriptional regulatory element induces or upregulates expression of the operably linked transgene following a simulation or activation signal in the T cell.

In some embodiments, a genetic disruption is targeted at, near, or within a T cell stimulation-associated locus. In some embodiments, the T cell stimulation-associated locus encodes molecule that is transiently upregulated or induced upon T cell. In some embodiments, exemplary T cell stimulation-associated locus includes PDCD1 (encoding PD-1), CD69, Nur77 (encoding NR4A1), FoxP3 or a HLA-DR locus. In some aspects, the target site is at or near the PDCD1 (encoding PD-1), CD69, Nur77 (encoding NR4A1), FoxP3 or a HLA-DR locus. Expression of the exemplary T cell stimulation-associated locus is described herein, e.g., in Section I.A.

Exemplary human PD-1 precursor polypeptide sequence is set forth in SEQ ID NO:79 (mature polypeptide includes residues 24-288 of SEQ ID NO:79; see Uniprot Accession No. Q15116-1; mRNA sequence set forth in SEQ ID NO:80, NCBI Reference Sequence: NM_005018.3). In humans, a genomic locus encoding PD-1, PDCD1, comprises an open reading frame that contains 5 exons and 4 introns. An exemplary mRNA transcript of PDCD1 spans the sequence corresponding to Chromosome 2: 241,849,884-241,858,894 reverse strand, with reference to human genome version GRCh38 (UCSC Genome Browser on Human December 2013 (GRCh38/hg38) Assembly). Table 1 sets forth the coordinates of the exons and introns of the open reading frames and the untranslated regions of an exemplary transcript encoding PD-1.

TABLE 1
Coordinates of exons and introns of human PDCD1
locus (GRCh38, Chromosome 2, reverse strand).

| | Start (GrCh38) | End (GrCh38) | Length |
|---|---|---|---|
| 5′ UTR and Exon 1 | 241,858,894 | 241,858,763 | 132 |
| Intron 1-2 | 241,858,762 | 241,852,981 | 5,782 |
| Exon 2 | 241,852,980 | 241,852,621 | 360 |
| Intron 2-3 | 241,852,620 | 241,852,354 | 267 |
| Exon 3 | 241,852,353 | 241,852,198 | 156 |
| Intron 3-4 | 241,852,197 | 241,851,984 | 214 |
| Exon 4 | 241,851,983 | 241,851,949 | 35 |
| Intron 4-5 | 241,851,948 | 241,851,298 | 651 |
| Exon 5 and 3′ UTR | 241,851,297 | 241,849,884 | 1,414 |

Exemplary human CD69 polypeptide sequence is set forth in SEQ ID NO:81 (see Uniprot Accession No. Q07108-1; mRNA sequence set forth in SEQ ID NO:82, NCBI Reference Sequence: NP_001772.1). In humans, a locus encoding CD69 comprises an open reading frame that contains 5 exons and 4 introns. An exemplary mRNA transcript of CD69 spans the sequence corresponding to Chromosome 12: 9,752,486-9,760,901 reverse strand, with reference to human genome version GRCh38 (UCSC Genome Browser on Human December 2013 (GRCh38/hg38) Assembly). Table 2 sets forth the coordinates of the exons and introns of the open reading frames and the untranslated regions of an exemplary transcript encoding CD69.

TABLE 2
Coordinates of exons and introns of human CD69
locus (GRCh38, Chromosome 12, reverse strand).

| | Start (GrCh38) | End (GrCh38) | Length |
|---|---|---|---|
| 5′ UTR and Exon 1 | 9,760,901 | 9,760,757 | 145 |
| Intron 1-2 | 9,760,756 | 9,756,420 | 4,337 |
| Exon 2 | 9,756,419 | 9,756,297 | 123 |
| Intron 2-3 | 9,756,296 | 9,755,262 | 1,035 |
| Exon 3 | 9,755,261 | 9,755,062 | 200 |
| Intron 3-4 | 9,755,061 | 9,754,691 | 371 |
| Exon 4 | 9,754,690 | 9,754,587 | 104 |
| Intron 4-5 | 9,754,586 | 9,753,590 | 997 |
| Exon 5 and 3′ UTR | 9,753,589 | 9,752,486 | 1,104 |

Exemplary human NR4A1 polypeptide sequence is set forth in SEQ ID NO:83 (isoform 1; see Uniprot Accession No. P22736-1; mRNA sequence set forth in SEQ ID NO:84, NCBI Reference Sequence: NP_002126.2). In humans, several different mRNA and protein isoforms are present for Nur77. An exemplary genomic locus encoding NR4A1, Nur77 (also known as NR4A1), comprises an open reading frame that contains 8 exons and 7 introns for the transcript variant that encodes isoform 1. An exemplary mRNA transcript of Nur77 encoding isoform 1 spans the sequence corresponding to Chromosome 12: 52,051,402-52,059,506 forward strand, with reference to human genome version GRCh38 (UCSC Genome Browser on Human December 2013 (GRCh38/hg38) Assembly). Table 3 sets forth the coordinates of the exons and introns of the open reading frames and the untranslated regions of an exemplary transcript isoform 1 encoding NR4A1.

TABLE 3
Coordinates of exons and introns of human Nur77
locus (GRCh38, Chromosome 12, forward strand).

| | Start (GrCh38) | End (GrCh38) | Length |
|---|---|---|---|
| 5′ UTR and Exon 1 | 52,051,402 | 52,051,568 | 167 |
| Intron 1-2 | 52,051,569 | 52,052,495 | 927 |
| Exon 2 | 52,052,496 | 52,052,645 | 150 |
| Intron 2-3 | 52,052,646 | 52,054,326 | 1,681 |
| Exon 3 | 52,054,327 | 52,055,204 | 878 |
| Intron 3-4 | 52,055,205 | 52,056,029 | 825 |
| Exon 4 | 52,056,030 | 52,056,159 | 130 |
| Intron 4-5 | 52,056,160 | 52,056,493 | 334 |
| Exon 5 | 52,056,494 | 52,056,645 | 152 |
| Intron 5-6 | 52,056,646 | 52,057,056 | 411 |
| Exon 6 | 52,057,057 | 52,057,259 | 203 |
| Intron 6-7 | 52,057,260 | 52,057,351 | 92 |
| Exon 7 | 52,057,352 | 52,057,530 | 179 |
| Intron 7-8 | 52,057,531 | 52,058,687 | 1,157 |
| Exon 8 and 3′ UTR | 52,058,688 | 52,059,506 | 819 |

Exemplary human FoxP3 polypeptide sequence is set forth in SEQ ID NO:85 (isoform 1; see Uniprot Accession No. Q9BZS1-1; mRNA sequence set forth in SEQ ID NO:86, NCBI Reference Sequence: NM_014009.3). In humans, several different mRNA and protein isoforms are present for FoxP3. An exemplary genomic locus encoding FoxP3, FOXP3, comprises an open reading frame that contains 12 exons and 11 introns for the transcript variant that encodes isoform 1. An exemplary mRNA transcript of FOXP3 encoding isoform 1 spans the sequence corresponding to Chromosome X: 49,250,436-49,264,924 reverse strand, with reference to human genome version GRCh38 (UCSC Genome Browser on Human December 2013 (GRCh38/hg38) Assembly). Table 4 sets forth the coordinates of the exons and introns of the open reading frames and the untranslated regions of an exemplary transcript isoform 1 encoding FoxP3.

TABLE 4
Coordinates of exons and introns of human FOXP3
locus (GRCh38, Chromosome X, reverse strand).

| | Start (GrCh38) | End (GrCh38) | Length |
|---|---|---|---|
| 5′ UTR and Exon 1 | 49,264,924 | 49,264,661 | 264 |
| Intron 1-2 | 49,264,660 | 49,258,528 | 6,133 |
| Exon 2 | 49,258,527 | 49,258,296 | 232 |
| Intron 2-3 | 49,258,295 | 49,257,769 | 527 |
| Exon 3 | 49,257,768 | 49,257,664 | 105 |
| Intron 3-4 | 49,257,663 | 49,257,566 | 98 |
| Exon 4 | 49,257,565 | 49,257,427 | 139 |
| Intron 4-5 | 49,257,426 | 49,257,013 | 414 |
| Exon 5 | 49,257,012 | 49,256,925 | 88 |
| Intron 5-6 | 49,256,924 | 49,256,856 | 69 |
| Exon 6 | 49,256,855 | 49,256,751 | 105 |
| Intron 6-7 | 49,256,750 | 49,255,803 | 948 |
| Exon 7 | 49,255,802 | 49,255,715 | 88 |
| Intron 7-8 | 49,255,714 | 49,255,510 | 205 |
| Exon 8 | 49,255,509 | 49,255,429 | 81 |
| Intron 8-9 | 49,255,428 | 49,254,068 | 1,361 |
| Exon 10 | 49,254,067 | 49,253,917 | 151 |
| Intron 9-10 | 49,253,916 | 49,253,203 | 714 |
| Exon 11 | 49,253,202 | 49,253,126 | 77 |
| Intron 10-11 | 49,253,125 | 49,251,766 | 1,360 |
| Exon 12 | 49,251,765 | 49,251,664 | 102 |
| Intron 11-12 | 49,251,663 | 49,251,484 | 180 |
| Exon 13 and 3′ UTR | 49,251,483 | 49,250,436 | 1,048 |

HLA-DR is a heterodimeric protein that includes an alpha (α) chain and a beta (β) chain. Each subunit of which contains two extracellular domains, a membrane-spanning domain and a cytoplasmic tail. Both α and β chains are anchored in the membrane. HLA-DR is encoded by several loci and several genes of different function at each locus. The DR α-chain is encoded by the HLA-DRA locus. The DR β-chain is encoded by several different loci, including HLA-DRB1 to HLA-DRB39, in which only some of them are present in each individual. The HLA-DRB1 locus is ubiquitous and encodes a very large number of functionally variable gene products (HLA-DR1 to HLA-DR17) (see, e.g., Marsh et al., Tissue Antigens. 2010 April; 75(4): 291-455).

Exemplary precursor human HLA-DR α-chain polypeptide sequence is set forth in SEQ ID NO:87 (mature polypeptide includes residues 26-254 of SEQ ID NO:87; see Uniprot Accession No. P01903-1; mRNA sequence set forth in SEQ ID NO:88, NCBI Reference Sequence: NM_019111.4). In humans, a locus encoding HLA-DR α-chain, HLA-DRA, comprises an open reading frame that contains 5 exons (4 coding exons) and 4 introns. An exemplary mRNA transcript of HLA-DRA spans the sequence corresponding to Chromosome 6: 32,439,887-32,445,046 forward strand, with reference to human genome version GRCh38 (UCSC Genome Browser on Human December 2013 (GRCh38/hg38) Assembly). Table 5 sets forth the coordinates of the exons and introns of the open reading frames and the untranslated regions of an exemplary transcript encoding HLA-DR α-chain.

TABLE 5
Coordinates of exons and introns of human HLA-
DRA locus (GRCh38, Chromosome 6, forward strand).

| | Start (GrCh38) | End (GrCh38) | Length |
|---|---|---|---|
| 5′ UTR and Exon 1 | 32,439,887 | 32,440,032 | 146 |
| Intron 1-2 | 32,440,033 | 32,442,447 | 2,415 |
| Exon 2 | 32,442,448 | 32,442,693 | 246 |
| Intron 2-3 | 32,442,694 | 32,443,184 | 491 |
| Exon 3 | 32,443,185 | 32,443,466 | 282 |
| Intron 3-4 | 32,443,467 | 32,443,755 | 289 |
| Exon 4 (including | 32,443,756 | 32,443,921 | 166 |
| non-coding) and 3′ UTR | | | |
| Intron 4-5 | 32,443,922 | 32,444,651 | 730 |
| Exon 5 (non-coding), 3′ UTR | 32,444,652 | 32,445,046 | 395 |

Exemplary precursor human HLA-DR a3-chain polypeptide sequence is set forth in SEQ ID NO: 89 (mature polypeptide includes residues 30-266 of SEQ ID NO: 89; see Uniprot Accession No. P04229-1; mRNA sequence set forth in SEQ ID NO:90, GenBank: X03069.1). In humans, an exemplary locus encoding HLA-DR β-chain, HLA-DRB1, comprises an open reading frame that contains 6 exons and 5 introns. An exemplary mRNA transcript of HLA-DRB1 spans the sequence corresponding to Chromosome 6: 32,578,769-32,589,848 reverse strand, with reference to human genome version GRCh38 (UCSC Genome Browser on Human December 2013 (GRCh38/hg38) Assembly). Table 6 sets forth the coordinates of the exons and introns of the open reading frames and the untranslated regions of an exemplary transcript encoding HLA-DR β-chain.

TABLE 6
Coordinates of exons and introns of human HLA-DRB1
locus (GRCh38, Chromosome 6, forward strand).

| | Start (GrCh38) | End (GrCh38) | Length |
|---|---|---|---|
| 5′ UTR and Exon 1 | 32,439,887 | 32,440,032 | 146 |
| Intron 1-2 | 32,440,033 | 32,442,447 | 2,415 |
| Exon 2 | 32,589,848 | 32,589,643 | 206 |
| Intron 2-3 | 32,589,642 | 32,584,379 | 5,264 |
| Exon 3 | 32,584,378 | 32,584,109 | 270 |
| Intron 3-4 | 32,584,108 | 32,581,839 | 2,270 |
| Exon 4 | 32,581,838 | 32,581,557 | 282 |
| Intron 4-5 | 32,581,556 | 32,580,857 | 700 |
| Exon 5 | 32,580,856 | 32,580,746 | 111 |
| Intron 5-6 | 32,580,745 | 32,580,271 | 475 |
| Exon 6 and 3′ UTR | 32,580,270 | 32,580,247 | 24 |

In some embodiments, the engineered cell, e.g., containing a modified T cell stimulation-associated locus comprising a transgene encoding a recombinant receptor or a portion thereof operably linked to an endogenous transcriptional regulatory element of the T cell stimulation-associated locus, also contains a genetic disruption at an endogenous T cell receptor alpha constant region (TRAC) gene and/or an endogenous T cell receptor beta constant region (TRBC) gene. In some aspects, the additional genetic disruption at an endogenous TRAC and/or TRBC loci prevents expression of the endogenous TCR in the engineered cell, thereby preventing expression of the transgene, e.g., encoding a recombinant receptor or a portion thereof, in the absence of a stimulation or activation signal via the recombinant receptor. In some aspects, the additional genetic disruption at an endogenous TRAC and/or TRBC loci prevents re-expression of the transgene encoding the recombinant receptor or a portion thereof in the absence of antigen-specific stimulation or activation signal, for example, when the tumor-specific antigen is not present after elimination of the tumor.

In some embodiments, the endogenous TCR Ca is encoded by the TRAC gene (IMGT nomenclature). Exemplary human TCR Ca polypeptide sequence is set forth in SEQ ID NO:91 or 92 (see UniProtKB Accession No. P01848 or Genbank Accession No. CAA26636.1; mRNA sequence set forth in SEQ ID NO:93, GenBank: X02592.1). In humans, an exemplary genomic locus of TRAC comprises an open reading frame that contains 4 exons and 3 introns. An exemplary mRNA transcript of TRAC can span the sequence corresponding to coordinates Chromosome 14: 22,547,506-22,552,154, on the forward strand, with reference to human genome version GRCh38 (UCSC Genome Browser on Human December 2013 (GRCh38/hg38) Assembly). Table 7 sets forth the coordinates of the exons and introns of the open reading frames and the untranslated regions of the transcript of an exemplary human TRAC locus.

TABLE 7
Coordinates of exons and introns of exemplary human
TRAC locus (GRCh38, Chromosome 14, forward strand).

| | Start (GrCh38) | End (GrCh38) | Length |
|---|---|---|---|
| 5′ UTR and Exon 1 | 22,547,506 | 22,547,778 | 273 |
| Intron 1-2 | 22,547,779 | 22,549,637 | 1,859 |
| Exon 2 | 22,549,638 | 22,549,682 | 45 |
| Intron 2-3 | 22,549,683 | 22,550,556 | 874 |
| Exon 3 | 22,550,557 | 22,550,664 | 108 |
| Intron 3-4 | 22,550,665 | 22,551,604 | 940 |
| Exon 4 and 3′ UTR | 22,551,605 | 22,552,154 | 550 |

In some embodiments, the endogenous TCR Cβ is encoded by TRBC1 or TRBC2 genes (IMGT nomenclature). Exemplary human TCR Cβ polypeptide sequence is set forth in SEQ ID NO:94, 95 or 96 (see UniProtKB Accession No. P01850, A0A5B9 or A0A0G2JNG9; mRNA sequence set forth in SEQ ID NO:97; GenBank: X00437.1).

In humans, an exemplary genomic locus of TRBC1 comprises an open reading frame that contains 4 exons and 3 introns. An exemplary mRNA transcript of TRBC1 can span the sequence corresponding to coordinates Chromosome 7: 142,791,694-142,793,368, on the forward strand, with reference to human genome version GRCh38 (UCSC Genome Browser on Human December 2013 (GRCh38/hg38) Assembly). Table 8 sets forth the coordinates of the exons and introns of the open reading frames and the untranslated regions of the transcript of an exemplary human TRBC1 locus.

TABLE 8
Coordinates of exons and introns of exemplary human
TRBC1 locus (GRCh38, Chromosome 7, forward strand).

| | Start (GrCh38) | End (GrCh38) | Length |
|---|---|---|---|
| 5′ UTR and Exon 1 | 142,791,694 | 142,792,080 | 387 |
| Intron 1-2 | 142,792,081 | 142,792,521 | 441 |
| Exon 2 | 142,792,522 | 142,792,539 | 18 |
| Intron 2-3 | 142,792,540 | 142,792,691 | 152 |
| Exon 3 | 142,792,692 | 142,792,798 | 107 |
| Intron 3-4 | 142,792,799 | 142,793,120 | 322 |
| Exon 4 and 3′ UTR | 142,793,121 | 142,793,368 | 248 |

In humans, an exemplary genomic locus of TRBC2 comprises an open reading frame that contains 4 exons and 3 introns. An exemplary mRNA transcript of TRBC2 can span the sequence corresponding to coordinates Chromosome 7: 142,801,041-142,802,748, on the forward strand, with reference to human genome version GRCh38 (UCSC Genome Browser on Human December 2013 (GRCh38/hg38) Assembly). Table 9 sets forth the coordinates of the exons and introns of the open reading frames and the untranslated regions of the transcript of an exemplary human TRBC2 locus.

TABLE 9
Coordinates of exons and introns of exemplary human
TRBC2 locus (GRCh38, Chromosome 7, forward strand).

| | Start (GrCh38) | End (GrCh38) | Length |
|---|---|---|---|
| 5′ UTR and Exon 1 | 142,801,041 | 142,801,427 | 387 |
| Intron 1-2 | 142,801,428 | 142,801,943 | 516 |
| Exon 2 | 142,801,944 | 142,801,961 | 18 |
| Intron 2-3 | 142,801,962 | 142,802,104 | 143 |
| Exon 3 | 142,802,105 | 142,802,211 | 107 |
| Intron 3-4 | 142,802,212 | 142,802,502 | 291 |
| Exon 4 and 3′ UTR | 142,802,503 | 142,802,748 | 246 |

In some of any embodiments, the genetic disruption is targeted at, near, or within an open reading frame of the T cell stimulation-associated locus, TRAC and/or TRBC (such as described in Tables 1-9 herein). In certain embodiments, the genetic disruption is targeted at, near, or within an open reading frame that encodes a TCRα constant domain. In some embodiments, the genetic disruption is targeted at, near, or within the T cell stimulation-associated locus, TRAC and/or TRBC (such as described in Tables 1-9 herein), or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the T cell stimulation-associated locus, TRAC and/or TRBC (such as described in Tables 1-9 herein).

In some aspects, the target site is within an exon of the open reading frame of the endogenous T cell stimulation-associated locus, TRAC and/or TRBC. In some aspects, the target site is within an intron of the open reading frame of the T cell stimulation-associated locus, TRAC and/or TRBC. In some aspects, the target site is within a regulatory or control element, e.g., a promoter, 5′ untranslated region (UTR) or 3′ UTR, of the T cell stimulation-associated locus, TRAC and/or TRBC. In some embodiments, the target site is within the T cell stimulation-associated locus, TRAC and/or TRBC genomic region sequence described in Tables 1-9 herein or any exon or intron of the T cell stimulation-associated locus, TRAC and/or TRBC genomic region sequence contained therein. In some aspects, the target site is at or near the junction or border between an exon and an intron, or an exon and a regulatory or control element, e.g., a promoter, 5′ untranslated region (UTR) or 3′ UTR, of the endogenous T cell stimulation-associated locus, TRAC and/or TRBC. In some aspects, the target site is within an intron of the open reading frame of the T cell stimulation-associated locus, TRAC and/or TRBC.

In some embodiments, the target site for a genetic disruption is selected such that after integration of the transgene, the cell is knocked out for, reduced and/or eliminated expression from the endogenous T cell stimulation-associated locus, TRAC and/or TRBC.

In some embodiments, a genetic disruption, e.g., DNA break, is targeted within an exon of the T cell stimulation-associated locus, TRAC and/or TRBC or open reading frame thereof. In certain embodiments, the genetic disruption is within the first exon, second exon, third exon, or forth exon of the T cell stimulation-associated locus, TRAC and/or TRBC or open reading frame thereof. In some of any embodiments, the genetic disruption is within the first exon of the T cell stimulation-associated locus, TRAC and/or TRBC or open reading frame thereof. In some embodiments, the genetic disruption is within 500 base pairs (bp) downstream from the 5′ end of the first exon in the T cell stimulation-associated locus, TRAC and/or TRBC or open reading frame thereof. In some of any embodiments, the genetic disruption is between the 5′ nucleotide of exon 1 and upstream of the 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the first exon in the T cell stimulation-associated locus, TRAC and/or TRBC or open reading frame thereof. In some of any embodiments, the genetic disruption is between 1 bp and 400 bp, between 50 and 300 bp, between 100 bp and 200 bp, or between 100 bp and 150 bp downstream from the 5′ end of the first exon in the T cell stimulation-associated locus, TRAC and/or TRBC or open reading frame thereof, each inclusive. In certain embodiments, the genetic disruption is between 100 bp and 150 bp downstream from the 5′ end of the first exon in the T cell stimulation-associated locus, TRAC and/or TRBC or open reading frame thereof, inclusive.

In some aspects, the target site is within an exon, such as exons corresponding to early coding regions. In some embodiments, the target site is within or in close proximity to exons corresponding to early coding region, e.g., exon 1, 2, 3, 4 or 5 of the open reading frame of the endogenous T cell stimulation-associated locus, TRAC and/or TRBC (such as described in Tables 1-9 herein), or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some aspects, the target site is within a regulatory or control element, e.g., a promoter, of the T cell stimulation-associated locus, TRAC and/or TRBC.

In certain embodiments, a genetic disruption is targeted at, near, or within a T cell stimulation-associated locus, TRAC and/or TRBC. In some of any embodiments, the genetic disruption is targeted at, near, or within an open reading frame of the T cell stimulation-associated locus, TRAC and/or TRBC (such as described in Tables 1-9 herein). In certain embodiments, the genetic disruption is targeted at, near, or within an open reading frame that encodes a T cell stimulation-associated locus, TRAC and/or TRBC. In some embodiments, the genetic disruption is targeted at, near, or within the T cell stimulation-associated locus, TRAC and/or TRBC (such as described in Tables 1-9 herein), or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the T cell stimulation-associated locus, TRAC and/or TRBC (such as described in Tables 1-9 herein).

In some aspects, the transgene (e.g., exogenous nucleic acid sequences) within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of a transgene (for example, encoding a recombinant receptor or a portion thereof). In some embodiments, the one or more homology arm sequences of the template polynucleotide is designed to surround the site of genetic disruption (target site). In some aspects, the genetic disruption is targeted such that upon integration of the transgene encoding the recombinant receptor, the expression of the endogenous T cell stimulation-associated locus, TRAC and/or TRBC is reduced or eliminated. In some aspects, the genetic disruption is targeted such that upon integration of the transgene encoding the recombinant receptor, all or a portion of the endogenous T cell stimulation-associated locus, TRAC and/or TRBC is expressed. In some aspects, the target site is placed within or near an exon of the endogenous T cell stimulation-associated locus, TRAC and/or TRBC, so that the transgene encoding recombinant receptor can be integrated in-frame with the coding sequence of the T cell stimulation-associated locus, TRAC and/or TRBC is expressed.

### 2. Methods of Genetic Disruption

In some aspects, the methods for generating the genetically engineered cells involve introducing a genetic disruption at one or more target site(s), e.g., one or more target sites at a T cell stimulation-associated locus, TRAC and/or TRBC. Methods for generating a genetic disruption, including those described herein, can involve the use of one or more agent(s) capable of inducing a genetic disruption, such as engineered systems to induce a genetic disruption, a cleavage and/or a double strand break (DSB) or a nick (e.g., a single strand break (SSB)) at a target site or target position in the endogenous or genomic DNA such that repair of the break by an error born process such as non-homologous end joining (NHEJ) or repair by HDR using repair template can result in the insertion of a sequence of interest (e.g., exogenous nucleic acid sequences or transgene encoding a recombinant receptor or a portion thereof) at or near the target site or position. Also provided are one or more agent(s) capable of inducing a genetic disruption, for use in the methods provided herein. In some aspects, the one or more agent(s) can be used in combination with the template nucleotides provided herein, for homology directed repair (HDR) mediated targeted integration of the transgene.

In some embodiments, the one or more agent(s) capable of inducing a genetic disruption comprises a DNA binding protein or DNA-binding nucleic acid that specifically binds to or hybridizes to a particular site or position in the genome, e.g., a target site or target position. In some aspects, the targeted genetic disruption, e.g., DNA break or cleavage, at the endogenous T cell stimulation-associated locus, TRAC and/or TRBC is achieved using a protein or a nucleic acid is coupled to or complexed with a gene editing nuclease, such as in a chimeric or fusion protein. In some embodiments, the one or more agent(s). capable of inducing a genetic disruption comprises an RNA-guided nuclease, or a fusion protein comprising a DNA-targeting protein and a nuclease.

In some embodiments, the agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system (including Cas and/or Cfp1). In some embodiments, the targeted genetic disruption is carried using agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Pub. No. 20110301073.

In some embodiments, the one or more agent(s) specifically targets the at least one target site(s) at or near a T cell stimulation-associated locus, TRAC and/or TRBC. In some embodiments, the agent comprises a ZFN, TALEN or a CRISPR/Cas9 combination that specifically binds to, recognizes, or hybridizes to the target site(s). In some embodiments, the CRISPR/Cas9 system includes an engineered crRNA/tracr RNA (“single guide RNA”) to guide specific cleavage. In some embodiments, the agent comprises nucleases based on the Argonaute system (e.g., from T. thermophilus, known as ‘TtAgo’ (Swarts et al., (2014) Nature 507(7491): 258-261). Targeted cleavage using any of the nuclease systems described herein can be exploited to insert the sequences of a transgene, e.g., nucleic acid sequence encoding a recombinant receptor or a portion thereof, into a specific target location at an endogenous T cell stimulation-associated locus, TRAC and/or TRBC, using either HDR or NHEJ-mediated processes.

In some embodiments, a “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994) Proc. Natl. Acad. Sci. USA 91:883-887; Kim et al. (1994) J. Biol. Chem. 269: 978-982. Some gene-specific engineered zinc fingers are available commercially. For example, a platform called CompoZr, for zinc-finger construction is available that provides specifically targeted zinc fingers for thousands of targets. See, e.g., Gaj et al., Trends in Biotechnology, 2013, 31(7), 397-405. In some cases, commercially available zinc fingers are used or are custom designed. In some embodiments, the one or more target site(s), e.g., within the T cell stimulation-associated locus, TRAC and/or TRBC can be targeted for genetic disruption by engineered ZFNs.

Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from different bacterial species. The new modular proteins have the advantage of displaying more sequence variability than TAL repeats. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, critical amino acids 12 and 13 can be mutated towards other amino acid residues in order to modulate their specificity towards nucleotides A, T, C and G and in particular to enhance this specificity.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a “TALE-nuclease” (TALEN) is a fusion protein comprising a nucleic acid binding domain typically derived from a Transcription Activator Like Effector (TALE) and a nuclease catalytic domain that cleaves a nucleic acid target sequence. The catalytic domain comprises a nuclease domain or a domain having endonuclease activity, like for instance I-TevI, ColE7, NucA and Fok-I. In a particular embodiment, the TALE domain can be fused to a meganuclease like for instance I-CreI and I-OnuI or functional variant thereof. In some embodiments, the TALEN is a monomeric TALEN. A monomeric TALEN is a TALEN that does not require dimerization for specific recognition and cleavage, such as the fusions of engineered TAL repeats with the catalytic domain of I-TevI described in WO2012138927. TALENs have been described and used for gene targeting and gene modifications (see, e.g., Boch et al. (2009) Science 326(5959): 1509-12; Moscou and Bogdanove (2009) Science 326(5959): 1501; Christian et al. (2010) Genetics 186(2): 757-61; Li et al. (2011) Nucleic Acids Res 39(1): 359-72). In some embodiments, one or more sites in the T cell stimulation-associated locus, TRAC and/or TRBC can be targeted for genetic disruption by engineered TALENs.

In some embodiments, a “TtAgo” is a prokaryotic Argonaute protein thought to be involved in gene silencing. TtAgo is derived from the bacteria Thermus thermophilus. See, e.g. Swarts et al., (2014) Nature 507(7491): 258-261, G. Sheng et al., (2013) Proc. Natl. Acad. Sci. U.S.A. 111, 652). A “TtAgo system” is all the components required including e.g. guide DNAs for cleavage by a TtAgo enzyme.

In some embodiments, an engineered zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

Zinc finger and TALE DNA-binding domains can be engineered to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

a. CRISPR/Cas9

In some embodiments, the targeted genetic disruption, e.g., DNA break, at the endogenous genes T cell stimulation-associated locus, TRAC and/or TRBC in humans is carried out using clustered regularly interspaced short palindromic repeats (CRISPR) and CRISPR-associated (Cas) proteins. See Sander and Joung (2014) Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracr RNA or an active partial tracr RNA), a tracr-mate sequence (encompassing a “direct repeat” and a tracr RNA-processed partial direct repeat in the context of an endogenous CRISPR system), a guide sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9), with nuclease functionality.

Also provided are one or more agents capable of introducing a genetic disruption. Also provided are polynucleotides (e.g., nucleic acid molecules) encoding one or more components of the one or more agent(s) capable of inducing a genetic disruption.

(i) Guide RNA (gRNA)

In some embodiments, the one or more agent(s) capable of inducing a genetic disruption comprises at least one of: a guide RNA (gRNA) having a targeting domain that is complementary with a target site at the T cell stimulation-associated locus, TRAC and/or TRBC or at least one nucleic acid encoding the gRNA.

In some aspects, a “gRNA molecule” is a nucleic acid that promotes the specific targeting or homing of a gRNA molecule/Cas9 molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a guide sequence, e.g., guide RNA, is any polynucleotide sequences comprising at least a sequence portion that has sufficient complementarity with a target polynucleotide sequence, such as the at the T cell stimulation-associated locus, TRAC and/or TRBC in humans, to hybridize with the target sequence at the target site and direct sequence-specific binding of the CRISPR complex to the target sequence. In some embodiments, in the context of formation of a CRISPR complex, “target sequence” is a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some embodiments, a guide RNA (gRNA) specific to a target locus of interest (e.g. at the T cell stimulation-associated locus, TRAC and/or TRBC in humans) is used to RNA-guided nucleases, e.g., Cas, to induce a DNA break at the target site or target position. Methods for designing gRNAs and exemplary targeting domains can include those described in, e.g., International PCT Pub. Nos. WO2015/161276, WO2017/193107 and WO2017/093969.

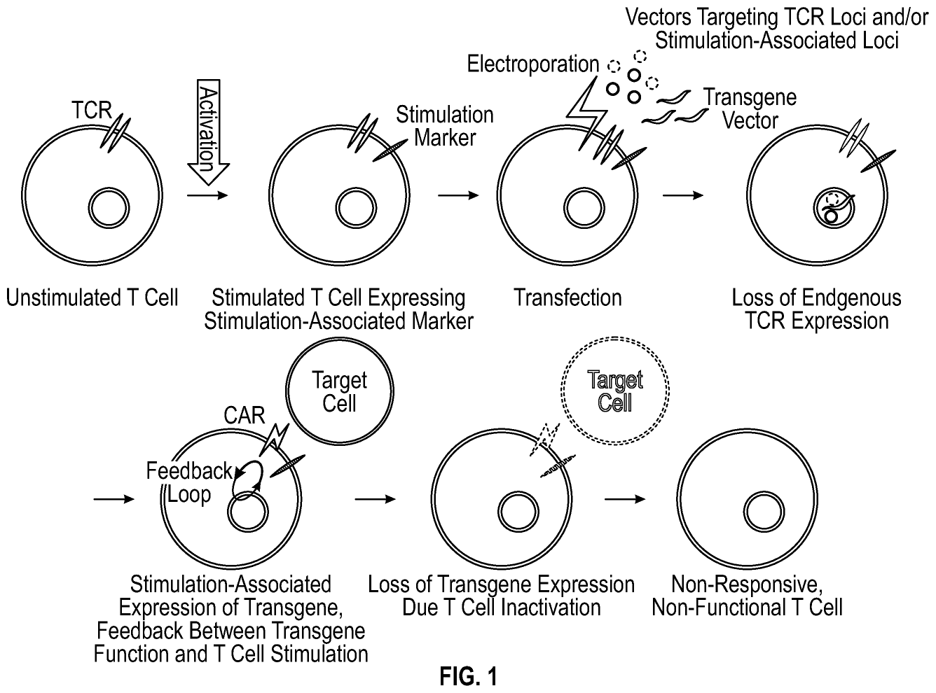

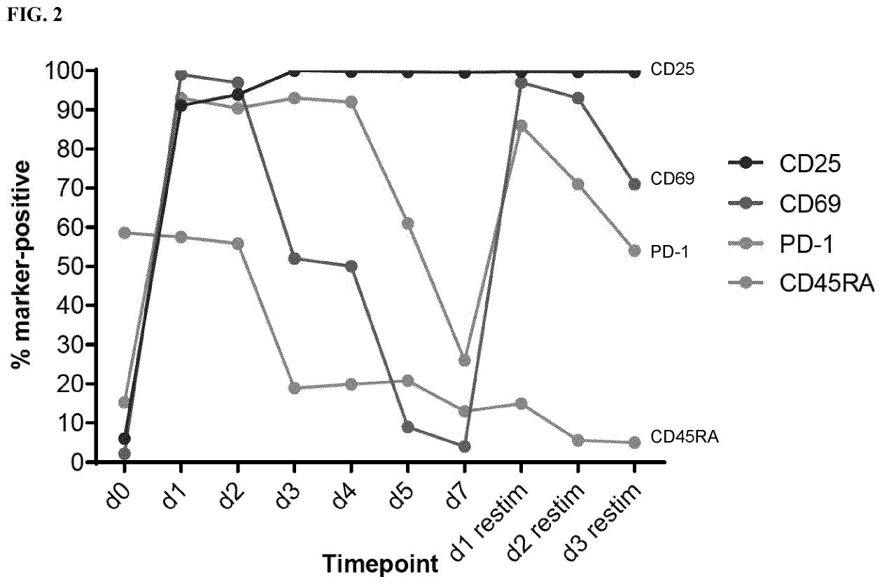

Several exemplary gRNA structures, with domains indicated thereon, are described in WO2015/161276, e.g., in FIGS. 1A-1G therein. While not wishing to be bound by theory, with regard to the three dimensional form, or intra- or inter-strand interactions of an active form of a gRNA, regions of high complementarity are sometimes shown as duplexes in WO2015/161276, e.g., in FIGS. 1A-1G therein and other depictions provided herein.

In some cases, the gRNA is a unimolecular or chimeric gRNA comprising, from 5′ to 3′: a targeting domain which is complementary to a target nucleic acid, such as a sequence from the T cell stimulation-associated locus, TRAC and/or TRBC gene; a first complementarity domain; a linking domain; a second complementarity domain (which is complementary to the first complementarity domain); a proximal domain; and optionally, a tail domain.

In some cases, the gRNA is a modular gRNA comprising first and second strands. In these cases, the first strand preferably includes, from 5′ to 3′: a targeting domain (which is complementary to a target nucleic acid, such as a sequence from the T cell stimulation-associated locus, TRAC and/or TRBC gene) and a first complementarity domain. The second strand generally includes, from 5′ to 3′: optionally, a 5′ extension domain; a second complementarity domain; a proximal domain; and optionally, a tail domain.

### (a) Targeting Domain

The targeting domain comprises a nucleotide sequence that is complementary, e.g., at least 80, 85, 90, 95, 98 or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting domains can be found, e.g., in Fu et al., Nat Biotechnol 2014 March; 32(3):279-284 and Sternberg et al., Nature 2014, 507:62-67. Examples of the placement of targeting domains include those described in WO2015/161276, e.g., in FIGS. 1A-1G therein.

The targeting domain is part of an RNA molecule and will therefore comprise the base uracil (U), while any DNA encoding the gRNA molecule will comprise the base thymine (T). While not wishing to be bound by theory, In some embodiments, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In some embodiments, the target domain itself comprises in the 5′ to 3′ direction, an optional secondary domain, and a core domain. In some embodiments, the core domain is fully complementary with the target sequence. In some embodiments, the targeting domain is 5 to 50 nucleotides in length. The strand of the target nucleic acid with which the targeting domain is complementary is referred to herein as the complementary strand. Some or all of the nucleotides of the domain can have a modification, e.g., to render it less susceptible to degradation, improve bio-compatibility, etc. By way of non-limiting example, the backbone of the target domain can be modified with a phosphorothioate, or other modification(s). In some cases, a nucleotide of the targeting domain can comprise a 2′ modification, e.g., a 2-acetylation, e.g., a 2′ methylation, or other modification(s).

In various embodiments, the targeting domain is 16-26 nucleotides in length (i.e. it is 16 nucleotides in length, or 17 nucleotides in length, or 18, 19, 20, 21, 22, 23, 24, 25 or 26 nucleotides in length.

### (b) Exemplary Targeting Domains

In some embodiments, gRNA sequences that is or comprises a targeting domain sequence targeting the target site in a particular gene, such as the T cell stimulation-associated locus, TRAC and/or TRBC, designed or identified. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the target sequence (target domain) is at or near the T cell stimulation-associated locus, TRAC and/or TRBC, such as any part of the T cell stimulation-associated locus, TRAC and/or TRBC. In some embodiments, the target nucleic acid complementary to the targeting domain is located at an early coding region of a gene of interest, such as T cell stimulation-associated locus, TRAC and/or TRBC. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target sequence on the target nucleic acid, such as the target nucleic acid in the T cell stimulation-associated locus, TRAC and/or TRBC. In some embodiments, the targeting domain is located downstream of and/or near the endogenous the endogenous transcriptional regulatory element, e.g., a promoter, of an endogenous T cell stimulation-associated locus, TRAC and/or TRBC.

In some embodiments, the gRNA can target a site at the T cell stimulation-associated locus, TRAC and/or TRBC near a desired site of targeted integration of a transgene, e.g., encoding a recombinant receptor. In some aspects, the gRNA can target a site based on the amount of sequences encoding the T cell stimulation-associated locus, TRAC and/or TRBC that is desired for regulation of expression of the recombinant receptor in a manner, time and extent similar to the regulation of the endogenous T cell stimulation-associated locus, TRAC and/or TRBC. In some aspects, the gRNA can target a site based on the amount of sequences encoding the T cell stimulation-associated locus, TRAC and/or TRBC that is desired for expression in the cell expressing the recombinant receptor. In some aspects, the gRNA can target a site such that upon integration of the transgene, e.g., encoding a recombinant receptor, the resulting T cell stimulation-associated locus, TRAC and/or TRBC retains expression of the endogenous gene product encoded by the T cell stimulation-associated locus, TRAC and/or TRBC. In some aspects, the endogenous gene product is not expressed (e.g., knocked-out) following targeting by the gRNA and subsequent HDR. In some aspects, the gRNA can target a site within an exon of the open reading frame of the endogenous T cell stimulation-associated locus, TRAC and/or TRBC. In some aspects, the gRNA can target a site within an intron of the open reading frame of the T cell stimulation-associated locus, TRAC and/or TRBC. In some aspects, the gRNA can target a site within or downstream of a regulatory or control element, e.g., a promoter, of the T cell stimulation-associated locus, TRAC and/or TRBC. In some aspects, the target site at the T cell stimulation-associated locus, TRAC and/or TRBC that is targeted by the gRNA can be any target sites described herein, e.g., in Section II.A.1. In some embodiments, the gRNA can target a site within or in close proximity to exons corresponding to early coding region, e.g., exon 1, 2, 3, 4 or 5 of the open reading frame of the endogenous T cell stimulation-associated locus, TRAC and/or TRBC, or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some embodiments, the gRNA can target a site at or near exon 2 of the endogenous T cell stimulation-associated locus, TRAC and/or TRBC, or within less than 500, 450, 400, 350, 300, 250, 200, 150, 100 or 50 bp of exon 2.

Exemplary target sequence for the T cell stimulation-associated locus PDCD1, include the sequence set forth in SEQ ID NO: 74, 78 or 98-103. Exemplary gRNAs can include a sequence of ribonucleic acids that can bind to or target or is complementary to or can bind to the complimentary strand sequence of the target site sequences set forth in SEQ ID NO: 74, 78 or 98-103. An exemplary PDCD1 gRNA sequence includes the sequence set forth in SEQ ID NO: 75 or 104-109. An exemplary PDCD1 gRNA sequence includes the sequence set forth in SEQ ID NO: 75. Any of the known methods can be used to target and generate a genetic disruption of the endogenous PDCD1 can be used in the embodiments provided herein. Exemplary target sequences or targeting domains contained within the gRNA for targeting the genetic disruption of the human PDCD1 locus include those described in, e.g., WO2015/161276, WO2017/093969, Schumann et al., PNAS Aug. 18, 2015 112 (33) 10437-10442, and Xu et al., Sci Rep. 2018; 8: 11649, which are incorporated by reference herein.

Exemplary target sequence for the exemplary T cell stimulation-associated locus CD69, include the sequence set forth in SEQ ID NO: 110-115. Exemplary gRNAs can include a sequence of ribonucleic acids that can bind to or target or is complementary to or can bind to the complimentary strand sequence of the target site sequences set forth in SEQ ID NO: 110-115. An exemplary CD69 gRNA sequence includes the sequence set forth in SEQ ID NO: 116-121. Any of the known methods can be used to target and generate a genetic disruption of the endogenous CD69 can be used in the embodiments provided herein. Exemplary target sequences or targeting domains contained within the gRNA for targeting the genetic disruption of the human CD69 locus include those described in, e.g., Simenov et al., Nature. 2017 Sep. 7; 549(7670): 111-115, which are incorporated by reference herein.

Exemplary target sequence for the exemplary T cell stimulation-associated locus Nur77 (NR4A1), include the sequence set forth in SEQ ID NO: 122-127 or 134-136. Exemplary gRNAs can include a sequence of ribonucleic acids that can bind to or target or is complementary to or can bind to the complimentary strand sequence of the target site sequences set forth in SEQ ID NO: 122-127 or 134-136. An exemplary Nur77 (NR4A1) gRNA sequence includes the sequence set forth in SEQ ID NO: 128-133 or 136-138. Any of the known methods can be used to target and generate a genetic disruption of the endogenous Nur77 (NR4A1) can be used in the embodiments provided herein. Exemplary target sequences or targeting domains contained within the gRNA for targeting the genetic disruption of the human Nur77 (NR4A1) locus include those described in, e.g., WO 2019/089982, WO 2019/104245 and Munnur et al., Cell Reports (2019) 26, 2028-2036, which are incorporated by reference herein.

Exemplary target sequence for the exemplary T cell stimulation-associated locus FoxP3, include the sequence set forth in SEQ ID NO: 140-147. Exemplary gRNAs can include a sequence of ribonucleic acids that can bind to or target or is complementary to or can bind to the complimentary strand sequence of the target site sequences set forth in SEQ ID NO: 140-147. An exemplary FoxP3 gRNA sequence includes the sequence set forth in SEQ ID NO: 148-155. Any of the known methods can be used to target and generate a genetic disruption of the endogenous FoxP3 can be used in the embodiments provided herein. Exemplary target sequences or targeting domains contained within the gRNA for targeting the genetic disruption of the human FoxP3 locus include those described in, e.g., Okada et al., Epigenetics Chromatin. 2017; 10: 24 and Holohan et al., bioRxiv 644229, which are incorporated by reference herein.

Exemplary target sequence for the exemplary T cell stimulation-associated locus HLA-DRA, include the sequence set forth in SEQ ID NO: 156-161. Exemplary gRNAs can include a sequence of ribonucleic acids that can bind to or target or is complementary to or can bind to the complimentary strand sequence of the target site sequences set forth in SEQ ID NO: 156-161. An exemplary HLA-DRA gRNA sequence includes the sequence set forth in SEQ ID NO: 162-167. Any of the known methods can be used to target and generate a genetic disruption of the endogenous HLA-DRA can be used in the embodiments provided herein. Exemplary target sequences or targeting domains contained within the gRNA for targeting the genetic disruption of the human HLA-DRA locus include those described in, e.g., WO 2016/021972 and WO 2017/093969, which are incorporated by reference herein.

Exemplary target sequence for the exemplary T cell stimulation-associated locus HLA-DRB1, include the sequence set forth in SEQ ID NO: 168-177. Exemplary gRNAs can include a sequence of ribonucleic acids that can bind to or target or is complementary to or can bind to the complimentary strand sequence of the target site sequences set forth in SEQ ID NO: 168-177. An exemplary HLA-DRB1 gRNA sequence includes the sequence set forth in SEQ ID NO: 178-187. Any of the known methods can be used to target and generate a genetic disruption of the endogenous HLA-DRB1 can be used in the embodiments provided herein. Exemplary target sequences or targeting domains contained within the gRNA for targeting the genetic disruption of the human HLA-DRB1 locus include those described in, e.g., WO 2016/021972 and WO 2017/093969, which are incorporated by reference herein.

Exemplary targeting domains contained within the gRNA for targeting the genetic disruption of the human TRAC, TRBC1 or TRBC2 include those described in, e.g., WO2015/161276, WO2017/193107, WO2017/093969, WO 2019/195492, US2016/272999 and US2015/056705 or a targeting domain that can bind to the target sequences described in the foregoing. Exemplary targeting domains contained within the gRNA for targeting the genetic disruption of the human TRAC locus using S. pyogenes or S. aureus Cas9 can include any of those set forth in SEQ ID NOS: 77 and 188-218. Exemplary targeting domains contained within the gRNA for targeting the genetic disruption of the human TRBC1 or TRBC2 locus using S. pyogenes or S. aureus Cas9 can include any of those set forth in SEQ ID NOS: 219-276.

In some embodiments, the gRNA for targeting TRAC, TRBC1 and/or TRBC2 include any that are described herein, or are described elsewhere e.g., in WO2015/161276, WO2017/193107, WO2017/093969, WO 2019/195492, US2016/272999 and US2015/056705 or a targeting domain that can bind to the target sequences described in the foregoing. In some embodiments, the gRNA for targeting the TRAC gene locus can be obtained by in vitro transcription of the sequence

AGCGCTCTCGTACAGAGTTGGCATTATAATACGACTCACTATAGGGGAG
AATCAAAATCGGTGAATGTTTTAGAGCTAGAAATAGCAAGTTAAAATAA
GGCTAGTCCGTTATCAACTTGAAAAAGTGGCACCGAGTCGGTGCTTTTT
TT (set forth in SEQ ID NO: 277;

bold and underlined portion is complementary to the target site in the TRAC locus), or chemically synthesized, where the gRNA had the sequence

5′-GAG AAU CAA AAU CGG UGA AUG UUU UAG AGC UAG
AAA UAG CAA GUU AAA AUA AGG CUA GUC CGU UAU CAA
CUU GAA AAA GUG GCA CCG AGU CGG UGC UUU U-
3′ (set forth in SEQ ID NO: 278;

see Osborn et al., Mol Ther. 24(3):570-581 (2016)). Other exemplary gRNA sequences to generate a genetic disruption of the endogenous genes encoding TCR domains or regions, e.g., TRAC, TRBC1 and/or TRBC2 are described, e.g., in WO2015/161276, WO2017/193107, WO2017/093969, WO 2019/195492, US2016/272999 and US2015/056705.

Exemplary methods for gene editing of the endogenous TCR loci include those described in, e.g. U.S. Publication Nos. US2011/0158957, US2014/0301990, US2015/0098954,US2016/0208243; US2016/272999 and US2015/056705; International PCT Publication Nos. WO2014/191128, WO2015/136001, WO2015/161276, WO2016/069283, WO2016/016341, WO2017/193107, and WO2017/093969; and Osborn et al. (2016) Mol. Ther. 24(3):570-581. Any of the known methods can be used to generate a genetic disruption of the endogenous genes encoding TCR domains or regions can be used in the embodiments provided herein.

In some embodiments, targeting domains include those for introducing a genetic disruption at the TRAC, TRBC1 and/or TRBC2 loci using S. pyogenes Cas9 or using N. meningitidis Cas9. In some embodiments, targeting domains include those for introducing a genetic disruption at the TRAC, TRBC1 and/or TRBC2 loci using S. pyogenes Cas9. Any of the targeting domains can be used with a S. pyogenes Cas9 molecule that generates a double stranded break (Cas9 nuclease) or a single-stranded break (Cas9 nickase).

In some embodiments, dual targeting is used to create two nicks on opposite DNA strands by using S. pyogenes Cas9 nickases with two targeting domains that are complementary to opposite DNA strands, e.g., a gRNA comprising any minus strand targeting domain may be paired with any gRNA comprising a plus strand targeting domain. In some embodiments, the two gRNAs are oriented on the DNA such that PAMs face outward and the distance between the 5′ ends of the gRNAs is 0-50 bp. In some embodiments, two gRNAs are used to target two Cas9 nucleases or two Cas9 nickases, for example, using a pair of Cas9 molecule/gRNA molecule complex guided by two different gRNA molecules to cleave the target domain with two single stranded breaks on opposing strands of the target domain. In some embodiments, the two Cas9 nickases can include a molecule having HNH activity, e.g., a Cas9 molecule having the RuvC activity inactivated, e.g., a Cas9 molecule having a mutation at D10, e.g., the D10A mutation, a molecule having RuvC activity, e.g., a Cas9 molecule having the HNH activity inactivated, e.g., a Cas9 molecule having a mutation at H840, e.g., a H840A, or a molecule having RuvC activity, e.g., a Cas9 molecule having the HNH activity inactivated, e.g., a Cas9 molecule having a mutation at N863, e.g., N863A. In some embodiments, each of the two gRNAs are complexed with a D10A Cas9 nickase.

In some embodiments, the target sequence (target domain) is at or near the T cell stimulation-associated locus, TRAC, TRBC1 and/or TRBC2 locus, such as any part of the T cell stimulation-associated locus, TRAC, TRBC1 and/or TRBC2 coding sequence, for example described in Tables 1-9 herein. In some embodiments, the target nucleic acid complementary to the targeting domain is located at an early coding region of a gene of interest, such as T cell stimulation-associated locus, TRAC, TRBC1 and/or TRBC2. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target sequence on the target nucleic acid, such as the target nucleic acid in the T cell stimulation-associated locus, TRAC, TRBC1 and/or TRBC2.

In some aspects, the gRNA can target a site within an exon of the open reading frame of the endogenous T cell stimulation-associated locus, TRAC, TRBC1 and/or TRBC2. In some aspects, the gRNA can target a site within an intron of the open reading frame of the T cell stimulation-associated locus, TRAC, TRBC1 and/or TRBC2. In some aspects, the gRNA can target a site within a regulatory or control element, e.g., a promoter, of the T cell stimulation-associated locus, TRAC, TRBC1 and/or TRBC2. In some aspects, the target site at the T cell stimulation-associated locus, TRAC, TRBC1 and/or TRBC2 that is targeted by the gRNA can be any target sites described herein, e.g., in Section II.A.1. In some embodiments, the gRNA can target a site within or in close proximity to exons corresponding to early coding region, e.g., exon 1, 2 or 3 of the open reading frame of the endogenous T cell stimulation-associated locus, TRAC, TRBC1 and/or TRBC2, or including sequence immediately following a transcription start site, within exon 1, 2, or 3, or within less than 500, 450, 400, 350, 300, 250, 200, 150, 100 or 50 bp of exon 1, 2, or 3. In some embodiments, the gRNA can target a site at or near exon 2 of the endogenous T cell stimulation-associated locus, TRAC, TRBC1 and/or TRBC2, or within less than 500, 450, 400, 350, 300, 250, 200, 150, 100 or 50 bp of exon 2.

In some embodiments, targeting domains include those for introducing a genetic disruption at the T cell stimulation-associated locus, TRAC and/or TRBC gene using S. pyogenes Cas9 or using N. meningitidis Cas9. In some embodiments, targeting domains include those for introducing a genetic disruption at the T cell stimulation-associated locus, TRAC and/or TRBC gene using S. pyogenes Cas9. Any of the targeting domains can be used with a S. pyogenes Cas9 molecule that generates a double stranded break (Cas9 nuclease) or a single-stranded break (Cas9 nickase).

In some embodiments, dual targeting is used to create two nicks on opposite DNA strands by using S. pyogenes Cas9 nickases with two targeting domains that are complementary to opposite DNA strands, e.g., a gRNA comprising any minus strand targeting domain may be paired with any gRNA comprising a plus strand targeting domain. In some embodiments, the two gRNAs are oriented on the DNA such that PAMs face outward and the distance between the 5′ ends of the gRNAs is 0-50 bp. In some embodiments, two gRNAs are used to target two Cas9 nucleases or two Cas9 nickases, for example, using a pair of Cas9 molecule/gRNA molecule complex guided by two different gRNA molecules to cleave the target domain with two single stranded breaks on opposing strands of the target domain. In some embodiments, the two Cas9 nickases can include a molecule having HNH activity, e.g., a Cas9 molecule having the RuvC activity inactivated, e.g., a Cas9 molecule having a mutation at D10, e.g., the D10A mutation, a molecule having RuvC activity, e.g., a Cas9 molecule having the HNH activity inactivated, e.g., a Cas9 molecule having a mutation at H840, e.g., a H840A, or a molecule having RuvC activity, e.g., a Cas9 molecule having the HNH activity inactivated, e.g., a Cas9 molecule having a mutation at N863, e.g., N863A. In some embodiments, each of the two gRNAs are complexed with a D10A Cas9 nickase.

Other domains of the gRNA, such as complementary domains, linking domains, 5′ extension domains, proximal domains and tail domains, and their structures, are described, for example, in WO2015/161276, e.g., in FIGS. 1A-1G therein.

Methods for designing gRNAs are described herein, including methods for selecting, designing and validating targeting domains. Exemplary targeting domains are also provided herein. Targeting domains discussed herein can be incorporated into the gRNAs described herein.

Methods for selection and validation of target sequences as well as off-target analyses are described, e.g., in Mali et al., 2013 Science 339 (6121): 823-826; Hsu et al. Nat Biotechnol, 31(9): 827-32; Fu et al., Nat Biotechnol 2014 March; 32(3):279-284; Heigwer et al., 2014 Nat Methods 11(2):122-3; Bae et al., Bioinformatics. 2014 May 15; 30(10):1473-5; Xiao A et al., Bioinformatics. 2014 Apr. 15; 30(8):1180-1182.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool. Candidate gRNA molecules can be evaluated by art-known methods or as described herein.

In some embodiments, gRNAs for use with S. pyogenes, S. aureus, and N. meningitidis Cas9s are identified using a DNA sequence searching algorithm, e.g., using a custom gRNA design software based on the public tool cas-offinder (Bae et al. Bioinformatics. 2014; 30(10): 1473-1475). The custom gRNA design software scores guides after calculating their genome-wide off-target propensity. Typically matches ranging from perfect matches to 7 mismatches are considered for guides ranging in length from 17 to 24. In some aspects, once the off-target sites are computationally determined, an aggregate score is calculated for each guide and summarized in a tabular output using a web-interface. In addition to identifying potential gRNA sites adjacent to PAM sequences, the software also can identify all PAM adjacent sequences that differ by 1, 2, 3 or more nucleotides from the selected gRNA sites. In some embodiments, genomic DNA sequences for each gene are obtained from the UCSC Genome browser and sequences can be screened for repeat elements using the publicly available RepeatMasker program. RepeatMasker searches input DNA sequences for repeated elements and regions of low complexity. The output is a detailed annotation of the repeats present in a given query sequence.

Following identification, gRNAs can be ranked into tiers based on one or more of their distance to the target site, their orthogonality and presence of a 5′ G (based on identification of close matches in the human genome containing a relevant PAM, e.g., in the case of S. pyogenes, a NGG PAM, in the case of S. aureus, NNGRR (e.g., a NNGRRT or NNGRRV) PAM, and in the case of N. meningtidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality are selected to minimize off-target DNA cleavage. It is to be understood that this is a non-limiting example and that a variety of strategies could be utilized to identify gRNAs for use with S. pyogenes, S. aureus and N. meningitidis or other Cas9 enzymes.

In some embodiments, gRNAs for use with the S. pyogenes Cas9 can be identified using the publicly available web-based ZiFiT server (Fu et al., Nat Biotechnol 2014 March; 32(3):279-284, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. In some aspects, genomic DNA sequences for each gene can be obtained from the UCSC Genome browser and sequences can be screened for repeat elements using the publicly available Repeat-Masker program. RepeatMasker searches input DNA sequences for repeated elements and regions of low complexity. The output is a detailed annotation of the repeats present in a given query sequence.

### (ii) Cas9

Cas9 molecules of a variety of species can be used in the methods and compositions described herein. While the S. pyogenes, S. aureus, N. meningitidis, and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes, S. aureus, N. meningitidis, and S. thermophilus Cas9 molecules, Cas9 molecules from the other species can replace them. Such species include: Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteo bacterium, Gluconacetobacter diazotrophicus, Haemophilus parainfiuenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria fiavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae. Examples of Cas9 molecules can include those described in, e.g., WO2015/161276, WO2017/193107, WO2017/093969, US2016/272999 and US2015/056705.

A Cas9 molecule, or Cas9 polypeptide, as that term is used herein, refers to a molecule or polypeptide that can interact with a gRNA molecule and, in concert with the gRNA molecule, homes or localizes to a site which comprises a target domain and PAM sequence. Cas9 molecule and Cas9 polypeptide, as those terms are used herein, refer to naturally occurring Cas9 molecules and to engineered, altered, or modified Cas9 molecules or Cas9 polypeptides that differ, e.g., by at least one amino acid residue, from a reference sequence, e.g., the most similar naturally occurring Cas9 molecule.

Crystal structures have been determined for two different naturally occurring bacterial Cas9 molecules (Jinek et al., Science, 343(6176):1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014 Sep. 25; 513(7519):569-73).

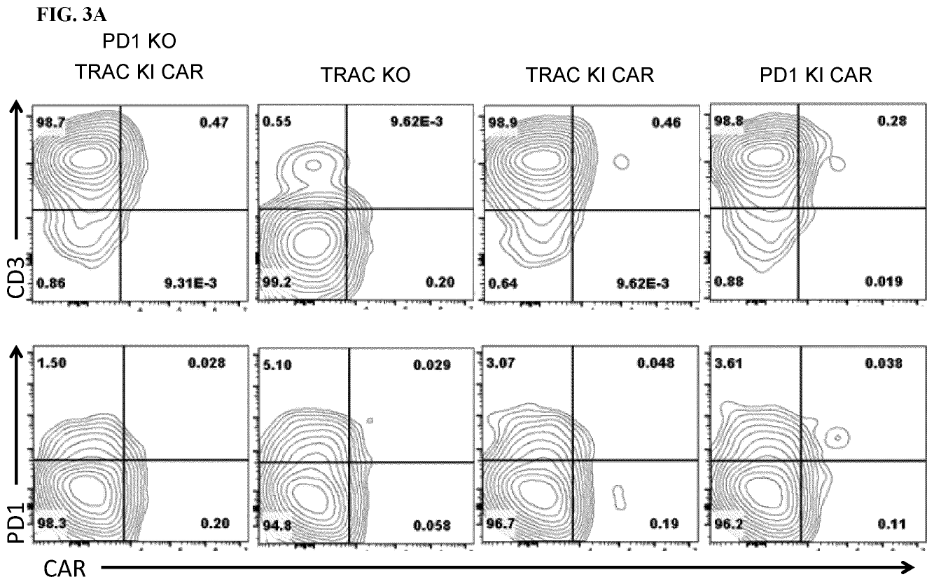

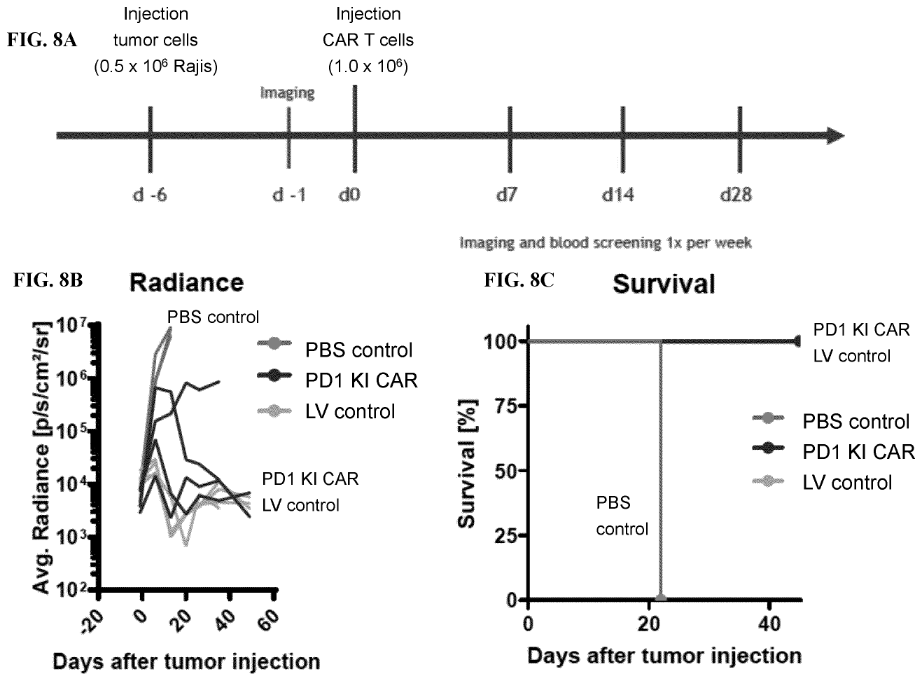

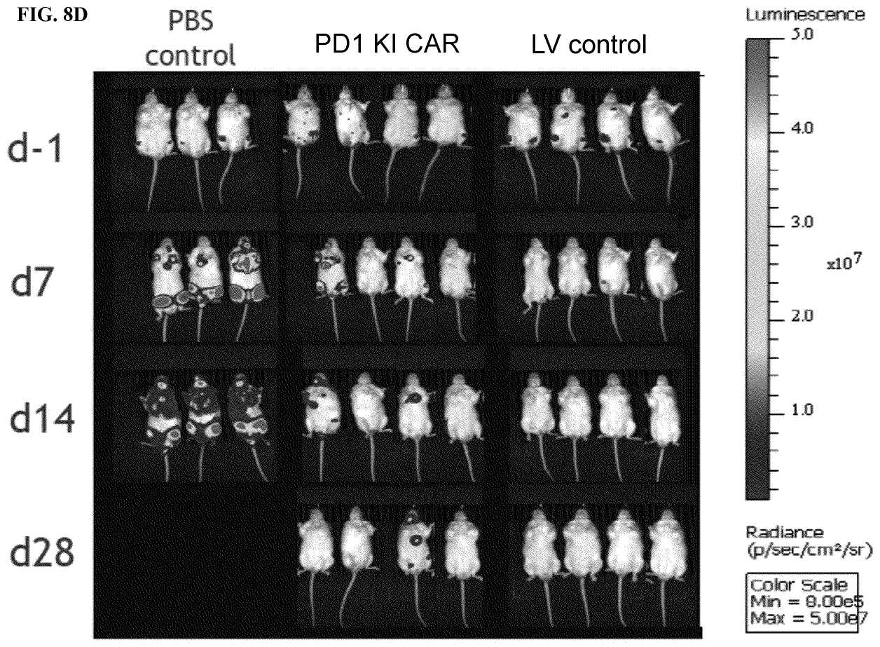

Exemplary Cas9 molecules, their structure and variants include those described in, e.g., WO2015/161276, e.g., in FIGS. 2A-2G and 8A-8B therein, and WO2017/193107, WO2017/093969, US2016/272999 and US2015/056705.

Nucleic acids encoding the Cas9 molecules or Cas9 polypeptides, e.g., an eaCas9 molecule or eaCas9 polypeptide, can be used in connection with any of the embodiments provided herein.

Exemplary nucleic acids encoding Cas9 molecules or Cas9 polypeptides are described in Cong et al., Science 2013, 399(6121):819-823; Wang et al., Cell 2013, 153(4):910-918; Mali et al., Science 2013, 399(6121):823-826; Jinek et al., Science 2012, 337(6096):816-821, and WO2015/161276, e.g., in FIG. 8 therein.

In some embodiments, a nucleic acid encoding a Cas9 molecule or Cas9 polypeptide can be a synthetic nucleic acid sequence. For example, the synthetic nucleic acid molecule can be chemically modified. In some embodiments, the Cas9 mRNA has one or more (e.g., all of the following properties: it is capped, polyadenylated, substituted with 5-methylcytidine and/or pseudouridine. In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. In addition, or alternatively, a nucleic acid encoding a Cas9 molecule or Cas9 polypeptide may comprise a nuclear localization sequence (NLS). Nuclear localization sequences are known.

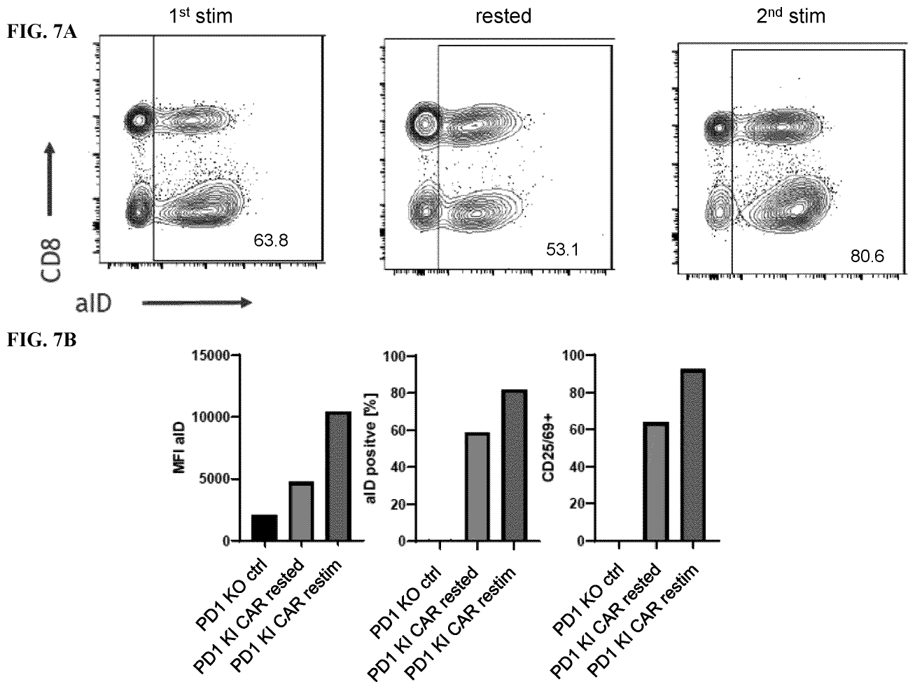

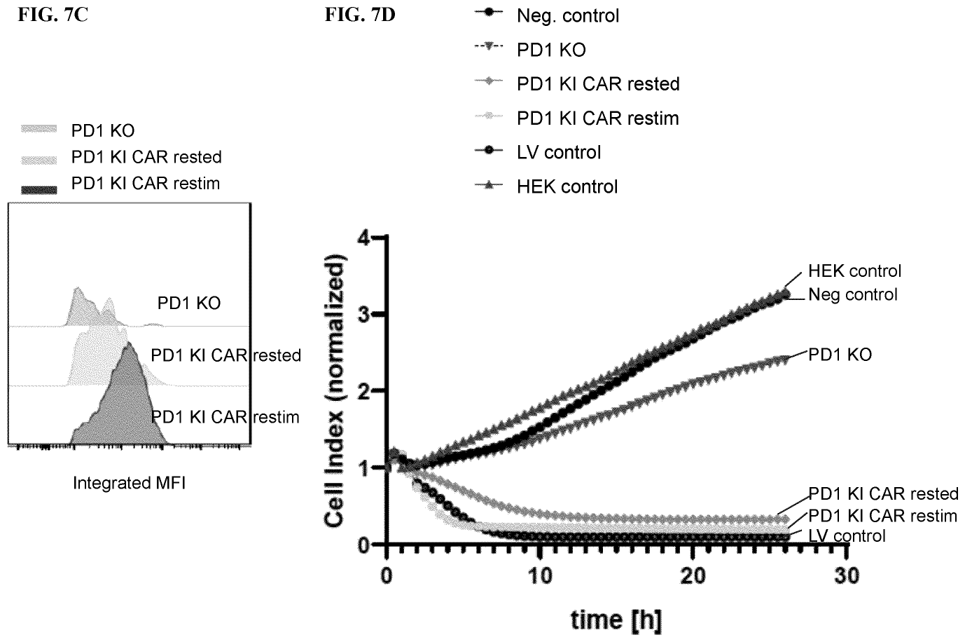

In some embodiments, the Cas9 molecule comprises by a sequence that is or comprises any of SEQ ID NOS: 279-287 or a sequence that exhibits at least 85%, 86%, 87%, 88%, 89%, 90%, 91%, 92%, 92%, 94%, 95%, 96%, 97%, 98%, 99% or more sequence identity to any of SEQ ID NOS: 112-117. Exemplary Cas9 molecule includes a Cas9 molecule of S. pyogenes, S. aureus or N. meningitidis. In some embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In some embodiments, each of regions 1-6, independently, have, 50%, 60%, 70%, or 80% homology with the corresponding residues of a Cas9 molecule or Cas9 polypeptide described herein, e.g., set forth in SEQ ID NOS: 279-287 or a sequence disclosed in WO2015/161276, e.g., from FIGS. 2A-2G or from FIGS. 7A-7B therein.

If any of the foregoing Cas9 sequences are fused with a peptide or polypeptide at the C-terminus, it is understood that the stop codon will be removed.

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., PLoS Computational Biology 2005, 1(6): e60 and Makarova et al., Nature Review Microbiology 2011, 9:467-477, the contents of both references are incorporated herein by reference in their entirety. Exemplary Cas molecules (and Cas systems) include those described in, e.g., WO2015/161276, WO2017/193107, WO2017/093969, US2016/272999 and US2015/056705.

(iii) Cpf1

In some embodiments, the guide RNA or gRNA promotes the specific association targeting of an RNA-guided nuclease such as a Cas9 or a Cpf1 to a target sequence such as a genomic or episomal sequence in a cell. In general, gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, in some embodiments by duplexing). gRNAs and their component parts are described throughout the literature, in some embodiments in Briner et al. Molecular Cell (2014) 56(2), 333-339, which is incorporated by reference.

Guide RNAs, whether unimolecular or modular, generally include a targeting domain that is fully or partially complementary to a target, and are typically 10-30 nucleotides in length, and in certain embodiments are 16-24 nucleotides in length (in some embodiments, 16, 17, 18, 19, 20, 21, 22, 23 or 24 nucleotides in length). In some aspects, the targeting domains are at or near the 5′ terminus of the gRNA in the case of a Cas9 gRNA, and at or near the 3′ terminus in the case of a Cpf1 gRNA. While the foregoing description has focused on gRNAs for use with Cas9, it should be appreciated that other RNA-guided nucleases have been (or may in the future be) discovered or invented which utilize gRNAs that differ in some ways from those described to this point. In some embodiments, Cpf1 (“CRISPR from Prevotella and Franciscella 1”) is a recently discovered RNA-guided nuclease that does not require a tracrRNA to function. (Zetsche et al., 2015, Cell 163, 759-771, incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, in some aspects in this disclosure, gRNAs may be described solely in terms of their targeting domain sequences.

More generally, some aspects of the present disclosure relate to systems, methods and compositions that can be implemented using multiple RNA-guided nucleases. Unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

While Cas9 and Cpf1 share similarities in structure and function, it should be appreciated that certain Cpf1 activities are mediated by structural domains that are not analogous to any Cas9 domains. In some embodiments, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Nucleic acids encoding RNA-guided nucleases, e.g., Cas9, Cpf1 or functional fragments thereof, are provided herein. Exemplary nucleic acids encoding RNA-guided nucleases include those described in, for example, Cong et al., Science 2013, 399(6121):819-823; Wang et al., Cell 2013, 153(4):910-918; Mali et al., Science 2013, 399(6121):823-826; Jinek et al., Science 2012, 337(6096):816-821.

b. Genome Editing Approaches

In general, it is to be understood that the alteration of any gene according to the methods described herein can be mediated by any mechanism and that any methods are not limited to a particular mechanism. Exemplary mechanisms that can be associated with the alteration of a gene include, but are not limited to, non-homologous end joining (e.g., classical or alternative), microhomology-mediated end joining (MMEJ), homology-directed repair (e.g., endogenous donor template mediated), synthesis dependent strand annealing (SDSA), single strand annealing, single strand invasion, single strand break repair (SSBR), mismatch repair (MMR), base excision repair (BER), Interstrand Crosslink (ICL) Translesion synthesis (TLS), or Error-free postreplication repair (PRR). Described herein are exemplary methods for targeted knockout of one or both alleles of the T cell stimulation-associated locus, TRAC and/or TRBC. Exemplary mechanisms include those described in, for example, U.S. Patent Pub. No. US20170349894, US20180362943 and US20180245079.

In some embodiments, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In some embodiments, the cleavage site is between 0-30 bp away from the target position (e.g., less than 30, 25, 20, 15, 10, 9, 8, 7, 6, 5, 4, 3, 2 or 1 bp from the target position).

In some embodiments, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In some embodiments, the gRNAs are configured to position cuts at the same position, or within a few nucleotides of one another, on different strands, essentially mimicking a double strand break. In some embodiments, the closer nick is between 0-30 bp away from the target position (e.g., less than 30, 25, 20, 15, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp). In some embodiments, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks in deleted). In some embodiments, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

Any of the Cas9 molecules, gRNA molecules, Cas9 molecule/gRNA molecule complexes, can be evaluated by art-known methods or as described herein. For example, exemplary methods for evaluating the endonuclease activity of Cas9 molecule are described, e.g., in Jinek et al., Science 2012, 337(6096):816-821, WO2015/161276, WO2017/193107, WO2017/093969, US2016/272999 and US2015/056705.

### 3. Delivery of Agents for Genetic Disruption

In some embodiments, the targeted genetic disruption, e.g., DNA break, of the endogenous T cell stimulation-associated locus, TRAC and/or TRBC in humans is carried out by delivering or introducing one or more agent(s) capable of inducing a genetic disruption, e.g., Cas9 and/or gRNA components, to a cell, using any of a number of known delivery method or vehicle for introduction or transfer to cells, for example, using viral, e.g., lentiviral, delivery vectors, or any of the known methods or vehicles for delivering Cas9 molecules and gRNAs. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequence encoding one or more components of one or more agent(s) capable of inducing a genetic disruption, e.g., DNA break, is introduced into the cells, e.g., by any methods for introducing nucleic acids into a cell described herein or known. In some embodiments, a vector encoding components of one or more agent(s) capable of inducing a genetic disruption such as a CRISPR guide RNA and/or a Cas9 enzyme can be delivered into the cell.

In some embodiments, the one or more agent(s) capable of inducing a genetic disruption, e.g., one or more agent(s) that is a Cas9/gRNA, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more agent(s) capable of inducing genetic disruption, e.g., CRISPR/Cas9, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the integration of transgene can be inherited by the progeny cells, but without the agent itself, which may further introduce off-target genetic disruptions, being passed on to the progeny cells.

In some embodiments, the RNP complexes include a gRNA that has been modified to include a 3′ poly-A tail and a 5′ Anti-Reverse Cap Analog (ARCA) cap.

Agent(s) and components capable of inducing a genetic disruption, e.g., a Cas9 molecule and gRNA molecule, can be introduced into target cells in a variety of forms using a variety of delivery methods and formulations, as set forth in Tables 10 and 11, or methods described in, e.g., WO 2015/161276; WO2017/193107, WO2017/093969, US 2015/0056705, US 2016/0272999, US 2017/0211075; or US 2017/0016027. As described further herein, the delivery methods and formulations can be used to deliver template polynucleotides and/or other agents to the cell (such as those required for engineering the cells) in prior or subsequent steps of the methods described herein. When a Cas9 or gRNA component is encoded as DNA for delivery, the DNA may typically but not necessarily include a control region, e.g., comprising a promoter, to effect expression. Useful promoters for Cas9 molecule sequences include, e.g., CMV, EF-1α, EFS, MSCV, PGK, or CAG promoters. Useful promoters for gRNAs include, e.g., H1, EF-1α, tRNA or U6 promoters. Promoters with similar or dissimilar strengths can be selected to tune the expression of components. Sequences encoding a Cas9 molecule may comprise a nuclear localization signal (NLS), e.g., an SV40 NLS. In some embodiments a promoter for a Cas9 molecule or a gRNA molecule may be, independently, inducible, tissue specific, or cell specific. In some embodiments, an agent capable of inducing a genetic disruption is introduced RNP complexes.

TABLE 10
Exemplary Delivery Methods

| Elements | | |
|---|---|---|
| Cas9 | gRNA | |
| Molecule(s) | molecule(s) | Comments |
| DNA | DNA | In this embodiment, a Cas9 molecule and a gRNA are transcribed |
| | | from DNA. In this embodiment, they are encoded on separate |
| | | molecules. |
| DNA | | In this embodiment, a Cas9 molecule and a gRNA are transcribed |
| | | from DNA, here from a single molecule. |
| DNA | RNA | In this embodiment, a Cas9 molecule is transcribed from DNA, |
| | | and a gRNA is provided as in vitro transcribed or synthesized |
| | | RNA |
| mRNA | RNA | In this embodiment, a Cas9 molecule is translated from in vitro |
| | | transcribed mRNA, and a gRNA is provided as in vitro |
| | | transcribed or synthesized RNA. |
| mRNA | DNA | In this embodiment, a Cas9 molecule is translated from in vitro |
| | | transcribed mRNA, and a gRNA is transcribed from DNA. |
| Protein | DNA | In this embodiment, a Cas9 molecule is provided as a protein, and |
| | | a gRNA is transcribed from DNA. |
| Protein | RNA | In this embodiment, a Cas9 molecule is provided as a protein, and |
| | | a gRNA is provided as transcribed or synthesized RNA. |

TABLE 11
Comparison of Exemplary Delivery Methods

| | | Delivery into | | | Type of |
|---|---|---|---|---|---|
| | | Non-Dividing | Duration of | Genome | Molecule |
| Delivery Vector/Mode | | Cells | Expression | Integration | Delivered |
| Physical (e.g., electroporation, | | YES | Transient | NO | Nucleic |
| particle gun, Calcium Phosphate | | | | | Acids and |
| transfection, cell compression or | | | | | Proteins |
| squeezing) | | | | | |
| Viral | Retrovirus | NO | Stable | YES | RNA |
| | Lentivirus | YES | Stable | YES/NO | RNA |
| | | | | with | |
| | | | | modifications | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex Virus | YES | Stable | NO | DNA |
| Non-Viral | Cationic Liposomes | YES | Transient | Depends on | Nucleic |
| | | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated Bacteria | YES | Transient | NO | Nucleic |
| Non-Viral | | | | | Acids |
| Delivery | Engineered | YES | Transient | NO | Nucleic |
| Vehicles | Bacteriophages | | | | Acids |
| | Mammalian Virus- | YES | Transient | NO | Nucleic |
| | like Particles | | | | Acids |
| | Biological liposomes: | YES | Transient | NO | Nucleic |
| | Erythrocyte Ghosts | | | | Acids |
| | and Exosomes | | | | |

In some embodiments, DNA encoding Cas9 molecules and/or gRNA molecules, or RNP complexes comprising a Cas9 molecule and/or gRNA molecules, can be delivered into cells by known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof. In some embodiments, the polynucleotide containing the agent(s) and/or components thereof is delivered by a vector (e.g., viral vector/virus or plasmid). The vector may be any described herein.

In some aspects, a CRISPR enzyme (e.g. Cas9 nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target locus (e.g., T cell stimulation-associated locus, TRAC and/or TRBC) are introduced into cells.

In some embodiments, the polynucleotide containing the agent(s) and/or components thereof or RNP complex is delivered by a non-vector based method (e.g., using naked DNA or DNA complexes). For example, the DNA or RNA or proteins or combination thereof, e.g., ribonucleoprotein (RNP) complexes, can be delivered, e.g., by organically modified silica or silicate (Ormosil), electroporation, transient cell compression or squeezing (such as described in Lee et al. (2012) Nano Lett 12: 6322-27, Kollmannsperger et al. (2016) Nat Comm 7, 10372), gene gun, sonoporation, magnetofection, lipid-mediated transfection, dendrimers, inorganic nanoparticles, calcium phosphates, or a combination thereof.

In some embodiments, delivery via electroporation comprises mixing the cells with the Cas9- and/or gRNA-encoding DNA or RNP complex in a cartridge, chamber or cuvette and applying one or more electrical impulses of defined duration and amplitude. In some embodiments, delivery via electroporation is performed using a system in which cells are mixed with the Cas9- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the delivery vehicle is a non-viral vector. In some embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In some embodiments, the non-viral vector is an organic nanoparticle. Exemplary organic nanoparticles include, e.g., SNALP liposomes that contain cationic lipids together with neutral helper lipids which are coated with polyethylene glycol (PEG), and protamine-nucleic acid complexes coated with lipid. Exemplary lipids for gene transfer include those described in, e.g., WO2015/161276, WO2017/193107, WO2017/093969, US2016/272999 and US2015/056705.

In some embodiments, the vehicle has targeting modifications to increase target cell update of nanoparticles and liposomes, e.g., cell specific antigens, monoclonal antibodies, single chain antibodies, aptamers, polymers, sugars, and cell penetrating peptides. In some embodiments, the vehicle uses fusogenic and endosome-destabilizing peptides/polymers. In some embodiments, the vehicle undergoes acid-triggered conformational changes (e.g., to accelerate endosomal escape of the cargo). In some embodiments, a stimulus-cleavable polymer is used, e.g., for release in a cellular compartment. For example, disulfide-based cationic polymers that are cleaved in the reducing cellular environment can be used.

In some embodiments, the delivery vehicle is a biological non-viral delivery vehicle. In some embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific cells, bacteria having modified surface proteins to alter target cell specificity). In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue-specificity. In some embodiments, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes-subject-derived membrane-bound nanovescicles (30-100 nm) of endocytic origin (e.g., can be produced from various cell types and can therefore be taken up by cells without the need for targeting ligands).

In some embodiments, RNA encoding Cas9 molecules and/or gRNA molecules, can be delivered into cells, e.g., target cells described herein, by known methods or as described herein. For example, Cas9-encoding and/or gRNA-encoding RNA can be delivered, e.g., by microinjection, electroporation, transient cell compression or squeezing (such as described in Lee et al. (2012) Nano Lett 12: 6322-27), lipid-mediated transfection, peptide-mediated delivery, e.g., cell-penetrating peptides, or a combination thereof.

In some embodiments, delivery via electroporation comprises mixing the cells with the RNA encoding Cas9 molecules and/or gRNA molecules in a cartridge, chamber or cuvette and applying one or more electrical impulses of defined duration and amplitude. In some embodiments, delivery via electroporation is performed using a system in which cells are mixed with the RNA encoding Cas9 molecules and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, Cas9 molecules can be delivered into cells by known methods or as described herein. For example, Cas9 protein molecules can be delivered, e.g., by microinjection, electroporation, transient cell compression or squeezing (such as described in Lee et al. (2012) Nano Lett 12: 6322-27), lipid-mediated transfection, peptide-mediated delivery, or a combination thereof. Delivery can be accompanied by DNA encoding a gRNA or by a gRNA.

In some embodiments, the one or more agent(s) capable of introducing a cleavage, e.g., a Cas9/gRNA system, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, calcium phosphate transfection, cell compression or squeezing.

In some embodiments, delivery via electroporation comprises mixing the cells with the Cas9 molecules with or without gRNA molecules in a cartridge, chamber or cuvette and applying one or more electrical impulses of defined duration and amplitude. In some embodiments, delivery via electroporation is performed using a system in which cells are mixed with the Cas9 molecules with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, delivery via electroporation comprises mixing the cells with the Cas9 molecules (e.g., eaCas9 molecules, eiCas9 molecules or eiCas9 fusion proteins) with or without gRNA molecules in a cartridge, chamber or cuvette and applying one or more electrical impulses of defined duration and amplitude. In some embodiments, delivery via electroporation is performed using a system in which cells are mixed with the Cas9 molecules (e.g., eaCas9 molecules, eiCas9 molecules or eiCas9 fusion proteins)

In some embodiments, the polynucleotide containing the agent(s) and/or components thereof is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, more than one agent(s) or components thereof are delivered to the cell. For example, in some embodiments, agent(s) capable of inducing a genetic disruption of two or more locations in the genome, such as at two or more sites within a T cell stimulation-associated locus, TRAC and/or TRBC, are delivered to the cell. In some embodiments, agent(s) and components thereof are delivered using one method. For example, in some embodiments, agent(s) for inducing a genetic disruption of the T cell stimulation-associated locus, TRAC and/or TRBC are delivered as polynucleotides encoding the components for genetic disruption. In some embodiments, one polynucleotide can encode agents that target the T cell stimulation-associated locus, TRAC and/or TRBC. In some embodiments, two or more different polynucleotides can encode the agents that target the T cell stimulation-associated locus, TRAC and/or TRBC. In some embodiments, the agents capable of inducing a genetic disruption can be delivered as ribonucleoprotein (RNP) complexes, and two or more different RNP complexes can be delivered together as a mixture, or separately.

In some embodiments, one or more nucleic acid molecules other than the one or more agent(s) capable of inducing a genetic disruption and/or component thereof, e.g., the Cas9 molecule component and/or the gRNA molecule component, such as a template polynucleotide for HDR-directed integration (such as any template polynucleotide described herein, e.g., in Section II.B.2), are delivered. In some embodiments, the nucleic acid molecule, e.g., template polynucleotide, is delivered at the same time as one or more of the components of the Cas system. In some embodiments, the nucleic acid molecule is delivered before or after (e.g., less than about 1 minute, 5 minutes, 10 minutes, 15 minutes, 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In some embodiments, the nucleic acid molecule, e.g., template polynucleotide, is delivered by a different means from one or more of the components of the Cas system, e.g., the Cas9 molecule component and/or the gRNA molecule component. The nucleic acid molecule, e.g., template polynucleotide, can be delivered by any of the delivery methods described herein. For example, the nucleic acid molecule, e.g., template polynucleotide, can be delivered by a viral vector, e.g., a retrovirus or a lentivirus, and the Cas9 molecule component and/or the gRNA molecule component can be delivered by electroporation. In some embodiments, the nucleic acid molecule, e.g., template polynucleotide, includes one or more exogenous sequences, e.g., sequences that encode a recombinant receptor or a portion thereof and/or other exogenous gene nucleic acid sequences.

### B. Targeted Integration Via Homology-Directed Repair (HDR)

In some aspects, the provided embodiments involve targeted integration of a specific part of a polynucleotide, such as the part of a template polynucleotide containing a transgene encoding a recombinant receptor or a portion thereof, at a particular location (such as target site or target location) in the genome at the endogenous T cell stimulation-associated locus. In some aspects, homology-directed repair (HDR) can mediate the site specific integration of the transgene at the target site. In some embodiments, the presence of a genetic disruption (e.g., a DNA break, such as described in Section II.A) and a template polynucleotide containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the 5′ and/or 3′ homology arms included in the template polynucleotide, cellular DNA repair machinery can use the template polynucleotide to repair the DNA break and resynthesize genetic information at the site of the genetic disruption, thereby effectively inserting or integrating the transgene in the template polynucleotide at or near the site of the genetic disruption. In some embodiments, the genetic disruption at an endogenous T cell stimulation-associated locus, can be generated by any of the methods for generating a targeted genetic disruption described herein.

Also provided are polynucleotides, e.g., template polynucleotides described herein, and kits that include such polynucleotides. In some embodiments, the provided polynucleotides and/or kits can be employed in the methods described herein, e.g., involving HDR, to target the transgene encoding a recombinant receptor or a portion thereof at the endogenous T cell stimulation-associated locus.

In some embodiments, the template polynucleotide is or comprises a polynucleotide containing a transgene, such as exogenous or heterologous nucleic acid sequences, encoding a recombinant receptor or a portion thereof (e.g., one or more region(s) or domain(s) of the recombinant receptor), and homology sequences (e.g., homology arms) that are homologous to sequences at or near the endogenous genomic site at the endogenous T cell stimulation-associated locus. In some aspects, the transgene in the template polynucleotide comprise sequence of nucleotides encoding a recombinant receptor or a portion thereof. In some aspects, upon targeted integration of the transgene, the T cell stimulation-associated locus in the engineered cell is modified such that the modified T cell stimulation-associated locus contains the transgene encoding a recombinant receptor, e.g., a chimeric antigen receptor (CAR).

In some aspects, the template polynucleotide is introduced as a linear DNA fragment or comprised in a vector. In some aspects, the step for inducing genetic disruption and the step for targeted integration (e.g., by introduction of the template polynucleotide) are performed simultaneously or sequentially.

### 1. Homology-Directed Repair (HDR)

In some embodiments, homology-directed repair (HDR) can be utilized for targeted integration or insertion of one or more nucleic acid sequences, e.g., a transgene, at one or more target site(s) at a T cell stimulation-associated locus. In some embodiments, the nuclease-induced HDR can be used to alter a target sequence, integrate the transgene at a particular target location, and/or to edit or repair a mutation in a particular target gene, for example, a T cell stimulation-associated locus.

Alteration of nucleic acid sequences at the target site can occur by HDR with an exogenously provided template polynucleotide (also referred to as “donor polynucleotide” or “template sequence”). For example, the template polynucleotide provides for alteration of the target sequence, such as insertion of the transgene contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Template polynucleotide-effected alteration of a target sequence depends on cleavage by a nuclease, e.g., a targeted nuclease such as CRISPR/Cas9. Cleavage by the nuclease can comprise a double strand break or two single strand breaks.

In some embodiments, “recombination” includes a process of exchange of genetic information between two polynucleotides. In some embodiments, “homologous recombination (HR)” includes a specialized form of such exchange that takes place, for example, during repair of double-strand breaks in cells via homology-directed repair mechanisms. This process requires nucleotide sequence homology, uses a template polynucleotide to template repair of a target DNA (i.e., the one that experienced the double-strand break, such as target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide.

In some embodiments, a template polynucleotide, e.g., polynucleotide containing transgene, is integrated into the genome of a cell via homology-independent mechanisms. The methods comprise creating a double-stranded break (DSB) in the genome of a cell and cleaving the template polynucleotide molecule using a nuclease, such that the template polynucleotide is integrated at the site of the DSB. In some embodiments, the template polynucleotide is integrated via non-homology dependent methods (e.g., NHEJ). Upon in vivo cleavage the template polynucleotides can be integrated in a targeted manner into the genome of a cell at the location of a DSB. The template polynucleotide can include one or more of the same target sites for one or more of the nucleases used to create the DSB. Thus, the template polynucleotide may be cleaved by one or more of the same nucleases used to cleave the endogenous gene into which integration is desired. In some embodiments, the template polynucleotide includes different nuclease target sites from the nucleases used to induce the DSB. As described herein, the genetic disruption of the target site or target position can be created by any know methods or any methods described herein, such as ZFNs, TALENs, CRISPR/Cas9 system, or TtAgo nucleases.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break. In some embodiments, a single-stranded template polynucleotide is used and the target site can be altered by alternative HDR.

Template polynucleotide-effected alteration of a target site depends on cleavage by a nuclease molecule. Cleavage by the nuclease can comprise a nick, a double strand break, or two single strand breaks, e.g., one on each strand of the DNA at the target site. After introduction of the breaks on the target site, resection occurs at the break ends resulting in single stranded overhanging DNA regions.

In canonical HDR, a double-stranded template polynucleotide is introduced, comprising homologous sequence to the target site that will either be directly incorporated into the target site or used as a template to insert the transgene or correct the sequence of the target site. After resection at the break, repair can progress by different pathways, e.g., by the double Holliday junction model (or double strand break repair, DSBR, pathway) or the synthesis-dependent strand annealing (SDSA) pathway.

In the double Holliday junction model, strand invasion by the two single stranded overhangs of the target site to the homologous sequences in the template polynucleotide occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the target site, e.g., insertion of the transgene in template polynucleotide. Crossover with the template polynucleotide may occur upon resolution of the junctions.

In the SDSA pathway, only one single stranded overhang invades the template polynucleotide and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

In alternative HDR, a single strand template polynucleotide, e.g., template polynucleotide, is introduced. A nick, single strand break, or double strand break at the target site, for altering a desired target site, is mediated by a nuclease molecule, and resection at the break occurs to reveal single stranded overhangs. Incorporation of the sequence of the template polynucleotide to correct or alter the target site of the DNA typically occurs by the SDSA pathway, as described herein.

“Alternative HDR”, or alternative homology-directed repair, in some embodiments, refers to the process of repairing DNA damage using a homologous nucleic acid (e.g., an endogenous homologous sequence, e.g., a sister chromatid, or an exogenous nucleic acid, e.g., a template polynucleotide). Alternative HDR is distinct from canonical HDR in that the process utilizes different pathways from canonical HDR, and can be inhibited by the canonical HDR mediators, RAD51 and BRCA2. Also, alternative HDR uses a single-stranded or nicked homologous nucleic acid for repair of the break. “Canonical HDR”, or canonical homology-directed repair, in some embodiments, refers to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2 and the homologous nucleic acid is typically double-stranded. Unless indicated otherwise, the term “HDR” in some embodiments encompasses canonical HDR and alternative HDR.

In some embodiments, double strand cleavage is effected by a nuclease, e.g., a Cas9 molecule having cleavage activity associated with an HNH-like domain and cleavage activity associated with a RuvC-like domain, e.g., an N-terminal RuvC-like domain, e.g., a wild type Cas9. Such embodiments require only a single gRNA.

In some embodiments, one single strand break, or nick, is effected by a nuclease molecule having nickase activity, e.g., a Cas9 nickase. A nicked DNA at the target site can be a substrate for alternative HDR.

In some embodiments, two single strand breaks, or nicks, are effected by a nuclease, e.g., Cas9 molecule, having nickase activity, e.g., cleavage activity associated with an HNH-like domain or cleavage activity associated with an N-terminal RuvC-like domain. Such embodiments usually require two gRNAs, one for placement of each single strand break. In some embodiments, the Cas9 molecule having nickase activity cleaves the strand to which the gRNA hybridizes, but not the strand that is complementary to the strand to which the gRNA hybridizes. In some embodiments, the Cas9 molecule having nickase activity does not cleave the strand to which the gRNA hybridizes, but rather cleaves the strand that is complementary to the strand to which the gRNA hybridizes. In some embodiments, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HNH activity and will cut on the strand to which the gRNA hybridizes (e.g., the complementary strand, which does not have the NGG PAM on it). In some embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In some embodiments, the Cas9 molecule is an N-terminal RuvC-like domain nickase, e.g., the Cas9 molecule comprises a mutation at N863, e.g., N863A.

In some embodiments, in which a nickase and two gRNAs are used to position two single strand nicks, one nick is on the + strand and one nick is on the − strand of the target DNA. The PAMs are outwardly facing. The gRNAs can be selected such that the gRNAs are separated by, from about 0-50, 0-100, or 0-200 nucleotides. In some embodiments, there is no overlap between the target sequences that are complementary to the targeting domains of the two gRNAs. In some embodiments, the gRNAs do not overlap and are separated by as much as 50, 100, or 200 nucleotides. In some embodiments, the use of two gRNAs can increase specificity, e.g., by decreasing off-target binding (Ran et al., Cell. 2013 Sep. 12; 154(6):1380-9).

In some embodiments, a single nick can be used to induce HDR, e.g., alternative HDR. It is contemplated herein that a single nick can be used to increase the ratio of HR to NHEJ at a given cleavage site, such as target site. In some embodiments, a single strand break is formed in the strand of the DNA at the target site to which the targeting domain of said gRNA is complementary. In some embodiments, a single strand break is formed in the strand of the DNA at the target site other than the strand to which the targeting domain of said gRNA is complementary.

In some embodiments, other DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

Targeted integration results in the transgene, e.g., sequences between the homology arms, being integrated into a T cell stimulation-associated locus in the genome. The transgene may be integrated anywhere at or near one of the at least one target site(s) or site in the genome. In some embodiments, the transgene is integrated at or near one of the at least one target site(s), for example, within 300, 250, 200, 150, 100, 50, 10, 5, 4, 3, 2, 1 or fewer base pairs upstream or downstream of the site of cleavage, such as within 100, 50, 10, 5, 4, 3, 2, 1 base pairs of either side of the target site, such as within 50, 10, 5, 4, 3, 2, 1 base pairs of either side of the target site. In some embodiments, the integrated sequence comprising the transgene does not include any vector sequences (e.g., viral vector sequences). In some embodiments, the integrated sequence includes a portion of the vector sequences (e.g., viral vector sequences).

The double strand break or single strand break (such as target site) in one of the strands should be sufficiently close to the target integration site, e.g., site for targeted integration, such that an alteration is produced in the desired region, such as insertion of transgene or correction of a mutation occurs. In some embodiments, the distance is not more than 10, 25, 50, 100, 200, 300, 350, 400 or 500 nucleotides. In some embodiments, it is believed that the break should be sufficiently close to the target integration site such that the break is within the region that is subject to exonuclease-mediated removal during end resection. In some embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 350, 400 or 500 nucleotides of the region desired to be altered, e.g., site for targeted insertion. The break, e.g., a double strand or single strand break, can be positioned upstream or downstream of the region desired to be altered, e.g., site for targeted insertion. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of target integration site.

In some embodiments, a single strand break is accompanied by an additional single strand break, positioned by a second gRNA molecule. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 350, 400 or 500 nucleotides of a target integration site. In some embodiments, the first and second gRNA molecules are configured such, that when guiding a Cas9 nickase, a single strand break will be accompanied by an additional single strand break, positioned by a second gRNA, sufficiently close to one another to result in alteration of the desired region. In some embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In some embodiments, the two gRNA molecules are configured to position cuts at the same position, or within a few nucleotides of one another, on different strands, e.g., essentially mimicking a double strand break.

In some embodiments, in which a gRNA (unimolecular, chimeric, or modular gRNA) and Cas9 nuclease induce a double strand break for the purpose of inducing HDR-mediated insertion of transgene or correction, the cleavage site, such as target site, is between 0-200 bp (e.g., 0-175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target integration site. In some embodiments, the cleavage site, such as target site such as target site, is between 0-100 bp (e.g., 0 to 75, 0 to 50, 0 to 25, 25 to 100, 25 to 75, 25 to 50, 50 to 100, 50 to 75 or 75 to 100 bp) away from the site for targeted integration.

In some embodiments, one can promote HDR by using nickases to generate a break with overhangs. In some embodiments, the single stranded nature of the overhangs can enhance the cell's likelihood of repairing the break by HDR as opposed to, e.g., NHEJ.

Specifically, in some embodiments, HDR is promoted by selecting a first gRNA that targets a first nickase to a first target site, and a second gRNA that targets a second nickase to a second target site which is on the opposite DNA strand from the first target site and offset from the first nick. In some embodiments, the targeting domain of a gRNA molecule is configured to position a cleavage event sufficiently far from a preselected nucleotide, e.g., the nucleotide of a coding region, such that the nucleotide is not altered. In some embodiments, the targeting domain of a gRNA molecule is configured to position an intronic cleavage event sufficiently far from an intron/exon border, or naturally occurring splice signal, to avoid alteration of the exonic sequence or unwanted splicing events. In some embodiments, the targeting domain of a gRNA molecule is configured to position in an early exon, to allow in-frame integration of the transgene at or near one of the at least one target site(s).

In some embodiments, a double strand break can be accompanied by an additional double strand break, positioned by a second gRNA molecule. In some embodiments, a double strand break can be accompanied by two additional single strand breaks, positioned by a second gRNA molecule and a third gRNA molecule. In some embodiments, two gRNAs, e.g., independently, unimolecular, chimeric, or modular gRNA, are configured to position a double-strand break on both sides of a target integration site, e.g., site for targeted integration.

### 2. Template Polynucleotide

In some embodiments, a template polynucleotide, e.g., a polynucleotide containing a transgene, such as exogenous or heterologous nucleic acid sequences, that includes a sequence of nucleotides encoding a recombinant receptor or a portion thereof, and homology sequences (e.g., homology arms) that are homologous to sequences at or near the endogenous genomic site for targeted integration, can be employed molecules and machinery involved in cellular DNA repair processes, such as homologous recombination, as a repair template. In some aspects, a template polynucleotide having homology with sequences at or near one or more target site(s) in the endogenous DNA can be used to alter the structure of a target DNA, such as target site at the endogenous T cell stimulation-associated locus, for targeted insertion of the transgenic or exogenous sequences, e.g., exogenous nucleic acid sequence encoding the recombinant receptor or portion thereof. Also provided are polynucleotides, e.g., template polynucleotides, for use in the methods provided herein, e.g., as templates for homology directed repair (HDR) mediated targeted integration of the transgene. In some embodiments, the polynucleotide includes a nucleic acid sequence encoding a recombinant receptor or a portion thereof; and one or more homology arm(s) linked to the nucleic acid sequence, wherein the one or more homology arm(s) comprise a sequence homologous to one or more region(s) of an open reading frame of a T cell stimulation-associated locus.

In some embodiments, the template polynucleotide contains one or more homology sequences (e.g., homology arms) linked to and/or flanking the transgene (exogenous or heterologous nucleic acids sequences) that includes a sequence of nucleotides encoding the recombinant receptor or portion thereof. In some embodiments, the homology sequences are used to target the exogenous sequences at the endogenous T cell stimulation-associated locus. In some embodiments, the template polynucleotide includes nucleic acid sequences, such as a transgene, between the homology arms, for insertion or integration into the genome of a cell. The transgene in the template polynucleotide may comprise one or more sequences encoding a functional polypeptide (for example, a recombinant receptor or a portion thereof), with or without a promoter or other regulatory elements.

In some embodiments, a template polynucleotide is a nucleic acid sequence which can be used in conjunction with one or more agent(s) capable of introducing a genetic disruption, to alter the structure of a target site. In some embodiments, the template polynucleotide alters the structure of the target site, e.g., insertion of transgene, by a homology directed repair event.

In some embodiments, the template polynucleotide alters the sequence of the target site, e.g., results in insertion or integration of the transgene between the homology arms, into the genome of the cell. In some aspects, targeted integration results in an in-frame integration of the coding portion of the transgene with one or more exons of the open reading frame of the endogenous T cell stimulation-associated locus, e.g., in-frame with the adjacent exon at the integration site. For example, in some cases, the in-frame integration results in a portion of the endogenous open reading frame and the recombinant receptor or portion thereof to be expressed, in some cases separated by a multicistronic element, such as a 2A element. Thus, the modified T cell stimulation-associated locus can express a polypeptide encoded by the endogenous T cell stimulation-associated locus and the recombinant receptor or portion thereof, which can be separated into 2 different polypeptides by virtue of the multicistronic element.

In some embodiments, the template polynucleotide includes sequences that correspond to or is homologous to a site on the target sequence that is cleaved, e.g., by one or more agent(s) capable of introducing a genetic disruption. In some embodiments, the template polynucleotide includes sequences that correspond to or is homologous to both, a first site on the target sequence that is cleaved in a first agent capable of introducing a genetic disruption, and a second site on the target sequence that is cleaved in a second agent capable of introducing a genetic disruption.

In some embodiments, a template polynucleotide comprises the following components: [5′ homology arm]-[a transgene (exogenous or heterologous nucleic acid sequences, e.g., encoding a recombinant receptor or a portion thereof)]-[3′ homology arm]. The homology arms provide for recombination into the chromosome, thus effectively inserting or integrating the transgene, e.g., that encodes a the recombinant receptor or portion thereof, into the genomic DNA at or near the cleavage site, such as target site(s). In some embodiments, the homology arms flank the sequences at the target site of genetic disruption.

In some embodiments, the template polynucleotide is double stranded. In some embodiments, the template polynucleotide is single stranded. In some embodiments, the template polynucleotide comprises a single stranded portion and a double stranded portion. In some embodiments, the template polynucleotide is comprised in a vector. In some embodiments, the template polynucleotide is DNA. In some embodiments, the template polynucleotide is RNA. In some embodiments, the template polynucleotide is double stranded DNA. In some embodiments, the template polynucleotide is single stranded DNA. In some embodiments, the template polynucleotide is double stranded RNA. In some embodiments, the template polynucleotide is single stranded RNA. In some embodiments, the template polynucleotide comprises a single stranded portion and a double stranded portion. In some embodiments, the template polynucleotide is comprised in a vector.

In certain embodiments, the polynucleotide, e.g., template polynucleotide contains and/or includes a transgene encoding a recombinant receptor or a portion thereof, e.g., a CAR or a portion thereof. In some of any embodiments, the transgene is targeted at a target site(s) that is within an endogenous gene, locus, or open reading frame that encodes the T cell stimulation-associated gene product. In some embodiments, the transgene is targeted for integration within the endogenous T cell stimulation-associated locus open reading frame, such as to result in the expression of all or a portion of the encoded T cell stimulation-associated gene product.

Polynucleotides for insertion can also be referred to as “transgene” or “exogenous sequences” or “donor” polynucleotides or molecules. The template polynucleotide can be DNA, single-stranded and/or double-stranded and can be introduced into a cell in linear or circular form. The template polynucleotide can be DNA, single-stranded and/or double-stranded and can be introduced into a cell in linear or circular form. The template polynucleotide can be RNA single-stranded and/or double-stranded and can be introduced as a RNA molecule (e.g., part of an RNA virus). See also, U.S. Patent Pub. Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by known methods. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Pub. No. 20130326645.

In some embodiments, the double-stranded template polynucleotide includes sequences (also referred to as transgene) greater than 1 kb in length, for example between 2 and 200 kb, between 2 and 10 kb (or any value therebetween).

In some embodiments, the template polynucleotide is a single stranded nucleic acid. In some embodiments, the template polynucleotide is a double stranded nucleic acid. In some embodiments, the template polynucleotide comprises a nucleotide sequence, e.g., of one or more nucleotides, that will be added to or will template a change in the target DNA. In some embodiments, the template polynucleotide comprises a nucleotide sequence that may be used to modify the target site, e.g., copying or inserting the transgene in the template polynucleotide into the genome of the cell. In some embodiments, the template polynucleotide comprises a nucleotide sequence, e.g., of one or more nucleotides, that corresponds to wild type sequence of the target DNA, e.g., of the target site.

In some embodiments, the template polynucleotide is linear double stranded DNA. The length may be, e.g., about 200-5000 base pairs, e.g., about 200, 300, 400, 500, 600, 700, 800, 900, 1000, 1200, 1400, 1600, 1800, 2000, 2500, 3000, 4000 or 5000 base pairs. The length may be, e.g., at least 200, 300, 400, 500, 600, 700, 800, 900, 1000, 1200, 1400, 1600, 1800, 2000, 2500, 3000, 4000 or 5000 base pairs. In some embodiments, the length is no greater than 200, 300, 400, 500, 600, 700, 800, 900, 1000, 1200, 1400, 1600, 1800, 2000, 2500, 3000, 4000 or 5000 base pairs. In some embodiments, a double stranded template polynucleotide has a length of more than at or about 160 base pairs, e.g., about 200-4000, 300-3500, 400-3000, 500-2500, 600-2000, 700-1900, 800-1800, 900-1700, 1000-1600, 1100-1500 or 1200-1400 base pairs.

In some embodiments, the template polynucleotide is at or about 1000, 1250, 1500, 1750, 2000, 2250, 2500, 2750, 3000, 3250, 3500, 3750 or 4000 nucleotides in length, or any value between any of the foregoing. In some embodiments, the polynucleotide is between at or about 1500 and at or about 2500 nucleotides or at or about 1750 and at or about 2250 nucleotides in length. In some embodiments, the template polynucleotide is about 2000±250, 2000±200, 2000±150, 2000±100 or 2000±50 nucleotides in length.

The transgene contained on the template polynucleotide described herein may be isolated from plasmids, cells or other sources using known standard techniques such as PCR. Template polynucleotide for use can include varying types of topology, including circular supercoiled, circular relaxed, linear and the like. Alternatively, they may be chemically synthesized using standard oligonucleotide synthesis techniques. In addition, template polynucleotides may be methylated or lack methylation. Template polynucleotides may be in the form of bacterial or yeast artificial chromosomes (BACs or YACs).

The template polynucleotide can be linear single stranded DNA In some embodiments, the template polynucleotide is (i) linear single stranded DNA that can anneal to the nicked strand of the target DNA, (ii) linear single stranded DNA that can anneal to the intact strand of the target DNA, (iii) linear single stranded DNA that can anneal to the transcribed strand of the target DNA, (iv) linear single stranded DNA that can anneal to the non-transcribed strand of the target DNA, or more than one of the preceding.

The length may be, e.g., about 200-5000 nucleotides, e.g., about 200, 300, 400, 500, 600, 700, 800, 900, 1000, 1200, 1400, 1600, 1800, 2000, 2500, 3000, 4000 or 5000 nucleotides. The length may be, e.g., at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a single stranded template polynucleotide has a length of about 160 nucleotides, e.g., about 200-4000, 300-3500, 400-3000, 500-2500, 600-2000, 700-1900, 800-1800, 900-1700, 1000-1600, 1100-1500 or 1200-1400 nucleotides.

In some embodiments, the template polynucleotide is circular double stranded DNA, e.g., a plasmid. In some embodiments, the template polynucleotide comprises about 500 to 1000 base pairs of homology on either side of the transgene and/or the target site. In some embodiments, the template polynucleotide comprises about 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene.

a. Transgene

In some embodiments, the template polynucleotide contains a transgene encoding a recombinant receptor or a portion thereof, such as any recombinant receptor described herein, e.g., in Section IV.B, or one or more regions, domains or chains of such recombinant receptor.

In some aspects, the transgene encodes a recombinant receptor that includes an extracellular binding domain, transmembrane domain and/or an intracellular region. In some aspects, the transgene can encode all or a portion of the recombinant receptor. In some embodiments, the transgene encodes any recombinant receptor described herein, for example in Section IV.B, or a one or more regions, domains or chains thereof. In some aspects, upon integration of the transgene into the endogenous T cell stimulation-associated locus, the resulting modified T cell stimulation-associated locus encodes a recombinant receptor, such as any recombinant receptor described herein, for example, in Section IV.B, or a one or more regions, domains or chains thereof. For example, the transgene can include sequence of nucleotides encoding one or more of extracellular regions, transmembrane domains, and intracellular regions that can comprise costimulatory signaling domains, and other domains or portions thereof.

In some aspects, the transgene, which are nucleic acid sequences of interest encoding a recombinant receptor or a portion thereof, including coding and/or non-coding sequences and/or partial coding sequences thereof, that are inserted or integrated at the target location in the genome can also be referred to as “transgene,” “transgene sequences,” “exogenous nucleic acids sequences,” “heterologous sequences” or “donor sequences.” In some aspects, the transgene is a nucleic acid sequence that is exogenous or heterologous to an endogenous genomic sequences, such as the endogenous genomic sequences at a specific target locus or target location in the genome, of a T cell, e.g., a human T cell. In some aspects, the transgene is a sequence that is modified or different compared to an endogenous genomic sequence at a target locus or target location of a T cell, e.g., a human T cell. In some aspects, the transgene is a nucleic acid sequence that originates from or is modified compared to nucleic acid sequences from different genes, species and/or origins. In some aspects, the transgene is a sequence that is derived from a sequence from a different locus, e.g., a different genomic region or a different gene, of the same species. In some aspects, exemplary recombinant receptors include any described herein, e.g., in Section IV.B.

In some embodiments, nuclease-induced HDR results in an insertion of a transgene (also called “exogenous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

In some aspects, the transgene is a sequence that is exogenous or heterologous to an open reading frame of the endogenous genomic T cell stimulation-associated locus a T cell, in some cases a human T cell. In some aspects, HDR in the presence of a template polynucleotide containing a transgene linked to one or more homology arm(s) that are homologous to sequences near a target site at an endogenous T cell stimulation-associated locus, results in a modified T cell stimulation-associated locus encoding a recombinant receptor or a portion thereof.

In some embodiments, the transgene encodes all or a portion of the various regions, domains or chains of a recombinant receptor, such as a recombinant receptor or various regions, domains or chains described in Section IV.B herein.

In some aspects, the transgene is a chimeric sequence, comprising a sequence generated by joining different nucleic acid sequences from different genes, species and/or origins. In some aspects, the transgene contains sequence of nucleotides encoding different regions or domains or portions thereof, from different genes, coding sequences or exons or portions thereof, that are joined or linked. In some aspects the transgene for targeted integration encode a polypeptide or a fragment thereof.

In some embodiments, the transgene can encode a recombinant receptor that is a chimeric receptor, such as a chimeric antigen receptor (CAR), or a portion thereof, such as a domain or region thereof. In some embodiments, the transgene encodes various regions or domains of the recombinant receptor, such as a chimeric antigen receptor (CAR). In some embodiments, the transgene encodes the entire CAR, or the full-length CAR, comprising all domains or regions of the CAR. In some embodiments, the transgene includes a sequence of nucleotides encoding an intracellular region, such as an intracellular region of a CAR, for example comprising an intracellular signaling domain. In some embodiments, the transgene also includes a sequence of nucleotides encoding a transmembrane region or a membrane association region, such as a transmembrane region of a CAR. In some embodiments, the transgene also includes a sequence of nucleotides encoding an extracellular region, such as an extracellular region of a CAR. In some embodiments, the transgene encodes a portion of a CAR, for example, one or more domains or regions of a CAR. In some embodiments, the CAR is a multi-chain CAR, and the transgene encodes one or more chains of the multi-chain CAR. In some embodiments, the CAR is a multi-chain CAR, and the transgene encodes one chain of the multi-chain CAR. In some embodiments, if the transgene that is integrated at the T cell stimulation-associated locus in the provided engineered cell encodes a portion of the recombinant receptor, e.g., a CAR, the remaining portion of the recombinant receptor can be encoded by a second transgene present at a different location in the genome of the engineered cell (e.g., a different T cell stimulation-associated locus, or a different location). Exemplary chimeric receptors include those described in Sections IV.B.1 and IV.B.3 below.

In some embodiments, the transgene can encode a recombinant receptor, such as a recombinant T cell receptor (TCR), or a portion thereof, such as a domain, region or chain thereof. In some embodiments, the recombinant receptor is a recombinant TCR. In some embodiments, the recombinant receptor, such as a recombinant TCR, comprises two or more separate polypeptide chains, such as TCR alpha (TCRα) and TCR beta (TCRβ) chains. In some aspects, the transgene can encode one or more chains of the recombinant TCR, such as a TCRα or a TCRβ or both. In some aspects, the transgene can encode the entire recombinant TCR, e.g., both chains of a recombinant TCR, such as both a TCRα chain and a TCRβ chain. In some aspects, the transgene can encode one chain of a recombinant TCR, such as a TCRα chain or a TCRβ chain. In some aspects, the transgene can encode one or more regions or domains of the recombinant TCR, such as intracellular region, transmembrane region and/or extracellular region of a TCRα or a TCRβ or both. In some embodiments, if the transgene that is integrated at the T cell stimulation-associated locus in the provided engineered cell encodes a portion of the recombinant receptor, e.g., one chain of a recombinant TCR, the remaining portion of the recombinant receptor, e.g., the other chain of the recombinant TCR, can be encoded by a second transgene present at a different location in the genome of the engineered cell (e.g., a different T cell stimulation-associated locus, or a different location). In some embodiments, if the transgene that is integrated at the T cell stimulation-associated locus in the provided engineered cell encodes a portion of the recombinant receptor, e.g., one domain of a recombinant TCR, the remaining portion of the recombinant receptor, e.g., other remaining domains of the recombinant TCR, can be encoded by a second transgene present at a different location in the genome of the engineered cell. In some aspects, the sequences encoding the TCRα and TCRβ are in some cases separated by a multicistronic element, such as a 2A element. Exemplary recombinant TCRs include those described in Section III.B.4 below.

In some aspects, the transgene also contains non-coding, regulatory or control sequences, e.g., sequences required for permitting, modulating and/or regulating expression of the encoded polypeptide or fragment thereof or sequences required to modify a polypeptide. In some embodiments, the transgene does not comprise an intron or lacks one or more introns as compared to a corresponding nucleic acid in the genome if the transgene is derived from a genomic sequence. In some embodiments, the transgene does not comprise an intron. In some of embodiments, the transgene contains sequences encoding a recombinant receptor or a portion thereof, wherein all or a portion of the transgene are codon-optimized, e.g., for expression in human cells.

In some embodiments, the length of the transgene, including coding and non-coding regions, is between or between about 100 to about 10,000 base pairs, such as about 100, 200, 300, 400, 500, 600, 700, 800, 900, 1000, 1500, 2000, 2500, 3000, 3500, 4000, 4500, 5000, 6000, 7000, 8000, 9000 or 10000 base pairs. In some embodiments, the length of the transgene is limited by the maximum length of polynucleotide that can be prepared, synthesized or assembled and/or introduced into the cell or the capacity of the viral vector. In some aspects, the length of the transgene can vary depending on the maximum length of the template polynucleotide and/or the length of the one or more homology arm(s) required.

In some embodiments, genetic disruption-induced HDR results in an insertion or integration of the transgene at a target location in the genome. The template polynucleotide sequence is typically not identical to the genomic sequence where it is targeted. A template polynucleotide sequence can contain a transgene flanked by two regions of homology to allow for efficient HDR at the location of interest. A template polynucleotide sequence can contain several, discontinuous regions of homology to the genomic DNA. For example, for targeted insertion of sequences not normally present in a region of interest, said sequences can be present in a transgene and flanked by regions of homology to sequence in the region of interest. In some embodiments, the transgene encode a recombinant receptor or a portion thereof, e.g., one or more of an extracellular binding domain, transmembrane domain and/or a portion of the intracellular region.

In some aspects, upon targeted integration of the transgene by HDR, the genome of the cell contains a modified T cell stimulation-associated locus, comprising a nucleic acid sequence encoding a recombinant receptor or a portion thereof. In some aspects, the entire recombinant receptor is encoded by the transgene. In some aspects, the transgene also contain sequence of nucleotides encoding other molecules and/or regulatory or control elements, e.g., exogenous promoter, and/or one or more multicistronic elements.

In some embodiments, the transgene also includes a signal sequence encoding a signal peptide, a regulatory or control elements, such as a promoter, and/or one or more multicistronic elements, e.g., a ribosome skip element or an internal ribosome entry site (IRES). In some embodiments, the signal sequence can be placed 5′ of the sequence of nucleotides encoding the recombinant receptor.

Exemplary regions, domains or chains encoded by the transgene are described below, and also can be any region or domain described in Section IV.B herein.

### (i) Signal Sequence

In some embodiments, the transgene includes a signal sequence that encodes a signal peptide. In some aspects, the signal sequence may encode a heterologous or non-native signal peptide, e.g., a signal peptide from a different gene or species or a signal peptide that is different from the signal peptide of the endogenous T cell stimulation-associated locus. In some aspects, exemplary signal sequence includes signal sequence of the GMCSFR alpha chain set forth in SEQ ID NO:24 or 288 and encoding the signal peptide set forth in SEQ ID NO:25; or the CD8 alpha signal peptide set forth in SEQ ID NO:26. In the mature form of an expressed recombinant receptor, the signal sequence is cleaved from the remaining portions of the polypeptide. In some aspects, the signal sequence is placed 3′ of a regulatory or control element, e.g., a promoter, such as a heterologous promoter, e.g., a promoter not derived from the T cell stimulation-associated locus. In some aspects, the signal sequence is placed 3′ of one or more multicistronic element(s), e.g., a sequence of nucleotides encoding a ribosome skip sequence and/or an internal ribosome entry site (IRES). In some aspects, the signal sequence can be placed 5′ of the sequence of nucleotides encoding the one or more components of the extracellular region in the transgene. In some embodiments, the signal sequence the most 5′ region present in the transgene, and is linked to one of the homology arms. In some aspects, the signal sequence encoded by the transgene include any signal sequence described herein.

### (ii) Exemplary Recombinant Receptor-Encoding Sequences

In some aspects the transgene for targeted integration include sequences encoding a recombinant receptor that is a chimeric receptor, such as a chimeric antigen receptor (CAR) or a chimeric auto antibody receptor (CAAR). In some aspects, the transgene contains sequence of nucleotides encoding different regions or domains or portions of the chimeric receptor, that can be from different genes, coding sequences or exons or portions thereof, that are joined or linked.

In some embodiments, the encoded recombinant receptor, such as a CAR, contains one or more regions or domains, such as one or more of extracellular region (e.g., containing one or more extracellular binding domain(s) and/or spacers), transmembrane domain and/or intracellular region (e.g., containing primary signaling region or domain and/or one or more costimulatory signaling domains). In some aspects, the encoded CAR further contains other domains, such multimerization domains or linkers.

In some aspects, in the transgene, the sequence of nucleotides encoding the extracellular region is placed between the signal sequence and the nucleotides encoding the spacer. In some aspects, in the transgene, the sequence of nucleotides encoding the extracellular multimerization domain is placed between the sequence of nucleotides encoding the binding domain and the sequence of nucleotides encoding the spacer. In some aspects, the sequence of nucleotides encoding the spacer is placed between the sequence of nucleotides encoding the binding domain and the sequence of nucleotides encoding the transmembrane domain. In some embodiments, the transgene includes, in 5′ to 3′ order, a sequence of nucleotides encoding an extracellular region, a sequence of nucleotides a transmembrane domain (or a membrane association domain) and a sequence of nucleotides an intracellular region.

In some embodiments, the encoded recombinant receptor is a CAR, and the transgene that encodes an extracellular region can include, in 5′ to 3′ order, a sequence of nucleotides encoding an extracellular binding domain and a sequence of nucleotides encoding a spacer. In some embodiments, the transgene also includes a sequence of nucleotides encoding one or more extracellular multimerization domain(s), which can be placed 5′ or 3′ of any of the sequence of nucleotides encoding binding domains and/or spacers, and/or 5′ of the sequence of nucleotides encoding a transmembrane domain. In some aspects, the transgene also includes a signal sequence, typically placed 5′ of the sequence of nucleotides encoding the extracellular region.

In some aspects, in the transgene, the sequence of nucleotides encoding the binding domain is placed between the signal sequence and the nucleotides encoding the spacer. In some aspects, in the transgene, the sequence of nucleotides encoding the extracellular multimerization domain is placed between the sequence of nucleotides encoding the binding domain and the sequence of nucleotides encoding the spacer. In some aspects, the sequence of nucleotides encoding the spacer is placed between the sequence of nucleotides encoding the binding domain and the sequence of nucleotides encoding the transmembrane domain.

In some embodiments, the transgene contains a sequence of nucleotides encoding an intracellular region, which can include a sequence of nucleotides encoding one or more costimulatory signaling domain(s) and/or a primary signaling domain or region.

In some embodiments, the transgene also comprises one or more multicistronic element(s), e.g., a ribosome skip sequence and/or an internal ribosome entry site (IRES). In some aspects, the transgene also includes regulatory or control elements, such as a promoter, typically at the most 5′ portion of the transgene, e.g., 5′ of the signal sequence. In some aspects, sequence of nucleotides encoding one or more additional molecule(s) or additional domains or regions can be included in the transgene portion of the polynucleotide. In some aspects, the sequence of nucleotides encoding one or more additional molecule(s) or additional domains or regions can be placed 5′ of the sequence of nucleotides encoding one or more region(s) or domain(s) or chain(s) of the CAR. In some aspects, the sequence of nucleotides encoding the one or more additional molecule(s) or additional domains, regions or chains is upstream of the sequence of nucleotides encoding one or more regions of the CAR.

Exemplary domains or regions of the chimeric receptor encoded by the transgene are described below, and also can include any region or domain of exemplary chimeric receptors described in Sections IV.B.1 and IV.B.3 below.

### (a) Binding Domain

In some embodiments, the transgene encodes a portion of a recombinant receptor, such as a CAR with specificity for a particular antigen (or ligand), such as an antigen expressed on the surface of a particular cell type. In some embodiments, the antigen is selectively expressed or overexpressed on cells of the disease or condition, e.g., the tumor or pathogenic cells, as compared to normal or non-targeted cells or tissues, e.g., in healthy cells or tissues.

In some aspects, the transgene encodes an extracellular region of a recombinant receptor. In some embodiments, the transgene encode extracellular binding domain, such as a binding domain that specifically binds an antigen or a ligand.

In some embodiments, the binding domain is or comprises a polypeptide, a ligand, a receptor, a ligand-binding domain, a receptor-binding domain, an antigen, an epitope, an antibody, an antigen-binding domain, an epitope-binding domain, an antibody-binding domain, a tag-binding domain or a fragment of any of the foregoing. In other embodiments, the antigen is expressed on normal cells and/or is expressed on the engineered cells. In some aspects, the antigen is recognized by a binding domain, such as a ligand binding domain or an antigen binding domain. In some aspects, the transgene encodes an extracellular region containing one or more binding domain(s). In some embodiments, exemplary binding domain encoded by the transgene include antibodies and antigen-binding fragments thereof, including scFv or sdAb. In some embodiments, an antigen-binding fragment comprises antibody variable regions joined by a flexible linker.

In some embodiments, the binding domain is or comprises a single chain variable fragment (scFv). In some embodiments, the binding domain is or comprises a single domain antibody (sdAb). In some embodiments, the binding domain is capable of binding to a target antigen that is associated with, specific to, and/or expressed on a cell or tissue of a disease, disorder or condition. In some embodiments, the disease, disorder or condition is an infectious disease or disorder, an autoimmune disease, an inflammatory disease, or a tumor or a cancer. In some embodiments, the target antigen is a tumor antigen.

Exemplary antigens and antigen- or ligand-binding domains encoded by the transgene include those described in Section IV.B.1 herein. In some aspects, the encoded recombinant receptor contains a binding domain that is or comprises a TCR-like antibody or a fragment thereof, such as an scFv that specifically recognizes an intracellular antigen, such as a tumor-associated antigen, presented on the cell surface as a major histocompatibility complex (MHC)-peptide complex. In some aspects, the transgene can encode a binding domain that is a TCR-like antibody or fragment thereof. Thus, the encoded recombinant receptor is a TCR-like CAR, such as any described herein in Section IV.B. In some embodiments, the binding domain is a multi-specific, such as a bi-specific, binding domain. In some embodiments, the encoded recombinant receptor contains a binding domain that is an antigen that binds to an autoantibody. In some embodiments, the recombinant receptor is a chimeric auto antibody receptor (CAAR), such as any described herein in Section IV.B.3.

In some aspects, sequence of nucleotides encoding the one or more binding domain(s) can be placed 3′ of a signal sequence, if present, in the transgene. In some aspects, sequence of nucleotides encoding the one or more binding domain(s) can be placed 3′ of the sequence of nucleotides encoding one or more regulatory or control element(s), in the transgene. In some aspects, sequence of nucleotides encoding the one or more binding domain(s) can be placed 5′ of the sequence of nucleotides encoding the spacer, if present, in the transgene. In some aspects, sequence of nucleotides encoding the one or more binding domain(s) can be placed 5′ of the sequence of nucleotides encoding transmembrane domain, in the transgene.

### (b) Spacer and Transmembrane Domain

In some embodiments, the encoded recombinant receptor is a CAR, and the transgene includes sequences encoding a spacer and/or sequences encoding a transmembrane domain or portion thereof. In some embodiments, the extracellular region of the encoded recombinant receptor comprises a spacer, in some cases wherein the spacer is operably linked between the binding domain and the transmembrane domain. In some aspects, the spacer and/or transmembrane domain can link the extracellular portion containing the ligand- (e.g., antigen-) binding domain and other regions or domains of the recombinant receptor, such as the intracellular region (e.g., containing one or more costimulatory signaling domain(s), intracellular multimerization domain and/or a primary signaling domain or region).

In some embodiments, the transgene further includes sequence of nucleotides encoding a spacer and/or a hinge region that separates the antigen-binding domain and transmembrane domain. In some aspects, the spacer may be or include at least a portion of an immunoglobulin constant region or variant or modified version thereof, such as a hinge region, e.g., an IgG4 hinge region, and/or a C_{H}1/C_{L} and/or Fc region. In some embodiments, the constant region or portion is of a human IgG, such as IgG4 or IgG1. In some aspects, the portion of the constant region serves as a spacer region between a binding domain, e.g., scFv, and a transmembrane domain. Exemplary spacers that can be encoded by the transgene include IgG4 hinge alone, IgG4 hinge linked to C_{H}2 and C_{H}3 domains, or IgG4 hinge linked to the C_{H}3 domain, and those described in Hudecek et al. (2013) Clin. Cancer Res., 19:3153, Hudecek et al. (2015) Cancer Immunol Res. 3(2): 125-135 or International Pat. App. Pub. No. WO2014031687, or any described in Section IV.B.1 herein.

In some aspects, the sequence of nucleotides encoding the spacer can be placed 3′ of the sequence of nucleotides encoding the one or more binding domains, in the transgene. In some aspects, the sequence of nucleotides encoding the spacer can be placed 5′ of the sequence of nucleotides encoding the transmembrane domain, in the transgene. In some embodiments, the sequence of nucleotides encoding the spacer is placed between the sequence of nucleotides encoding one or more binding domains and the sequence of nucleotides encoding the transmembrane domain.

In some embodiments, the transgene encodes a transmembrane domain, which can link the extracellular region, e.g., containing one or more binding domains and/or spacers, with the intracellular region, e.g., containing one or more costimulatory signaling domain(s), intracellular multimerization domain and/or a primary signaling domain or region. In some embodiments, the transgene comprises a sequence of nucleotides encoding a transmembrane domain, in some cases wherein the transmembrane domain is human or comprises a sequence from a human protein. In some embodiments, the transmembrane domain is or comprises a transmembrane domain derived from CD4, CD28, or CD8, in some cases derived from human CD4, human CD28 or human CD8. In some embodiments, the transmembrane domain is or comprises a transmembrane domain derived from a CD28, in some cases derived from human CD28.

In some embodiments, the sequence of nucleotides encoding transmembrane domain is fused to the sequence of nucleotides encoding the extracellular region. In some embodiments, the sequence of nucleotides encoding transmembrane domain is fused to the sequence of nucleotides encoding the intracellular region. In some aspects, sequence of nucleotides encoding the transmembrane domain can be placed 3′ of the sequence of nucleotides encoding the one or more binding domains and/or the spacer in the transgene. In some aspects, the sequence of nucleotides encoding the transmembrane domain can be placed 5′ of the sequence of nucleotides encoding the intracellular region, e.g., containing one or more costimulatory signaling domain(s), intracellular multimerization domain and/or a primary signaling domain or region, in the transgene. In some aspects, the transmembrane domain encoded by the transgene include any transmembrane domain described herein, for example, in Section IV.B.1.

In some embodiments, in cases where the encoded recombinant receptor comprises an intracellular region comprising a primary signaling domain or region but does not comprise a transmembrane domain and/or an extracellular region, the transgene can include a sequence of nucleotides encoding a membrane association domain, such as any described herein, e.g., in Section IV.B.

### (c) Intracellular Region

In some embodiments, the transgene includes a sequence of nucleotides encoding an intracellular region. In some embodiments, the transgene encodes a CAR, and in some aspects, the intracellular region comprises one or more secondary or co-stimulatory signaling region. In some aspects, the sequence of nucleotides encoding the transmembrane domain can be placed 3′ of the sequence of nucleotides encoding the one or more binding domains and/or the spacer in the transgene, in the transgene. In some aspects, the sequence of nucleotides encoding the one or more costimulatory signaling domain can be placed 5′ of the sequence of nucleotides encoding a primary signaling domain or region. In some aspects, the sequence of nucleotides encoding the one or more costimulatory signaling domain can be placed 3′ of the sequence of nucleotides encoding a primary signaling domain or region. In some aspects, the sequence of nucleotides encoding intracellular region is the most 3′ region in the transgene, which is then linked to one of the homology arm sequences, e.g., the 3′ homology arm sequence. In some aspects, the sequence of nucleotides encoding the one or more costimulatory signaling domain can be placed 3′ of the sequence of nucleotides encoding the transmembrane domain, in the transgene. In some aspects, the costimulatory signaling region or the primary signaling domain or region encoded by the transgene include any costimulatory signaling region or any primary signaling domain or region described herein, for example, in Section IV.B.1.

### (1) Costimulatory Signaling Domain

In some embodiments, the transgene comprises a sequence of nucleotides encoding a portion of the intracellular region, which can include one or more costimulatory signaling domain(s). In some embodiments, the one or more costimulatory signaling domain comprises an intracellular signaling domain of a T cell costimulatory molecule or a signaling portion thereof, optionally wherein the T cell costimulatory molecule or a signaling portion thereof is human.

In some embodiments, the one or more costimulatory signaling domain comprises an intracellular signaling domain of a T cell costimulatory molecule or a signaling portion thereof. In some embodiments, the T cell costimulatory molecule or a signaling portion thereof is human. In some embodiments, exemplary costimulatory signaling domain encoded by the transgene include signaling regions or domains from one or more costimulatory receptor such as CD28, CD137 (4-1BB), OX40 (CD134), CD27, DAP10, DAP12, NKG2D, ICOS and/or other costimulatory receptors, such as any described herein in Section IV.B herein. In some embodiments, the one or more costimulatory signaling domain comprises an intracellular signaling domain of a CD28, a 4-1BB or an ICOS or a signaling portion thereof. In some embodiments, the one or more costimulatory signaling domain comprises a signaling domain of human CD28, human 4-1BB, human ICOS or a signaling portion thereof. In some embodiments, the one or more costimulatory signaling domain comprises an intracellular signaling domain of human 4-1BB.

### (2) Primary Signaling Region or Domain

In some embodiments, the transgene encoding a recombinant receptor, e.g., CAR, includes a sequence of nucleotides encoding a primary signaling region or domain, such as the cytoplasmic domain of CD3zeta (CD3ζ). In some embodiments, the primary signaling region is or comprises a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof) and/or a signaling domain comprising an immunoreceptor tyrosine-based activation motif (ITAM). In some embodiments, the encoded recombinant receptor is any describe herein, for example, in Section IV.B.

In some aspects, the transgene includes a sequence of nucleotides encoding a primary cytoplasmic signaling region that regulates primary stimulation and/or activation of the TCR complex. Primary cytoplasmic signaling region(s) that act in a stimulatory manner may contain signaling motifs which are known as immunoreceptor tyrosine-based activation motifs or ITAMs. Examples of ITAM containing primary cytoplasmic signaling region(s) include those derived from TCR or CD3 zeta (CD3ζ), Fc receptor (FcR) gamma or FcR beta. In some embodiments, cytoplasmic signaling regions or domains in the CAR contain(s) a cytoplasmic signaling domain, portion thereof, or sequence derived from CD3 zeta. In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the primary signaling domain or region encoded by the transgene include any primary signaling domain or region described herein, for example, in Section IV.B.1.

### (d) Additional Domains, e.g., Multimerization Domains

In some embodiments, the transgene also includes a sequence of nucleotides encoding one or more multimerization domain(s), e.g., a dimerization domain. In some aspects, the encoded multimerization domain can be extracellular or intracellular. In some embodiments, the encoded multimerization domain is extracellular. In some embodiments, the encoded multimerization domain is intracellular. In some embodiments, the portion of the intracellular region encoded by the transgene comprises a multimerization domain, optionally a dimerization domain. In some embodiments, the transgene comprises a sequence of nucleotides encoding an extracellular region. In some embodiments, the extracellular region comprises a multimerization domain, optionally a dimerization domain. In some embodiments, the multimerization domain is capable of dimerization upon binding to an inducer.

In some aspects, the recombinant receptor is a multi-chain recombinant receptor, such as a multi-chain CAR. In some embodiments, one or more chains of the multi-chain recombinant receptor or a portion thereof is encoded by the transgene. In some embodiments, one or more chains of the multi-chain recombinant receptor can together form a functional or active recombinant receptor, by virtue of multimerization of the multimerization domain included in each chain of the recombinant receptor.

In some aspects, the sequence of nucleotides encoding a multimerization domain is 5′ or 3′ of other domains. For example, in some embodiments, the encoded multimerization domain is extracellular, and the sequence encoding the multimerization domain is 5′ of the sequence encoding the spacer. In some embodiments, the encoded multimerization domain is intracellular, and the sequence encoding the multimerization domain is 5′ of the sequence encoding the primary signaling region or domain. In some embodiments, the multimerization domain is intracellular, and the sequence encoding the multimerization domain is 5′ or 3′ of the sequence encoding one or more costimulatory signaling domain(s). In some embodiments, the encoded multimerization domain can multimerize (e.g., dimerize), upon binding of an inducer. Exemplary encoded multimerization domain includes any multimerization domain described herein, e.g., in Section IV.B herein.

(iii) Exemplary T Cell Receptor (TCR)-Encoding Sequences

In some embodiments, the recombinant receptor encoded by the transgene is a recombinant T cell receptor (TCR). In some aspects, the transgene can encode all or a portion of the recombinant TCR. In some embodiments, the transgene comprises a sequence of nucleotides encoding one or more chains, regions or domains of a recombinant TCR. Exemplary recombinant TCR encoded by the transgene are described below, and also can include any chains, region or domain of exemplary recombinant TCRs described in Sections IV.B.4 below.

In some embodiments, the TCR, comprises two or more separate polypeptide chains such as TCR alpha (TCRα) and TCR beta (TCRβ) chains. In some aspects, the transgene can encode one or more chains of the recombinant TCR, such as a TCRα or a TCRβ or both. In some aspects, the transgene can encode both TCRα and TCRβ chains. In some aspects, the transgene can encode one of a TCRα chain or a TCRβ chain, and a second transgene present in the engineered cell can encode the other chain of the TCR. In some aspects, the sequences encoding the TCRα and TCRβ are optionally separated by a multicistronic element, such as a 2A element.

In certain embodiments, the transgene includes nucleic acid sequence encoding recombinant receptor is a recombinant TCR or an antigen-binding fragment thereof. In some aspects, the transgene can encode a chain if the recombinant TCR, containing a variable domain and a constant domain. In some aspects, the transgene encodes a chain of a recombinant TCR that contains one or more variable domains and one or more constant domains. In some of embodiments, the transgene contains a sequence encoding a TCRα and a TCRβ chain.

In some embodiments, the encoded TCRα chain and TCRβ chain are separated by a linker region. In some embodiments, a linker sequence is included that links the TCRα and TCRβ chains to form the single polypeptide strand. In some embodiments, the linker is of sufficient length to span the distance between the C terminus of the α chain and the N terminus of the β chain, or vice versa, while also ensuring that the linker length is not so long so that it blocks or reduces bonding to a target peptide-MHC complex. In some embodiments, the linker may be any linker capable of forming a single polypeptide strand, while retaining TCR binding specificity. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)n-P-, wherein n is 5 or 6 and P is proline, G is glycine and S is serine (SEQ ID NO: 22). In some embodiments, the linker has the sequence GSADDAKKDAAKKDGKS (SEQ ID NO: 23). In some embodiments, the linker between the TCRα chain or portion thereof and the TCRβ chain or portion thereof that is recognized by and/or is capable of being cleaved by a protease. In certain embodiments, the linker between the nucleic acid sequence encoding a TCRα chain or portion thereof and the nucleic acid sequence encoding a TCRβ chain or portion thereof contains a multicistronic element.

In some embodiments, the transgene is or include a sequence of nucleotides that is or includes the structure [TCRβ chain]-[linker or multicistronic element]-[TCRα chain]. In some of any embodiments, the transgene is or include a sequence of nucleotides that is or includes the structure [TCRα chain]-[linker or multicistronic element]-[TCRβ chain]. In some aspects, the multicistronic element includes a ribosome skipping element/self-cleavage element (e.g., a 2A element or an internal ribosome entry site (IRES), such as any described herein.

### (iv) Additional Molecules, e.g., Markers

In some embodiments, the transgene also includes a sequence of nucleotides encoding one or more additional molecules, such as an antibody, an antigen, an additional chimeric or additional polypeptide chains of a multi-chain recombinant receptor (e.g., multi-chain CAR, chimeric co-stimulatory receptor, inhibitory receptor, regulatable chimeric antigen receptor or other components of multi-chain recombinant receptor systems described herein, for example, in Section IV.B.2 or a recombinant T cell receptor (TCR) described in Section IV.B.3), a transduction marker or a surrogate marker (e.g., truncated cell surface marker), an enzyme, an factors, a transcription factor, an inhibitory peptide, a growth factor, a nuclear receptor, a hormone, a lymphokine, a cytokine, a chemokine, a soluble receptor, a soluble cytokine receptor, a soluble chemokine receptor, a reporter, functional fragments or functional variants of any of the foregoing and combinations of the foregoing. In some aspects, such sequence of nucleotides encoding one or more additional molecules can be placed 5′ of the sequence of nucleotides encoding regions or domains of the recombinant receptor. In some aspects, the sequences encoding one or more other molecules and the sequence of nucleotides encoding regions or domains of the recombinant receptor are separated by regulatory sequences, such as a 2A ribosome skipping element and/or promoter sequences.

In some embodiments, the transgene also includes a sequence of nucleotides encoding one or more additional molecules. In some aspects, one or more additional molecules include one or more marker(s). In some embodiments, the one or more marker(s) includes a transduction marker, a surrogate marker and/or a selection marker. In some embodiments, the transgene also includes nucleic acid sequences that can improve the efficacy of therapy, such as by promoting viability and/or function of transferred cells; nucleic acid sequences to provide a genetic marker for selection and/or evaluation of the cells, such as to assess in vivo survival or localization; nucleic acid sequences to improve safety, for example, by making the cell susceptible to negative selection in vivo as described by Lupton et al., Mol. and Cell Biol., 11:6 (1991); and Riddell et al., Human Gene Therapy 3:319-338 (1992); see also WO 1992008796 and WO 1994028143 describing the use of bifunctional selectable fusion genes derived from fusing a dominant positive selectable marker with a negative selectable marker, and U.S. Pat. No. 6,040,177. In some aspects, the markers include any markers described herein, for example, in this section or Section II or III, or any additional molecules and/or receptor polypeptides described herein, for example, in Section IV.B.2. In some embodiments, the additional molecule is a surrogate marker, optionally a truncated receptor, optionally wherein the truncated receptor lacks an intracellular signaling domain and/or is not capable of mediating intracellular signaling when bound by its ligand.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR or a TCR. In some of any embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the transgene includes sequences encoding one or more additional molecule that is an immunomodulatory agent. In some embodiments, the immunomodulatory molecule is selected from an immune checkpoint modulator, an immune checkpoint inhibitor, a cytokine or a chemokine. In some embodiments, the immunomodulatory agent is an immune checkpoint inhibitor capable of inhibiting or blocking a function of an immune checkpoint molecule or a signaling pathway involving an immune checkpoint molecule. In some embodiments, the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing. Other exemplary additional molecules include epitope tags, detectable molecules such as fluorescent or luminescent proteins, or molecules that mediate enhanced cell growth and/or gene amplification (e.g., dihydrofolate reductase). Epitope tags include, for example, one or more copies of FLAG, His, myc, Tap, HA or any detectable amino acid sequence. In some embodiments, additional molecules can include non-coding sequences, inhibitory nucleic acid sequences, such as antisense RNAs, RNAi, shRNAs and micro RNAs (miRNAs), or nuclease recognition sequences.

In some aspects, the additional molecule can include any additional receptor polypeptides described herein, such as any additional polypeptide chain of the multi-chain recombinant receptor, e.g., as described in Section IV.B.2.

### (v) Multicistronic Elements and Regulatory or Control Elements

In some aspects, the transgene, including the transgene encoding a recombinant receptor or a portion thereof, can be inserted so that its expression is driven by the endogenous promoter at the integration site, namely the promoter that drives expression of the endogenous T cell stimulation-associated locus gene. In some embodiments in which the polypeptide encoding sequences are promoterless, the expression of the integrated transgene is then ensured by transcription driven by an endogenous promoter or other control element in the region of interest. For example, the transgene encoding a portion of the recombinant receptor can be inserted without a promoter, but in-frame with the coding sequence of the endogenous T cell stimulation-associated locus, such that expression of the integrated transgene is controlled by the transcription of the endogenous promoter and/or other regulatory elements at the integration site. In some embodiments, a multicistronic element such as a ribosome skipping element/self-cleavage element (e.g., a 2A element or an internal ribosome entry site (IRES)), is placed upstream of the transgene encoding a portion of the recombinant receptor, such that the multicistronic element is placed in-frame with one or more exons of the endogenous open reading frame at the T cell stimulation-associated locus, such that the expression of the transgene encoding the recombinant receptor is operably linked to the endogenous T cell stimulation-associated locus promoter. In some embodiments, the transgene does not comprise a sequence encoding a 3′ UTR. In some embodiments, upon integration of the transgene into the endogenous T cell stimulation-associated locus, the transgene is integrated upstream of the 3′ UTR of the endogenous T cell stimulation-associated locus, such that the message encoding the recombinant receptor contains a 3′ UTR of the endogenous T cell stimulation-associated locus, e.g., from the open reading frame or partial sequence thereof of the endogenous T cell stimulation-associated locus. In some embodiments, the open reading frame or a partial sequence thereof encoding the remaining portion of the recombinant receptor comprises a 3′ UTR of the endogenous T cell stimulation-associated locus.

In some embodiments, a “tandem” cassette is integrated into the selected site. In some embodiments, one or more of the “tandem” cassettes encode one or more polypeptide or factors, each independently controlled by a regulatory element or all controlled as a multi-cistronic expression system. In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows co-expression of gene products by a message from a single promoter. Alternatively, in some cases, a single promoter may direct expression of an RNA that contains, in a single open reading frame (ORF), two or three polypeptides separated from one another by sequences encoding a self-cleavage peptide (e.g., 2A sequences) or a protease recognition site (e.g., furin), as described herein. The ORF thus encodes a single polypeptide, which, either during (in the case of 2A) or after translation, is processed into the individual proteins. In some embodiments, the “tandem cassette” includes the first component of the cassette comprising a promoterless sequence, followed by a transcription termination sequence, and a second sequence, encoding an autonomous expression cassette or a multi-cistronic expression sequence. In some embodiments, the tandem cassette encodes two or more different polypeptides or factors, e.g., two or more chains or domains of a recombinant receptor. In some embodiments, nucleic acid sequence encoding two or more chains or domains of the recombinant receptor are introduced as tandem expression cassettes or bi- or multi-cistronic cassettes, into one target DNA integration site.

In some embodiments, the transgene (e.g., exogenous nucleic acid sequences) also contains one or more heterologous or exogenous regulatory or control elements, e.g., cis-regulatory elements, that are not, or are different from the regulatory or control elements of the endogenous T cell stimulation-associated locus. In some embodiments, the heterologous or exogenous regulatory or control element is operably linked to nucleic acid sequence encoding an additional component of the transgene, e.g., a nucleic acid sequence encoding an additional polypeptide, apart from the nucleic acid sequence encoding the recombinant receptor.

In some aspects, the heterologous regulatory or control elements include such as a promoter, an enhancer, an intron, an insulator, a polyadenylation signal, a transcription termination sequence, a Kozak consensus sequence, a multicistronic element (e.g., internal ribosome entry sites (IRES), a 2A sequence), sequences corresponding to untranslated regions (UTR) of a messenger RNA (mRNA), and splice acceptor or donor sequences, such as those that are not, or are different from the regulatory or control element at the T cell stimulation-associated locus. In some embodiments, the heterologous regulatory or control elements include a promoter, an enhancer, an intron, a polyadenylation signal, a Kozak consensus sequence, a splice acceptor sequence and/or a splice donor sequence. In some embodiments, the transgene comprises a promoter that is heterologous and/or not typically present at or near the target site, for example, to control the expression of additional components in the transgene.

In some cases, the multicistronic element, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004); also referred to as a self-cleavage element). This allows the inserted transgene to be controlled by the transcription of the endogenous promoter at the integration site such as a T cell stimulation-associated locus promoter. Exemplary multicistronic element include 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18, 19 or 61) as described in U.S. Patent Pub. No. 20070116690. In some embodiments, the template polynucleotide includes a P2A ribosome skipping element (sequence set forth in SEQ ID NO: 18, 19 or 61) upstream of the transgene, e.g., nucleic acids encoding the recombinant receptor or portion thereof.

In some embodiments, the transgene encoding the one or more chains of a recombinant receptor or portion thereof and/or the sequences encoding an additional molecule independently comprises one or more multicistronic element(s). In some embodiments, the one or more multicistronic element(s) are upstream of the transgene encoding the recombinant receptor portion thereof and/or the sequences encoding an additional molecule. In some embodiments, the multicistronic element(s) is positioned between the transgene encoding the recombinant receptor portion thereof and/or the sequences encoding an additional molecule. In some embodiments, the multicistronic element(s) is positioned between the nucleic acid sequence encoding portions or chains of the recombinant receptor.

In some embodiments, the sequence encoding an additional molecule is operably linked to a heterologous regulatory or control element. In some aspects, the heterologous regulatory or control element comprises a heterologous promoter. In some embodiments, the heterologous promoter is selected from among a constitutive promoter, an inducible promoter, a repressible promoter, and/or a tissue-specific promoter. In some embodiments, regulatory or control element is a promoter and/or enhancer, for example a constitutive promoter or an inducible or tissue-specific promoter. In some embodiments, the promoter is selected from among an RNA pol I, pol II or pol III promoter. In some embodiments, the promoter is recognized by RNA polymerase II (e.g., a CMV, SV40 early region or adenovirus major late promoter). In some embodiments, the promoter is recognized by RNA polymerase III (e.g., a U6 or H1 promoter). In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the heterologous promoter is or comprises a human elongation factor 1 alpha (EF1α) promoter or an MND promoter or a variant thereof.

In some embodiments, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence or is an analog thereof or is capable of being bound by or recognized by a Lac repressor or an analog thereof. In some embodiments, the promoter is a tissue-specific promoter. In some instances, the promoter is only expressed in a specific cell type (e.g., a T cell or B cell or NK cell specific promoter).

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In some instances, the promoter drives expression only in a specific cell type (e.g., a T cell or B cell or NK cell specific promoter).

In some embodiments, the promoter is a viral promoter. In some embodiments, the promoter is a non-viral promoter. In some cases, the promoter is selected from among human elongation factor 1 alpha (EF1α) promoter or a modified form thereof (EF1α promoter with HTLV1 enhancer) or the MND promoter. In some embodiments, the polynucleotide does not include a heterologous or exogenous regulatory element, e.g., a promoter. In some embodiments, the promoter is a bidirectional promoter (see, e.g., WO2016/022994).

In some embodiments, the transgene may also include splice acceptor sequences. Exemplary known splice acceptor site sequences include, e.g., CTGACCTCTTCTCTTCCTCCCACAG (SEQ ID NO:289) (from the human HBB gene) and TTTCTCTCCACAG (SEQ ID NO:290) (from the human IgG gene).

In some embodiments, the transgene may also include sequences required for transcription termination and/or polyadenylation signal. In some aspects, exemplary polyadenylation signal is selected from SV40, hGH, BGH, and rbGlob transcription termination sequence and/or polyadenylation signal. In some embodiments, the transgene includes an SV40 polyadenylation signal. In some embodiments, if present within the transgene, the transcription termination sequence and/or polyadenylation signal is typically the most 3′ sequence within the transgene, and is linked to one of the homology arm. In some aspects, the transgene does not comprise a sequence encoding a 3′ UTR or a transcription terminator. In some embodiments, upon integration of the transgene into the endogenous T cell stimulation-associated locus, the transgene is integrated upstream of the 3′ UTR and/or the transcription terminator of the endogenous T cell stimulation-associated locus, such that the message encoding the recombinant receptor contains a 3′ UTR of the endogenous T cell stimulation-associated locus, e.g., from the open reading frame or partial sequence thereof of the endogenous T cell stimulation-associated locus. Thus, in some embodiments, upon integration of the transgene encoding a portion of the recombinant receptor, the nucleic acid sequence encoding the recombinant receptor is operably linked to be under the control of 3′ UTR, transcription terminator and/or other regulatory elements of the endogenous T cell stimulation-associated locus.

### (vi) Exemplary Transgene Sequences

In some embodiments, an exemplary transgene includes, in 5′ to 3′ order, sequence of nucleotides each encoding: a transmembrane domain (or a membrane association domain) and an intracellular region. In some embodiments, an exemplary transgene includes, in 5′ to 3′ order, sequence of nucleotides each encoding: an extracellular region, a transmembrane domain and an intracellular region.

In some embodiments, the encoded recombinant receptor is a CAR, and an exemplary transgene comprises, in 5′ to 3′ direction, sequence of nucleotides each encoding: a signal peptide, an extracellular binding domain, a spacer, a transmembrane domain and an intracellular region comprising a primary signaling domain or region and/or a co-stimulatory signaling domain. In some embodiments, an exemplary transgene comprises, in 5′ to 3′ direction, sequence of nucleotides each encoding: a signal peptide, an extracellular binding domain, a spacer, a transmembrane domain and one or more costimulatory signaling domains. In some embodiments, an exemplary transgene comprises, in 5′ to 3′ direction, sequence of nucleotides each encoding: a signal peptide, an extracellular binding domain, a spacer, a transmembrane domain and one or more costimulatory signaling domains and primary signaling domain or region.

In some embodiments, an exemplary transgene comprises, in 5′ to 3′ direction, sequence of nucleotides each encoding: a transmembrane domain (or a membrane association domain), an intracellular multimerization domain, optionally one or more costimulatory signaling domain(s), and a primary signaling domain or region. In some embodiments, an exemplary transgene comprises, in 5′ to 3′ direction, sequence of nucleotides each encoding: an extracellular multimerization domain, a transmembrane domain, optionally one or more costimulatory signaling domain(s), and a primary signaling domain or region.

In some embodiments, the transgene comprises, in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof. In some embodiments, the encoded intracellular region of the recombinant receptor comprises, from its N to C terminus in order: the one or more costimulatory signaling domain(s) and a primary signaling domain or region, such as containing a CD3zeta chain or a fragment thereof.

In some embodiments, an exemplary transgene includes, in 5′ to 3′ order, sequence of nucleotides each encoding: a transmembrane domain (or a membrane association domain) and an intracellular region. In some embodiments, an exemplary transgene includes, in 5′ to 3′ order, sequence of nucleotides each encoding: an extracellular region, a transmembrane domain and an intracellular region.

In some embodiments, an exemplary transgene encodes all or a portion of a TCRα chain. In some embodiments, an exemplary transgene encodes all or a portion of a TCRβ chain. In some embodiments, an exemplary transgene encodes all or a portion of both a TCRα chain and a TCRβ chain. In some embodiments, the encoded recombinant receptor is a recombinant T cell receptor (TCR) and an exemplary transgene includes, in 5′ to 3′ order, [TCRβ chain]-[linker or multicistronic element]-[TCRα chain]. In some embodiments, the encoded recombinant receptor is a recombinant TCR and an exemplary transgene includes, in 5′ to 3′ order, [TCRα chain]-[linker or multicistronic element]-[TCRβ chain].

In some embodiments, the exemplary transgene can also comprise a multicistronic element, e.g., a 2A element or an internal ribosome entry site (IRES), and/or a regulatory or control element, e.g., a promoter, placed 5′ of the sequences encoding the signal peptide and/or the extracellular region. In some embodiments, the exemplary transgene can also comprise additional sequences, e.g., sequence of nucleotides encoding one or more additional molecules, such as a marker, an additional recombinant receptor, an antibody or an antigen-binding fragment thereof, an immunomodulatory molecule, a ligand, a cytokine or a chemokine. In some aspects, the sequences encoding one or more other molecules and the sequence of nucleotides encoding regions or domains of the recombinant receptor are separated by regulatory sequences, such as a 2A ribosome skipping element and/or promoter sequences. In some aspects, in the exemplary transgene, the sequence of nucleotides encoding one or more additional molecules is placed 5′ of the sequences encoding the signal peptide and/or the extracellular region. In some embodiments, the sequence of nucleotides encoding one or more additional molecules is placed between the multicistronic element and/or regulatory or control element, and the sequence of nucleotides encoding regions or domains of the recombinant receptor. In some embodiments, the sequence of nucleotides encoding one or more additional molecules is placed between two elements and/or regulatory or control elements. In some embodiments, an exemplary transgene comprises, in 5′ to 3′ direction: a multicistronic element and/or a regulatory element, a sequence of nucleotides encoding an additional molecule, a multicistronic element and/or a regulatory element, a signal peptide, nucleic acid sequence encoding regions or domains of the recombinant receptor (e.g., extracellular region, transmembrane domain, intracellular region).

b. Homology Arms

In some embodiments, the template polynucleotide contains one or more homology sequences (also called “homology arms”) on the 5′ and 3′ ends, linked to or surrounding the transgene encoding a recombinant receptor or a portion thereof. The homology arms allow the DNA repair mechanisms, e.g., homologous recombination machinery, to recognize the homology and use the template polynucleotide as a template for repair, and the nucleic acid sequence between the homology arms are copied into the DNA being repaired, effectively inserting or integrating the transgene into the target site of integration in the genome between the location of the homology.

In some aspects, upon integration of the transgene, the entire recombinant receptor is encoded by the transgene, and the entire coding sequence or a portion of the coding sequences of the endogenous T cell stimulation-associated locus is deleted. In some embodiments, the transgene comprises a sequence of nucleotides that is in-frame with one or more exons of the open reading frame of the T cell stimulation-associated locus comprised in the one or more homology arm(s). In some aspects, the entire recombinant receptor is encoded by the transgene, and only a portion of the T cell stimulation-associated locus is deleted, and the remaining portion of the endogenous T cell stimulation-associated locus is expressed.

In some embodiments, the homology arm sequences include sequences that are homologous to the genomic sequences surrounding the genetic disruption, e.g., a target site within the T cell stimulation-associated locus. In some embodiments, the template polynucleotide comprises the following components: [5′ homology arm]-[a transgene (exogenous or heterologous nucleic acid sequences, e.g., encoding a recombinant receptor or a portion thereof)]-[3′ homology arm]. In some embodiments, the 5′ homology arm sequences include contiguous sequences that are homologous to sequences located near the genetic disruption on the 5′ side. In some embodiments, the 3′ homology arm sequences include contiguous sequences that are homologous to sequences located near the genetic disruption on the 3′ side. In some aspects, the target site is determined by targeting of the one or more agent(s) capable of introducing a genetic disruption, e.g., Cas9 and gRNA targeting a specific site within the T cell stimulation-associated locus.

In some aspects, the transgene within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of the transgene. In some aspects, the homology arms are designed to target integration within an exon of the open reading frame of the endogenous T cell stimulation-associated locus, and the homology arm sequences are determined based on the desired location of integration surrounding the genetic disruption, including exon and intron sequences surrounding the genetic disruption. In some embodiments, the location of the target site, relative location of the one or more homology arm(s), and the transgene (exogenous nucleic acid sequence) for insertion can be designed depending on the requirement for efficient targeting and the length of the template polynucleotide or vector that can be used. In some aspects, the homology arms are designed to target integration within an intron of the open reading frame of the T cell stimulation-associated locus. In some aspects, the homology arms are designed to target integration within an exon of the open reading frame of the T cell stimulation-associated locus.

In some aspects, the target integration site (site for targeted integration) within the T cell stimulation-associated locus is located within an open reading frame at the endogenous T cell stimulation-associated locus. In some embodiments, the target integration site is at or near any of the target sites described herein, e.g., in Section II.A. In some aspects, the target location for integration is at or around the target site for genetic disruption, e.g., within less than 500, 450, 400, 350, 300, 250, 200, 150, 100 or 50 bp of the target site for genetic disruption.

In some aspects, the target integration site is within an exon of the open reading frame of the endogenous T cell stimulation-associated locus. In some aspects, the target integration site is within an intron of the open reading frame of the T cell stimulation-associated locus. In some aspects, the target integration site is within a regulatory or control element, e.g., a promoter, of the T cell stimulation-associated locus. In some embodiments, the target integration site is within or in close proximity to exons corresponding to early coding region, e.g., exon 1, 2, 3, 4 or 5 of the open reading frame of the endogenous T cell stimulation-associated locus, or including sequence immediately following a transcription start site, within exon 1, 2, 3, 4 or 5 (such as described in Tables 1-9 herein), or within less than 500, 450, 400, 350, 300, 250, 200, 150, 100 or 50 bp of exon 1, 2, 3, 4 or 5. In some embodiments, the integration is targeted at or near exon 2 of the endogenous T cell stimulation-associated locus, or within less than 500, 450, 400, 350, 300, 250, 200, 150, 100 or 50 bp of exon 2. In some aspects, the target integration site is at or near exon 1 of the endogenous T cell stimulation-associated locus, e.g., within less than 500, 450, 400, 350, 300, 250, 200, 150, 100 or 50 bp of exon 1. In some embodiments, the target integration site is at or near exon 2 of the endogenous T cell stimulation-associated locus, or within less than 500, 450, 400, 350, 300, 250, 200, 150, 100 or 50 bp of exon 2. In some aspects, the target integration site is at or near exon 3 of the endogenous T cell stimulation-associated locus, e.g., within less than 500, 450, 400, 350, 300, 250, 200, 150, 100 or 50 bp of exon 3. In some aspects, the target integration site is at or near exon 4 of the endogenous T cell stimulation-associated locus, e.g., within less than 500, 450, 400, 350, 300, 250, 200, 150, 100 or 50 bp of exon 4. In some aspects, the target integration site is at or near exon 5 of the endogenous T cell stimulation-associated locus, e.g., within less than 500, 450, 400, 350, 300, 250, 200, 150, 100 or 50 bp of exon 5. In some aspects, the target integration site is within a regulatory or control element, e.g., a promoter, of the T cell stimulation-associated locus.

In some embodiments, the 5′ homology arm sequences include contiguous sequences of approximately 10, 20, 30, 40, 50, 100, 200, 300, 400, 500, 600, 700, 800, 900, 1000, 1500, 2000, 3000, 4000, or 5000 base pairs 5′ of the target site for genetic disruption, starting near the target site at the endogenous T cell stimulation-associated locus. In some embodiments, the 3′ homology arm sequences include contiguous sequences of approximately 10, 20, 30, 40, 50, 100, 200, 300, 400, 500, 600, 700, 800, 900, 1000, 1500, 2000, 3000, 4000, or 5000 base pairs 3′ of the target site for genetic disruption, starting near the target site at the endogenous T cell stimulation-associated locus. Thus, upon integration via HDR, the transgene is targeted for integration at or near the target site for genetic disruption, e.g., a target site within an exon or intron of the endogenous T cell stimulation-associated locus.

In some aspects, the homology arms contain sequences that are homologous to a portion of an open reading frame sequence at the endogenous T cell stimulation-associated locus. In some aspects, the homology arm sequences contain sequences homologous to contiguous portion of an open reading frame sequence, including exons and introns, at the endogenous T cell stimulation-associated locus. In some aspects, the homology arm contains sequences that are identical to a contiguous portion of an open reading frame sequence, including exons and introns, at the endogenous T cell stimulation-associated locus.

In some embodiments, the template polynucleotide contains homology arms for targeting integration of the transgene at the endogenous T cell stimulation-associated locus (exemplary genomic locus sequence described in Tables 1-9 herein; exemplary human mRNA sequence described in Section II.A.1 above) In some embodiments, the genetic disruption is introduced using any of the agents for genetic disruption, e.g., targeted nucleases and/or gRNAs described herein. In some embodiments, the template polynucleotide comprises about 500 to 1000, e.g., 500 to 900 or 600 to 700, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequences, which is homologous to 500, 600, 700, 800, 900 or 1000 base pairs of sequences 5′ of the genetic disruption at a T cell stimulation-associated locus, the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption at a T cell stimulation-associated locus.

In some aspects, the boundary between the transgene and the one or more homology arm sequences, is designed such that upon HDR and targeted integration of the transgene, the sequences within the transgene that encode one or more polypeptide, e.g., chain(s), domain(s) or region(s) of a recombinant receptor, is integrated in-frame with one or more exons of the open reading frame sequence at the endogenous T cell stimulation-associated locus, and/or generates an in-frame fusion of the transgene that encode a polypeptide and one or more exons of the open reading frame sequence at the endogenous T cell stimulation-associated locus. In some embodiments, all or a portion of the gene product of the T cell stimulation-associated locus is encoded by the nucleic acid sequences of the endogenous open reading frame, and a polypeptide of the recombinant receptor or a portion thereof is encoded by the integrated transgene, optionally, separated by a multicistronic element, such as a 2A element.

In some embodiments, the one or more homology arm sequences include sequences that are homologous, substantially identical or identical to sequences that surround or flank the target site that are within an open reading frame sequence at the endogenous T cell stimulation-associated locus. In some aspects, the one or more homology arm sequences contain introns and exons of a partial sequence of an open reading frame at the endogenous T cell stimulation-associated locus. In some aspects, the boundary of the 5′ homology arm sequence and the transgene is such that, in a case of a transgene that does not contain a heterologous promoter, the coding portion of the transgene is fused in-frame with an upstream exon or a portion thereof, e.g., exon 1, 2, 3, 4 or 5, depending on the location of targeted integration, of the open reading frame of the endogenous T cell stimulation-associated locus.

In some aspects, the boundary of the 5′ homology arm sequence and the transgene is such that, the upstream exons or a portion thereof, e.g., exons 1, 2, 3, 4, or 5, of the open reading frame of the endogenous T cell stimulation-associated locus, is fused in-frame with the coding portions of the transgene. Thus, upon targeted integration, transcription and translation, the encoded recombinant receptor that is a contiguous polypeptide is produced, from a fusion DNA sequence of an open reading frame sequence of the endogenous T cell stimulation-associated locus and the transgene. In some aspects, the upstream exons or a portion thereof encode all or a portion of the gene product of the T cell stimulation-associated locus. In some aspects, upon targeted integration, a multicistronic element, e.g., a 2A element or an internal ribosome entry site (IRES) separates the open reading frame sequence of the endogenous T cell stimulation-associated locus and the transgene encoding the recombinant receptor. In some aspects, when expressed and translated from the modified T cell stimulation-associated locus, the polypeptide is cleaved to generate all or a portion of the polypeptide encoded by the endogenous T cell stimulation-associated locus and a recombinant receptor.

In some embodiments, exemplary 5′ homology arm for targeting integration at the endogenous T cell stimulation-associated locus PDCD1 comprises the sequence set forth in SEQ ID NO:66, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 66 or a partial sequence thereof.

In some embodiments, exemplary 3′ homology arm for targeting integration at the endogenous T cell stimulation-associated locus PDCD1 comprises the sequence set forth in SEQ ID NO:67, or a sequence that exhibits at least 85%, 86%, 87%, 88%, 89%, 90%, 91%, 92%, 93%, 94%, 95%, 96%, 97%, 98%, 99% or more sequence identity to SEQ ID NO:67 or a partial sequence thereof.

In some embodiments, exemplary 5′ homology arm for targeting integration at the endogenous TRAC locus comprises the sequence set forth in SEQ ID NO:68, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 68 or a partial sequence thereof.

In some embodiments, exemplary 3′ homology arm for targeting integration at the endogenous TRAC locus comprises the sequence set forth in SEQ ID NO:69, or a sequence that exhibits at least 85%, 86%, 87%, 88%, 89%, 90%, 91%, 92%, 93%, 94%, 95%, 96%, 97%, 98%, 99% or more sequence identity to SEQ ID NO:69 or a partial sequence thereof.

In some aspects, the target site can determine the relative location and sequences of the homology arms. The homology arm can typically extend at least as far as the region in which end resection by the DNA repair mechanism can occur after the genetic disruption, e.g., DSB, is introduced, e.g., in order to allow the resected single stranded overhang to find a complementary region within the template polynucleotide. The overall length could be limited by parameters such as plasmid size, viral packaging limits or construct size limit.

In some embodiments, the homology arm comprises about 500 to 1000, e.g., 600 to 900 or 700 to 800, base pairs of homology on either side of the target site at the endogenous gene. In some embodiments, the homology arm comprises about at least or less than or about 200, 300, 400, 500, 600, 700, 800, 900 or 1000 base pairs homology 5′ of the target site, 3′ of the target site, or both 5′ and 3′ of the target site at T cell stimulation-associated locus.

In some embodiments, the homology arm comprises at or about 10, 20, 30, 40, 50, 100, 200, 300, 400, 500, 600, 700, 800, 900, 1000, 1500, 2000, 3000, 4000, or 5000 base pairs homology 3′ of the target site at T cell stimulation-associated locus. In some embodiments, the homology arm comprises at or about 100 to 500, 200 to 400 or 250 to 350, base pairs homology 3′ of the transgene and/or target site at T cell stimulation-associated locus. In some embodiments, the homology arm comprises less than about 100, 90, 80, 70, 60, 50, 40, 30, 20, 15, or 10 base pairs homology 5′ of the target site at T cell stimulation-associated locus.

In some embodiments, the homology arm comprises at or about 10, 20, 30, 40, 50, 100, 200, 300, 400, 500, 600, 700, 800, 900, 1000, 1500, 2000, 3000, 4000, or 5000 base pairs homology 5′ of the target site at T cell stimulation-associated locus. In some embodiments, the homology arm comprises at or about 100 to 500, 200 to 400 or 250 to 350, base pairs homology 5′ of the transgene and/or target site at T cell stimulation-associated locus. In some embodiments, the homology arm comprises less than about 100, 90, 80, 70, 60, 50, 40, 30, 20, 15, or 10 base pairs homology 3′ of the target site at T cell stimulation-associated locus.

In some embodiments, the 3′ end of the 5′ homology arm is the position next to the 5′ end of the transgene. In some embodiments, the 5′ homology arm can extend at least at or about 10, 20, 30, 40, 50, 100, 200, 300, 400, 500, 600, 700, 800, 900, 1000, 1500, 2000, 3000, 4000, or 5000 nucleotides 5′ from the 5′ end of the transgene.

In some embodiments, the 5′ end of the 3′ homology arm is the position next to the 3′ end of the transgene. In some embodiments, the 3′ homology arm can extend at least at or about 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ the homology arms, may each comprise about 1000 base pairs (bp) of sequence flanking the most distal target sites (e.g., 1000 bp of sequence on either side of the mutation).

Exemplary homology arm lengths include at least at or about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include less than at or about or is or is about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or 750 to at or about 1000 nucleotides.

In some of any such embodiments, the transgene is integrated by a template polynucleotide introduced into each of a plurality of T cells. In some of any embodiments, the template polynucleotide comprises the structure [5′ homology arm]-[transgene]-[3′ homology arm]. In certain embodiments, the 5′ homology arm and the 3′ homology arm comprises nucleic acid sequences homologous to nucleic acid sequences surrounding the at least at or about one target site. In some embodiments, the 5′ homology arm comprises nucleic acid sequences that are homologous to nucleic acid sequences 5′ of the target site. In some of any embodiments, the 3′ homology arm comprises nucleic acid sequences that are homologous to nucleic acid sequences 3′ of the target site. In certain embodiments, the 5′ homology arm and the 3′ homology arm independently are at least at or about or at least at or about 10, 20, 30, 40, 50, 100, 200, 300, 400, 500, 600, 700, 800, 900, 1000, 1500, or 2000 nucleotides, or less than at or about 10, 20, 30, 40, 50, 100, 200, 300, 400, 500, 600, 700, 800, 900, 1000, 1500, or 2000 nucleotides. In some embodiments, the 5′ homology arm and the 3′ homology arm independently are between at or about 50 and at or about 100, 100 and at or about 250, 250 and at or about 500, 500 and at or about 750, 750 and at or about 1000, 1000 and at or about 2000 nucleotides. In some of any such embodiments, the 5′ homology arm and the 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

In some of any embodiments, the 5′ homology arm and the 3′ homology arm independently are from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides. In some of any embodiments, the 5′ homology arm and the 3′ homology arm independently are from at or about 100 to at or about at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length. In some embodiments, the 5′ homology arm and the 3′ homology arm independently are at or about 200, 300, 400, 500, 600, 700 or 800 nucleotides in length, or any value between any of the foregoing. In some embodiments, the 5′ homology arm and the 3′ homology arm independently are greater than at or about 300 nucleotides in length, optionally wherein the 5′ homology arm and the 3′ homology arm independently are at or about 400, 500 or 600 nucleotides in length or any value between any of the foregoing. In some embodiments, the 5′ homology arm and the 3′ homology arm independently are greater than at or about 300 nucleotides in length.

In some embodiments, one or more of the homology arms contain a sequence of nucleotides are homologous to sequences that encode a gene product of the T cell stimulation-associated locus or a fragment thereof. In some embodiments, one or more homology arms are connected or linked in frame with the transgene encoding a recombinant receptor or a portion thereof.

In some embodiments, alternative HDR is employed. In some embodiments, alternative HDR proceeds more efficiently when the template polynucleotide has extended homology 5′ to the target site (i.e., in the 5′ direction of the target site strand). Accordingly, in some embodiments, the template polynucleotide has a longer homology arm and a shorter homology arm, wherein the longer homology arm can anneal 5′ of the target site. In some embodiments, the arm that can anneal 5′ to the target site is at least 25, 50, 75, 100, 125, 150, 175, or 200, 300, 400, 500, 600, 700, 800, 900, 1000, 1500, 2000, 3000, 4000, or 5000 nucleotides from the target site or the 5′ or 3′ end of the transgene. In some embodiments, the arm that can anneal 5′ to the target site is at least 10%, 20%, 30%, 40%, or 50% longer than the arm that can anneal 3′ to the target site. In some embodiments, the arm that can anneal 5′ to the target site is at least 2×, 3×, 4×, or 5× longer than the arm that can anneal 3′ to the target site. Depending on whether a ssDNA template can anneal to the intact strand or the targeted strand, the homology arm that anneals 5′ to the target site may be at the 5′ end of the ssDNA template or the 3′ end of the ssDNA template, respectively.

Similarly, in some embodiments, the template polynucleotide has a 5′ homology arm, a transgene, and a 3′ homology arm, such that the template polynucleotide contains extended homology to the 5′ of the target site. For example, the 5′ homology arm and the 3′ homology arm may be substantially the same length, but the transgene may extend farther 5′ of the target site than 3′ of the target site. In some embodiments, the homology arm extends at least 10%, 20%, 30%, 40%, 50%, 2×, 3×, 4×, or 5× further to the 5′ end of the target site than the 3′ end of the target site.

In some embodiments alternative HDR proceeds more efficiently when the template polynucleotide is centered on the target site. Accordingly, in some embodiments, the template polynucleotide has two homology arms that are essentially the same size. In some embodiments, the first homology arm (e.g., 5′ homology arm) of a template polynucleotide may have a length that is within 10%, 9%, 8%, 7%, 6%, 5%, 4%, 3%, 2%, or 1% of the second homology arm (e.g., 3′ homology arm) of the template polynucleotide.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

In some embodiments, the length of the template polynucleotide, including the transgene and the one or more homology arms, is between or between about 1000 to about 20,000 base pairs, such as about 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000 or 20000 base pairs. In some embodiments, the length of the template polynucleotide is limited by the maximum length of polynucleotide that can be prepared, synthesized or assembled and/or introduced into the cell or the capacity of the viral vector, and the type of polynucleotide or vector. In some aspects, the limited capacity of the template polynucleotide can determine the length of the transgene and/or the one or more homology arms. In some aspects, the combined total length of the transgene and the one or more homology arms must be within the maximum length or capacity of the polynucleotide or vector. For example, in some aspects, the transgene portion of the template polynucleotide is about 1000, 1500, 2000, 2500, 3000, 3500 or 4000 base pairs, and if the maximum length of the template polynucleotide is about 5000 base pairs, the remaining portion of the sequence can be divided among the one or more homology arms, e.g., such that the 3′ or 5′ homology arms can be approximately 500, 750, 1000, 1250, 1500, 1750 or 2000 base pairs.

### 3. Delivery of Template Polynucleotides

In some embodiments, the polynucleotide, e.g., a polynucleotide such as a template polynucleotide encoding a recombinant receptor or a portion thereof (for example, described in Section II.B.2 herein), are introduced into the cells in nucleotide form, e.g., as a polynucleotide or a vector. In some of any embodiments, the polynucleotide contains a transgene that encodes the recombinant receptor or a portion thereof and one or more homology arms, and can be introduced into the cell for homology-directed repair (HDR)-mediated integration of the transgene.

In some aspects, the provided embodiments genetic engineering of cells, by the introduction of one or more agent(s) or components thereof capable of inducing a genetic disruption and a template polynucleotide, to induce (HDR and targeted integration of the transgene. In some aspects, the one or more agent(s) and the template polynucleotide are delivered simultaneously. In some aspects, the one or more agent(s) and the template polynucleotide are delivered sequentially. In some embodiments, the one or more agent(s) are delivered prior to the delivery of the polynucleotide.

In some embodiments, the template polynucleotide is introduced into the cell for engineering, in addition to the agent(s) capable of inducing a targeted genetic disruption, e.g., nuclease and/or gRNAs. In some embodiments, the template polynucleotide(s) may be delivered prior to, simultaneously or after one or more components of the agent(s) capable of inducing a targeted genetic disruption is introduced into a cell. In some embodiments, the template polynucleotide(s) are delivered simultaneously with the agents. In some embodiments, the template polynucleotide(s) and the one or more agent(s) are delivered simultaneously, e.g., in one reaction, using a physical delivery means. In some embodiments, the template polynucleotide(s) and the one or more agent(s) are delivered simultaneously, via electroporation.

In some embodiments, the template polynucleotides are delivered prior to the agents, for example, seconds to hours to days before the template polynucleotides, including, but not limited to, 1 to 60 minutes (or any time therebetween) before the agents, 1 to 24 hours (or any time therebetween) before the agents or more than 24 hours before the agents. In some embodiments, the template polynucleotides are delivered after the agents, seconds to hours to days after the template polynucleotides, including immediately after delivery of the agent, e.g., between 30 seconds to 4 hours, such as about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after delivery of the agents and/or preferably within 4 hours of delivery of the agents. In some embodiments, the template polynucleotide is delivered more than 4 hours after delivery of the agents.

In some embodiments, the template polynucleotides may be delivered using the same delivery systems as the agent(s) capable of inducing a targeted genetic disruption, e.g., nuclease and/or gRNAs. In some embodiments, the template polynucleotides may be delivered using different same delivery systems as the agent(s) capable of inducing a targeted genetic disruption, e.g., nuclease and/or gRNAs. In some embodiments, the template polynucleotide is delivered simultaneously with the agent(s). In other embodiments, the template polynucleotide is delivered at a different time, before or after delivery of the agent(s). Any of the delivery method described herein in Section II.A.3 (e.g., in Tables 10 and 11) for delivery of nucleic acids in the agent(s) capable of inducing a targeted genetic disruption, e.g., nuclease and/or gRNAs, can be used to deliver the template polynucleotide.

In some embodiments, the one or more agent(s) and the template polynucleotide are delivered in the same format or method. For example, in some embodiments, the one or more agent(s) and the template polynucleotide are both comprised in a vector, e.g., viral vector. In some embodiments, the template polynucleotide is encoded on the same vector backbone, e.g. AAV genome, plasmid DNA, as the Cas9 and gRNA. In some aspects, the one or more agent(s) and the template polynucleotide are in different formats, e.g., ribonucleic acid-protein complex (RNP) for the Cas9-gRNA agent and a linear DNA for the template polynucleotide, but they are delivered using the same method.

In some embodiments, the template polynucleotide is a linear or circular nucleic acid molecule, such as a linear or circular DNA or linear RNA, and can be delivered using any of the methods described in Section II.A.3 herein (e.g., Tables 10 and 11 herein) for delivering nucleic acid molecules into the cell.

In some of any embodiments, the polynucleotide, e.g., the template polynucleotide, are introduced into the cells in nucleotide form, e.g., as or within a non-viral vector. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (such as described in Lee et al. (2012) Nano Lett 12: 6322-27), lipid-mediated transfection, peptide-mediated delivery, e.g., cell-penetrating peptides, or a combination thereof. In some embodiments, the non-viral polynucleotide is delivered into the cell by a non-viral method described herein, such as a non-viral method listed in Table 11 herein.

In some embodiments, the template polynucleotide sequence can be comprised in a vector molecule containing sequences that are not homologous to the region of interest in the genomic DNA. In some embodiments, the virus is a DNA virus (e.g., dsDNA or ssDNA virus). In some embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein. A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In some embodiments, the template polynucleotide can be transferred into cells using recombinant infectious virus particles, such as, e.g., vectors derived from simian virus 40 (SV40), adenoviruses, adeno-associated virus (AAV). In some embodiments, the template polynucleotide are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557 or HIV-1 derived lentiviral vectors.

In other aspects, the template polynucleotide is delivered by viral and/or non-viral gene transfer methods. In some embodiments, the template polynucleotide is delivered to the cell via an adeno associated virus (AAV). The template polynucleotide may be delivered using the same gene transfer system as used to deliver the nuclease (including on the same vector) or may be delivered using a different delivery system that is used for the nuclease. In some embodiments, the template polynucleotide is delivered using a viral vector (e.g., AAV) and the nuclease(s) is(are) delivered in mRNA form. The cell may also be treated with one or more molecules that inhibit binding of the viral vector to a cell surface receptor as described herein prior to, simultaneously and/or after delivery of the viral vector (e.g., carrying the nuclease(s) and/or template polynucleotide).

In some embodiments, the retroviral vector has a long terminal repeat sequence (LTR), e.g., a recombinant retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, the template polynucleotides are delivered using an AAV vector and the agent(s) capable of inducing a targeted genetic disruption, such as nuclease and/or gRNAs are delivered as a different form, such as mRNAs encoding the nucleases and/or gRNAs. In some embodiments, the template polynucleotides and nucleases are delivered using the same type of method, such as a viral vector, but on separate vectors. In some embodiments, the template polynucleotides are delivered in a different delivery system as the agents capable of inducing a genetic disruption, such as nucleases and/or gRNAs. Types or nucleic acids and vectors for delivery include any of those described in Section II.B or III herein.

In some embodiments, the template polynucleotides and nucleases may be on the same vector, for example an AAV vector (such as AAV6). In some embodiments, the template polynucleotides are delivered using an AAV vector and the agent(s) capable of inducing a targeted genetic disruption, such as nuclease and/or gRNAs are delivered as a different form, such as mRNAs encoding the nucleases and/or gRNAs. In some embodiments, the template polynucleotides and nucleases are delivered using the same type of method, such as a viral vector, but on separate vectors. In some embodiments, the template polynucleotides are delivered in a different delivery system as the agents capable of inducing a genetic disruption, such as nucleases and/or gRNAs. In some embodiments, the template polynucleotide is excised from a vector backbone in vivo, such as it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, such as in a vector or a linear nucleic acid molecule, such as linear DNA. Types or nucleic acids and vectors for delivery include any of those described in Section II.B or III herein.

In some embodiments, the template polynucleotide is comprised in an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In some embodiments, the template polynucleotide comprises about 150 to 1000 nucleotides of homology on either side of the transgene and/or the target site. In some embodiments, the template polynucleotide comprises about 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at least 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at most 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene.

In some embodiments, the template polynucleotide is a lentiviral vector, e.g., an IDLV (integration deficiency lentivirus). In some embodiments, the template polynucleotide comprises about 500 to 1000 base pairs of homology on either side of the transgene and/or the target site. In some embodiments, the template polynucleotide comprises about 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises one or more mutations, e.g., silent mutations, that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered. In some embodiments, the cDNA comprises one or more mutations, e.g., silent mutations that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered.

Double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

### III. NUCLEIC ACIDS, VECTORS AND DELIVERY

In some embodiments, the polynucleotide, such as a template polynucleotide comprising a transgene encoding a recombinant receptor or a portion thereof, are introduced into the cells in nucleotide form, such as a polynucleotide or a vector. In some of any embodiments, the polynucleotide contains a transgene that encodes the recombinant receptor or a portion thereof. In certain embodiments, the one or more agent(s) or components thereof for genetic disruption are introduced into the cells in nucleic acid form, such as polynucleotides and/or vectors. In some embodiments, the components for engineering can be delivered in various forms using various delivery methods, including any suitable methods used for delivery of agent(s) as described in Section II.B.3 and Tables 10 and 11 herein. Also provided are one or more polynucleotides (such as nucleic acid molecules) encoding one or more components of the one or more agent(s) capable of inducing a genetic disruption, and/or one or more template polynucleotides containing transgene (for example, any described in Section II.B.2 herein), and vectors for genetically engineering cells for targeted integration of the transgene, such as a template polynucleotide or a polynucleotide encoding one or more components of the one or more agent(s) capable of inducing a genetic disruption.

In some embodiments, provided are polynucleotides, such as template polynucleotides for targeting transgene at a specific genomic target location, such as at the T cell stimulation-associated locus. In some embodiments, provided are any template polynucleotides described in Section II.B.2 herein. In some embodiments, the template polynucleotide contains transgene that include nucleic acid sequences that encode a recombinant receptor or a portion thereof or other polypeptides and/or factors, and homology arms for targeted integration. In some embodiments, the template polynucleotide can be contained in a vector.

In some embodiments, agents capable of inducing a genetic disruption can be encoded in one or more polynucleotides. In some embodiments, the component of the agents, such as Cas9 molecule and/or a gRNA molecule, can be encoded in one or more polynucleotides, and introduced into the cells. In some embodiments, the polynucleotide encoding one or more component of the agents can be included in a vector.

In some embodiments, a vector may comprise a sequence that encodes a Cas9 molecule and/or a gRNA molecule and/or template polynucleotides. A vector may also comprise a sequence encoding a signal peptide (such as for nuclear localization, nucleolar localization, mitochondrial localization), fused, such as to a Cas9 molecule sequence. For example, a vector may comprise a nuclear localization sequence (such as from SV40) fused to the sequence encoding the Cas9 molecule.

In some of any embodiments, one or more regulatory/control elements, such as a promoter, an enhancer, an intron, a polyadenylation signal, a Kozak consensus sequence, internal ribosome entry sites (IRES), a 2A sequence, and splice acceptor or donor can be included in the vectors. In some embodiments, the promoter is selected from among an RNA pol I, pol II or pol III promoter. In some embodiments, the promoter is recognized by RNA polymerase II (such as a CMV, SV40 early region or adenovirus major late promoter). In another embodiment, the promoter is recognized by RNA polymerase III (such as a U6 or H1 promoter).

In certain embodiments, the promoter is a regulated promoter (such as inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter or a modified form thereof (e.g., EF1α promoter with HTLV1 enhancer) or the MND promoter. In some embodiments, the polynucleotide and/or vector does not include a regulatory element, e.g. promoter.

In some of any embodiments, the polynucleotide, e.g., the polynucleotide encoding the recombinant receptor or a portion thereof, are introduced into the cells in nucleotide form, e.g., as or within a non-viral vector. In some embodiments, the polynucleotide is a DNA or an RNA polynucleotide. In some embodiments, the polynucleotide is a double-stranded or single-stranded polynucleotide. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (such as described in Lee et al. (2012) Nano Lett 12: 6322-27), lipid-mediated transfection, peptide-mediated delivery, or a combination thereof. In some embodiments, the non-viral polynucleotide is delivered into the cell by a non-viral method described herein, such as a non-viral method listed in Table 11.

In some embodiments, the vector or delivery vehicle is a viral vector (e.g., for generation of recombinant viruses). In some embodiments, the virus is a DNA virus (e.g., dsDNA or ssDNA virus). In some embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein.

In some embodiments, the virus infects dividing cells. In another embodiment, the virus infects non-dividing cells. In another embodiment, the virus infects both dividing and non-dividing cells. In another embodiment, the virus can integrate into the host genome. In another embodiment, the virus is engineered to have reduced immunity, e.g., in human. In another embodiment, the virus is replication-competent. In another embodiment, the virus is replication-defective, e.g., having one or more coding regions for the genes necessary for additional rounds of virion replication and/or packaging replaced with other genes or deleted. In another embodiment, the virus causes transient expression of the Cas9 molecule and/or the gRNA molecule for the purposes of transient induction of genetic disruption. In another embodiment, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the polynucleotide containing the agent(s) and/or template polynucleotide is delivered by a recombinant retrovirus. In another embodiment, the retrovirus (e.g., Moloney murine leukemia virus) comprises a reverse transcriptase, e.g., that allows integration into the host genome. In some embodiments, the retrovirus is replication-competent. In another embodiment, the retrovirus is replication-defective, e.g., having one of more coding regions for the genes necessary for additional rounds of virion replication and packaging replaced with other genes, or deleted.

In some embodiments, the polynucleotide containing the agent(s) and/or template polynucleotide is delivered by a recombinant lentivirus. For example, the lentivirus is replication-defective, e.g., does not comprise one or more genes required for viral replication.

In some embodiments, the polynucleotide containing the agent(s) and/or template polynucleotide is delivered by a recombinant adenovirus. In another embodiment, the adenovirus is engineered to have reduced immunity in humans.

In some embodiments, the polynucleotide containing the agent(s) and/or template polynucleotide is delivered by a recombinant AAV. In some embodiments, the AAV can incorporate its genome into that of a host cell, e.g., a target cell as described herein. In another embodiment, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV7, AAV8, AAV 8.2, AAV9, AAV.rh10, modified AAV.rh10, AAV.rh32/33, modified AAV.rh32/33, AAV.rh43, modified AAV.rh43, AAV.rh64R1, modified AAV.rh64R1, and pseudotyped AAV, such as AAV2/8, AAV2/5 and AAV2/6 can also be used in the disclosed methods.

In some embodiments, the polynucleotide containing the agent(s) and/or template polynucleotide is delivered by a hybrid virus, e.g., a hybrid of one or more of the viruses described herein.

A packaging cell is used to form a virus particle that is capable of infecting a target cell. Such a cell includes a 293 cell, which can package adenovirus, and a ψ2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., Cas9. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the viral vector has the ability of cell type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In some embodiments, the viral vector achieves cell type specific expression. For example, a tissue-specific promoter can be constructed to restrict expression of the transgene (Cas9 and gRNA) in only a specific target cell. The specificity of the vector can also be mediated by microRNA-dependent control of transgene expression. In some embodiments, the viral vector has increased efficiency of fusion of the viral vector and a target cell membrane. For example, a fusion protein such as fusion-competent hemagglutinin (HA) can be incorporated to increase viral uptake into cells. In some embodiments, the viral vector has the ability of nuclear localization. For example, a virus that requires the breakdown of the nuclear membrane (during cell division) and therefore will not infect a non-diving cell can be altered to incorporate a nuclear localization peptide in the matrix protein of the virus thereby enabling the transduction of non-proliferating cells.

### IV. ENGINEERED CELLS

Provided herein are genetically engineered cells comprising a modified T cell stimulation-associated locus that comprises nucleic acid sequences (e.g., a transgene) encoding a recombinant receptor, such as a chimeric antigen receptor (CAR), or a portion thereof or a recombinant T cell receptor (TCR) or a portion or a chain thereof. In some aspects, the modified T cell stimulation-associated locus in the genetically engineered cell comprises exogenous nucleic acid sequences (e.g., transgene) encoding a recombinant receptor or portion thereof, integrated into the endogenous T cell stimulation-associated locus. In some aspects, the provided engineered cells are produced using methods described herein, e.g., involving homology-dependent repair (HDR) by employing agent(s) for inducing a genetic disruption and template polynucleotides containing the transgene for repair. In some aspects, a part, e.g., a contiguous segment of the provided polynucleotides, such as any template polynucleotides described in Section II.B.2, can be targeted for integration at the endogenous T cell stimulation-associated locus, to generate a cell containing a modified T cell stimulation-associated locus comprising a nucleic acid sequence encoding a recombinant receptor or a portion thereof. In some embodiments, the part of the template polynucleotide that is integrated by HDR into the endogenous T cell stimulation-associated locus includes the transgene portion, such as any described herein, for example in Section II.B.2, of the template polynucleotide.

In some aspects, the cells are engineered to express a recombinant receptor, such as a CAR or a recombinant T cell receptor (TCR). In some aspects, the recombinant receptor is encoded by the nucleic acid sequences present at the modified T cell stimulation-associated locus in the engineered cells. In some aspects, the cells are generated by integrating the transgene encoding all or a portion of the recombinant receptor, via HDR. In some embodiments, the recombinant receptor contains a binding domain that binds to or recognizes a ligand or an antigen, e.g., an antigen associated with a disease or disorder.

In some aspects, the engineered cells are immune cells, such as T cells. In some embodiments, the engineered cells are T cells. In some embodiments, the engineered cells are human T cells. In some of any embodiments, the T cell is a T cell derived from a subject. In some of any embodiments, the subject is a human. In some embodiments, the T cells are primary T cells. In some of any embodiments, the engineered cells are primary human T cells. In some aspects, the immune cells are engineered to express a recombinant receptor, e.g., chimeric antigen receptor or modified recombinant receptors, such as any described herein.

In some embodiments, the methods, compositions, articles of manufacture, and/or kits provided herein are useful to generate, manufacture, or produce genetically engineered cells, e.g., genetically engineered immune cells and/or T cells, that have or contain a modified T cell stimulation-associated locus. In some of any embodiments, the methods provided herein result in genetically engineered cells that have or contain a modified T cell stimulation-associated locus. In some embodiments, the modified locus is or contains a transgene, e.g., a transgene described in Section II.B.2, integrated in an open reading frame of the endogenous T cell stimulation-associated locus gene. In certain embodiments, the transgene is inserted in-frame into the open reading frame of the endogenous T cell stimulation-associated locus gene, resulting in a modified T cell stimulation-associated locus that encodes all or a portion of the gene product encoded by the endogenous T cell stimulation-associated locus. In some embodiments, the recombinant receptor is a chimeric antigen receptor (CAR). In some aspects, the recombinant receptor is a recombinant T cell receptor (TCR). In some aspects, the modified T cell stimulation-associated locus comprises a transgene encoding the entire recombinant receptor or a full length recombinant receptor, such as a full length CAR or both chains of recombinant TCR comprising two chains. In some aspects, the modified T cell stimulation-associated locus comprises a transgene encoding a portion of the recombinant receptor, for example one chain of a multi-chain CAR or one chain of a recombinant TCR comprising two chains, or a domain or a region of the recombinant receptor; and the engineered cell comprises a second transgene encoding a remaining portion of the recombinant receptor, for example another chain of a multi-chain CAR or the other chain of the recombinant TCR, present at a different location in the genome of the engineered cell.

In some cases, the cell is engineered to express one or more additional molecules, e.g., an additional factors and/or an accessory molecule, such as any additional molecules, including therapeutic molecules, described herein. In some embodiments, the additional molecules can include a marker, an additional recombinant receptor polypeptide chain, an antibody or an antigen-binding fragment thereof, an immunomodulatory molecule, a ligand, a cytokine or a chemokine. In some embodiments, the additional factors is a soluble molecule. In some embodiments, the additional factors is a membrane-bound molecule. In some aspects, the additional factors can be used to overcome or counteract the effect of an immunosuppressive environment, such as a tumor microenvironment (TME). In some aspects, exemplary additional molecule includes a cytokine, a cytokine receptor, a chimeric co-stimulatory receptor, a co-stimulatory ligand and other modulators of T cell function or activity. In some embodiments, the additional molecules expressed by the engineered cell include IL-7, IL-12, IL-15, CD40 ligand (CD40L), and 4-1BB ligand (4-1BBL). In some aspects, the additional molecule is an additional receptor, e.g., a membrane-bound receptor, that binds a different molecule. For example, in some embodiments, the additional molecule is a cytokine receptor or a chemokine receptor, e.g., IL-4 receptor or CCL2 receptor. In some cases, the engineered cells are called “armored CARs” or T cells redirected for universal cytokine killing (TRUCKs).

Also provided are compositions containing a plurality of the engineered cells. In some aspects, the compositions containing the engineered cells exhibit improved, uniform, homogeneous and/or stable expression and/or antigen binding by the recombinant receptor, compared to cells or cell compositions generated using other methods of engineering, such as methods in which the recombinant receptor is introduced randomly into the genome of a cell. In some embodiments, the engineered cells or the composition comprising the engineered cells can be used in therapy, e.g., adoptive cell therapy. In some embodiments, the provided cells or cell compositions can be used in any of the methods of treatment described herein or for therapeutic uses described herein.

### A. Modified Loci

In some aspects, provided are genetically engineered cells, such as modified T cells, comprising a modified T cell stimulation-associated locus. In some embodiments, the modified T cell stimulation-associated locus comprises a nucleic acid sequence encoding a recombinant receptor or a portion thereof. In some embodiments, the nucleic acid sequence comprises a transgene encoding a recombinant receptor or a portion thereof, the transgene having been integrated at the endogenous T cell stimulation-associated locus, optionally via homology directed repair (HDR). In some aspects, the modified T cell stimulation-associated locus can encode any one or more of the recombinant receptors described herein, for example in Section IV.B, or a portion thereof, such as a domain or region thereof, or one or more chains of a multi-chain recombinant receptor described herein.

In some embodiments, provided are engineered cells containing a modified PDCD1 locus comprising a transgene encoding a recombinant receptor or a portion thereof, such as a chimeric antigen receptor (CAR) or a recombinant T cell receptor (TCR), operably linked to an endogenous transcriptional regulatory element of the PDCD1 locus, wherein the endogenous transcriptional regulatory element PDCD1 induces or upregulates, such as transiently induces or upregulates, expression of the operably linked transgene following a simulation or activation signal in the T cell.

In some embodiments, provided are engineered cells containing a modified CD69 locus comprising a transgene encoding a recombinant receptor or a portion thereof, such as a CAR or a TCR, operably linked to an endogenous transcriptional regulatory element of the CD69 locus, wherein the endogenous transcriptional regulatory element CD69 induces or upregulates, such as transiently induces or upregulates, expression of the operably linked transgene following a simulation or activation signal in the T cell.

In some embodiments, provided are engineered cells containing a modified Nur77 locus comprising a transgene encoding a recombinant receptor or a portion thereof, such as a CAR or a TCR, operably linked to an endogenous transcriptional regulatory element of the Nur77 (encoding NR4A1) locus, wherein the endogenous transcriptional regulatory element Nur77 (encoding NR4A1) induces or upregulates, such as transiently induces or upregulates, expression of the operably linked transgene following a simulation or activation signal in the T cell.

In some embodiments, provided are engineered cells containing a modified FoxP3 locus comprising a transgene encoding a recombinant receptor or a portion thereof, such as a CAR or a TCR, operably linked to an endogenous transcriptional regulatory element of the FoxP3 locus, wherein the endogenous transcriptional regulatory element FoxP3 induces or upregulates, such as transiently induces or upregulates, expression of the operably linked transgene following a simulation or activation signal in the T cell.

In some embodiments, provided are engineered cells containing a modified a HLA-DR locus comprising a transgene encoding a recombinant receptor or a portion thereof, such as a CAR or a TCR, operably linked to an endogenous transcriptional regulatory element of the a HLA-DR locus, wherein the endogenous transcriptional regulatory element a HLA-DR induces or upregulates, such as transiently induces or upregulates, expression of the operably linked transgene following a simulation or activation signal in the T cell.

In some embodiments, the modified T cell stimulation-associated locus is generated as a result of genetic disruption and integration of the transgene (e.g. exogenous or heterologous nucleic acid sequences) that includes a sequence of nucleotides encoding a recombinant receptor or a portion thereof, such as via HDR methods. In some aspects, the nucleic acid sequence present at the modified T cell stimulation-associated locus includes the transgene(s), such as an exogenous sequence, integrated at a region in the endogenous T cell stimulation-associated locus that normally would include an open reading frame encoding full length gene product of the T cell stimulation-associated locus. In some aspects, upon targeted integration of the transgene by HDR, the genome of the cell contains a modified T cell stimulation-associated locus, comprising a nucleic acid sequence encoding a recombinant receptor or a portion thereof.

In some embodiments, following integration of the transgene encoding the recombinant receptor or a portion thereof, the endogenous gene product of the T cell stimulation-associated locus is expressed in full, in addition to the recombinant receptor that is expressed from the modified T cell stimulation-associated locus. In some embodiments, following integration of the transgene encoding the recombinant receptor or a portion thereof, all or a portion of the endogenous gene product of the T cell stimulation-associated locus is not expressed. In some embodiments, the modified T cell stimulation-associated locus encodes a portion of the full-length endogenous gene product of the T cell stimulation-associated locus, e.g., the endogenous gene product contains a deletion. In some embodiments, the modified T cell stimulation-associated locus does not encode the endogenous gene product of the T cell stimulation-associated locus, i.e., the endogenous gene product is knocked out. In some embodiments, upon targeted integration, the modified T cell stimulation-associated locus contains the transgene integrated into a site within the open reading frame of the endogenous T cell stimulation-associated locus, such that the recombinant receptor is expressed from the engineered cell, and, in some cases, also a portion of gene product of the T cell stimulation-associated locus, e.g. a partial or truncated gene product of the T cell stimulation-associated locus.

In some aspects, the T cell stimulation-associated locus is PDCD1, and the endogenous gene product of the locus, PD-1, is not expressed or is not functional. In some aspects, the T cell stimulation-associated locus is PDCD1, and the endogenous gene product of the locus, PD-1, is expressed in full length or is functional. In some aspects, both the PD1 polypeptide and the recombinant receptor or a portion thereof are co-expressed in the cell comprising the modified T cell stimulation-associated locus PDCD1.

In some aspects, the T cell stimulation-associated locus is CD69, and the endogenous gene product of the locus, CD69, is not expressed or is not functional. In some aspects, the T cell stimulation-associated locus is CD69, and the endogenous gene product of the locus, CD69, is expressed in full length or is functional. In some aspects, both the CD69 polypeptide and the recombinant receptor or a portion thereof are co-expressed in the cell comprising the modified T cell stimulation-associated locus CD69.

In some aspects, the T cell stimulation-associated locus is Nur77, and the endogenous gene product of the locus, NR4A1, is not expressed or is not functional. In some aspects, the T cell stimulation-associated locus is Nur77, and the endogenous gene product of the locus, NR4A1, is expressed in full length or is functional. In some aspects, both the Nur77 polypeptide and the recombinant receptor or a portion thereof are co-expressed in the cell comprising the modified T cell stimulation-associated locus Nur77.

In some aspects, the T cell stimulation-associated locus is FoxP3, and the endogenous gene product of the locus, FoxP3, is not expressed or is not functional. In some aspects, the T cell stimulation-associated locus is FoxP3, and the endogenous gene product of the locus, FoxP3, is expressed in full length or is functional. In some aspects, both the FoxP3 polypeptide and the recombinant receptor or a portion thereof are co-expressed in the cell comprising the modified T cell stimulation-associated locus FoxP3.

In some aspects, the T cell stimulation-associated locus is HLA-DRA, and the endogenous gene product of the locus, HLA-DRA, is not expressed or is not functional. In some aspects, the T cell stimulation-associated locus is HLA-DRA, and the endogenous gene product of the locus, HLA-DRA, is expressed in full length or is functional. In some aspects, both the HLA-DRA polypeptide and the recombinant receptor or a portion thereof are co-expressed in the cell comprising the modified T cell stimulation-associated locus HLA-DRA.

In some aspects, the T cell stimulation-associated locus is HLA-DRB1, and the endogenous gene product of the locus, HLA-DRB1, is not expressed or is not functional. In some aspects, the T cell stimulation-associated locus is HLA-DRB1, and the endogenous gene product of the locus, HLA-DRB1, is expressed in full length or is functional. In some aspects, both the HLA-DRB1 polypeptide and the recombinant receptor or a portion thereof are co-expressed in the cell comprising the modified T cell stimulation-associated locus HLA-DRB1.

In some embodiments, upon integration of the transgene, the endogenous sequences of the T cell stimulation-associated locus comprise a genetic disruption, such as a deletion of nucleic acid sequence encoding one or more amino acids and/or a mutation introducing a stop codon. In some embodiments, upon integration of the transgene, the endogenous sequences of the T cell stimulation-associated locus do not encode a functional gene product of the T cell stimulation-associated locus polypeptide. In some embodiments, upon integration of the transgene, the endogenous sequences of the T cell stimulation-associated locus encode a partial gene product of the T cell stimulation-associated locus polypeptide or a truncated gene product of the T cell stimulation-associated locus polypeptide.

In certain embodiments, the transgene encodes a recombinant receptor and is inserted in-frame within an endogenous open reading frame of the T cell stimulation-associated locus. In particular embodiments, the transcription of the modified locus results in an mRNA that encodes the recombinant receptor, such as a CAR. In some aspects, the nucleic acid sequence nucleic acid sequence present in the open reading frame of the endogenous T cell stimulation-associated locus can encode a partial or a truncated gene product of the T cell stimulation-associated locus polypeptide, such as a dominant negative form of gene product of the T cell stimulation-associated locus. In some embodiments, the transgene is integrated at a target site immediately downstream of and in frame with one or more exons of open reading frame of the endogenous T cell stimulation-associated locus. In some embodiments, the transgene is integrated or inserted downstream of exon 1, 2, 3 or 4 and upstream of exon 6, 7 or 8 of the open reading frame of the endogenous T cell stimulation-associated locus (such as described in Tables 1-9 herein). In some embodiments, the transgene is integrated or inserted downstream of exon 1, 2, 3 or 4 and upstream of exon 6 of the open reading frame of the endogenous T cell stimulation-associated locus (such as described in Tables 1-9 herein). In some embodiments, the transgene is downstream of exon 1 and upstream of exon 8 of the open reading frame of the endogenous T cell stimulation-associated locus. In some embodiments, the transgene is downstream of exon 3 and upstream of exon 5 of the open reading frame of the endogenous T cell stimulation-associated locus. In some embodiments, the transgene is downstream of exon 4 and upstream of exon 6 of the open reading frame of the endogenous T cell stimulation-associated locus.

In some embodiments, the mRNA transcribed from the modified locus contains a 3′UTR that is encoded by the endogenous T cell stimulation-associated locus and/or is identical to a 3′UTR of an mRNA that is transcribed from the endogenous T cell stimulation-associated locus. In some embodiments, the transgene contains a ribosomal skipping element upstream, e.g., immediately upstream, of the sequence of nucleic acids encoding the portion of the CAR. In some embodiments, the mRNA encoding the CAR contains a 5′UTR that is encoded by the endogenous T cell stimulation-associated locus and/or is identical to a 5′UTR of an mRNA that is transcribed from the endogenous T cell stimulation-associated locus.

In some embodiments, the recombinant receptor encoded from the modified T cell stimulation-associated locus is a CAR. In some embodiments, the CAR encoded by the modified T cell stimulation-associated locus binds to and/or is capable of binding to a target antigen. In some embodiments, the target antigen is associated with, specific to, and/or expressed on a cell or tissue that is associated with a disease, disorder, or condition. In some embodiments, the CAR is capable of stimulating and/or inducing a primary activation signal in a T cell, a signaling domain of a T cell receptor (TCR) component and/or a signaling domain comprising an immunoreceptor tyrosine-based activation motif (ITAM), such as via an intracellular signaling domain or region of a CD3-zeta (CD3ζ) chain or a functional variant or signaling portion thereof.

In some embodiments, the recombinant receptor encoded from the modified T cell stimulation-associated locus is a is a recombinant TCR. In some aspects, the recombinant TCR comprises two polypeptide chains, for example, a TCR alpha (TCRα) and a TCR beta (TCRβ) chain; or a TCR gamma (TCRγ) and a TCR delta (TCRδ) chain. In some aspects, the modified T cell stimulation-associated locus encodes one or more chains of the recombinant TCR. In some embodiments, the modified T cell stimulation-associated locus encodes a TCRα. In some embodiments, the modified T cell stimulation-associated locus encodes a TCRβ. In some aspects, if the modified T cell stimulation-associated locus encodes only one chain of the recombinant TCR, the other chain of the TCR can be encoded by a second transgene present in the engineered cell, e.g., at a different genomic location. In some embodiments, the modified T cell stimulation-associated locus encodes a TCRα and a TCRβ, optionally separated by a multicistronic element such as a 2A element.

### B. Encoded Recombinant Receptors

In some embodiments, the recombinant receptor encoded by the engineered cells provided herein, or the engineered cells generated according to the methods provided herein, include a chimeric antigen receptor (CAR) or a portion thereof, or a recombinant T cell receptor (TCR) or a portion thereof. Among the recombinant receptors are chimeric receptors, antigen receptors and receptors containing one or more component of chimeric receptors or antigen receptors. The recombinant receptors may include those containing ligand-binding domains or binding fragments thereof and intracellular signaling domains or regions. In some embodiments, the recombinant receptors encoded by the engineered cells include functional non-TCR antigen receptors, chimeric antigen receptors (CARs), chimeric autoantibody receptor (CAAR), recombinant T cell receptors (TCRs) and region(s), chain(s), domain(s) or component(s) of any of the foregoing. In some aspects, the recombinant receptor or a portion thereof is encoded by transgene present in the polynucleotides provided herein, such as any template polynucleotides described in Section II.B.2 above. In some aspects, the transgene encoding the recombinant receptor or a portion thereof contained in the polynucleotides, is integrated at the endogenous T cell stimulation-associated locus of the engineered cell, to result in a modified T cell stimulation-associated locus that encodes a recombinant receptor or a portion thereof, such as any recombinant receptor described herein, including one or more polypeptide chains of a multi-chain recombinant receptor.

In some embodiments, exemplary recombinant receptors expressed from the engineered cell include multi-chain receptors that contain two or more receptor polypeptides, which, in some cases, contain different components, domains or regions. In some aspects, the recombinant receptor contains two or more polypeptides that together comprise a functional recombinant receptor. In some aspects, the multi-chain receptor is a dual-chain receptor, comprising two polypeptides that together comprise a functional recombinant receptor. In some embodiments, the recombinant receptor is a TCR comprising two different receptor polypeptides, for example, a TCR alpha (TCRα) and a TCR beta (TCRβ) chain; or a TCR gamma (TCRγ) and a TCR delta (TCRδ) chain. In some embodiments, the recombinant receptor is a CAR comprising two or more different receptor polypeptides, such as a multi-chain CAR. In some embodiments, the recombinant receptor is a multi-chain receptor in which one or more of the polypeptides regulates, modifies or controls the expression, activity or function of another receptor polypeptide. In some aspects, multi-chain receptors allows spatial or temporal regulation or control of specificity, activity, antigen (or ligand) binding, function and/or expression of the receptor. In some aspects, the entire recombinant receptor, such as all chains of the multi-chain recombinant receptor, can be encoded by the transgene present in the modified T cell stimulation-associated locus. In some aspects, one chain of the multi-chain recombinant receptor can be encoded by the transgene present in the T cell stimulation-associated locus, and the other chain(s) are encoded by a second transgene present at a different location in the genome (e.g., a different T cell stimulation-associated locus, or a different location).

In some embodiments, the recombinant receptor, encoded in the genetically engineered cells provided herein, contains a transmembrane domain or a membrane association domain. In some aspects, the recombinant receptor also contains an extracellular region. In some aspects, the recombinant receptor also contains an intracellular region. In some embodiments, the recombinant receptor encoded in the genetically engineered cells provided herein contains various regions or domains such as one or more of extracellular region (e.g., containing one or more extracellular binding domain(s) and/or spacers), transmembrane domain and intracellular region (e.g., containing an intracellular signaling region and/or one or more costimulatory signaling domains). In some aspects, the encoded recombinant receptor further contains other domains, such as multimerization domains, linkers and/or regulatory elements.

In some embodiments, an exemplary encoded recombinant receptor comprises, in its N- to C-terminus order: a transmembrane domain (or a membrane association domain) and an intracellular region. In some embodiments, an exemplary encoded recombinant receptor comprises, in its N- to C-terminus order: an extracellular region, a transmembrane domain and an intracellular region. In some embodiments, the extracellular region is or comprises an extracellular binding domain and, in some aspects, the encoded recombinant receptor comprises, from its N to C terminus in order: an extracellular binding domain, a transmembrane domain and an intracellular region. In some cases, a spacer that separates or is positioned between the extracellular region, e.g. extracellular binding domain, and the transmembrane domain. In some embodiments, the encoded recombinant receptor comprises, from its N to C terminus in order: an extracellular binding domain, a spacer, a transmembrane domain and an intracellular region. In some embodiments, the intracellular signaling region present in a recombinant receptor contains an immunoreceptor tyrosine-based activation motif (ITAM) and/or one or more costimulatory signaling domains, such as one, two or three costimulatory signaling domains

In some embodiments, the recombinant receptor contains a multimerization domain, which in some aspects, is able to effect formation of a multi-chain polypeptide thereof. In some embodiments, an exemplary encoded recombinant receptor comprises, in its N- to C-terminus order: a transmembrane domain (or a membrane association domain), an intracellular multimerization domain, optionally one or more costimulatory signaling domain(s), and an intracellular signaling region. In some embodiments, an exemplary recombinant receptor polypeptide comprises, in its N- to C-terminus order: an extracellular multimerization domain, a transmembrane domain, optionally one or more costimulatory signaling domain(s), and an intracellular signaling region.

In some embodiments, the encoded recombinant receptor is a chimeric receptor, such as a CAR. An exemplary encoded CAR sequence comprises: an extracellular binding domain, a spacer, a transmembrane domain and an intracellular region comprising a primary signaling domain or region and one or more co-stimulatory signaling domain. In some embodiments, an exemplary encoded CAR sequence comprises: an extracellular binding domain, a spacer, a transmembrane domain and one or more costimulatory signaling domains and primary signaling domain or region.

In some embodiments, an exemplary encoded polypeptide, such as a polypeptide chain of a multi-chain CAR, sequence comprises: a transmembrane domain (or a membrane association domain), an intracellular multimerization domain, optionally one or more costimulatory signaling domain(s), and a primary signaling domain or region. In some embodiments, an exemplary encoded polypeptide, such as a polypeptide chain of a multi-chain CAR, sequence comprises: an extracellular multimerization domain, a transmembrane domain, optionally one or more costimulatory signaling domain(s), and a primary signaling domain or region.

In some embodiments, an exemplary encoded CAR sequence comprises, in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof. In some embodiments, the encoded intracellular region of the recombinant receptor comprises, from its N to C terminus in order: the one or more costimulatory signaling domain(s) and a primary signaling domain or region, such as containing a CD3zeta chain or a fragment thereof.

In some embodiments, the encoded recombinant receptor is a recombinant TCR and an exemplary encoded TCR includes, a TCRα chain or a TCRβ chain or both. In some embodiments, an exemplary encoded polypeptide, such as a polypeptide of a recombinant receptor, comprises all or a portion of a TCRα chain. In some embodiments, an exemplary encoded polypeptide, such as a polypeptide of a recombinant receptor, comprises all or a portion of a TCRβ chain. In some aspects, an exemplary encoded recombinant receptor is a recombinant TCR comprising a TCRα chain and a TCRβ chain.

### 1. Chimeric Antigen Receptors (CARs)

In some embodiments, the recombinant receptor encoded by the modified T cell stimulation-associated locus is a chimeric antigen receptor (CAR). In some embodiments, the engineered cells, such as T cells, express a recombinant receptor such as a CAR, with specificity for a particular antigen (or marker or ligand), such as an antigen expressed on the surface of a particular cell type. In some aspects, at least a portion of any of the CARs described herein, including multi-chain or regulatable CAR, is encoded in the transgene. In some aspects, the transgene encoding the CARs described herein or a portion thereof, can be any described in Section II.B.2. In some aspects, upon integration of the transgene via HDR, the resulting modified T cell stimulation-associated locus contains nucleic acid sequence encoding a CAR, such as any CAR described herein, including multi-chain or regulatable CAR.

In some embodiments, the recombinant receptor, e.g., CAR, encoded by the modified T cell stimulation-associated locus, contains one or more of extracellular region (e.g., containing one or more extracellular binding domain(s) and/or spacers), transmembrane domain and/or intracellular region (e.g., containing a primary signaling region or domain and/or one or more costimulatory signaling domains). In some aspects, the encoded recombinant receptor further contains other domains, such as multimerization domains. In some aspects, the modified T cell stimulation-associated locus contains sequences encoding linkers and/or regulatory elements. In some embodiments, the encoded recombinant receptor comprises, from its N to C terminus in order: an extracellular binding domain, a transmembrane domain and an intracellular region, e.g., comprising a primary signaling region or domain or a portion thereof and/or a costimulatory signaling domain. In some embodiments, the encoded recombinant receptor comprises, from its N to C terminus in order: an extracellular binding domain, a spacer, a transmembrane domain and an intracellular region, e.g., comprising a primary signaling region or domain or a portion thereof and/or a costimulatory signaling domain.

a. Binding Domain

In some embodiments, the extracellular region of the encoded recombinant receptor comprises a binding domain. In some embodiments, the binding domain is an extracellular binding domain. In some embodiments, the binding domain is or comprises a polypeptide, a ligand, a receptor, a ligand-binding domain, a receptor-binding domain, an antigen, an epitope, an antibody, an antigen-binding domain, an epitope-binding domain, an antibody-binding domain, a tag-binding domain or a fragment of any of the foregoing. In some embodiments, the binding domain is a ligand- or antigen-binding domain.

In some aspects, the extracellular binding domain, such as a ligand- (e.g., antigen-) binding region or domain(s) and the intracellular region or domain(s) are linked or connected via one or more linkers and/or transmembrane domain(s). In some embodiments, the chimeric antigen receptor includes a transmembrane domain disposed between the extracellular region and the intracellular region.

In some embodiments, the antigen, e.g., an antigen that binds the binding domain of the recombinant receptor, is a polypeptide. In some embodiments, the antigen is a carbohydrate or other molecule. In some embodiments, the antigen is selectively expressed or overexpressed on cells of the disease, disorder or condition, e.g., the tumor or pathogenic cells, as compared to normal or non-targeted cells or tissues, e.g., in healthy cells or tissues. In some embodiments, the disease, disorder or condition is an infectious disease or disorder, an autoimmune disease, an inflammatory disease, or a tumor or a cancer. In some embodiments, the antigen is expressed on normal cells and/or is expressed on the engineered cells. In some aspects, the recombinant receptor, e.g., a CAR, includes one or more regions or domains selected from an extracellular ligand- (e.g., antigen-) binding or region or domains, e.g., any of the antibody or fragment described herein, and an intracellular region. In some embodiments, the ligand- (e.g., antigen-) binding region or domain is or includes an scFv or a single-domain V_{H} antibody and the intracellular region comprises an intracellular signaling region or domain comprising an immunoreceptor tyrosine-based activation motif (ITAM).

Exemplary encoded recombinant receptors, including CARs, include those described, for example, in International Pat. App. Pub. Nos. WO2000/14257, WO2013/126726, WO2012/129514, WO2014/031687, WO2013/166321, WO2013/071154, WO2013/123061, U.S. Pat. App. Pub. Nos. US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European Pat. App. No. EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; and Wu et al., Cancer, 2012 Mar. 18(2): 160-75. In some aspects, the antigen receptors include a CAR as described in U.S. Pat. No. 7,446,190, and those described in International Pat. App. Pub. No. Pub. No. WO 2014/055668. Examples of the CARs include CARs as disclosed in any of the aforementioned references, such as WO2014/031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177).

In some embodiments, the encoded recombinant receptor, e.g., antigen receptor contains an extracellular binding domain, such as an antigen- or ligand-binding domain that binds, e.g., specifically binds, to an antigen, a ligand and/or a marker. Among the antigen receptors are functional non-TCR antigen receptors, such as chimeric antigen receptors (CARs).In some embodiments, the antigen receptor is a CAR that contains an extracellular antigen-recognition domain that specifically binds to an antigen. In some embodiments, the CAR is constructed with a specificity for a particular antigen, marker or ligand, such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more ligand- (e.g., antigen-) binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb), or a single domain antibody (sdAb), such as sdFv, nanobody, V_{H}H and V_{NAR}. In some embodiments, an antigen-binding fragment comprises antibody variable regions joined by a flexible linker.

In some embodiments, the encoded CAR contains an antibody or an antigen-binding fragment (e.g. scFv) that specifically recognizes an antigen or ligand, such as an intact antigen, expressed on the surface of a cell. In some embodiments, the antigen or ligand, is a protein expressed on the surface of cells. In some embodiments, the antigen or ligand is a polypeptide. In some embodiments, it is a carbohydrate or other molecule. In some embodiments, the antigen or ligand is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, among the antigens targeted by the recombinant receptors are those expressed in the context of a disease, condition, or cell type to be targeted via the adoptive cell therapy. Among the diseases and conditions are proliferative, neoplastic, and malignant diseases and disorders, including cancers and tumors, including hematologic malignancy, cancers of the immune system, such as lymphomas, leukemias, and/or myelomas, such as B, T, and myeloid leukemias, lymphomas, and multiple myelomas.

In some embodiments, the antigen or ligand is a tumor antigen or cancer marker. In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19.

In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NOS: 38 and 39, respectively, and CDR-H3 set forth in SEQ ID NO: 40 or 54; and CDR-L1 set forth in SEQ ID NO: 35 and CDR-L2 set forth in SEQ ID NO: 36 or 55 and CDR-L3 set forth in SEQ ID NO: 37 or 34. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40. In some embodiments, the scFv comprises a variable heavy chain region set forth in SEQ ID NO:41 and a variable light chain region set forth in SEQ ID NO:42. In some embodiments, the variable heavy and variable light chains are connected by a linker. In some embodiments, the linker is set forth in SEQ ID NO:56. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51.

In some embodiments, the scFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49. In some embodiments, the scFv comprises a variable heavy chain region set forth in SEQ ID NO:50 and a variable light chain region set forth in SEQ ID NO:51. In some embodiments, the variable heavy and variable light chain are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as is m971 scFv.

In some embodiments, the antigen or antigen binding domain is BCMA. In some embodiments, the scFv contains a V_{H} and a V_{L} derived from an antibody or an antibody fragment specific to BCMA. In some embodiments, the antibody or antibody fragment that binds BCMA is or contains a V_{H} and a V_{L} from an antibody or antibody fragment set forth in International Patent Applications, Publication Number WO 2016/090327 and WO 2016/090320.

In some embodiments, the antigen or antigen binding domain is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Patent Applications, Publication Number WO 2016/090329 and WO 2016/090312.

In some aspects, the encoded CAR contains a ligand- (e.g., antigen-) binding domain that binds or recognizes, e.g., specifically binds, a universal tag or a universal epitope. In some aspects, the binding domain can bind a molecule, a tag, a polypeptide and/or an epitope that can be linked to a different binding molecule (e.g., antibody or antigen-binding fragment) that recognizes an antigen associated with a disease or disorder. Exemplary tag or epitope includes a dye (e.g., fluorescein isothiocyanate) or a biotin. In some aspects, a binding molecule (e.g., antibody or antigen-binding fragment) linked to a tag, that recognizes the antigen associated with a disease or disorder, e.g., tumor antigen, with an engineered cell expressing a CAR specific for the tag, to effect cytotoxicity or other effector function of the engineered cell. In some aspects, the specificity of the CAR to the antigen associated with a disease or disorder is provided by the tagged binding molecule (e.g., antibody), and different tagged binding molecule can be used to target different antigens. Exemplary CARs specific for a universal tag or a universal epitope include those described, e.g., in U.S. Pat. No. 9,233,125, WO 2016/030414, Urbanska et al., (2012) Cancer Res 72: 1844-1852, and Tamada et al., (2012) Clin Cancer Res 18:6436-6445.

In some embodiments, the encoded CAR contains a TCR-like antibody, such as an antibody or an antigen-binding fragment (e.g. scFv) that specifically recognizes an intracellular antigen, such as a tumor-associated antigen, presented on the cell surface as a major histocompatibility complex (MHC)-peptide complex. In some embodiments, an antibody or antigen-binding portion thereof that recognizes an MHC-peptide complex can be expressed on cells as part of a recombinant receptor, such as an antigen receptor. Among the antigen receptors are functional non-T cell receptor (TCR) antigen receptors, such as chimeric antigen receptors (CARs). In some embodiments, a CAR containing an antibody or antigen-binding fragment that exhibits TCR-like specificity directed against peptide-MHC complexes also may be referred to as a TCR-like CAR. In some embodiments, the CAR is a TCR-like CAR and the antigen is a processed peptide antigen, such as a peptide antigen of an intracellular protein, which, like a TCR, is recognized on the cell surface in the context of an MHC molecule. In some embodiments, the extracellular antigen-binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, Major histocompatibility complex (MHC) includes a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning α chain, in some cases with three a domains, and a non-covalently associated β2 microglobulin. Generally, MHC class II molecules are composed of two transmembrane glycoproteins, α and β3, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4⁺ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g., US Pat. App. Pub. Nos. US 2002/0150914; US 2003/0223994; US 2004/0191260; US 2006/0034850; US 2007/00992530; US20090226474; US20090304679; and International App. Pub. No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described herein. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US Pat. App. Pub. No. US20020150914, US20140294841; and Cohen CJ. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody, V_{H}H or V_{NAR}) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some aspects, the CAR is a bispecific CAR, e.g., containing two antigen-binding domains with different specificities.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In some embodiments, the antibody light chain constant region is chosen from, e.g., kappa or lambda, particularly kappa.

Among the binding domains of the encoded recombinant receptors are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In some of any embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the encoded chimeric antigen receptor, including TCR-like CARs, includes an extracellular portion containing an antibody or antibody fragment. In some embodiments, the antibody or fragment includes an scFv. In some aspects, the antibody or antigen-binding fragment can be obtained by screening a plurality, such as a library, of antigen-binding fragments or molecules, such as by screening an scFv library for binding to a specific antigen or ligand.

In some embodiments, the encoded CAR is a multi-specific CAR, e.g., contains a plurality of ligand- (e.g., antigen-) binding domains that can bind and/or recognize, e.g., specifically bind, a plurality of different antigens. In some aspects, the encoded CAR is a bispecific CAR, for example, targeting two antigens, such as by containing two antigen-binding domains with different specificities. In some embodiments, the CAR contains a bispecific binding domain, e.g., a bispecific antibody or fragment thereof, containing at least one antigen-binding domain binding to different surface antigens on a target cell, e.g., selected from any of the listed antigens as described herein, e.g. CD19 and CD22 or CD19 and CD20. In some embodiments, binding of the bispecific binding domain to each of its epitope or antigen can result in stimulation of function, activity and/or responses of the T cell, e.g., cytotoxic activity and subsequent lysis of the target cell. Among such exemplary bispecific binding domain can include tandem scFv molecules, in some cases fused to each other via, e.g. a flexible linker; diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; bispecific T cell engager (BiTE) molecules, which contain tandem scFv molecules fused by a flexible linker (see e.g. Nagorsen and Bauerle, Exp Cell Res 317, 1255-1260 (2011); or triomabs that include whole hybrid mouse/rat IgG molecules (Seimetz et al, Cancer Treat Rev 36, 458-467 (2010). Any of such binding domains can be contained in any of the CARs described herein.

b. Spacer and Transmembrane Domain

In some aspects, the encoded recombinant receptor, e.g., a chimeric antigen receptor, includes an extracellular portion containing one or more ligand- (e.g., antigen-) binding domains, such as an antibody or fragment thereof, and one or more intracellular signaling region or domain (also interchangeably called a cytoplasmic signaling domain or region). In some aspects, the recombinant receptor, e.g., CAR, further includes a spacer and/or a transmembrane domain or portion. In some aspects, the spacer and/or transmembrane domain can link the extracellular portion containing the ligand- (e.g., antigen-) binding domain and the intracellular signaling region(s) or domain(s).

In some embodiments, the encoded recombinant receptor such as the CAR further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4, IgG2 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is less than 250 amino acids in length, less than 200 amino acids in length, less than 150 amino acids in length, less than 100 amino acids in length, less than 75 amino acids in length, less than 50 amino acids in length, less than 25 amino acids in length, less than 20 amino acids in length, less than 15 amino acids in length, less than 12 amino acids in length, or less than 10 amino acids in length. In some embodiments, the spacer is from or from about 10 to 250 amino acids in length, 10 to 150 amino acids in length, 10 to 100 amino acids in length, 10 to 50 amino acids in length, 10 to 25 amino acids in length, 10 to 15 amino acids in length, 15 to 250 amino acids in length, 15 to 150 amino acids in length, 15 to 100 amino acids in length, 15 to 50 amino acids in length, 15 to 25 amino acids in length, 25 to 250 amino acids in length, 25 to 100 amino acids in length, 25 to 50 amino acids in length, 50 to 250 amino acids in length, 50 to 150 amino acids in length, 50 to 100 amino acids in length, 100 to 250 amino acids in length, 100 to 150 amino acids in length, or 150 to 250 amino acids in length. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al. (2013) Clin. Cancer Res., 19:3153, Hudecek et al. (2015) Cancer Immunol Res. 3(2): 125-135 or International Pat. App. Pub. No. WO2014031687.

In some embodiments, the spacer can be derived all or in part from IgG4 and/or IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the spacer can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N to Q substitution at a position corresponding to position 177 in the C_{H}2 region of the IgG4 heavy chain constant region sequence set forth in SEQ ID NO: 60 (Uniprot Accession No. P01861; position corresponding to position 297 by EU numbering and position 79 of the hinge-C_{H}2-C_{H}3 spacer sequence set forth in SEQ ID NO:4) or an N to Q substitution at a position corresponding to position 176 in the C_{H}2 region of the IgG2 heavy chain constant region sequence set forth in SEQ ID NO: 59 (Uniprot Accession No. P01859; position corresponding to position 297 by EU numbering).

In some aspects, the spacer contains only a hinge region of an IgG, such as only a hinge of IgG4, IgG2 or IgG1, such as the hinge only spacer set forth in SEQ ID NO:1, and is encoded by the sequence set forth in SEQ ID NO: 2. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:3. In some embodiments, the spacer is an Ig hinge, e.g., an IgG4 hinge, linked to a C_{H}3 domain only, such as set forth in SEQ ID NO:4. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer such as one or more selected from: (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5 or 27-34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

Exemplary spacers include those containing portion(s) of an immunoglobulin constant region such as those containing an Ig hinge, such as an IgG hinge domain. In some aspects, the spacer includes an IgG hinge alone, an IgG hinge linked to one or more of a C_{H}2 and C_{H}3 domain, or IgG hinge linked to the C_{H}3 domain. In some embodiments, the IgG hinge, C_{H}2 and/or C_{H}3 can be derived all or in part from IgG4 or IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fe sequence set forth in SEQ ID NO: 60 or an N176Q. at position 176, in the C_{H}2 region, of the full-length IgG2 Fe sequence set forth in SEQ ID NO: 59. In some embodiments, the spacer is or comprises an IgG4/2 chimeric hinge or a modified IgG4 hinge; an IgG2/4 chimeric C_{H}2 region; and an IgG4 C_{H}3 region and optionally is about 228 amino acids in length; or a spacer set forth in SEQ ID NO: 291. In some embodiments, the ligand- (e.g., antigen-) binding or recognition domain of the CAR is linked to an intracellular region, e.g., containing one or more intracellular signaling components, such as an intracellular signaling region or domain, and/or signaling components that mimic activation through an antigen receptor complex, such as a TCR complex, and/or signal via another cell surface receptor. Thus, in some embodiments, the extracellular region, e.g., containing a binding domain such as an antigen binding component (e.g., antibody), is linked to one or more transmembrane and intracellular region(s) or domain(s). In some embodiments, the transmembrane domain is fused to the extracellular region. In some embodiments, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular region and transmembrane can be linked directly or indirectly. In some embodiments, the extracellular region and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8; in some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

c. Intracellular Region

In some aspects, the recombinant receptor, e.g., CAR, encoded in the modified T cell stimulation-associated locus, includes an intracellular region (also called cytoplasmic region) that comprises a signaling region or domain. In some embodiments, the intracellular region comprises an intracellular signaling region or domain. In some embodiments, the intracellular signaling region or domain is or comprises a primary signaling region, a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the recombinant receptor, e.g., CAR, includes at least one intracellular signaling component or components, such as an intracellular signaling region or domain. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic (or intracellular) domain or regions, e.g., intracellular signaling region, of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region or domain of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

### (i) Costimulatory Signaling Domain

In some embodiments, to promote full stimulation and/or activation, one or more components for generating secondary or costimulatory signal is included in the encoded CAR. In other embodiments, the encoded CAR does not include a component for generating a costimulatory signal. In some aspects, an additional receptor polypeptide or portion thereof is expressed in the same cell and provides the component for generating the secondary or costimulatory signal.

In some embodiments, the encoded CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components.

In some embodiments, one or more different recombinant receptors can contain one or more different intracellular signaling region(s) or domain(s). In some embodiments, the primary cytoplasmic signaling region is included within one encoded CAR, whereas the costimulatory component is provided by another receptor, e.g., another CAR recognizing another antigen. In some embodiments, the encoded CARs include activating or stimulatory CARs, and costimulatory CARs, both expressed on the same cell (see WO2014/055668).

In certain embodiments, the intracellular signaling region comprises a CD28 transmembrane and signaling domain linked to a CD3 (e.g., CD3ζ) intracellular region or domain. In some embodiments, the intracellular region comprises a chimeric CD28 and CD137 (4-1BB, TNFRSF9) co-stimulatory domains, linked to a CD3ζ intracellular region or domain.

In some embodiments, the encoded CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS. In some embodiments, the costimulatory molecule is a human costimulatory molecule.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain or region of CD137(4-1BB) or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some cases, the encoded CARs are referred to as first, second, third or fourth generation CARs. In some aspects, a first generation CAR is one that solely provides a primary stimulation or activation signal, e.g., via CD3-chain induced signal upon antigen binding; in some aspects, a second-generation CAR is one that provides such a signal and costimulatory signal, such as one including an intracellular signaling region(s) or domain(s) from one or more costimulatory receptor such as CD28, CD137 (4-1BB), OX40 (CD134), CD27, DAP10, DAP12, NKG2D, ICOS and/or other costimulatory receptors; in some aspects, a third generation CAR is one that includes multiple costimulatory domains of different costimulatory receptors, e.g., selected from CD28, CD137 (4-1BB), OX40 (CD134), CD27, DAP10, DAP12, NKG2D, ICOS and/or other costimulatory receptors; in some aspects, a fourth generation CAR is one that includes three or more costimulatory domains of different costimulatory receptors, e.g., selected from CD28, CD137 (4-1BB), OX40 (CD134), CD27, DAP10, DAP12, NKG2D, ICOS and/or other costimulatory receptors.

### (ii) Primary Signaling Region, e.g., CD3ζ Chain

In some embodiments, the encoded recombinant receptor includes an intracellular component of a TCR complex, such as a TCR CD3 chain that mediates T-cell activation and cytotoxicity, e.g., CD3 zeta chain. Thus, in some aspects, the antigen-binding or antigen-recognition domain is linked to one or more cell signaling modules. In some embodiments, cell signaling modules include CD3 transmembrane domain, CD3 intracellular signaling domains, and/or other CD transmembrane domains. In some embodiments, the encoded recombinant receptor, e.g., CAR, further includes one or more additional molecules such as Fc receptor gamma (FcRγ), CD8 alpha, CD8 beta, CD4, CD25 or CD16. For example, in some aspects, the CAR includes a chimeric molecule between CD3 zeta (CD3ζ) and one or more of CD8 alpha, CD8 beta, CD4, CD25 or CD16.

In the context of a natural TCR, full stimulation generally requires not only signaling through the TCR, but also a costimulatory signal. T cell stimulation is in some aspects can be mediated by two classes of cytoplasmic signaling sequences: those that initiate antigen-dependent primary activation through the TCR (primary cytoplasmic signaling region(s) or domain(s)), and those that act in an antigen-independent manner to provide a secondary or co-stimulatory signal (secondary cytoplasmic signaling region(s) or domain(s)). In some aspects, the CAR includes one or both of such signaling components.

In some aspects, the encoded CAR includes an intracellular region comprising a primary cytoplasmic signaling region that regulates primary stimulation and/or activation of the TCR complex. Primary cytoplasmic signaling region(s) that act in a stimulatory manner may contain signaling motifs which are known as immunoreceptor tyrosine-based activation motifs or ITAMs, e.g., derived from CD3 zeta (CD3ζ). In some embodiments, the CAR contain(s) a cytoplasmic signaling domain, fragment or portion thereof, or sequence derived from CD3ζ. In some embodiments, the intracellular (or cytoplasmic) signaling region comprises a human CD3 zeta chain or a fragment or portion thereof, including the intracellular or cytoplasmic stimulatory signaling domain of CD3ζ or functional variant thereof, such as an 112 AA cytoplasmic domain of isoform 3 of human CD3ζ (Accession No.: P20963.2) or a CD3ζ signaling domain as described in U.S. Pat. No. 7,446,190 or U.S. Pat. No. 8,911,993. In some embodiments, the intracellular region of the encoded recombinant receptor comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15 or a partial sequence thereof. In some embodiments, exemplary CD3ζ chain or a fragment thereof encoded by the modified T cell stimulation-associated locus include the ITAM domains of the CD3ζ chain, e.g., amino acid residues 61-89, 100-128 or 131-159 of the human CD3ζ chain precursor sequence set forth in SEQ ID NO:292 or a sequence of amino acids that containing one or more ITAM domains from the CD3ζ chain and exhibits at least or at least about 85%, 86%, 87%, 88%, 89%, 90%, 91%, 92%, 93%, 94%, 95%, 96%, 97%, 98%, 99% or more sequence identity to SEQ ID NO: 292.

In some embodiments, the cell is engineered to express one or more additional molecules (e.g., polypeptides, such as an additional recombinant receptor polypeptides or portion thereof) are used to regulate, control, or modulate function and/or activity of the encoded CAR. Exemplary multi-chain recombinant receptors, such as multi-chain CARs, and are described herein, for example, in Section IV.B.2.

In some embodiments, the encoded CAR contains an antibody, e.g., an antibody fragment, a transmembrane domain that is or contains a transmembrane portion of CD28 or a functional variant thereof, and an intracellular signaling region containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer. In some embodiments, the recombinant receptor comprises a CD3 zeta (CD3ζ) at the C-terminus of the receptor.

### 2. Multi-Chain CARs

In some embodiments, the recombinant receptor encoded by the nucleic acid sequences of the modified T cell stimulation-associated locus can be a multi-chain CAR. In some embodiments, if the multi-chain CAR comprising two or more polypeptide chains is expressed in the cell, at least one of the polypeptide chains encoded by the modified T cell stimulation-associated locus. In some aspects, the polynucleotide used to introduce nucleic acid sequence encoding one or more chains of the multi-chain CAR can include any described in Section II.B herein. In some aspects, a polynucleotide, e.g., template polynucleotide, contains a transgene encoding at least one chain of the multi-chain CAR or a portion thereof, such as at least a portion of at least one polypeptide of a multi-chain CAR. In some aspects, the transgene also includes sequences encoding a different or additional polypeptide, e.g., the other or additional chain of the multi-chain CAR, or additional molecules, such as those described in Section IV.B.2 herein. In some aspects, an additional polynucleotide, e.g., an additional template polynucleotide, can be introduced, that encodes additional components of the multi-chain CAR. In some aspects, the additional polynucleotide can be any polynucleotide described herein, e.g., in Section II.B.2, or a modified form thereof, such as one comprising different homology arms for targeting the nucleic acid for integration at a distinct genomic locus.

In some embodiments, the provided engineered cells include cells that express multi-chain receptors, such as multi-chain CARs In some embodiments, exemplary multi-chain CARs can contain two or more genetically engineered receptors on the cell, which together can comprise a functional recombinant receptor. In some aspects, the various polypeptide chains in combination can perform functions or activities of a CAR, and/or regulate, control, or modulate function and/or activity of the CAR. In some aspects, a multi-chain CAR can contain two or more polypeptide chains, each recognizing the same of a different antigen and typically each including different regions or domains, such as a different intracellular signaling component. In some aspects, the modified T cell stimulation-associated locus can include nucleic acid sequence encoding at least one chain of a multi-chain receptor, such as a multi-chain CAR.

In some embodiments, the chimeric receptor is multi-chain CAR or a dual-chain CAR, that comprises two or more polypeptide chains. In some embodiments, the multi-chain receptor is a regulatable CAR, a conditionally active CAR or an inducible CAR. In some aspects, two or more polypeptides of the recombinant receptor, such as a dual-chain CAR, allows spatial or temporal regulation or control of specificity, activity, antigen (or ligand) binding, function and/or expression of the recombinant receptors. In some of such embodiments, the recombinant receptor encoded by the nucleic acid sequences at the modified T cell stimulation-associated locus can include one or more chains of the dual-chain or multi-chain receptors. In some aspects, in cases where only one of the dual-chain CAR is encoded by the modified T cell stimulation-associated locus, the other chain can be encoded by a separate nucleic acid molecule that is integrated at a different genomic location or is episomal.

In some embodiments, the multi-chain CARs can include combinations of activating and costimulatory CARs. For example, in some embodiments, the multi-chain CAR can include two polypeptides encoding CARs targeting two different antigens present individually on non-target cells, e.g., normal cells, but present together only on cells of the disease or condition to be treated. In some embodiments, the multi-chain CARs can include an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat. In some aspects, multi-chain CARs can include one or more polypeptides encoding CARs that are capable of being regulated, modulated or controlled.

In some embodiments, the multi-chain CAR includes one or more polypeptide chains encode one or more domains or regions of a CAR. In some aspects, various polypeptide chains in combination can comprise a CAR. In some embodiments, one or more additional domains or regions are present in the CAR. In some embodiments, various domains or regions present in one or more polypeptide chains of the multi-chain CAR are used to regulate, control, or modulate function and/or activity of the CAR. In some embodiments, the engineered cells express two or more polypeptide chains that contain different components, domains or regions. In some aspects, two or more polypeptide chains allows spatial or temporal regulation or control of specificity, activity, antigen (or ligand) binding, function and/or expression of the recombinant receptors. In some embodiments of the multi-chain CAR involving more than one polypeptides, e.g., 2 or more polypeptides, the nucleic acid sequence encoding at least one polypeptide, is targeted for integration at the endogenous T cell stimulation-associated locus. In some embodiments, the nucleic acid sequence encoding an additional molecule or polypeptide, e.g., additional polypeptide chain of the multi-chain CAR or an additional molecule, can be targeted at the same locus, e.g. by virtue of placement on the same polynucleotide used for targeting. In some nucleic acid sequence encoding an additional molecule or polypeptide is targeted at a different locus or is delivered by different methods.

In some aspects, one or more polypeptide chain encoding domains or regions of a CAR can target one or more antigens or molecules. Exemplary multi-chain CARs include those described in, for example, in International Pat. App. Pub. No. WO 2014055668 or Fedorov et al., Sci. Transl. Medicine, Sci Transl Med. (2013) 5(215):215ra172; Sadelain, Curr Opin Immunol. (2016) 41: 68-76; Wang et al. (2017) Front. Immunol. 8:1934; Mirzaei et al. (2017) Front. Immunol. 8:1850; Marin-Acevedo et al. (2018) Journal of Hematology & Oncology 11:8; Fesnak et al. (2016) Nat Rev Cancer. 16(9): 566-581; and Abate-Daga and Davila, (2016) Molecular Therapy—Oncolytics 3, 16014.

In some embodiments, the engineered cells can express a first polypeptide chain of the recombinant receptor, e.g., CAR, which is capable of inducing an activating or stimulating signal to the cell, generally upon specific binding to the antigen recognized by the first polypeptide chain e.g., the first antigen. In some embodiments, the cell can further express a second polypeptide chain of the recombinant receptor, e.g., CAR, in some cases called a chimeric costimulatory receptor, which is capable of inducing a costimulatory signal to the immune cell, generally upon specific binding to a second antigen recognized by the second polypeptide chain. In some embodiments, the first antigen and second antigen are the same. In some embodiments, the first antigen and second antigen are different.

In some embodiments, the first and/or second polypeptide chain is capable of inducing an activating or stimulating signal to the cell. In some embodiments, the receptor includes an intracellular signaling component containing ITAM or ITAM-like motifs. In some embodiments, the activation induced by the first polypeptide chain involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g., CD4 or CD8, etc.), activation of one or more transcription factors, such as NF-κB and/or AP-1, and/or induction of gene expression of factors such as cytokines, proliferation, and/or survival. In some embodiments, the activating domain is included within at least one of the multi-chain CAR, such as the polypeptide chain that is encoded by the modified T cell stimulation-associated locus, whereas the costimulatory component is provided by another polypeptide recognizing another antigen. In some embodiments, the engineered cells can include multi-chain CARs, including activating or stimulatory CARs, costimulatory CARs, both expressed on the same cell (see WO2014/055668). In some aspects, the cells express one or more stimulatory or activating CAR (such as those encoded by the modified T cell stimulation-associated locus as described herein, e.g., in Section IV.A) and/or a costimulatory CAR.

In some embodiments, the first and/or second polypeptide chain, includes intracellular signaling regions or domains of costimulatory receptors such as CD28, CD137 (4-1BB), OX40 (CD134), CD27, DAP10, DAP12, NKG2D, ICOS and/or other costimulatory receptors. In some embodiments, the first and second polypeptide chains can contain intracellular signaling domain(s) of a costimulatory receptor that are different. In one embodiment, the first polypeptide chain contains a CD28 costimulatory signaling domain and the second polypeptide chain contain a 4-1BB co-stimulatory signaling region or vice versa.

In some embodiments, the first and/or second polypeptide chain includes both an intracellular signaling domain containing ITAM or ITAM-like motifs, such as those from a CD3zeta (CD3ζ) chain or a fragment or portion thereof, such as the CD3ζ intracellular signaling domain and an intracellular signaling domain of a costimulatory receptor. In some embodiments, the first polypeptide chain contains an intracellular signaling domain containing ITAM or ITAM-like motifs and the second polypeptide chain contains an intracellular signaling domain of a costimulatory receptor. The costimulatory signal in combination with the activating or stimulating signal induced in the same cell is one that results in an immune response, such as a robust and sustained immune response, such as increased gene expression, secretion of cytokines and other factors, and T cell mediated effector functions such as cell killing.

In some embodiments, neither ligation of the first polypeptide chain alone nor ligation of the second polypeptide chain alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the multiple polypeptide chains are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, one or more chain of the multi-chain CAR can include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects. In some embodiments, the inhibitory CAR can be encoded by the same polynucleotide as the stimulating or activating CAR (e.g., containing a CD3zeta (CD3ζ) chain or a fragment or portion thereof), or by a different polynucleotide.

In some embodiments, the two polypeptide chains of the multi-chain CAR induce, respectively, an activating and an inhibitory signal to the cell, such that ligation of one polypeptide chain to its antigen activates the cell or induces a response, but ligation of the second polypeptide chain, e.g., an inhibitory receptor, to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, an additional receptor polypeptide expressed in the cell further includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PDCD1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some embodiments, a multi-chain CAR can be employed where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific polypeptides, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

In some embodiments, one of the first and/or second polypeptide chains can regulate the expression, antigen binding and/or activity of the other polypeptide chain.

In some aspects, a two polypeptide chain system can be used to regulate the expression of at least one of the polypeptide chains. In some embodiments, the first polypeptide chain contains a first ligand- (e.g., antigen-) binding domain linked to a regulatory molecule, such as a transcription factor, linked via a regulatable cleavage element. In some aspects, the regulatable cleavage element is derived from a modified Notch receptor (e.g., synNotch), which is capable of cleaving and releasing an intracellular domain upon engagement of the first ligand- (e.g., antigen-) biding domain. In some aspects, the second polypeptide chain contains a second ligand- (e.g., antigen-) binding domain linked to an intracellular signaling component capable of inducing an activating or stimulating signal to the cell, such as an ITAM-containing intracellular signaling domain. In some aspects, the nucleic acid sequence encoding the second polypeptide chain is operably linked to transcriptional regulatory elements, e.g., promoter, that is capable of being regulated by a particular transcription factor, e.g., transcription factor encoded by the first polypeptide chain. In some aspects, engagement of a ligand or an antigen to the first ligand- (e.g., antigen-) binding domain leads to proteolytic release of the transcription factor, which in turn can induce the expression of the second polypeptide chain (see Roybal et al. (2016) Cell 164:770-779; Morsut et al. (2016) Cell 164:780-791). In some embodiments, the first antigen and second antigen are different.

In some instances, the recombinant receptor, e.g., CAR, is capable of being regulated, controlled, induced or inhibited, can be desirable to optimize the safety and efficacy of a therapy with the recombinant receptor. In some embodiments, the multi-chain CAR is a regulatable CAR. In some aspects, provided herein is an engineered cell comprising a CAR that is capable of being regulated. A recombinant receptor that is capable of being regulated, also referred to herein as a “regulatable recombinant receptor,” or a “regulatable CAR” refers to multiple polypeptides, such as a set of at least two polypeptide chains, which when expressed in an engineered cell (e.g., engineered T cell), provides the engineered cell with the ability to generate an intracellular signal under the control of an inducer.

In some embodiments, the polypeptides of the regulatable CAR contain multimerization domains that are capable of multimerization with another multimerization domain. In some embodiments, the multimerization domain is capable of multimerization upon binding to an inducer. For example, the multimerization domain can bind an inducer, such as a chemical inducer, which results in multimerization of the polypeptides of the regulatable CAR by virtue of multimerization of the multimerization domain, thereby producing the regulatable CAR.

In some embodiments, one polypeptide of the regulatable CAR comprises a ligand- (e.g., antigen-) binding domain and a different polypeptide of the regulatable CAR comprises an intracellular signaling region, wherein multimerization of the two polypeptides by virtue of multimerization of the multimerization domain produces a regulatable CAR comprising a ligand-binding domain and an intracellular signaling region. In some embodiments, multimerization can induce, modulate, activate, mediate and/or promote signals in the engineered cell containing the regulatable CAR. In some embodiments, an inducer binds to a multimerization domain at least one polypeptide of a regulatable CAR and induces a conformational change of the regulatable CAR, wherein the conformational change activates signaling. In some embodiments, binding of a ligand to such chimeric receptors induces conformational changes in the polypeptide chain, including, in some cases, polypeptide chain oligomerization, which can render the receptors competent for intracellular signaling.

In some embodiments, an inducer functions to couple or multimerize (e.g., dimerize) a set of at least two polypeptide chains of a regulatable CAR expressed in an engineered cell in order for the regulatable CAR to produce a desired intracellular signal such as during interaction of the regulatable CAR with a target antigen. Coupling or multimerization of at least two polypeptides of a regulatable CAR by an inducer is achieved upon binding of an inducer to a multimerization domain. For example, in some embodiments, a first polypeptide and a second polypeptide in an engineered cell may each comprise a multimerization domain capable of binding an inducer. Upon binding of the multimerization domain by the inducer, the first polypeptide and the second polypeptide are coupled together to produce the desired intracellular signal. In some embodiments, a multimerization domain is located on an intracellular portion of a polypeptide. In some embodiments, a multimerization domain is located on an extracellular portion of a polypeptide.

In some embodiments, a set of at least two polypeptides of a regulatable CAR comprises two, three, four, or five or more polypeptides. In some embodiments, the set of at least two polypeptides are the same polypeptides, for example, two, three, or more of the same polypeptides comprising an intracellular signaling region, and a multimerization domain. In some embodiments, the set of at least two polypeptides are different polypeptides, for example, a first polypeptide comprising an ligand- (e.g., antigen-) binding domain and a multimerization domain and a second polypeptide comprising an intracellular signaling region and a multimerization domain. In some embodiments, the intercellular signal is generated in the presence of an inducer. In some embodiments, the intracellular signal is generated in the absence of an inducer, e.g., an inducer interferes with multimerization of at least two polypeptides of a regulatable CAR thereby preventing intracellular signaling by the regulatable CAR.

In some embodiments, the multi-chain CAR, the nucleic acid sequence encoding at least one of the polypeptide chains, is integrated into the endogenous T cell stimulation-associated locus, e.g., by HDR. In some embodiments, the nucleic acid sequence encoding the other of the two or more separate polypeptide chains, can be targeted within the same locus (e.g., within the same transgene, and can be placed 5′ or 3′ of the nucleic acid sequence encoding the other polypeptide chain), or at a different locus. In some aspects, the introduction of the nucleic acid sequence encoding the other of the two or more separate polypeptide chains may be via different delivery methods, e.g., by transient delivery methods or as an episomal nucleic acid molecule.

In some embodiments, one or more of the polypeptide chains of a multi-chain CAR, can include a multimerization domain. In some embodiments, the multimerization domain can multimerize (e.g., dimerize), upon binding of an inducer. An inducer contemplated herein includes, but is not limited to, a chemical inducer or a protein (e.g., a caspase). In some embodiments, the inducer is selected from an estrogen, a glucocorticoid, a vitamin D, a steroid, a tetracycline, a cyclosporine, Rapamycin, Coumermycin, Gibberellin, FK1012, FK506, FKCsA, rimiducid or HaXS, or analogs or derivatives thereof. In some embodiments, the inducer is AP20187 or an AP20187 analog, such as, AP1510.

In some embodiments, the multimerization domain can multimerize (e.g., dimerize), upon binding of an inducer such as an inducer provided herein. In some embodiments, the multimerization domain can be from an FKBP, a cyclophilin receptor, a steroid receptor, a tetracycline receptor, an estrogen receptor, a glucocorticoid receptor, a vitamin D receptor, Calcineurin A, CyP-Fas, FRB domain of mTOR, GyrB, GAI, GID1, Snap-tag and/or HaloTag, or portions or derivatives thereof. In some embodiments, the multimerization domain is an FK506 binding protein (FKBP) or derivative thereof, or fragment and/or multimer thereof, such as FKBP12v36. In some embodiments, FKBP comprises the amino acid sequence GVQVETISPGDGRTFPKRGQTCVVHYTGMLEDGKKMDSSRDRNKPFKFMLGKQEVIRGWEEG VAQMSVGQRAKLTISPDYAYGATGHPGIIPPHATLVFDVELLKLE (SEQ ID NO:293). In some embodiments, FKBP12v36 comprises the amino acid sequence GVQVETISPGDGRTFPKRGQTCVVHYTGMLEDGKKVDSSRDRNKPFKFMLGKQEVIRGWEEGV AQMSVGQRAKLTISPDYAYGATGHPGIIPPHATLVFDVELLKLE (SEQ ID NO:294).

Exemplary inducers and corresponding multimerization domains are known, e.g., as described in U.S. Pat. App. Pub. No. 2016/0046700, Clackson et al. (1998) Proc Natl Acad Sci USA. 95(18):10437-42; Spencer et al. (1993) Science 262(5136):1019-24; Farrar et al. (1996) Nature 383 (6596):178-81; Miyamoto et al. (2012) Nature Chemical Biology 8(5): 465-70; Erhart et al. (2013) Chemistry and Biology 20(4): 549-57). In some embodiments, the inducer is rimiducid (also known as AP1903; CAS Index Name: 2-Piperidinecarboxylic acid, 1-[(2S)-1-oxo-2-(3,4,5-trimethoxyphenyl)butyl]-, 1,2-ethanediylbis [imino (2-oxo-2, 1-ethanediyl)oxy-3,1-phenylene[(1R)-3-(3,4-Dimethoxyphenyl)propylidene]]ester, [2S-[1(R*),2R*[S*[S*[1(R*),2R]]]]]-(9Cl); CAS Registry Number: 195514-63-7; Molecular Formula: C₇₈H₉₈N₄O₂₀; Molecular Weight: 1411.65), and the multimerization domain is an FK506 binding protein (FKBP).

In some embodiments, the cell membrane of the engineered cell is impermeable to the inducer. In some embodiments, the cell membrane of the engineered cell is permeable to the inducer.

In some embodiments, the regulatable CAR are not part of a multimer or a dimer in the absence of the inducer. Upon the binding of the inducer, the multimerization domains can multimerize, e.g., dimerize. In some aspects, multimerization of the multimerization domain results in multimerization of a polypeptide of the regulatable CAR with another polypeptide of the regulatable CAR, e.g. multimeric complex of at least two polypeptides of the regulatable CARs. In some embodiments, multimerization of the multimerization domain can induce, modulate, activate, mediate and/or promote signal transduction by virtue of inducing physical proximity of signaling components or formation of the multimer or dimer. In some embodiments, upon the binding of an inducer, multimerization of the multimerization domain also induces multimerization of signaling domains linked, directly or indirectly, to the multimerization domain. In some embodiments, the multimerization induces, modulates, activates, mediates and/or promotes signaling through the signaling domain or region. In some embodiments, the signaling domain or region linked to the multimerization domain is an intracellular signaling region.

In some embodiments, the multimerization domain is intracellular or is associated with the cell membrane on the intracellular or cytoplasmic side of the engineered cell (e.g., engineered T cell). In some aspects, the intracellular multimerization domain is linked, directly or indirectly, to a membrane association domain (e.g., a lipid linking domain), such as a myristoylation domain, palmitoylation domain, prenylation domain, or a transmembrane domain. In some embodiments, the multimerization domain is intracellular, and is linked to the extracellular ligand- (e.g., antigen-) binding domain via a transmembrane domain. In some embodiments, the intracellular multimerization domain is linked, directly or indirectly, to the intracellular signaling region. In some aspects, induced multimerization of the multimerization domain also brings the intracellular signaling regions in proximity with one another, to allow multimerization, e.g., dimerization, and stimulate intracellular signaling. In some embodiments, a polypeptide of the regulatable CAR comprises a transmembrane domain, one or more intracellular signaling region(s), and one or more multimerization domain(s), each of which are linked directly or indirectly.

In some embodiments, the multimerization domain is extracellular or is associated with the cell membrane on the extracellular side of the engineered cell (e.g., engineered T cell). In some aspects, the extracellular multimerization domain is linked, directly or indirectly, to a membrane association domain (e.g., a lipid linking domain), such as a myristoylation domain, palmitoylation domain, prenylation domain, or a transmembrane domain. In some embodiments, the extracellular multimerization domain is linked, directly or indirectly, to a ligand-binding domain, e.g., an antigen-binding domain such as for binding to an antigen associated with a disease. In some embodiments, the multimerization domain is extracellular, and is linked to an intracellular signaling region via a transmembrane domain.

In some aspects, the membrane association domain is a transmembrane domain of an existing transmembrane protein. In some examples, the membrane association domain is any of the transmembrane domains described herein. In some aspects, the membrane association domain contains protein-protein interaction motifs or transmembrane sequences.

In some aspects, the membrane association domain is an acylation domain, such as a myristoylation domain, palmitoylation domain, prenylation domain (i.e., farnesylation, geranyl-geranylation, CAAX Box). For example, the membrane association domain can be an acylation sequence motif present in N-terminus or C-terminus of a protein. Such domains contain particular sequence motifs that can be recognized by acyltransferases that transfer acyl moieties to the polypeptide that contains the domain. For example, the acylation motifs can be modified with a single acyl moiety (in some cases, followed by several positively charged residues (e.g. human c-Src: MGSNKSKPKDASQRRR (SEQ ID NO:295) to improve association with anionic lipid head groups). In other aspects, the acetylation motif is capable of being modified with multiple acyl moieties. For example, dual acylation regions are located within the N-terminal regions of certain protein kinases, such as a subset of Src family members (e.g., Yes, Fyn, Lek) and G-protein alpha subunits. Exemplary dual acylation regions contain the sequence motif Met-Gly-Cys-Xaa-Cys, (SEQ ID NO:296) where the Met is cleaved, the Gly is N-acylated and one of the Cys residues is S-acylated. The Gly often is myristoylated and a Cys can be palmitoylated.

Other exemplary acylation regions include sequence motif Cys-Ala-Ala-Xaa (so called “CAAX boxes”; SEQ ID NO:297) that can modified with C15 or 010 isoprenyl moieties, and are known (see, e.g., Gauthier-Campbell et al. (2004) Molecular Biology of the Cell 15:2205-2217; Glabati et al. (1994) Biochem. J. 303: 697-700 and Zlakine et al. (1997) J. Cell Science 110:673-679; ten Klooster et al. (2007) Biology of the Cell 99:1-12; Vincent et al. (2003) Nature Biotechnology 21:936-40). In some embodiments, the acyl moiety is a C1-C20 alkyl, C2-C20 alkenyl, C2-C20 alkynyl, C3-C6 cycloalkyl, C1-C4 haloalkyl, C4-C12 cycloalkylalkyl, aryl, substituted aryl, or aryl (C1-C4) alkyl. In some embodiments, the acyl-containing moiety is a fatty acid, and examples of fatty acid moieties are propyl (C3), butyl (C4), pentyl (C5), hexyl (C6), heptyl (C7), octyl (C8), nonyl (C9), decyl (C10), undecyl (C11), lauryl (C12), myristyl (C14), palmityl (C16), stearyl (C18), arachidyl (C20), behenyl (C22) and lignoceryl moieties (C24), and each moiety can contain 0, 1, 2, 3, 4, 5, 6, 7 or 8 unsaturated bonds (i.e., double bonds). In some examples, the acyl moiety is a lipid molecule, such as a phosphatidyl lipid (e.g., phosphatidyl serine, phosphatidyl inositol, phosphatidyl ethanolamine, phosphatidyl choline), sphingolipid (e.g., shingomyelin, sphingosine, ceramide, ganglioside, cerebroside), or modified versions thereof. In certain embodiments, one, two, three, four or five or more acyl moieties are linked to a membrane association domain.

In some aspects, the membrane association domain is a domain that promotes an addition of a glycolipid (also known as glycosyl phosphatidylinositols or GPIs). In some aspects, a GPI molecule is post-translationally attached to a protein target by a transamidation reaction, which results in the cleavage of a carboxy-terminal GPI signal sequence (see, e.g., White et al. (2000) J. Cell Sci. 113:721) and the simultaneous transfer of the already synthesized GPI anchor molecule to the newly formed carboxy-terminal amino acid (See, e.g., Varki A, et al., editors. Essentials of Glycobiology. Cold Spring Harbor (NY): Cold Spring Harbor Laboratory Press; 1999. Chapter 10, Glycophospholipid Anchors. Available from: https://www.ncbi.nlm.nih.gov/books/NBK20711/). In certain embodiments, the membrane association domain is a GPI signal sequence.

In some embodiments, a multimerization domain as provided herein is linked to an intracellular signaling regions, e.g., a primary signaling region and/or costimulatory signaling domains. In some embodiments, the multimerization domain is extracellular, and is linked to the intracellular signaling region via a transmembrane domain. In some embodiments, the multimerization domain is intracellular, and is linked to the ligand- (e.g., antigen-) binding domain via a transmembrane domain. The ligand-binding domain and transmembrane domain can be linked directly or indirectly. In some embodiments, the ligand-binding domain and transmembrane are linked by a spacer, such as any described herein. In some embodiments, the multimerization domain is an FK506 binding protein (FKBP) or derivative or fragment thereof, such as FKBP12v36. In some examples, upon the introduction of an inducer, such as a rimiducid, the polypeptides of the regulatable CAR multimerize, e.g., dimerize, thereby stimulating the signaling domains associated with the multimerization domain and forming a multimeric complex. Formation of the multimeric complex results in inducing, modulating, stimulating, activating, mediating and/or promoting signals through intracellular signaling region.

In some embodiments, signaling through the regulatable CAR can be modulated in a conditional manner through conditional multimerization. For example, the multimerization domain of the polypeptides of the regulatable CAR can bind an inducer to multimerize, and the inducer can be provided exogenously. In some aspects, upon binding of the inducer, the multimerization domain multimerizes and induces, modulates, activates, mediates and/or promotes signaling through the signaling domain. For example, the inducer can be exogenously administered, thereby controlling the location and duration of the signal provided to the engineered cell containing the regulatable CAR. In some embodiments, the multimerization domain of the polypeptides of the regulatable CAR can bind an inducer to multimerize, and the inducer can be provided endogenously. For example, the inducer can be produced endogenously by the engineered cell (e.g., engineered T cell) from a recombinant expression vector or from the genome of the engineered cell under the control of an inducible or conditional promoter, thereby controlling the location and duration of the signal provided to the engineered cell containing the regulatable CAR.

In some embodiments, the regulatable CAR is controlled using a suicide switch. Exemplary chimeric receptors utilize an inducible caspase-9 (iCasp9) system, comprising a fusion of human caspase-9 and a modified FKBP dimerization domain, allowing conditional dimerization upon binding with an inducer, e.g., AP1903. Upon dimerization by binding of the inducer, caspase-9 becomes activated and results in apoptosis and cell death of the cells expressing the chimeric receptor (see, e.g., Di Stasi et al. (2011) N. Engl. J. Med. 365:1673-1683).

In some embodiments, exemplary regulatable CAR includes: (1) a first polypeptide of a regulatable CAR comprising: (i) intracellular signaling region; and (ii) at least one multimerization domain capable of binding an inducer; and (2) a second polypeptide of a regulatable CAR comprising: (i) a ligand- (e.g., antigen-) binding domain; (ii) a transmembrane domain; and (iii) at least one multimerization domain capable of binding an inducer. In some embodiments, exemplary regulatable CAR includes: (1) a first polypeptide of a regulatable CAR comprising: (i) a transmembrane domain or an acylation domain; (ii) intracellular signaling region; and (iii) at least one multimerization domain capable of binding an inducer; and (2) a second polypeptide of a regulatable CAR comprising: (i) a ligand- (e.g., antigen-) binding domain; (ii) a transmembrane domain; and (iii) at least one multimerization domain capable of binding an inducer. In some embodiments, the intracellular signaling region further comprises a costimulatory signaling domain. In some embodiments, the second polypeptide further comprises a costimulatory signaling domain. In some embodiments, the at least one multimerization domain(s) on both polypeptides is intracellular. In some embodiments, the at least one multimerization domain(s) on both polypeptides is extracellular.

In some embodiments, exemplary regulatable CAR includes: (1) a first polypeptide of a regulatable CAR comprising: (i) at least one extracellular multimerization domain capable of binding an inducer; (ii) a transmembrane domain; and (iii) intracellular signaling region; and (2) a second polypeptide of a regulatable CAR comprising: (i) a ligand- (e.g., antigen-) binding domain; (ii) at least one extracellular multimerization domain capable of binding an inducer and (iii) a transmembrane domain, an acylation domain or a GPI signal sequence. In some embodiments, the intracellular signaling region further comprises a costimulatory signaling domain. In some embodiments, the second polypeptide further comprises a costimulatory signaling domain.

In some embodiments, exemplary regulatable CAR includes: (1) a first polypeptide of a regulatable CAR comprising: (i) a transmembrane domain or an acylation domain; (ii) at least one costimulatory domain; (iii) a multimerization domain capable of binding an inducer and (iv) intracellular signaling region; and (iii) at least one costimulatory domain; and (2) a second polypeptide of a regulatable CAR comprising: (i) a ligand- (e.g., antigen-) binding domain; (ii) a transmembrane domain; (iii) at least one costimulatory domain; and (iv) at least one extracellular multimerization domain capable of binding an inducer.

In some aspects, any of the regions and/or domains described in the exemplary regulatable CARs can be ordered in various different orders. In some aspects, the various polypeptides of the regulatable CAR(s) contain the multimerization domain on the same side of the cell membrane, e.g., the multimerization domain in the two or more polypeptides are all intracellular or all extracellular.

Variations of regulatable CARs are known, for example, described in U.S. Pat. App. Pub. No. 2014/0286987, U.S. Pat. App. Pub. No. 2015/0266973, International Pat. App. Pub. No. WO2014/127261, and International Pat. App. Pub. No. WO2015/142675.

### 3. Chimeric Auto-Antibody Receptor (CAAR)

In some embodiments, the recombinant receptor encoded by the modified T cell stimulation-associated locus is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling region, a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

### 4. T Cell Receptors (TCRs)

In some embodiments, the encoded recombinant receptor is a T cell receptor (TCR) or antigen-binding portion thereof, such as a recombinant TCR, that recognizes an intracellular and/or a peptide epitope or T cell epitope of a target polypeptide, such as an antigen of a tumor, viral or autoimmune protein. In some aspects, the encoded receptor is or includes a recombinant TCR. In some aspects, the recombinant TCR is a single-chain TCR or a multi-chain TCR, such as a dual-chain TCR.

In some embodiments, a “T cell receptor” or “TCR” is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. In some embodiments, TCRs that exist in αβ and γδ forms are generally structurally similar, but T cells expressing them may have distinct anatomical locations or functions. A TCR can be found on the surface of a cell or in soluble form. In some embodiments, the TCR is a dual-chain TCR, comprising a TCRα and a TCRβ; or a TCRγ and a TCRδ chain. In some aspects, a TCR is found on the surface of T cells (or T lymphocytes) where it is generally responsible for recognizing antigens bound to major histocompatibility complex (MHC) molecules.

In some embodiments, a TCR encompasses a full-length TCRs or antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α (Vα) chain and variable β (Vβ) chain of a TCR, or antigen-binding fragments thereof sufficient to form a binding site for binding to a specific MHC-peptide complex.

In some embodiments, the encoded recombinant receptor is a TCR, and the modified locus encodes a chain of the TCR. In some embodiments, the encoded recombinant receptor is a dual-chain TCR, and the modified locus encodes one chain of the dual-chain TCR. In some embodiments, the encoded recombinant receptor is a dual-chain TCR, and the modified locus encodes both chains of the dual-chain TCR. In some embodiments, the encoded recombinant receptor is a TCR comprising an alpha chain and a beta chain, and the modified locus encodes both alpha and beta chains of a TCR. In some embodiments, the nucleic acid sequence encoding the alpha chain of the TCR and the beta chain of the TCR are separated by a multicistronic element.

In some embodiments, the variable domains of the encoded TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, the encoded TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, the encoded TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the encoded TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the encoded TCR contains various domains or regions. In some cases, the exact domain or region can vary depending on the particular structural or homology modeling or other features used to describe a particular domain. It is understood that reference to amino acids, including to a specific sequence set forth as a SEQ ID NO used to describe domain organization of a recombinant receptor, e.g., TCR, are for illustrative purposes and are not meant to limit the scope of the embodiments provided. In some cases, the specific domain (e.g. variable or constant) can be several amino acids (such as one, two, three or four) longer or shorter. In some aspects, residues of a TCR are known or can be identified according to the International Immunogenetics Information System (IMGT) numbering system (see e.g. www.imgt.org; see also, Lefranc et al. (2003) Developmental and Comparative Immunology, 27; 55-77; and The T Cell Factsbook 2nd Edition, Lefranc and LeFranc Academic Press 2001). Using this system, the CDR1 sequences within a TCR Vα chains and/or Vβ chain correspond to the amino acids present between residue numbers 27-38, inclusive, the CDR2 sequences within a TCR Vα chain and/or Vβ chain correspond to the amino acids present between residue numbers 56-65, inclusive, and the CDR3 sequences within a TCR Vα chain and/or Vβ chain correspond to the amino acids present between residue numbers 105-117, inclusive.

In some embodiments, the α chain and β chain of a TCR each further contain a constant domain. In some embodiments, the α chain constant domain (Cα) and β chain constant domain (Cβ) individually are mammalian, such as is a human or murine constant domain. In some embodiments, the constant domain is adjacent to the cell membrane. For example, in some cases, the extracellular portion of the encoded TCR formed by the two chains contains two membrane-proximal constant domains, and two membrane-distal variable domains, which variable domains each contain CDRs.

In some embodiments, each of the Cα and Cβ domains is human. In some embodiments, the Cα is encoded by the TRAC gene (IMGT nomenclature) or is a variant thereof. In some embodiments, the Cα has or comprises the sequence of amino acids set forth in SEQ ID NO: 91 or 92 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 91 or 92. In some embodiments, the Cα has or comprises the sequence of amino acids set forth in any of SEQ ID NO:91 or 92. In some embodiments, the Cα has or comprises the sequence of amino acids, e.g., mature polypeptide, encoded by the nucleic acid sequence set forth in SEQ ID NO:93 or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids, e.g., mature polypeptide, encoded by the nucleic acid sequence set forth in SEQ ID NO:93. In some embodiments, the Cβ is encoded by TRBC1 or TRBC2 genes (IMGT nomenclature) or is a variant thereof. In some embodiments, the Cβ has or comprises the sequence of amino acids set forth in SEQ ID NO:94, 95 or 96 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 94, 95 or 96. In some embodiments, the CP has or comprises the sequence of amino acids set forth in SEQ ID NO: 94, 95 or 96. In some embodiments, the CP has or comprises the sequence of amino acids, e.g., mature polypeptide, encoded by the nucleic acid sequence set forth in SEQ ID NO:97 or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids, e.g., mature polypeptide, encoded by the nucleic acid sequence set forth in SEQ ID NO:97.

In some embodiments, any of the provided TCRs or antigen-binding fragments thereof can be a human/mouse chimeric TCR. In some cases, the TCR or antigen-binding fragment thereof have α chain and/or a β chain comprising a mouse constant region. In some aspects, the Cα and/or Cβ regions are mouse constant regions.

In some of any such embodiments, the TCR or antigen-binding fragment thereof containing one or more modifications in the α chain and/or β chain such that when the TCR or antigen-binding fragment thereof is expressed in a cell, the frequency of mispairing between the TCR α chain and β chain and an endogenous TCR α chain and β chain is reduced, the expression of the TCR α chain and β chain is increased and/or the stability of the TCR α chain and β chain is increased. In some embodiments, the one or more modifications is a replacement, deletion, or insertion of one or more amino acids in the Cα region and/or the Cβ region. In some aspects, the one or more modifications contain replacement(s) to introduce one or more cysteine residues that are capable of forming one or more non-native disulfide bridges between the α chain and β chain.

In some of any such embodiments, the TCR or antigen-binding fragment thereof containing a Cα region containing a cysteine at a position corresponding to position 48 with numbering as set forth in SEQ ID NO: 92 and/or a Cβ region containing a cysteine at a position corresponding to position 57 with numbering as set forth in SEQ ID NO: 96. In some embodiments, said Cα region contains the amino acid sequence set forth in any of SEQ ID NOS: 91 or 92, or a sequence of amino acids that has at least 90% sequence identity thereto containing one or more cysteine residues capable of forming a non-native disulfide bond with the β chain; and/or said Cβ region contains the amino acid sequence set forth in any of SEQ ID NOS:94, 95 or 96, or a sequence of amino acids that has at least 90% sequence identity thereto that contains one or more cysteine residues capable of forming a non-native disulfide bond with the α chain.

In some of any such embodiments, the encoded TCR or antigen-binding fragment thereof is encoded by a nucleotide sequence that has been codon-optimized.

In some of any such embodiments, the binding molecule or TCR or antigen-binding fragment thereof is isolated or purified or is recombinant. In some of any such embodiments, the binding molecule or TCR or antigen-binding fragment thereof is human.

In some embodiments, the encoded TCR may be a heterodimer of two chains α and β that are linked, such as by a disulfide bond or disulfide bonds. In some embodiments, the constant domain of the encoded TCR may contain short connecting sequences in which a cysteine residue forms a disulfide bond, thereby linking the two chains of the encoded TCR. In some embodiments, a TCR may have an additional cysteine residue in each of the α and β chains, such that the encoded TCR contains two disulfide bonds in the constant domains. In some embodiments, each of the constant and variable domains contains disulfide bonds formed by cysteine residues.

In some embodiments, the encoded TCR may be a heterodimer of two chains α and β or γ and δ, such as a dual-chain TCR, or it may be a single chain TCR construct. In some embodiments, the TCR is a heterodimer containing two separate chains (dual-chain TCR, α and β chains or γ and δ chains) that are linked, such as by a disulfide bond or disulfide bonds.

In some embodiments, the encoded TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the encoded recombinant receptors include recombinant TCRs and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some aspects, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the encoded TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the encoded TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e., diseased TCR libraries. In some embodiments, degenerate primers are used to amplify the gene repertoire of Vα and Vβ, such as by RT-PCR in samples, such as T cells, obtained from humans. In some embodiments, libraries, such as single-chain TCR (scTv) libraries, can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule.

In some aspects, the encoded TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, encoded TCRs, e.g. present on the antigen-specific T cells, may be selected, such as by binding activity, e.g., particular affinity or avidity for the antigen.

In some embodiments, the encoded TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, the antigen is a tumor antigen that can be a glioma-associated antigen, β-human chorionic gonadotropin, alphafetoprotein (AFP), B-cell maturation antigen (BCMA, BCM), B-cell activating factor receptor (BAFFR, BR3), and/or transmembrane activator and CAML interactor (TACI), Fe Receptor-like 5 (FCRL5, FcRH5), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, and melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, beta-catenin, Bcr-abl, E2A-PRL, H4-RET, IGH-IGK, MYL-RAR, Caspase 8 or a B-Raf antigen. Other tumor antigens can include any derived from FRa, CD24, CD44, CD133, CD 166, epCAM, CA-125, HE4, Oval, estrogen receptor, progesterone receptor, uPA, PAI-1, CD19, CD20, CD22, ROR1, mesothelin, CD33/IL3Ra, c-Met, PSMA, Glycolipid F77, GD-2, insulin growth factor (IGF)-I, IGF-II and IGF-I receptor. Specific tumor-associated antigens or T cell epitopes are known (see e.g. van der Bruggen et al. (2013) Cancer Immun, available at www.cancerimmunity.org/peptide/; Cheever et al. (2009) Clin Cancer Res, 15, 5323-37).

In some embodiments, the antigen is a viral antigen. Many viral antigen targets have been identified and are known, including peptides derived from viral genomes in HIV, HTLV and other viruses (see e.g., Addo et al. (2007) PLoS ONE, 2, e321; Tsomides et al. (1994) J Exp Med, 180, 1283-93; Utz et al. (1996) J Virol, 70, 843-51). Exemplary viral antigens include, but are not limited to, an antigen selected from hepatitis A, hepatitis B (e.g., HBV core and surface antigens (HBVc, HBVs)), hepatitis C (HCV), Epstein-Barr virus (e.g. EBVA), human papillomavirus (HPV; e.g. E6 and E7), human immunodeficiency type-1 virus (HIV1), Kaposi's sarcoma herpes virus (KSHV), human papilloma virus (HPV), influenza virus, Lassa virus, HTLN-1, HIN-1, HIN-II, CMN, EBN or HPN. In some embodiments, the target protein is a bacterial antigen or other pathogenic antigen, such as Mycobacterium tuberculosis (MT) antigens, trypanosome, e.g., Trypanosoma cruzi (T. cruzi), antigens such as surface antigen (TSA), or malaria antigens. Specific viral antigen or epitopes or other pathogenic antigens or T cell epitopes are known (see e.g., Addo et al. (2007) PLoS ONE, 2:e321; Anikeeva et al. (2009) Clin Immunol, 130:98-109).

In some embodiments, the antigen is an antigen derived from a virus associated with cancer, such as an oncogenic virus. For example, an oncogenic virus is one in which infection from certain viruses are known to lead to the development of different types of cancers, for example, hepatitis A, hepatitis B (e.g., HBV core and surface antigens (HBVc, HBVs)), hepatitis C (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV) antigen.

In some embodiments, the viral antigen is an HPV antigen, which, in some cases, can lead to a greater risk of developing cervical cancer. In some embodiments, the antigen can be a HPV-16 antigen, and HPV-18 antigen, and HPV-31 antigen, an HPV-33 antigen or an HPV-35 antigen. In some embodiments, the viral antigen is an HPV-16 antigen (e.g., seroreactive regions of the E1, E2, E6 and/or E7 proteins of HPV-16, see e.g., U.S. Pat. No. 6,531,127) or an HPV-18 antigen (e.g., seroreactive regions of the L1 and/or L2 proteins of HPV-18, such as described in U.S. Pat. No. 5,840,306). In some embodiments, the viral antigen is an HPV-16 antigen that is from the E6 and/or E7 proteins of HPV-16. In some embodiments, the TCR is a TCR directed against an HPV-16 E6 or HPV-16 E7. In some embodiments, the TCR is a TCR described in, e.g., WO 2015/184228, WO 2015/009604 and WO 2015/009606.

In some embodiments, the viral antigen is a HBV or HCV antigen, which, in some cases, can lead to a greater risk of developing liver cancer than HBV or HCV negative subjects. For example, in some embodiments, the heterologous antigen is an HBV antigen, such as a hepatitis B core antigen or a hepatitis B envelope antigen (US2012/0308580).

In some embodiments, the viral antigen is an EBV antigen, which, in some cases, can lead to a greater risk for developing Burkitt's lymphoma, nasopharyngeal carcinoma and Hodgkin's disease than EBV negative subjects. For example, EBV is a human herpes virus that, in some cases, is found associated with numerous human tumors of diverse tissue origin. While primarily found as an asymptomatic infection, EBV-positive tumors can be characterized by active expression of viral gene products, e.g., EBNA-1, LMP-1 and LMP-2A. In some aspects, the heterologous antigen is an EBV antigen that can include Epstein-Barr nuclear antigen (EBNA)-1, EBNA-2, EBNA-3A, EBNA-3B, EBNA-3C, EBNA-leader protein (EBNA-LP), latent membrane protein (LMP)-1, LMP-2A and LMP-2B, EBV-EA, EBV-MA or EBV-VCA.

In some embodiments, the viral antigen is an HTLV-1 or HTLV-2 antigen, which, in some cases, can lead to a greater risk for developing T-cell leukemia than HTLV-1 or HTLV-2 negative subjects. For example, in some embodiments, the heterologous antigen is an HTLV-antigen, such as TAX.

In some embodiments, the viral antigen is a HHV-8 antigen, which, in some cases, can lead to a greater risk for developing Kaposi's sarcoma than HHV-8 negative subjects. In some embodiments, the heterologous antigen is a CMV antigen, such as pp65 or pp64 (see U.S. Pat. No. 8,361,473).

In some embodiments, the antigen is an autoantigen, such as an antigen of a polypeptide associated with an autoimmune disease or disorder. In some embodiments, the autoimmune disease or disorder can be multiple sclerosis (MS), rheumatoid arthritis (RA), Sjogren syndrome, scleroderma, polymyositis, dermatomyositis, systemic lupus erythematosus, juvenile rheumatoid arthritis, ankylosing spondylitis, myasthenia gravis (MG), bullous pemphigoid (antibodies to basement membrane at dermal-epidermal junction), pemphigus (antibodies to mucopolysaccharide protein complex or intracellular cement substance), glomerulonephritis (antibodies to glomerular basement membrane), Goodpasture's syndrome, autoimmune hemolytic anemia (antibodies to erythrocytes), Hashimoto's disease (antibodies to thyroid), pernicious anemia (antibodies to intrinsic factor), idiopathic thrombocytopenic purpura (antibodies to platelets), Grave's disease, or Addison's disease (antibodies to thyroglobulin). In some embodiments, the autoantigen, such as an autoantigen associated with one of the foregoing autoimmune disease, can be collagen, such as type II collagen, mycobacterial heat shock protein, thyroglobulin, acetyl choline receptor (AcHR), myelin basic protein (MBP) or proteolipid protein (PLP). Specific autoimmune associated epitopes or antigens are known (see e.g., Bulek et al. (2012) Nat Immunol, 13:283-9; Harkiolaki et al. (2009) Immunity, 30:348-57; Skowera et al. (2008) J Clin Invest, 1(18): 3390-402).

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some examples, HLA-A0201-binding motifs and the cleavage sites for proteasomes and immune-proteasomes using computer prediction models are known. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the encoded recombinant TCR is a full-length TCR. In some embodiments, the recombinant TCR is an antigen-binding portion. In some embodiments, the TCR is a dimeric TCR (dTCR). In some embodiments, the TCR is a single-chain TCR (scTCR). In some embodiments, a dTCR or scTCR have the structures as described in, e.g., International Pat. App. Pub. No. WO 03/020763, WO 04/033685 and WO 2011/044186.

In some embodiments, the encoded recombinant TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the recombinant TCRs, including a dTCR or scTCR, can be linked to signaling domains that yield an active TCR on the surface of a T cell. In some embodiments, the recombinant TCR is expressed on the surface of cells. In some embodiments of the dTCR or scTCR containing introduced or engineered inter-chain disulfide bonds, the native disulfide bonds are not present.

In certain embodiments, the encoded TCR contains one or more modifications(s) to introduce one or more cysteine residues that are capable of forming one or more non-native disulfide bridges between the TCRα chain and TCRβ chain. In some embodiments, the encoded TCR contains a TCRα chain or a portion thereof containing a TCRα constant domain containing one or more cysteine residues capable of forming a non-native disulfide bond with a TCRβ chain. In some embodiments, the transgene encodes a TCRβ chain or a portion thereof containing a TCRβ constant domain containing one or more cysteine residues capable of forming a non-native disulfide bond with a TCRα chain. In some embodiments, the encoded TCR comprises a TCRα and/or TCRβ chain and/or a TCRα and/or TCRβ chain constant domains containing one or more modifications to introduce one or more disulfide bonds. In some embodiments, the transgene encodes a TCRα and/or TCRβ chain and/or a TCRα and/or TCRβ with one or more modifications to remove or prevent a native disulfide bond, e.g., between the TCRα encoded by the transgene and the endogenous TCRβ chain, or between the TCRβ encoded by the transgene and the endogenous TCR α chain. In some embodiments, one or more native cysteines that form and/or are capable of forming a native inter-chain disulfide bond are substituted to another residue, e.g., serine or alanine. In some embodiments, the cysteine is introduced at one or more of residue Thr48, Thr45, Tyr10, Thr45, and Ser15 with reference to numbering of a TCRα constant domain. In certain embodiments, cysteines can be introduced at residue Ser57, Ser77, Ser17, Asp59, of Glu15 of the TCRβ chain constant domain. Exemplary non-native disulfide bonds of a TCR are described in published International PCT No. WO2006/000830, WO 2006/037960 and Kuball et al. (2007) Blood, 109:2331-2338. In some embodiments, cysteines can be introduced or substituted at a residue corresponding to Thr48 of the Cα chain and Ser57 of the Cβ chain, at residue Thr45 of the Cα chain and Ser77 of the CP chain, at residue Tyr10 of the Cα chain and Ser17 of the Cβ chain, at residue Thr45 of the Cα chain and Asp59 of the Cβ chain and/or at residue Ser15 of the Cα chain and Glu15 of the Cβ chain. In some embodiments, any of the cysteine mutations can be made at a corresponding position in another sequence, for example, in a human or mouse Cα and Cβ sequence described above. The term “corresponding” with reference to positions of a protein, such as recitation that amino acid positions “correspond to” amino acid positions in an exemplary Cα and Cβ refers to amino acid positions identified upon alignment with the disclosed sequence based on structural sequence alignment or using a standard alignment algorithm, such as the GAP algorithm.

In some embodiments, the one or more of the native cysteines forming a native inter-chain disulfide bonds are substituted to another residue, such as to a serine or alanine. In some embodiments, an introduced or engineered disulfide bond can be formed by mutating non-cysteine residues on the first and second segments to cysteine. Exemplary non-native disulfide bonds of a TCR are described in published International PCT No. WO2006/000830.

In some embodiments, the encoded recombinant TCR is a dimeric TCR (dTCR). In some embodiments, the dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the inter-chain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, the dTCR contains a TCR α chain containing a variable a domain, a constant α domain and a first dimerization motif attached to the C-terminus of the constant α domain, and a TCR β chain comprising a variable β domain, a constant β domain and a first dimerization motif attached to the C-terminus of the constant 3 domain, wherein the first and second dimerization motifs interact to form a covalent bond between an amino acid in the first dimerization motif and an amino acid in the second dimerization motif linking the TCR α chain and TCR β chain together.

In some embodiments, the encoded recombinant TCR is a single-chain TCR (scTCR or scTv). Typically, a scTCR can be generated using known methods, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International Pat. App. Pub. Nos. WO 96/13593, WO 96/18105, WO 99/60120, WO 99/18129, WO 03/020763, WO 2011/044186; and Schlueter, C. J. et al. J. Mol. Biol. 256, 859 (1996). In some embodiments, the scTCR contains an introduced non-native disulfide inter-chain bond to facilitate the association of the TCR chains (see e.g. International Pat. App. Pub. No. WO 03/020763). In some embodiments, the scTCR is a non-disulfide linked truncated TCR in which heterologous leucine zippers fused to the C-termini thereof facilitate chain association (see e.g. International Pat. App. Pub. No. WO 99/60120). In some embodiments, the scTCR contains a TCRα variable domain covalently linked to a TCRβ variable domain via a peptide linker (see e.g., International Pat. App. Pub. No. WO 99/18129).

In some embodiments, the scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment. In some embodiments, the scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment. In some embodiments, the scTCR contains a first segment constituted by a TCR β chain variable region sequence fused to the N terminus of a β chain extracellular constant domain sequence, and a second segment constituted by an α chain variable region sequence fused to the N terminus of a sequence a chain extracellular constant and transmembrane sequence, and, optionally, a linker sequence linking the C terminus of the first segment to the N terminus of the second segment.

In some embodiments, the linker of the scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO:22). In some embodiments, the linker has the sequence GSADDAKKDAAKKDGKS (SEQ ID NO:23)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments, the encoded TCR or antigen-binding fragment thereof exhibits an affinity with an equilibrium dissociation constant (K_{D}) for a target antigen of between or between about 10⁻⁵ and 10⁻¹² M and all individual values and ranges therein. In some embodiments, the target antigen is an MHC-peptide complex or ligand.

### C. Cells and Preparation of Cells for Genetic Engineering

In some embodiments, provided are engineered cells, e.g., genetically engineered or modified cells, and methods of engineering cells. In some embodiments, polynucleotides, e.g., template polynucleotides containing nucleic acid sequence encoding a recombinant receptor or a portion thereof and/or additional molecule(s), are introduced into one a cell for engineering, e.g., according to the methods of engineering described herein. In some aspects, the transgene (exogenous or heterologous nucleic acid sequences) in the polynucleotides and/or portions thereof are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acid sequences are not naturally occurring, such as a nucleic acid sequences not found in nature or is modified from a nucleic acid sequence found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as iPSCs. In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils. In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. In some embodiments, the cells are human T cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Pat. App. Pub. No. WO2009/072003 or US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Various methods for the introduction of genetically engineered components, e.g., agents for inducing a genetic disruption and/or nucleic acids encoding recombinant receptors, e.g., CARs or TCRs, are known and may be used with the provided methods and compositions. Exemplary methods include those for transfer of nucleic acids encoding the polypeptides or receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell, or any delivery methods described in Section II.A.3 or II.B.3 herein. Other approaches and vectors for transfer of the nucleic acids encoding the recombinant products are those described, e.g., in WO2014055668 and U.S. Pat. No. 7,446,190.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (such as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, it may be desired to safeguard against the potential that overexpression of a stimulatory factor (for example, a lymphokine or a cytokine) could potentially result in an unwanted outcome or lower efficacy in a subject, such as a factor associated with toxicity in a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 11:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, the cells, e.g., T cells, may be engineered either during or after expansion. This engineering for the introduction of the gene of the desired polypeptide or receptor can be carried out with any suitable retroviral vector, for example. The genetically modified cell population can then be liberated from the initial stimulus (the CD3/CD28 stimulus, for example) and subsequently be stimulated with a second type of stimulus (e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

As described herein, in some embodiments, the cells are incubated and/or cultured prior to or in connection with genetic engineering. The incubation steps can include culture, cultivation, stimulation, activation, propagation and/or freezing for preservation, e.g. cryopreservation.

### D. Composition of Cells Expressing Recombinant Receptor

Also provided are plurality or populations of engineered cells, compositions containing such cells and/or enriched for such cells. In some aspects, the provided engineered cells and/or composition of engineered cells are produced by the methods described herein.

In some embodiments, the provided cell population and/or compositions containing engineered cells include a cell population that exhibits more improved, uniform, homogeneous and/or stable expression and/or antigen binding by the recombinant receptor, e.g., exhibit reduced coefficient of variation, compared to the expression and/or antigen binding of cell populations and/or compositions generated using other methods. In some embodiments, the cell population and/or compositions exhibit at least 100%, 95%, 90%, 80%, 70%, 60%, 50%, 40%, 30%, 20% or 10% lower coefficient of variation of expression of the recombinant receptor and/or antigen binding by the recombinant receptor compared to a respective population generated using other methods, e.g., random integration of sequences encoding the recombinant receptor. The coefficient of variation is defined as standard deviation of expression of the nucleic acid of interest (e.g., a transgene encoding a recombinant receptor or a portion thereof) within a population of cells, for example CD4+ and/or CD8+ T cells, divided by the mean of expression of the respective nucleic acid of interest in the respective population of cells. In some embodiments, the cell population and/or compositions exhibit a coefficient of variation that is lower than 0.70, 0.65, 0.60, 0.55, 0.50, 0.45, 0.40, 0.35 or 0.30 or less, when measured among CD4+ and/or CD8+ T cell populations that have been engineered using the methods provided herein.

In some embodiments, the provided cell population and/or compositions containing engineered cells include a cell population that exhibits minimal or reduced random integration of the transgene encoding a recombinant receptor or a portion thereof. In some aspects, random integration of transgene into the genome of the cell can result in adverse effects or cell death due to integration of the transgene into undesired location in the genome, e.g., into an essential gene or a gene critical in regulating the activity of the cell, and/or unregulated or uncontrolled expression of the receptor. In some aspects, random integration of the transgene is reduced by at least or greater than 50%, 60%, 70%, 80%, 90%, 91%, 92%, 93%, 94%, 95%, 96%, 97%, 98%, 99%, or more compared to cell populations generated using other methods.

In some embodiments, provided are cell population and/or compositions that include a plurality of engineered immune cells expressing a recombinant receptor, wherein the nucleic acid sequence encoding the recombinant receptor is present at the T cell stimulation-associated locus, e.g., by integration of a transgene encoding recombinant receptor or a portion thereof at the T cell stimulation-associated locus via homology directed repair (HDR). In some embodiments, at least or greater than 30%, 35%, 40%, 45%, 50%, 55%, 60%, 65%, 70%, 75%, 80%, or 90% of the cells in the composition and/or cells in the composition that contains a genetic disruption at the T cell stimulation-associated locus comprise integration of the transgene encoding recombinant receptor or a portion thereof at the T cell stimulation-associated locus.

In some embodiments, the provided compositions containing cells such as in which cells expressing the recombinant receptor make up at least 30%, 40%, 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. In some embodiments, the provided compositions containing cells such as in which cells expressing the recombinant receptor make up at least 30%, 40%, 50%, 60%, 70%, 80%, 90%, 91%, 92%, 93%, 94%, 95%, 96%, 97%, 98%, 99%, or more of the total cells in the composition that contains a genetic disruption at the T cell stimulation-associated locus.

### V. METHODS OF TREATMENT AND USE IN ADOPTIVE CELL THERAPY

Provided herein are methods of treatment, e.g., including administering any of the engineered cells or compositions containing engineered cells described herein. In some aspects, also provided are methods of administering any of the engineered cells or compositions containing engineered cells described herein to a subject, such as a subject that has a disease or disorder. The engineered cells expressing a recombinant receptor, such as a chimeric antigen receptor (CAR) or a T cell receptor (TCR), or compositions comprising the same, described herein are useful in a variety of therapeutic, diagnostic and prophylactic indications. For example, the engineered cells or compositions comprising the engineered cells are useful in treating a variety of diseases and disorders in a subject. Such methods and uses include therapeutic methods and uses, for example, involving administration of the engineered cells, or compositions containing the same, to a subject having a disease, condition, or disorder, such as a tumor or cancer. In some embodiments, the engineered cells or compositions comprising the same are administered in an effective amount to effect treatment of the disease or disorder. Uses include uses of the engineered cells or compositions in such methods and treatments, and in the preparation of a medicament in order to carry out such therapeutic methods. In some embodiments, the methods are carried out by administering the engineered cells, or compositions comprising the same, to the subject having or suspected of having the disease or condition. In some embodiments, the methods thereby treat the disease or condition or disorder in the subject. Also provided are therapeutic methods for administering the cells and compositions to subjects, e.g., patients.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described herein. In some of any embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histiocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or disorder is a multiple myeloma (MM). In some embodiments, administration of the provided cells, e.g., engineered cells with a modified T cell stimulation-associated locus, can result in treatment of and/or amelioration of a disease or condition, such as a MM in the subject. In some embodiments, the subject has or is suspected of having a MM that is associated with expression of a tumor-associated antigen, such as a B cell maturation antigen (BCMA).

In some embodiments, the disease or disorder is a chronic lymphocytic leukemia (CLL). In some embodiments, administration of the provided cells, e.g., engineered cells with a modified T cell stimulation-associated locus, can result in treatment of and/or amelioration of a disease or condition, such as a CLL in the subject. In some embodiments, the subject has or is suspected of having a CLL that is associated with expression of a tumor-associated antigen, such as a Receptor Tyrosine Kinase Like Orphan Receptor 1 (ROR1).

In some embodiments, the disease or disorder is a solid tumor, or a cancer associated with a non-hematological tumor. In some embodiments, the disease or disorder is a solid tumor, or a cancer associated with a solid tumor. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma. In some embodiments, the disease or disorder is a bladder, lung, brain, melanoma (e.g. small-cell lung, melanoma), breast, cervical, ovarian, colorectal, pancreatic, endometrial, esophageal, kidney, liver, prostate, skin, thyroid, or uterine cancers. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma.

In some embodiments, the disease or disorder is a non-small cell lung cancer (NSCLC). In some embodiments, administration of the provided cells, e.g., engineered cells with a modified T cell stimulation-associated locus, can result in treatment of and/or amelioration of a disease or condition, such as a NSCLC in the subject. In some embodiments, the subject has or is suspected of having a NSCLC that is associated with expression of a tumor-associated antigen, such as a Receptor Tyrosine Kinase Like Orphan Receptor 1 (ROR1).

In some embodiments, the disease or disorder is a head and neck squamous cell carcinoma (HNSCC). In some embodiments, administration of the provided cells, e.g., engineered cells with a modified T cell stimulation-associated locus, can result in treatment of and/or amelioration of a disease or condition, such as a HNSCC in the subject. In some embodiments, the subject has or is suspected of having a HNSCC that is associated with expression of a tumor-associated antigen, such as a human papilloma virus (HPV) 16 E6 or E7. In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some aspects, the recombinant receptor, such as a CAR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30, or combinations thereof.

In some embodiments, the disease or condition is a myeloma, such as a multiple myeloma. In some aspects, the recombinant receptor, such as a CAR, specifically binds to an antigen associated with the disease or condition or expressed in cells of the environment of a lesion associated with the multiple myeloma. Antigens targeted by the receptors in some embodiments include antigens associated with multiple myeloma. In some aspects, the antigen, e.g., the second or additional antigen, such as the disease-specific antigen and/or related antigen, is expressed on multiple myeloma, such as B cell maturation antigen (BCMA), G protein-coupled receptor class C group 5 member D (GPRC5D), CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, β2-Microglobulin, HM1.24, IGF-1R, IL-6R, TRAIL-R1, and the activin receptor type IIA (ActRIIA). See Benson and Byrd, J. Clin. Oncol. (2012) 30(16): 2013-15; Tao and Anderson, Bone Marrow Research (2011):924058; Chu et al., Leukemia (2013) 28(4):917-27; Garfall et al., Discov Med. (2014) 17(91):37-46. In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603,477, 8,008,450; U.S. Pub. No. US20120189622 or US20100260748; and/or International PCT Publication Nos. WO2006099875, WO2009080829 or WO2012092612 or WO2014210064. In some embodiments, such antibodies or antigen-binding fragments thereof (e.g. scFv) are contained in multispecific antibodies, multispecific chimeric receptors, such as multispecific CARs, and/or multispecific cells.

In some embodiments, the disease or disorder is associated with expression of G protein-coupled receptor class C group 5 member D (GPRC5D) and/or expression of B cell maturation antigen (BCMA).

In some embodiments, the disease or disorder is a B cell-related disorder. In some of any of the provided embodiments of the provided methods, the disease or disorder associated with BCMA is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

In some embodiments, the disease or disorder is a cancer. In some embodiments, the cancer is a GPRC5D-expressing cancer. In some embodiments, the cancer is a plasma cell malignancy and the plasma cell malignancy is multiple myeloma (MM) or plasmacytoma. In some embodiments, the cancer is multiple myeloma (MM). In some embodiments, the cancer is a relapsed/refractory multiple myeloma.

In some embodiments, the antigen is associated a virus, such as a human papilloma virus (HPV), and the disease or disorder is a cancer, such as a HNSCC. In some embodiments, the antigen is ROR1, and the disease or disorder is CLL. In some embodiments, the antigen is ROR1, and the disease or disorder is NSCLC.

In some embodiments, the antibody or an antigen-binding fragment (e.g. scFv or V_{H} domain) specifically recognizes an antigen, such as CD19, BCMA, GPRC5D or ROR1. In some embodiments, the antibody or antigen-binding fragment is derived from, or is a variant of, antibodies or antigen-binding fragment that specifically binds to CD19, BCMA, GPRC5D or ROR1.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described herein, and fludarabine at any dose or administration schedule, such as those described herein. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR expressed by the population can be conjugated either directly or indirectly through a linker to a targeting moiety. The practice of conjugating compounds, e.g., the CAR, to targeting moieties is known. See, e.g., Wadwa et al., J. Drug Targeting 3: 111 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total recombinant receptor (e.g., CAR)-expressing cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁸, 8×10⁸,or 1.2×10⁹ total such cells, or the range between any two of the foregoing values.

In some embodiments, the dose of genetically engineered cells comprises from at or about 1×10⁵ to at or about 5×10⁸ total CAR-expressing (CAR⁻) T cells, from at or about 1×10⁵ to at or about 2.5×10⁸ total CAR⁺ T cells, from at or about 1×10⁵ to at or about 1×10⁸ total CAR⁺ T cells, from at or about 1×10⁵ to at or about 5×10⁷ total CAR⁺ T cells, from at or about 1×10⁵ to at or about 2.5×10⁷ total CAR⁺ T cells, from at or about 1×10⁵ to at or about 1×10⁷ total CAR⁺ T cells, from at or about 1×10⁵ to at or about 5×10⁶ total CAR⁺ T cells, from at or about 1×10⁵ to at or about 2.5×10⁶ total CAR⁺ T cells, from at or about 1×10⁵ to at or about 1×10⁶ total CAR⁺ T cells, from at or about 1×10⁶ to at or about 5×10⁸ total CAR⁺ T cells, from at or about 1×10⁶ to at or about 2.5×10⁸ total CAR⁺ T cells, from at or about 1×10⁶ to at or about 1×10⁸ total CAR⁺ T cells, from at or about 1×10⁶ to at or about 5×10⁷ total CAR⁺ T cells, from at or about 1×10⁶ to at or about 2.5×10⁷ total CAR⁺ T cells, from at or about 1×10⁶ to at or about 1×10⁷ total CAR⁺ T cells, from at or about 1×10⁶ to at or about 5×10⁶ total CAR⁺ T cells, from at or about 1×10⁶ to at or about 2.5×10⁶ total CAR⁺ T cells, from at or about 2.5×10⁶ to at or about 5×10⁸ total CAR⁺ T cells, from at or about 2.5×10⁶ to at or about 2.5×10⁸ total CAR⁺ T cells, from at or about 2.5×10⁶ to at or about 1×10⁸ total CAR⁺ T cells, from at or about 2.5×10⁶ to at or about 5×10⁷ total CAR⁺ T cells, from at or about 2.5×10⁶ to at or about 2.5×10⁷ total CAR⁺ T cells, from at or about 2.5×10⁶ to at or about 1×10⁷ total CAR⁺ T cells, from at or about 2.5×10⁶ to at or about 5×10⁶ total CAR⁺ T cells, from at or about 5×10⁶ to at or about 5×10⁸ total CAR⁺ T cells, from at or about 5×10⁶ to at or about 2.5×10⁸ total CAR⁺ T cells, from at or about 5×10⁶ to at or about 1×10⁸ total CAR⁺ T cells, from at or about 5×10⁶ to at or about 5×10⁷ total CAR⁺ T cells, from at or about 5×10⁶ to at or about 2.5×10⁷ total CAR⁺ T cells, from at or about 5×10⁶ to at or about 1×10⁷ total CAR⁺ T cells, from at or about 1×10⁷ to at or about 5×10⁸ total CAR⁺ T cells, from at or about 1×10⁷ to at or about 2.5×10⁸ total CAR⁺ T cells, from at or about 1×10⁷ to at or about 1×10⁸ total CAR⁺ T cells, from at or about 1×10⁷ to at or about 5×10⁷ total CAR⁺ T cells, from at or about 1×10⁷ to at or about 2.5×10⁷ total CAR⁺ T cells, from at or about 2.5×10⁷ to at or about 5×10⁸ total CAR⁺ T cells, from at or about 2.5×10⁷ to at or about 2.5×10⁸ total CAR⁺ T cells, from at or about 2.5×10⁷ to at or about 1×10⁸ total CAR⁺ T cells, from at or about 2.5×10⁷ to at or about 5×10⁷ total CAR⁺ T cells, from at or about 5×10⁷ to at or about 5×10⁸ total CAR⁺ T cells, from at or about 5×10⁷ to at or about 2.5×10⁸ total CAR⁺ T cells, from at or about 5×10⁷ to at or about 1×10⁸ total CAR⁺ T cells, from at or about 1×10⁸ to at or about 5×10⁸ total CAR⁺ T cells, from at or about 1×10⁸ to at or about 2.5×10⁸ total CAR⁺ T cells, from at or about or 2.5×10⁸ to at or about 5×10⁸ total CAR⁺ T cells. In some embodiments, the dose of genetically engineered cells comprises from or from about 2.5×10⁷ to at or about 1.5×10⁸ total CAR⁺ T cells, such as from or from about 5×10⁷ to or to about 1×10⁸ total CAR⁺ T cells.

In some embodiments, the dose of genetically engineered cells comprises at least at or about 1×10⁵ CAR⁺ cells, at least at or about 2.5×10⁵ CAR⁺ cells, at least at or about 5×10⁵ CAR⁺ cells, at least at or about 1×10⁶ CAR⁺ cells, at least at or about 2.5×10⁶ CAR⁺ cells, at least at or about 5×10⁶ CAR⁺ cells, at least at or about 1×10⁷ CAR⁺ cells, at least at or about 2.5×10⁷ CAR⁺ cells, at least at or about 5×10⁷ CAR⁺ cells, at least at or about 1×10⁸ CAR⁺ cells, at least at or about 1.5×10⁸ CAR⁺ cells, at least at or about 2.5×10⁸ CAR⁺ cells, or at least at or about 5×10⁸ CAR⁺ cells.

In some embodiments, the cell therapy comprises administration of a dose comprising a number of cell from or from about 1×10⁵ to or to about 5×10⁸ total recombinant receptor-expressing cells, total T cells, or total peripheral blood mononuclear cells (PBMCs), from or from about 5×10⁵ to or to about 1×10⁷ total recombinant receptor-expressing cells, total T cells, or total peripheral blood mononuclear cells (PBMCs) or from or from about 1×10⁶ to or to about 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3⁺ or CD8⁺, in some cases also recombinant receptor-expressing (e.g. CAR⁺) cells. In some embodiments, the cell therapy comprises administration of a dose comprising a number of cell from or from about 1×10⁵ to or to about 5×10⁸ CD3⁺ or CD8⁺ total T cells or CD3⁺ or CD8⁺ recombinant receptor-expressing cells, from or from about 5×10⁵ to or to about 1×10⁷ CD3⁺ or CD8⁺ total T cells or CD3⁺ or CD8⁺ recombinant receptor-expressing cells, or from or from about 1×10⁶ to or to about 1×10⁷ CD3⁺ or CD8⁺ total T cells or CD3⁺ or CD8⁺ recombinant receptor-expressing cells, each inclusive. In some embodiments, the cell therapy comprises administration of a dose comprising a number of cell from or from about 1×10⁵ to or to about 5×10⁸ total CD3⁺/CAR⁺ or CD8⁺/CAR⁺ cells, from or from about 5×10⁵ to or to about 1×10⁷ total CD3⁺/CAR⁺ or CD8⁺/CAR⁺ cells, or from or from about 1×10⁶ to or to about 1×10⁷ total CD3⁺/CAR⁺ or CD8⁺/CAR⁺ cells, each inclusive.

In some embodiments, the T cells of the dose include CD4⁺ T cells, CD8⁺ T cells or CD4⁺ and CD8⁺ T cells. In some embodiments, the T cells of the dose include human CD4⁺ T cells, human CD8⁺ T cells or human CD4⁺ T cells and human CD8⁺ T cells.

In some embodiments, for example, where the subject is human, the CD8⁺ T cells of the dose, including in a dose including CD4⁺ and CD8⁺ T cells, includes between at or about 1×10⁶ and at or about 5×10⁸ total recombinant receptor (e.g., CAR)-expressing CD8⁺ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8⁺ T cells, from or from about 1×10⁷ to or to about 5×10⁷ total recombinant receptor-expressing CD8⁺ T cells, from or from about 1×10⁷ to or to about 0.25×10⁸ total recombinant receptor-expressing CD8⁺ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD8⁺ T cells.

In some embodiments, the dose of cells, e.g., recombinant receptor-expressing T cells, is administered to the subject as a single dose or is administered only one time within a period of two weeks, one month, three months, six months, 1 year or more. In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some composition, the first composition, e.g., first composition of the dose, comprises CD4⁺ T cells. In some composition, the first composition, e.g., first composition of the dose, comprises CD8⁺ T cells. In some embodiments, the first composition is administered prior to the second composition.

In some embodiments, the dose or composition of cells includes a defined or target ratio of CD4⁺ cells expressing a recombinant receptor to CD8⁺ cells expressing a recombinant receptor and/or of CD4⁺ cells to CD8⁺ cells, which ratio optionally is approximately 1:1 or is between approximately 1:3 and approximately 3:1, such as approximately 1:1. In some aspects, the administration of a composition or dose with the target or desired ratio of different cell populations (such as CD4⁺:CD8⁺ ratio or CAR⁺CD4⁺:CAR⁺CD8⁺ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some aspects, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some of any embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells being administered.

In some embodiments, in a subject administered the engineered cells or the composition provided herein, the volume or the size of the cancer or the tumor is reduced compared to in a subject that was not administered the engineered cells or the composition. In some embodiments, in a subject administered the engineered cells or the composition provided herein, survival of the subject is lengthened compared to in a subject that was not administered the engineered cells or the composition.

### VI. PHARMACEUTICAL COMPOSITION AND FORMULATION

Among the compositions are pharmaceutical compositions and formulations for administration, such as for adoptive cell therapy. In some embodiments, the dose of cells comprising cells engineered with a recombinant antigen receptor, e.g. CAR, is provided as a composition or formulation, such as a pharmaceutical composition or formulation. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. In some aspects, the cells are isolated from a subject, engineered, and administered to the same subject. In other aspects, they are isolated from one subject, engineered, and administered to another subject. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### VII. KITS AND ARTICLES OF MANUFACTURE

Also provided are articles of manufacture, systems, apparatuses, and kits useful in performing the provided embodiments. In some embodiments, the provided articles of manufacture or kits contain one or more components of the one or more agent(s) capable of inducing genetic disruption and/or template polynucleotide(s), e.g., template polynucleotides containing a transgene encoding a recombinant receptor or a portion thereof. In some embodiments, the articles of manufacture or kits can be used in methods for engineering T cells to express a recombinant receptor and/or other molecules.

In some embodiments, the articles of manufacture or kits include polypeptides, nucleic acids, vectors and/or polynucleotides useful in performing the provided methods. In some embodiments, the articles of manufacture or kits include one or more nucleic acid molecules, e.g., a plasmid or a DNA fragment, that encodes one or more components of the one or more agent(s) capable of inducing genetic disruption and/or comprises template polynucleotide(s), e.g., for use in targeting the transgene into the cell via HDR. In some embodiments, the articles of manufacture or kits provided herein contain control vectors.

In some embodiments, the articles of manufacture or kits provided herein contain one or more agent(s), wherein each of the one or more agent is independently capable of inducing a genetic disruption of a target site within a T cell stimulation-associated locus; and a template polynucleotide comprising a transgene encoding a recombinant receptor or a portion thereof, wherein the transgene is targeted for integration at or near the target site via homology directed repair (HDR). In some aspects, the one or more agent(s) capable of inducing a genetic disruption is any described herein. In some aspects, the one or more agent(s) is a ribonucleoprotein (RNP) complex comprising a Cas9/gRNA complex. In some aspects, the gRNA included in the RNP targets a target site in the T cell stimulation-associated locus, such as any target site described herein. In some aspects, the template polynucleotide is any of the template polynucleotide described herein.

In some embodiments, the articles of manufacture or kits include one or more containers, typically a plurality of containers, packaging material, and a label or package insert on or associated with the container or containers and/or packaging, generally including instructions for use, e.g., instructions for introducing the components into the cells for engineering.

The articles of manufacture provided herein contain packaging materials. Packaging materials for use in packaging the provided materials are well known. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the one or more agent(s) capable of inducing genetic disruption and/or template polynucleotide(s) are packaged separately. In some embodiments, each container can have a single compartment. In some embodiments, other components of the articles of manufacture or kits are packaged separately, or together in a single compartment.

Also provided are articles of manufacture, systems, apparatuses, and kits useful in administering the provided cells and/or cell compositions, e.g., for use in therapy or treatment. In some embodiments, the articles of manufacture or kits provided herein contain T cells and/or T cell compositions, such as any T cells and/or T cell compositions described herein. In some aspects, the articles of manufacture or kits provided herein can be used for administration of the T cells or T cell compositions, and can include instructions for use.

In some embodiments, the articles of manufacture or kits provided herein contain T cells, and/or T cell compositions, such as any T cells, and/or T cell compositions described herein. In some embodiments, the T cells, and/or T cell compositions any of the modified T cells used the screening methods described herein. In some embodiments, the articles of manufacture or kits provided herein contain control or unmodified T cells and/or T cell compositions. In some embodiments, the article of manufacture or kits include one or more instructions for administration of the engineered cells and/or cell compositions for therapy.

The articles of manufacture and/or kits containing cells or cell compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may include (a) a first container with a composition contained therein, wherein the composition includes engineered cells expressing a recombinant receptor; and (b) a second container with a composition contained therein, wherein the composition includes the second agent. In some embodiments, the article of manufacture may include (a) a first container with a first composition contained therein, wherein the composition includes a subtype of engineered cells expressing a recombinant receptor; and (b) a second container with a composition contained therein, wherein the composition includes a different subtype of engineered cells expressing a recombinant receptor. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VIII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1%.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

In some cases, a “T cell stimulation-associated locus” refers to a gene locus whose expression is induced, increased or upregulated following a stimulation or activation signal in T cells. In some aspects, the T cell stimulation-associated locus is an endogenous gene locus that is responsive to a signal transduced through a components of the TCR complex of a T cell, or a recombinant receptor comprising intracellular signaling regions that comprise a component of the TCR complex or a portion thereof, and/or antigen or epitope binding by a receptor, e.g. T cell receptor (TCR) or a recombinant receptor, present or expressed on a T cell. In some aspects, the T cell stimulation-associated locus can be regulated by a canonical factor that is part of the normal downstream signaling pathway of T cells. In some aspects, antigen or epitope binding and/or signal or activity through the intracellular signaling region of the encoded recombinant receptor, e.g., CAR or a TCR, induces signaling that induces the T cell stimulation-associated locus to express the transgene, e.g., encoding the recombinant receptor. Detectable expression of the endogenous gene product and/or the transgene can then be monitored as an indicator of T cell activation.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### IX. EXEMPLARY EMBODIMENTS

Among the provided embodiments are:
1. An engineered T cell comprising a modified T cell stimulation-associated locus comprising a transgene encoding a recombinant receptor or a portion thereof, wherein the transgene is operably linked to an endogenous transcriptional regulatory element of the T cell stimulation-associated locus, wherein the endogenous transcriptional regulatory element induces or upregulates expression of the operably linked transgene following a simulation or activation signal in the T cell.
2. The engineered T cell of embodiment 1, wherein the endogenous transcriptional regulatory element is a promoter of an endogenous T cell stimulation-associated locus.
3. The engineered T cell of embodiment 1 or 2, wherein the transgene encoding the recombinant receptor or a portion thereof is present downstream of the promoter.
4. The engineered T cell of any of embodiments 1-3, wherein the expression of the operably linked transgene is upregulated or induced within less than at or about 6, 12, 18, 24, 36 or 48 hours following the stimulation or activation signal in the T cells.
5. The engineered T cell of any of embodiments 1-4, wherein, following the upregulation or induction of expression or following a reduction or an absence of the simulation or activation signal in the T cells, the expression of the operably linked transgene is reduced or downregulated.
6. The engineered T cell of any of embodiments 1-5, wherein, following the upregulation or induction of expression, or following the reduction or the absence of the simulation or activation signal in the T cells, the expression of the operably linked transgene is reduced or downregulated by greater than at or about 30%, 40%, 50%, 60%, 70%, 75%, 80%, 85%, 90% or 95% or more.
7. The engineered T cell of embodiment 5 or 6, wherein, following the upregulation or induction of expression, the expression of the operably linked transgene is reduced or downregulated after at or about 1, 2, 3, 4, 5, 6, 7 or 8 days or more after the stimulation or activation signal in the T cells.
8. The engineered T cell of any of embodiments 5-7, wherein the expression of the operably linked transgene is reduced or downregulated within less than at or about 6, 12, 18, 24, 36 or 48 hours following the reduction or the absence of the simulation or activation signal in the T cell.
9. The engineered T cell of any of embodiments 5-8, wherein the expression of the operably linked transgene is capable of being induced or upregulated again following a further simulation or activation signal in the T cells after the reduction or the absence of the simulation or activation signal.
10. The engineered T cell of embodiment 9, wherein the expression of the operably linked transgene is upregulated or induced within less than at or about 6, 12, 18, 24, 36 or 48 hours following the further simulation or activation signal in the T cells after the reduction or the absence of the simulation or activation signal.
11. The engineered T cell of any of embodiments 1-10, wherein a translated product from an open reading frame of the endogenous T cell stimulation-associated locus is not expressed in the cell or a functional endogenous gene product of the endogenous T cell stimulation-associated locus is not expressed, following the simulation or activation signal in the T cell.
12. The engineered T cell of embodiment 11, wherein the modified T cell stimulation-associated locus comprises a deletion, an insertion, a frameshift mutation or a nonsense mutation in the open reading frame of the endogenous T cell stimulation-associated locus.
13. The engineered T cell of any of embodiments 1-12, wherein the endogenous T cell stimulation-associated locus is selected from among PDCD1, CD69, Nur77, FoxP3 and a HLA-DR locus.
14. The engineered T cell of any of embodiments 1-13, wherein: the endogenous transcriptional regulatory element comprises one or more response elements recognized by a transcription factor that is activated following the stimulation or activation signal.
15. The engineered T cell of any of embodiments 1-14, wherein the recombinant receptor or portion thereof is capable of inducing or transmitting the stimulation or activation signal in the T cell.
16. The engineered T cell of any of embodiments 1-15, wherein:
the recombinant receptor comprises an intracellular region comprising an intracellular signaling domain of a component of the T cell receptor (TCR) complex and the stimulation or activation signal in the T cells comprises a signal through the intracellular signaling domain present in the recombinant receptor, or
the recombinant receptor comprises an intracellular region comprising an intracellular signaling domain comprising an immunoreceptor tyrosine-based activation motif (ITAM) and the stimulation or activation signal in the T cells comprises a signal through the intracellular signaling domain present in the recombinant receptor.
17. The engineered T cell of any of embodiments 1-16, wherein the recombinant receptor comprises an extracellular region comprising a binding domain that is capable of binding to or recognizing an agent.
18. The engineered T cell of embodiment 17, wherein a stimulation or activation signal is induced in the T cell upon binding of the agent.
19. The engineered T cell of embodiment 17 or 18, wherein the agent is a target antigen, optionally wherein the target antigen is a recombinant protein or is an antigen expressed on the surface of a cell.
20. The engineered T cell of embodiment 19, wherein the target antigen is associated with, specific to, or expressed on a cell or tissue of a disease, disorder or condition.
21. The engineered T cell of embodiment 20, wherein the disease, disorder or condition is an infectious disease or disorder, an autoimmune disease, an inflammatory disease, or a tumor or a cancer, optionally, wherein the target antigen is a tumor antigen.
22. The engineered T cell of any of embodiments 17-21, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.
23. The engineered T cell of embodiment 17, wherein the agent is an anti-idiotypic antibody.
24. The engineered T cell of any of embodiments 1-23, wherein the recombinant receptor is a chimeric antigen receptor (CAR).
25. The engineered T cell of embodiment 24, wherein the CAR comprises an extracellular region, a transmembrane domain, and an intracellular region.
26. The engineered T cell of embodiment 25, wherein the extracellular region comprises a spacer, optionally wherein the spacer is operably linked between the binding domain and the transmembrane domain.
27. The engineered T cell of embodiment 25 or 26, wherein the extracellular region comprises a binding domain that is or comprises an antibody or an antigen-binding fragment thereof.
28. The engineered T cell of any of embodiments 25-27, wherein the intracellular region comprises an intracellular signaling domain.
29. The engineered T cell of embodiment 28, wherein the intracellular signaling domain is or comprises an intracellular signaling domain of a CD3 chain, optionally a CD3-zeta (CD3ζ) chain, or a signaling portion thereof.
30. The engineered T cell of embodiment 28 or 29, wherein the intracellular region comprises one or more costimulatory signaling domain(s).
31. The engineered T cell of embodiment 30, wherein the one or more costimulatory signaling domain comprises an intracellular signaling domain of a CD28, a 4-1BB or an ICOS or a signaling portion thereof.
32. The engineered T cell of embodiment 30 or 31, wherein the costimulatory signaling region comprises an intracellular signaling domain of 4-1BB.
33. The engineered T cell of any of embodiments 1-32, wherein the modified T cell stimulation-associated locus encodes a recombinant receptor that is a CAR, wherein the CAR comprises, from its N to C terminus in order: the extracellular binding domain, the spacer, the transmembrane domain and an intracellular region.
34. The engineered T cell of any of embodiments 1-33, wherein
the transgene comprises in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a CH2 region and/or a CH3 region; and a transmembrane domain, optionally from human CD28; a costimulatory signaling domain, optionally from human 4-1BB; and an intracellular signaling domain, optionally a CD3ζ chain or a portion thereof; and/or
the modified T cell stimulation-associated locus comprises in order: a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a CH2 region and/or a CH3 region; and a transmembrane domain, optionally from human CD28; a costimulatory signaling domain, optionally from human 4-1BB; and an intracellular signaling domain, optionally a CD3ζ chain or a portion thereof.
35. The engineered T cell of any of embodiments 1-34, wherein the transgene encodes the recombinant receptor.
36. The engineered T cell of any of embodiments 1-34, wherein the transgene encodes a portion of the recombinant receptor.
37. The engineered T cell of embodiment 36, wherein the recombinant receptor contains two separate polypeptide chains, wherein the portion of the recombinant receptor encoded by the transgene is one chain of the recombinant receptor, and the engineered T cell further expresses the other chain of the recombinant receptor.
38. The engineered T cell of embodiment 37, wherein the other chain of the recombinant receptor is encoded by a second transgene.
39. The engineered T cell of any of embodiments 24-38, wherein the CAR is a multi-chain CAR.
40. The engineered T cell of embodiment 39, wherein transgene encodes one chain of the multi-chain CAR.
41. The engineered T cell of any of embodiments 1-23 and 35-38, wherein the recombinant receptor is a recombinant T cell receptor (TCR).
42. The engineered T cell of embodiment 41, wherein recombinant TCR comprises an alpha (TCRα) chain and a beta (TCRβ) chain and the transgene comprises a nucleic acid sequence encoding the TCRα chain and/or a nucleic acid sequence encoding the TCRβ chain.
43. The engineered T cell of embodiment 42, wherein the transgene encodes one of the TCRα chain or the TCRβ chain.
44. The engineered T cell of embodiment 42 or 43, wherein the TCRα chain comprises a constant (Cα) region comprising one or more introduced cysteine residues and/or the TCRβ chain comprises a Cβ region comprising one or more introduced cysteine residues, wherein the one or more introduced cysteine residues are capable of forming one or more non-native disulfide bridges between the alpha chain and beta chain, optionally wherein the one or more introduced cysteine residues comprises replacement of a non-cysteine residue with a cysteine residue.
45. The engineered T cell of embodiment 44, wherein the Cα region comprises a cysteine at a position corresponding to position 48 with numbering as set forth in SEQ ID NO: 92; and/or the CP region comprises a cysteine at a position corresponding to position 57 with numbering as set forth in SEQ ID NO: 96.
46. The engineered T cell of any of embodiments 1-45, wherein the transgene comprises a sequence of nucleotides encoding at least one further protein.
47. The engineered T cell of embodiment 46, wherein the at least one further protein is a surrogate marker, optionally wherein the surrogate marker is a truncated receptor, optionally wherein the truncated receptor lacks an intracellular signaling domain and/or is not capable of mediating intracellular signaling when bound by its ligand.
48. The engineered T cell of any of embodiments 1-47, wherein the transgene further comprises a multicistronic element(s).
49. The engineered T cell of embodiment 48, wherein the multicistronic element(s) comprises a sequence encoding a ribosome skip element selected from among a T2A, a P2A, a E2A or a F2A or an internal ribosome entry site (IRES).
50. The engineered T cell of embodiment 48 or 49, wherein:
the multicistronic element is positioned between the sequence of nucleotides encoding the CAR and the sequence of nucleotides encoding the at least one further protein;
the recombinant receptor is a recombinant TCR, and the multicistronic element is positioned between a sequence of nucleotides encoding the TCRα and a sequence of nucleotides encoding the TCRβ;
the recombinant receptor is a multi-chain CAR, and the multicistronic element is positioned between a sequence of nucleotides encoding one chain of the multi-chain CAR and a sequence of nucleotides encoding another chain of the multi-chain CAR; and/or
the multicistronic element(s) are upstream of the sequence of nucleotides encoding the recombinant receptor.
51. The engineered T cell of any of embodiments 1-50, wherein the modified T cell stimulation-associated locus is produced by integration of the transgene encoding the recombinant receptor into the endogenous to cell stimulation-associated locus by:
a) inducing a genetic disruption at one or more target site(s) at or near the endogenous T cell stimulation-associated locus; and
b) introducing a polynucleotide for homology directed repair (HDR).
52. The engineered T cell of embodiment 51, wherein the transgene encoding the recombinant receptor is integrated at or near the at least one target site in the T cell stimulation-associated locus.
53. The engineered T cell of embodiment 51 or 52, wherein the genetic disruption is effected by a zinc finger nuclease (ZFN), a TAL-effector nuclease (TALEN), or a CRISPR-Cas9 combination that specifically binds to, recognizes, or hybridizes to the target site.
54. The engineered T cell of any of embodiments 51-53, wherein the genetic disruption is effected by a CRISPR-Cas9 combination and the CRISPR-Cas9 combination comprises a guide RNA (gRNA) having a targeting domain that is complementary to the at least one target site.
55. The engineered T cell of embodiment 54, wherein the CRISPR-Cas9 combination is a ribonucleoprotein (RNP) complex comprising the gRNA and a Cas9 protein.
56. The engineered T cell of embodiment 55, wherein the genetic disruption of is effected by the RNP introduced into a plurality of T cells via electroporation.
57. The engineered T cell of any of embodiments 1-56, wherein the T cell stimulation-associated locus is PDCD1.
58. The engineered T cell of embodiment 57, wherein the genetic disruption is effected by a CRISPR-Cas9 combination comprising a gRNA and the gRNA has a targeting domain that is complementary to a target site in a PDCD1 gene.
59. The engineered T cell of embodiment 58, wherein the gRNA comprises the sequence set forth in in any one of SEQ ID NOS: 75 and 104-109.
60. The engineered T cell of embodiment 58 or 59, wherein the gRNA comprises the sequence set forth in SEQ ID NO:75.
61. The engineered T cell of any of embodiments 1-56, wherein the T cell stimulation-associated locus is CD69.
62. The engineered T cell of embodiment 61, wherein the genetic disruption is effected by a CRISPR-Cas9 combination comprising a gRNA and the gRNA has a targeting domain that is complementary to a target site in a CD69 gene.
63. The engineered T cell of embodiment 62, wherein the gRNA comprises the sequence set forth in any one of SEQ ID NOS: 116-121.
64. The engineered T cell of any of embodiments 1-56, wherein the T cell stimulation-associated locus is Nur77.
65. The engineered T cell of embodiment 64, wherein the genetic disruption is effected by a CRISPR-Cas9 combination comprising a gRNA and the gRNA has a targeting domain that is complementary to a target site in a Nur77 gene.
66. The engineered T cell of embodiment 65, wherein the gRNA comprises the sequence set forth in SEQ ID NO: 122-127 and 134-136.
67. The engineered T cell of any of embodiments 1-56, wherein the T cell stimulation-associated locus is FoxP3.
68. The engineered T cell of any of embodiments 1-56, wherein the T cell stimulation-associated locus is a HLA-DR locus.
69. The engineered T cell of any of embodiments 1-68, wherein the T cell further comprises a genetic disruption at an endogenous T cell receptor alpha constant region (TRAC) gene and/or an endogenous T cell receptor beta constant region (TRBC) gene.
70. The engineered T cell of embodiment 69, wherein the genetic disruption is effected by a zinc finger nuclease (ZFN), a TAL-effector nuclease (TALEN), or a CRISPR-Cas9 combination that specifically binds to, recognizes, or hybridizes to at least one target site is within the TRAC, TRBC1 and/or TRBC2 gene.
71. The engineered T cell of embodiment 69 or 70, wherein the genetic disruption is effected by a CRISPR-Cas9 combination and the CRISPR-Cas9 combination comprises a guide RNA (gRNA) having a targeting domain that is complementary to the at least one target site, optionally wherein the CRISPR-Cas9 combination is a ribonucleoprotein (RNP) complex comprising the gRNA and a Cas9 protein, optionally wherein the genetic disruption of is effected by the RNP introduced into a plurality of T cells via electroporation.
72. The engineered T cell of any of embodiments 69-71, wherein the genetic disruption is effected by a CRISPR-Cas9 combination comprising a gRNA and the gRNA has a targeting domain that is complementary to at least one target site is within the TRAC, TRBC1 and/or TRBC2 gene.
73. The engineered T cell of embodiment 72, wherein the gRNA has a targeting domain that is complementary to a target site in a TRAC gene.
74. The engineered T cell of embodiment 73, wherein the gRNA comprises the sequence set forth in any one of SEQ ID NOS: 77 and 188-218.
75. The engineered T cell of embodiment 73 or 74, wherein the gRNA comprises the sequence set forth in SEQ ID NO:77.
76. The engineered T cell of embodiment 72, wherein the gRNA has a targeting domain that is complementary to a target site in a TRBC gene.
77. The engineered T cell of embodiment 76, wherein the gRNA comprises the sequence set forth in any one of SEQ ID NOS: 219-276.
78. The engineered T cell of any of embodiments 1-77, wherein signaling activity through the intracellular signaling domain of the encoded recombinant receptor in the absence of a simulation or activation signal in the T cells is reduced by greater than at or about 10%, 15%, 20%, 25%, 30%, 40%, 50% or more compared to an engineered T cell comprising a transgene encoding the same recombinant receptor present at a different location in the genome of the T cell or present at random locations in the genome of the T cell.
79. The engineered T cell of any of embodiments 1-78, wherein the T cell is a CD8+ T cell or a CD4+ T cell or subtypes thereof.
80. The engineered T cell of any of embodiments 1-79, wherein the T cell is a primary T cell derived from a subject, optionally wherein the subject is a human.
81. The engineered T cell of any of embodiments 1-79, wherein the T cell is derived from a multipotent or pluripotent cell, which optionally is an iPSC.
82. A composition comprising a plurality of the engineered cell of any of embodiments 1-81.
83. The composition of embodiment 82, wherein the expression of the operably linked transgene is upregulated or induced in one or more cells in the composition within less than at or about 6, 12, 18, 24, 36 or 48 hours following the stimulation or activation signal in the T cells.
84. The composition of embodiment 82 or 83, wherein, following a simulation or activation signal in the T cells, the frequency of cells expressing the operably linked transgene among the cells in the composition is greater than at or about 50%, 60%, 70%, 75%, 80%, 85%, 90% or 95% or more.
85. The composition of any of embodiments 82-84, wherein, following the upregulation or induction of expression or following a reduction or an absence of the simulation or activation signal in the T cells, the expression of the operably linked transgene is reduced or downregulated in one or more cells in the composition.
86. The composition of any of embodiments 82-84, wherein, following the upregulation or induction of expression, or following the reduction or the absence of the simulation or activation signal in the T cells, the frequency of cells expressing the operably linked transgene among the cells in the composition is reduced by greater than at or about 30%, 40%, 50%, 60%, 70%, 75%, 80%, 85%, 90% or 95% or more.
87. The composition of any of embodiments 82-86, wherein, following the upregulation or induction of expression, and the expression of the operably linked transgene is reduced or downregulated in one or more cells in the composition after at or about 1, 2, 3, 4, 5, 6, 7 or 8 days or more after the stimulation or activation signal in the T cells.
88. The composition of any of embodiments 82-87, wherein the expression of the operably linked transgene in one or more of the cells in the composition is reduced or downregulated within less than at or about 6, 12, 18, 24, 36 or 48 hours following the reduction or an absence of the simulation or activation signal in the T cell.
89. The composition of embodiment 88, wherein, following a reduction or an absence of the simulation or activation signal in the T cells, the frequency of cells expressing the recombinant receptor among the cells in the composition is less than at or about 50%, 40%, 30%, 25%, 20%, 15%, 10% or 5% or less.
90. The composition of any of embodiments 82-89, wherein the composition comprises CD4+ T cells and/or CD8+ T cells.
91. The composition of any of embodiments 82-90, wherein the composition comprises CD4+ and CD8+ T cells and the ratio of CD4+ to CD8+ T cells is from or from about 1:3 to 3:1, optionally 1:1.
92. A polynucleotide, comprising:
(a) a transgene encoding a recombinant receptor or a portion thereof, and
(b) one or more homology arms linked to the transgene, wherein the one or more homology arms comprise a sequence homologous to one or more region(s) of an endogenous T cell stimulation-associated locus in a T cell.
93. The polynucleotide of embodiment 92, wherein the recombinant receptor or a portion thereof is encoded by a modified T cell stimulation-associated locus comprising the transgene encoding the recombinant receptor or a portion thereof, when the recombinant receptor is expressed from a cell introduced with the polynucleotide.
94. The polynucleotide of embodiment 92 or 93, wherein the transgene is a sequence that is exogenous or heterologous to an open reading frame of the endogenous T cell stimulation-associated locus of a T cell, optionally a human T cell.
95. The polynucleotide of any of embodiments 92-94, wherein the one or more homology arm comprises a 5′ homology arm and/or a 3′ homology arm, optionally wherein the 5′ homology arm and 3′ homology arm comprises nucleic acid sequences homologous to nucleic acid sequences surrounding a target site, wherein the target site is within the T cell stimulation-associated locus.
96. The polynucleotide of embodiment 95, wherein the target site is downstream of an endogenous transcriptional regulatory element of the T cell stimulation-associated locus.
97. The polynucleotide of embodiment 95 or 96, wherein the polynucleotide comprises the structure [5′ homology arm]-[transgene]-[3′ homology arm].
98. The polynucleotide of embodiment 97, wherein the 5′ homology arm and 3′ homology arm comprises nucleic acid sequences homologous to nucleic acid sequences surrounding the at least one target site.
99. The polynucleotide of any of embodiments 95-98, wherein the 5′ homology arm and 3′ homology arm independently are between at or about 50 and at or about 750 nucleotides, at or about 50 and at or about 500 nucleotides, at or about 50 and at or about 250 nucleotides, at or about 50 and at or about 100 nucleotides, at or about 100 and at or about 750 nucleotides, at or about 100 and at or about 500 nucleotides, at or about 100 and at or about 250 nucleotides, at or about 250 and at or about 750 nucleotides, at or about 250 and at or about 500 nucleotides, in length.
100. The polynucleotide of any of embodiments 95-99, wherein the 5′ homology arm and 3′ homology arm independently are at or about 50, 60, 70, 80, 90, 100, 150, 200, 250, 300, 400 or 500 nucleotides, or any value between any of the foregoing, in length.
101. The polynucleotide of any of embodiments 95-100, wherein the 5′ homology arm and 3′ homology arm independently are less than at or about 100 nucleotides, in length, optionally at or about 50, 60, 70, 80 or 90 nucleotides, or any value between any of the foregoing, in length.
102. The polynucleotide of any of embodiments 95-101, wherein the T cell stimulation-associated locus is selected from among PDCD1, CD69, Nur77, FoxP3 and a HLA-DR locus.
103. The polynucleotide of embodiment 102, wherein the T cell stimulation-associated locus is PDCD1.
104. The polynucleotide of embodiment 103, wherein the 5′ homology arm and 3′ homology arm comprise a sequence homologous to one or more region(s) of PDCD1.
105. The polynucleotide of embodiment 104, wherein the 5′ homology arm comprises:
a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 66;
b) a sequence comprising at or at least at or at least 150, 200, 250, 300, 350, 400, 450, 500, 550, or 600 contiguous nucleotides of the sequence set forth in SEQ ID NO:66; or
c) the sequence set forth in SEQ ID NO: 66.
106. The polynucleotide of embodiment 104 or 105, wherein the 3′ homology arm comprises:
a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 67;
b) a sequence comprising at or at least at or at least 150, 200, 250, 300, 350, 400, 450, 500, 550, or 600 contiguous nucleotides of the sequence set forth in SEQ ID NO:67; or
c) the sequence set forth in SEQ ID NO: 67.
107. The polynucleotide of embodiment 102, wherein the T cell stimulation-associated locus is CD69.
108. The polynucleotide of embodiment 107, wherein the 5′ homology arm and 3′ homology arm comprise a sequence homologous to one or more region(s) of CD69.
109. The polynucleotide of embodiment 102, wherein the T cell stimulation-associated locus is Nur77.
110. The polynucleotide of embodiment 109, wherein the 5′ homology arm and 3′ homology arm comprise a sequence homologous to one or more region(s) of Nur77.
111. The polynucleotide of embodiment 102, wherein the T cell stimulation-associated locus is FoxP3.
112. The polynucleotide of embodiment 111, wherein the 5′ homology arm and 3′ homology arm comprise a sequence homologous to one or more region(s) of FoxP3.
113. The polynucleotide of embodiment 102, wherein the T cell stimulation-associated locus is a HLA-DR locus.
114. The polynucleotide of embodiment 113, wherein the 5′ homology arm and 3′ homology arm comprise a sequence homologous to one or more region(s) of a HLA-DR locus.
115. The polynucleotide of any of embodiments 92-114, wherein the recombinant receptor or portion thereof is capable of inducing or transmitting the stimulation or activation signal in the T cell.
116. The polynucleotide of any of embodiments 92-115, wherein:
the recombinant receptor comprises an intracellular region comprising an intracellular signaling domain of a component of the T cell receptor (TCR) complex and the stimulation or activation signal in the T cells comprises a signal through the intracellular signaling domain present in the recombinant receptor, or
the recombinant receptor comprises an intracellular region comprising an intracellular signaling domain comprising an immunoreceptor tyrosine-based activation motif (ITAM) and the stimulation or activation signal in the T cells comprises a signal through the intracellular signaling domain present in the recombinant receptor.
117. The polynucleotide of any of embodiments 92-116, wherein the recombinant receptor comprises an extracellular region comprising a binding domain that is capable of binding to or recognizing an agent.
118. The polynucleotide of embodiment 117, wherein a stimulation or activation signal is induced in the T cell upon binding of the agent.
119. The polynucleotide of embodiment 117 or 118, wherein the agent is a target antigen, optionally wherein the target antigen is a recombinant protein or is an antigen expressed on the surface of a cell.
120. The polynucleotide of embodiment 119, wherein the target antigen is associated with, specific to, or expressed on a cell or tissue of a disease, disorder or condition.
121. The polynucleotide of embodiment 120, wherein the disease, disorder or condition is an infectious disease or disorder, an autoimmune disease, an inflammatory disease, or a tumor or a cancer, optionally, wherein the target antigen is a tumor antigen.
122. The polynucleotide of any of embodiments 117-121, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-11 and LAGE-12), carcinoembryonic antigen (CEA), a cyclin, cyclin A2, C—C Motif Chemokine Ligand 1 (CCL-11), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-12), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-122 receptor alpha (IL-122Rα), IL-113 receptor alpha 2 (IL-113Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-11), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-11), a pathogen-specific or pathogen-expressed antigen, or an antigen associated with a universal tag, and/or biotinylated molecules, and/or molecules expressed by HIV, HCV, HBV or other pathogens.
123. The polynucleotide of embodiment 117, wherein the agent is an anti-idiotypic antibody.
124. The polynucleotide of any of embodiments 92-123, wherein the recombinant receptor is a chimeric antigen receptor (CAR).
125. The polynucleotide of embodiment 124, wherein the CAR comprises an extracellular region, a transmembrane domain, and an intracellular region.
126. The polynucleotide of any of embodiments 125, wherein the extracellular region comprises a spacer, optionally wherein the spacer is operably linked between the binding domain and the transmembrane domain.
127. The polynucleotide of embodiment 125 or 126, wherein the extracellular region comprises a binding domain that is or comprises an antibody or an antigen-binding fragment thereof.
128. The polynucleotide of any of embodiments 125-127, wherein the intracellular region comprises an intracellular signaling domain.
129. The polynucleotide of embodiment 128, wherein the intracellular signaling domain is or comprises an intracellular signaling domain of a CD3 chain, optionally a CD3-zeta (CD3ζ) chain, or a signaling portion thereof.
130. The polynucleotide of embodiment 128 or 129, wherein the intracellular region comprises one or more costimulatory signaling domain(s).
131. The polynucleotide of embodiment 130, wherein the one or more costimulatory signaling domain comprises an intracellular signaling domain of a CD28, a 4-1BB or an ICOS or a signaling portion thereof.
132. The polynucleotide of embodiment 130 or 131, wherein the costimulatory signaling region comprises an intracellular signaling domain of 4-1BB.
133. The polynucleotide of any of embodiments 92-132, wherein the modified T cell stimulation-associated locus encodes a recombinant receptor that is a CAR, wherein the CAR comprises, from its N to C terminus in order: the extracellular binding domain, the spacer, the transmembrane domain and an intracellular region.
134. The polynucleotide of any of embodiments 92-133, wherein
the transgene comprises in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a CH2 region and/or a CH3 region; and a transmembrane domain, optionally from human CD28; a costimulatory signaling domain, optionally from human 4-1BB; and an intracellular signaling domain, optionally a CD3ζ chain or a portion thereof; and/or
the modified T cell stimulation-associated locus comprises in order: a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a CH2 region and/or a CH3 region; and a transmembrane domain, optionally from human CD28; a costimulatory signaling domain, optionally from human 4-1BB; and an intracellular signaling domain, optionally a CD3ζ chain or a portion thereof.
135. The polynucleotide of any of embodiments 92-134, wherein the transgene encodes the recombinant receptor.
136. The polynucleotide of any of embodiments 92-134, wherein the transgene encodes a portion of the recombinant receptor.
137. The polynucleotide of embodiment 136, wherein the recombinant receptor contains two separate polypeptide chains, wherein the portion of the recombinant receptor encoded by the transgene is one chain of the recombinant receptor.
138. The polynucleotide of embodiment 137, wherein the other chain of the recombinant receptor is encoded by a second transgene.
139. The polynucleotide of any of embodiments 124-138, wherein the CAR is a multi-chain CAR.
140. The polynucleotide of embodiment 139, wherein transgene encodes one chain of the multi-chain CAR.
141. The polynucleotide of any of embodiments 92-123 and 135-138, wherein the recombinant receptor is a recombinant T cell receptor (TCR).
142. The polynucleotide of embodiment 141, wherein recombinant TCR comprises an alpha (TCRα) chain and a beta (TCRβ) chain and the transgene comprises a nucleic acid sequence encoding the TCRα chain and/or a nucleic acid sequence encoding the TCRβ chain.
143. The polynucleotide of embodiment 142, wherein the transgene encodes one of the TCRα chain or the TCRβ chain.
144. The polynucleotide of embodiment 142 or 143, wherein the TCRα chain comprises a constant (Cα) region comprising one or more introduced cysteine residues and/or the TCRβ chain comprises a Cβ region comprising one or more introduced cysteine residues, wherein the one or more introduced cysteine residues are capable of forming one or more non-native disulfide bridges between the alpha chain and beta chain, optionally wherein the one or more introduced cysteine residues comprises replacement of a non-cysteine residue with a cysteine residue.
145. The polynucleotide of embodiment 144, wherein the Cα region comprises a cysteine at a position corresponding to position 48 with numbering as set forth in SEQ ID NO: 92; and/or the CP region comprises a cysteine at a position corresponding to position 57 with numbering as set forth in SEQ ID NO: 96.
146. The polynucleotide of any of embodiments 92-145, wherein the transgene comprises a sequence of nucleotides encoding at least one further protein.
147. The polynucleotide of embodiment 146, wherein the at least one further protein is a surrogate marker, optionally wherein the surrogate marker is a truncated receptor, optionally wherein the truncated receptor lacks an intracellular signaling domain and/or is not capable of mediating intracellular signaling when bound by its ligand.
148. The polynucleotide of any of embodiments 92-147, wherein the transgene further comprises a multicistronic element(s).
149. The polynucleotide of embodiment 148, wherein the multicistronic element(s) comprises a sequence encoding a ribosome skip element selected from among a T2A, a P2A, a E2A or a F2A or an internal ribosome entry site (IRES).
150. The polynucleotide of embodiment 148 or 149, wherein:
the multicistronic element is positioned between the sequence of nucleotides encoding the CAR and the sequence of nucleotides encoding the at least one further protein;
the recombinant receptor is a recombinant TCR, and the multicistronic element is positioned between a sequence of nucleotides encoding the TCRα and a sequence of nucleotides encoding the TCRβ;
the recombinant receptor is a multi-chain CAR, and the multicistronic element is positioned between a sequence of nucleotides encoding one chain of the multi-chain CAR and a sequence of nucleotides encoding another chain of the multi-chain CAR; and/or
the multicistronic element(s) are upstream of the sequence of nucleotides encoding the recombinant receptor.
151. The polynucleotide of any of embodiments 92-150, that is a linear polynucleotide.
152. The polynucleotide of embodiment 151, that is a double-stranded polynucleotide.
153. The polynucleotide of embodiment 151, that is a single-stranded polynucleotide.
154. The polynucleotide of any of embodiments 92-150, wherein the polynucleotide is comprised in a viral vector.
155. The polynucleotide of embodiment 154, wherein the viral vector is an AAV vector.
156. The polynucleotide of embodiment 154, wherein the viral vector is a retroviral vector, optionally a lentiviral vector.
157. The polynucleotide of any of embodiments 92-156, wherein the polynucleotide is at or about 1000, 1250, 1500, 1750, 2000, 2250, 2500, 2750, 3000, 3250, 3500, 3750 or 4000 nucleotides in length, or any value between any of the foregoing.
158. The polynucleotide of any of embodiments 92-157, wherein the polynucleotide is between at or about 1500 and at or about 2500 nucleotides or at or about 1750 and at or about 2250 nucleotides in length.
159. A method of producing a genetically engineered T cell, the method comprising:
(a) introducing, into a T cell, one or more agent(s) capable of inducing a genetic disruption at a target site within an endogenous T cell stimulation-associated locus of the T cell; and
(b) introducing the polynucleotide of any of embodiments 92-158 into a T cell comprising a genetic disruption at a T cell stimulation-associated locus, wherein the method produces a modified T cell stimulation-associated locus, said modified T cell stimulation-associated locus comprising a transgene encoding the recombinant receptor or a portion thereof.
160. The method of embodiment 159, wherein the transgene encoding a recombinant receptor or a portion thereof is integrated within the endogenous T cell stimulation-associated locus via homology directed repair (HDR).
161. A method of producing a genetically engineered T cell, the method comprising introducing, into a T cell, a polynucleotide comprising a transgene encoding a recombinant receptor or a portion thereof, said T cell having a genetic disruption within a T cell stimulation-associated locus of the T cell, wherein the transgene encoding the recombinant receptor or a portion thereof is integrated within the endogenous T cell stimulation-associated locus via homology directed repair (HDR).
162. The method of embodiment 159 or 161, wherein the genetic disruption is carried out by introducing, into a T cell, one or more agent(s) capable of inducing a genetic disruption at a target site within an endogenous T cell stimulation-associated locus of the T cell.
163. The method of any of embodiments 159-162, wherein the method produces a modified T cell stimulation-associated locus, said modified T cell stimulation-associated locus comprising a transgene encoding a recombinant receptor or a portion thereof.
164. The method of any of embodiments 159-163, wherein the polynucleotide further comprises one or more homology arm(s) linked to the nucleic acid sequence, wherein the one or more homology arm(s) comprise a sequence homologous to one or more region(s) of an endogenous T cell stimulation-associated locus in a T cell.
165. The method of embodiment 164, wherein the one or more homology arm comprises a 5′ homology arm and/or a 3′ homology arm, optionally wherein the 5′ homology arm and 3′ homology arm comprises nucleic acid sequences homologous to nucleic acid sequences surrounding a target site, wherein the target site is within the T cell stimulation-associated locus.
166. The method of embodiment 165, wherein the target site is downstream of an endogenous transcriptional regulatory element of the T cell stimulation-associated locus.
167. The method of embodiment 165 or 166, wherein the polynucleotide comprises the structure [5′ homology arm]-[transgene]-[3′ homology arm].
168. The method of embodiment 167, wherein the 5′ homology arm and 3′ homology arm comprises nucleic acid sequences homologous to nucleic acid sequences surrounding the at least one target site.
169. The method of any of embodiments 165-168, wherein the 5′ homology arm and 3′ homology arm independently are between at or about 50 and at or about 750 nucleotides, at or about 50 and at or about 500 nucleotides, at or about 50 and at or about 250 nucleotides, at or about 50 and at or about 100 nucleotides, at or about 100 and at or about 750 nucleotides, at or about 100 and at or about 500 nucleotides, at or about 100 and at or about 250 nucleotides, at or about 250 and at or about 750 nucleotides, at or about 250 and at or about 500 nucleotides, in length.
170. The method of any of embodiments 165-169, wherein the 5′ homology arm and 3′ homology arm independently are at or about 50, 60, 70, 80, 90, 100, 150, 200, 250, 300, 400 or 500 nucleotides, or any value between any of the foregoing, in length.
171. The method of any of embodiments 165-170, wherein the 5′ homology arm and 3′ homology arm independently are less than at or about 100 nucleotides, in length, optionally at or about 50, 60, 70, 80 or 90 nucleotides, or any value between any of the foregoing, in length.
172. The method of any of embodiments 165-171, wherein the T cell stimulation-associated locus is selected from among PDCD1, CD69, Nur77, FoxP3 and a HLA-DR locus.
173. The method of embodiment 172, wherein the T cell stimulation-associated locus is PDCD1.
174. The method of embodiment 173, wherein the 5′ homology arm and 3′ homology arm comprise a sequence homologous to one or more region(s) of PDCD1.
175. The method of embodiment 174, wherein the 5′ homology arm comprises:
a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 66;
b) a sequence comprising at or at least at or at least 150, 200, 250, 300, 350, 400, 450, 500, 550, or 600 contiguous nucleotides of the sequence set forth in SEQ ID NO:66; or
c) the sequence set forth in SEQ ID NO: 66.
176. The method of embodiment 174 or 175, wherein the 3′ homology arm comprises:
a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 67;
b) a sequence comprising at or at least at or at least 150, 200, 250, 300, 350, 400, 450, 500, 550, or 600 contiguous nucleotides of the sequence set forth in SEQ ID NO:67; or
c) the sequence set forth in SEQ ID NO: 67.
177. The method of embodiment 172, wherein the T cell stimulation-associated locus is CD69.
178. The method of embodiment 177, wherein the 5′ homology arm and 3′ homology arm comprise a sequence homologous to one or more region(s) of CD69.
179. The method of embodiment 172, wherein the T cell stimulation-associated locus is Nur77.
180. The method of embodiment 179, wherein the 5′ homology arm and 3′ homology arm comprise a sequence homologous to one or more region(s) of Nur77.
181. The method of embodiment 172, wherein the T cell stimulation-associated locus is FoxP3.
182. The method of embodiment 181, wherein the 5′ homology arm and 3′ homology arm comprise a sequence homologous to one or more region(s) of FoxP3.
183. The method of embodiment 172, wherein the T cell stimulation-associated locus is a HLA-DR locus.
184. The method of embodiment 183, wherein the 5′ homology arm and 3′ homology arm comprise a sequence homologous to one or more region(s) of a HLA-DR locus.
185. The method of any of embodiments 159-184, wherein the recombinant receptor or portion thereof is capable of inducing or transmitting the stimulation or activation signal in the T cell.
186. The method of any of embodiments 159-185, wherein:
the recombinant receptor comprises an intracellular region comprising an intracellular signaling domain of a component of the T cell receptor (TCR) complex and the stimulation or activation signal in the T cells comprises a signal through the intracellular signaling domain present in the recombinant receptor, or
the recombinant receptor comprises an intracellular region comprising an intracellular signaling domain comprising an immunoreceptor tyrosine-based activation motif (ITAM) and the stimulation or activation signal in the T cells comprises a signal through the intracellular signaling domain present in the recombinant receptor.
187. The method of any of embodiments 159-186, wherein the recombinant receptor comprises an extracellular region comprising a binding domain that is capable of binding to or recognizing an agent.
188. The method of embodiment 187, wherein a stimulation or activation signal is induced in the T cell upon binding of the agent.
189. The method of embodiment 187 or 188, wherein the agent is a target antigen, optionally wherein the target antigen is a recombinant protein or is an antigen expressed on the surface of a cell.
190. The method of embodiment 189, wherein the target antigen is associated with, specific to, or expressed on a cell or tissue of a disease, disorder or condition.
191. The method of embodiment 190, wherein the disease, disorder or condition is an infectious disease or disorder, an autoimmune disease, an inflammatory disease, or a tumor or a cancer, optionally wherein the target antigen is a tumor antigen.
192. The method of any of embodiments 187-191, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-11 and LAGE-12), carcinoembryonic antigen (CEA), a cyclin, cyclin A2, C—C Motif Chemokine Ligand 1 (CCL-11), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-12), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-122 receptor alpha (IL-122Rα), IL-113 receptor alpha 2 (IL-113Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-11), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-11), a pathogen-specific or pathogen-expressed antigen, or an antigen associated with a universal tag, and/or biotinylated molecules, and/or molecules expressed by HIV, HCV, HBV or other pathogens.
193. The method of embodiment 191 or 192, wherein the genetic disruption is effected by a zinc finger nuclease (ZFN), a TAL-effector nuclease (TALEN), or a CRISPR-Cas9 combination that specifically binds to, recognizes, or hybridizes to the target site.
194. The method of any of embodiments 159-193, wherein the genetic disruption is effected by a CRISPR-Cas9 combination and the CRISPR-Cas9 combination comprises a guide RNA (gRNA) having a targeting domain that is complementary to the at least one target site.
195. The method of embodiment 194, wherein the CRISPR-Cas9 combination is a ribonucleoprotein (RNP) complex comprising the gRNA and a Cas9 protein.
196. The method of embodiment 195, wherein the genetic disruption of is effected by the RNP introduced into a plurality of T cells via electroporation.
197. The method of any of embodiments 159-196, wherein the T cell stimulation-associated locus is PDCD1.
198. The method of embodiment 197, wherein the genetic disruption is effected by a CRISPR-Cas9 combination comprising a gRNA and the gRNA has a targeting domain that is complementary to a target site in a PDCD1 gene.
199. The method of embodiment 198, wherein the gRNA comprises the sequence set forth in in any one of SEQ ID NOS: 75 and 104-109.
200. The method of embodiment 198 or 199, wherein the gRNA comprises the sequence set forth in SEQ ID NO:75.
201. The method of any of embodiments 159-196, wherein the T cell stimulation-associated locus is CD69.
202. The method of embodiment 201, wherein the genetic disruption is effected by a CRISPR-Cas9 combination comprising a gRNA and the gRNA has a targeting domain that is complementary to a target site in a CD69 gene.
203. The method of embodiment 202, wherein the gRNA comprises the sequence set forth in any one of SEQ ID NOS: 116-121.
204. The method of any of embodiments 159-196, wherein the T cell stimulation-associated locus is Nur77.
205. The method of embodiment 204, wherein the genetic disruption is effected by a CRISPR-Cas9 combination comprising a gRNA and the gRNA has a targeting domain that is complementary to a target site in a Nur77 gene.
206. The method of embodiment 205, wherein the gRNA comprises the sequence set forth in SEQ ID NO: 122-127 and 134-136.
207. The method of any of embodiments 159-196, wherein the T cell stimulation-associated locus is FoxP3.
208. The method of any of embodiments 159-196, wherein the T cell stimulation-associated locus is a HLA-DR locus.
209. The method of any of embodiments 159-208, wherein the T cell further comprises a genetic disruption at an endogenous T cell receptor alpha constant region (TRAC) gene and/or an endogenous T cell receptor beta constant region (TRBC) gene.
210. The method of embodiment 209, wherein the genetic disruption is effected by a zinc finger nuclease (ZFN), a TAL-effector nuclease (TALEN), or a CRISPR-Cas9 combination that specifically binds to, recognizes, or hybridizes to at least one target site is within the TRAC, TRBC1 and/or TRBC2 gene.
211. The method of embodiment 209 or 210, wherein the genetic disruption is effected by a CRISPR-Cas9 combination and the CRISPR-Cas9 combination comprises a guide RNA (gRNA) having a targeting domain that is complementary to the at least one target site, optionally wherein the CRISPR-Cas9 combination is a ribonucleoprotein (RNP) complex comprising the gRNA and a Cas9 protein, optionally wherein the genetic disruption of is effected by the RNP introduced into a plurality of T cells via electroporation.
212. The method of any of embodiments 209-211, wherein the genetic disruption is effected by a CRISPR-Cas9 combination comprising a gRNA and the gRNA has a targeting domain that is complementary to at least one target site is within the TRAC, TRBC1 and/or TRBC2 gene.
213. The method of embodiment 212, wherein the gRNA has a targeting domain that is complementary to a target site in a TRAC gene.
214. The method of embodiment 213, wherein the gRNA comprises the sequence set forth in any one of SEQ ID NOS: 77 and 188-218.
215. The method of embodiment 213 or 214, wherein the gRNA comprises the sequence set forth in SEQ ID NO:77.
216. The method of embodiment 212, wherein the gRNA has a targeting domain that is complementary to a target site in a TRBC gene.
217. The method of embodiment 216, wherein the gRNA comprises the sequence set forth in any one of SEQ ID NOS: 219-276.
218. The method of any of embodiments 159-217, wherein the recombinant receptor is a chimeric antigen receptor (CAR).
219. The method of any of embodiments 159-217, wherein the encoded recombinant receptor is or comprises recombinant T cell receptor (TCR).
220. The method of any of embodiments 211-219, wherein the RNP is introduced via electroporation, particle gun, calcium phosphate transfection, cell compression or squeezing, optionally via electroporation.
221. The method of embodiment 220, wherein the RNP introduced into a plurality of T cells via electroporation.
222. The method of embodiment 220 or 221, wherein the concentration of the RNP is from at or about 1 μM to at or about 5 μM, optionally wherein the concentration of the RNP is at or about 2 μM.
223. The method of any of embodiments 159-222, wherein T cells comprise CD8+ T cell and/or CD4+ T cells or subtypes thereof.
224. The method of any of embodiments 159-223, wherein the T cells are autologous to the subject.
225. The method of any of embodiments 159-224, wherein the T cell is a primary T cell derived from a subject, optionally wherein the subject is a human.
226. The method of any of embodiments 159-225, wherein the T cells are allogeneic to the subject.
227. The method of any of embodiments 159-224, wherein the T cell is derived from a multipotent or pluripotent cell, which optionally is an iPSC.
228. The method of any of embodiments 159-227, wherein the polynucleotide is a linear polynucleotide.
229. The method of embodiment 228, wherein the polynucleotide is a double-stranded polynucleotide.
230. The method of embodiment 228, wherein the polynucleotide is a single-stranded polynucleotide.
231. The method of any of embodiments 159-227, wherein the polynucleotide is comprised in a viral vector.
232. The method of embodiment 231, wherein the viral vector is an AAV vector.
233. The method of embodiment 231, wherein the viral vector is a retroviral vector, optionally a lentiviral vector.
234. The method of any of embodiments 159-233, wherein the polynucleotide is at or about 1000, 1250, 1500, 1750, 2000, 2250, 2500, 2750, 3000, 3250, 3500, 3750 or 4000 nucleotides in length, or any value between any of the foregoing.
235. The method of any of embodiments 159-234, wherein the polynucleotide is between at or about 1500 and at or about 2500 nucleotides or at or about 1750 and at or about 2250 nucleotides in length.
236. The method of any of embodiments 159, 160 and 162-235, wherein the one or more agent(s) and the polynucleotide are introduced simultaneously or sequentially, in any order.
237. The method of any of embodiments 159, 160 and 162-236, wherein the one or more agent(s) and the polynucleotide are introduced simultaneously.
238. The method of any of embodiments 159, 160 and 162-236, wherein the polynucleotide is introduced after the introduction of the one or more agent(s).
239. The method of embodiment 238, wherein the polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the agent.
240. The method of any of embodiments 159, 160 and 162-239, wherein prior to the introducing of the one or more agent(s) and/or the polynucleotide, the method comprises incubating the cells, in vitro with a stimulatory agent(s) under conditions to stimulate or activate the one or more immune cells.
241. The method of embodiment 240, wherein the stimulatory agent(s) comprises and anti-CD3 and/or anti-CD28 antibodies.
242. The method of embodiment 240 and 241, wherein the stimulatory agent(s) comprises an oligomeric particle reagent comprising anti-CD3 and/or anti-CD28 antibodies.
243. The method of any of embodiments 240-242, wherein the stimulatory agent(s) comprises beads coated with anti-CD3 and/or anti-CD28 antibodies.
244. The method of any of embodiments 159, 160 and 162-243, wherein the method further comprises incubating the cells prior to, during or subsequent to the introducing of the one or more agents and/or the introducing of the polynucleotide with one or more recombinant cytokines, optionally wherein the one or more recombinant cytokines are selected from the group consisting of IL-2, IL-7, and IL-15.
245. The method of embodiment 244, wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.
246. The method of embodiment 244 or 245, wherein the incubation is carried out subsequent to the introducing of the one or more agents and the introducing of the polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.
247. An engineered T cell or a plurality of engineered T cells generated using the method of any of embodiments 159-246.
248. A composition, comprising the engineered T cell of embodiment 247 or a plurality of the engineered T cell of embodiment 247.
249. The composition of embodiment 248, wherein the expression of the operably linked transgene is upregulated or induced in one or more cells in the composition within less than at or about 6, 12, 18, 24, 36 or 48 hours following the stimulation or activation signal in the T cells.
250. The composition of embodiment 248 or 249 wherein, following a simulation or activation signal in the T cells, the frequency of cells expressing the operably linked transgene among the cells in the composition is greater than at or about 50%, 60%, 70%, 75%, 80%, 85%, 90% or 95% or more.
251. The composition of any of embodiments 248-250, wherein, following the upregulation or induction of expression or following a reduction or an absence of the simulation or activation signal in the T cells, the expression of the operably linked transgene is reduced or downregulated in one or more cells in the composition.
252. The composition of any of embodiments 248-251, wherein, following the upregulation or induction of expression, or following the reduction or the absence of the simulation or activation signal in the T cells, the frequency of cells expressing the operably linked transgene among the cells in the composition is reduced by greater than at or about 30%, 40%, 50%, 60%, 70%, 75%, 80%, 85%, 90% or 95% or more.
253. The composition of any of embodiments 248-252, wherein, following the upregulation or induction of expression, and the expression of the operably linked transgene is reduced or downregulated in one or more cells in the composition after at or about 1, 2, 3, 4, 5, 6, 7 or 8 days or more after the stimulation or activation signal in the T cells.
254. The composition of any of embodiments 248-253, wherein the expression of the operably linked transgene in one or more of the cells in the composition is reduced or downregulated within less than at or about 6, 12, 18, 24, 36 or 48 hours following the reduction or an absence of the simulation or activation signal in the T cell.
255. The composition of embodiment 254, wherein, following a reduction or an absence of the simulation or activation signal in the T cells, the frequency of cells expressing the recombinant receptor among the cells in the composition is less than at or about 50%, 40%, 30%, 25%, 20%, 15%, 10% or 5% or less.
256. The composition of any of embodiments 248-255, wherein the composition comprises CD4+ T cells and/or CD8+ T cells.
257. The composition of any of embodiments 248-256, wherein the composition comprises CD4+ and CD8+ T cells and the ratio of CD4+ to CD8+ T cells is from or from about 1:3 to 3:1, optionally 1:1.
258. A method of treatment comprising administering the engineered cell of any of embodiments 1-81 and 247 or the composition of any of embodiments 82-91 and 248-257 to a subject having a disease or disorder.
259. Use of the engineered cell of any of embodiments 1-81 and 247 or the composition of any of embodiments 82-91 and 248-257 for the treatment of a disease or disorder.
260. Use of the engineered cell of any of embodiments 1-81 and 247 or the composition of any of embodiments 82-91 and 248-257 in the manufacture of a medicament for treating a disease or disorder.
261. The engineered cell of any of embodiments 1-81 and 247 or the composition of any of embodiments 82-91 and 248-257 for use in the treatment of a disease or disorder.
262. The method, use or the engineered cell, plurality of engineered cells or composition for use of any of embodiments 258-261, wherein the disease or disorder is a cancer or a tumor.
263. The method, use or the engineered cell, plurality of engineered cells or composition for use of embodiment 262, wherein the cancer or the tumor is a hematologic malignancy, optionally a lymphoma, a leukemia, or a plasma cell malignancy.
264. The method, use or the engineered cell, plurality of engineered cells or composition for use of embodiment 262 or 263, wherein the cancer is a lymphoma and the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL).
265. The method, use or the engineered cell, plurality of engineered cells or composition for use of any of embodiments 262-264, wherein the cancer is a leukemia and the leukemia is chronic lymphocytic leukemia (CLL), plasma cell leukemia or acute lymphocytic leukemia (ALL).
266. The method, use or the engineered cell, plurality of engineered cells or composition for use of any of embodiments 262-265, wherein the cancer is a plasma cell malignancy and the plasma cell malignancy is multiple myeloma (MM).
267. The method, use or the engineered cell, plurality of engineered cells or composition for use of embodiment 262, wherein the tumor is a solid tumor.
269. The method, use or the engineered cell, plurality of engineered cells or composition for use of embodiment 267, wherein the solid tumor is a non-small cell lung cancer (NSCLC) or a head and neck squamous cell carcinoma (HNSCC).
270. A kit comprising:
one or more agent(s) capable of inducing a genetic disruption at a target site within a T cell stimulation-associated locus; and
the polynucleotide of any of embodiments 92-158.
271. A kit, comprising:
one or more agent(s) capable of inducing a genetic disruption at a target site within a T cell stimulation-associated locus; and
a polynucleotide comprising a nucleic acid sequence encoding recombinant receptor or a portion thereof, wherein the transgene encoding the recombinant receptor or antigen-binding fragment or chain thereof is targeted for integration at or near the target site via homology directed repair (HDR); and
instructions for carrying out the method of any of embodiments 159-246.

### X. EXAMPLES

The following examples are included for illustrative purposes only and are not intended to limit the scope of the invention.

### Example 1: Expression Kinetics of T Cell Stimulation-Associated Molecules

The expression of various T cell stimulation signal-associated markers was assessed over time, to identify candidate loci for targeting integration of nucleic acid sequences encoding a chimeric antigen receptor (CAR). Nucleic acid sequences encoding a CAR were to be integrated into target loci in the genome of the cell that encode stimulation-associated markers, such that the expression of the CAR is under operable control of the promoters of the genes encoding the stimulation-associated markers. In some cases, the encoded CAR contains a signaling domain containing an immunoreceptor tyrosine-based activation motif (ITAM), for example, from a CD3-zeta (CD3ζ chain, or a signaling portion thereof. As depicted in FIG. 1, in some cases, the expression of the CAR can be regulated by a feedback loop, for example, expressed after transmission of T cell stimulation signal in the engineered T cell via the ITAM domain present in the CAR. The expression can be reduced or turned off if T cell stimulation signal is not present, for example, in the absence of stimulatory signal from binding of the CAR by the target antigen.

Primary T cells obtained from a human donor were stimulated by incubating with soluble multimeric reagent loaded with anti-CD3/anti-CD28 Fab antibody fragments or anti-CD3/anti-CD28 antibody conjugated beads on day 0, and cultured for 7 days, then subject to a re-stimulation after 7 days using the same reagent. The expression of cluster of differentiation 25 (CD25), CD69, CD45RA and programmed cell death protein 1 (PD-1), candidate stimulation-associated markers, were monitored over time, by flow cytometry.

As shown in FIG. 2, the expression of CD25, CD69 and PD-1 increased after stimulation. Around day 7 after the initial stimulation, the expression of CD69 and PD-1 decreased, consistent with downregulation of stimulation-associated markers. After the expression of CD29 and PD-1 decreased, the cells were re-stimulated using the same reagent. After re-stimulation, the expression of CD69 and PD-1 was observed to rapidly increase again. The expression of CD25 remained high around day 7 and after re-stimulation. The expression of CD45RA was reduced after around day 3, and did not increase after re-stimulation. PD-1 was selected as one of the location for targeted integration of nucleic acid sequences encoding a CAR.

### Example 2: Expression of Exemplary Chimeric Antigen Receptor (CAR) Under the Control of the Promoter of Stimulation-Associated Marker Programmed Cell Death Protein 1 (PD-1)

Nucleic acid sequences encoding an exemplary chimeric antigen receptor (CAR) were introduced into T cells with a genetic disruption at the endogenous gene loci that encodes the T cell receptor alpha (TCRα) chain and/or programmed cell death protein 1 (PD-1), by CRISPR/Cas9 mediated gene editing and targeted integration at the site of genetic disruption via homology-dependent repair (HDR).

### A. Generation of Engineered T Cells

Linear double stranded template polynucleotides containing nucleic acid sequences encoding an exemplary anti-CD19 CAR, flanked by 5′ and 3′ homology sequences for targeted for integration at the human TCR a constant region (TRAC) gene or the gene encoding PD-1 (PDCD1) were generated for HDR-mediated targeting. The encoded anti-CD19 CAR contained an scFv derived from a murine antibody (variable region derived from FMC63, V_{L}-linker-V_{H} orientation), three (3) Strep-tag II sequences, a transmembrane domain derived from CD28, a costimulatory region derived from 4-1BB, and a CD3-zeta intracellular signaling domain. The polynucleotides also contained P2A ribosome skip sequence upstream of the CAR-encoding sequences to allow the inserted nucleic acid sequences to be expressed under the control of the endogenous promoter at the insertion site and a polyadenylation signal for transcription termination and mRNA maturation. An exemplary nucleic acid sequence encoding an anti-CD19 CAR is set forth in SEQ ID NO:64. For targeting at the PDCD1 locus, the nucleic acid sequences were flanked by 5′ and 3′ homology arms of approximately 100 to 200 base pairs (set forth in SEQ ID NOS: 66 and 67, respectively), and were amplified using primers set forth in SEQ ID NOS: 68 and 69. For targeting at the TRAC locus, the nucleic acid sequences were flanked by 5′ and 3′ homology arms of approximately 50 to 70 base pairs (set forth in SEQ ID NOS: 68 and 69, respectively), and were amplified using primers set forth in SEQ ID NOS: 72 and 73.

Primary human CD4+ and CD8+ T cells were stimulated, cultured and subject to a one-step electroporation, with ribonucleoprotein (RNP) complexes containing PDCD1-targeting gRNA (set forth in SEQ ID NO: 75) or TRAC-targeting gRNA (set forth in SEQ ID NO:77), and linear polynucleotides for HDR-mediated targeting of the CAR-encoding nucleic acid at the PDCD1 or TRAC locus. T cells were stimulated by incubation with soluble multimeric reagent loaded with anti-CD3/anti-CD28 Fab antibody fragments or rested without reagent for about 48 hours. Cells were washed and suspended in electroporation mix. Pre-assembled RNP complexes containing TRAC-targeting gRNA and a Cas9 protein were mixed with linear polynucleotides, then added to the cell suspension. The cells were subject to electroporation, followed by incubation in culture medium for 5 days. As controls, cells were subject to electroporation with RNP complexes only (without template polynucleotide) or without RNP complexes. Five (5) days after electroporation (7 days after initial stimulation), the cells were subject to re-stimulation using the same reagent, or rested without re-stimulation. The cells were assessed by flow cytometry after staining with an anti-CD3 antibody, an anti-Strep-tag reagent to assess expression of the CAR, an anti-PD1 antibody and an anti-CD69 antibody, to monitor the expression of the CAR and the markers over time.

### B. Expression of Exemplary CAR

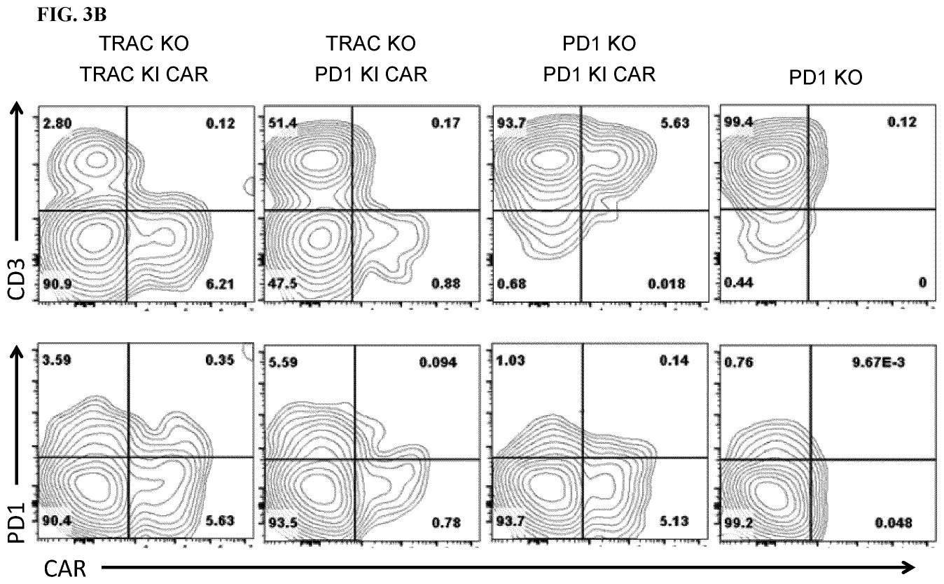

As shown in FIGS. 3A-3B, cells introduced with RNP complexes targeting the PDCD1 and PDCD1-targeting template polynucleotide resulted in CAR-expressing cells with a knock-out of PD-1 (as observed by the presence of PD1-CAR⁺ cells in PD-1 KO PD-1 CAR group; FIG. 3B); and cells introduced with RNP complexes targeting the TRAC and TRAC-targeting template polynucleotide resulted in CAR-expressing cells with a knock-out of endogenous CD3 (as observed by the presence of CD3-CAR⁺ cells in TRAC KO TRAC KI CAR group; FIG. 3B), at 7 days after initial stimulation.

### C. Stimulation-Associated Control of CAR Expression

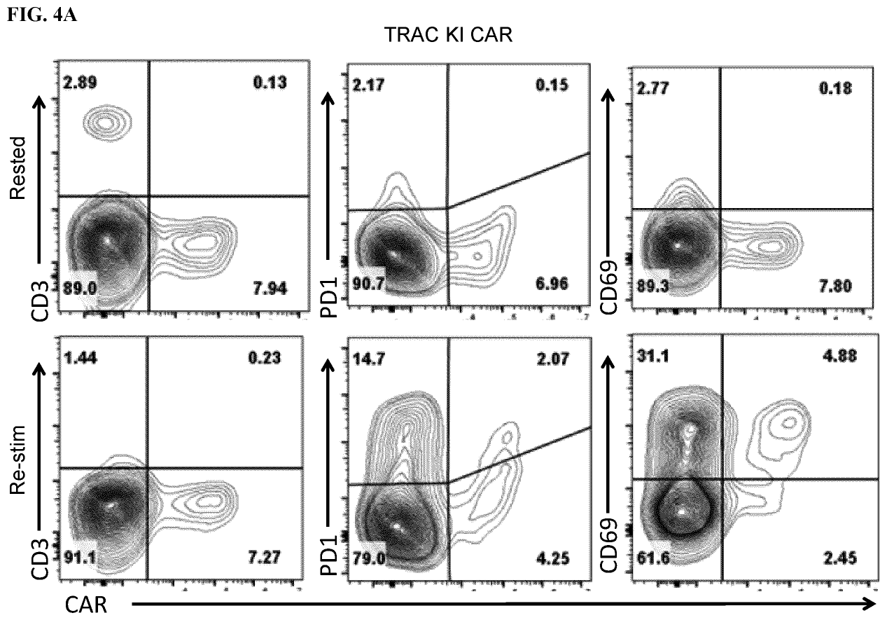

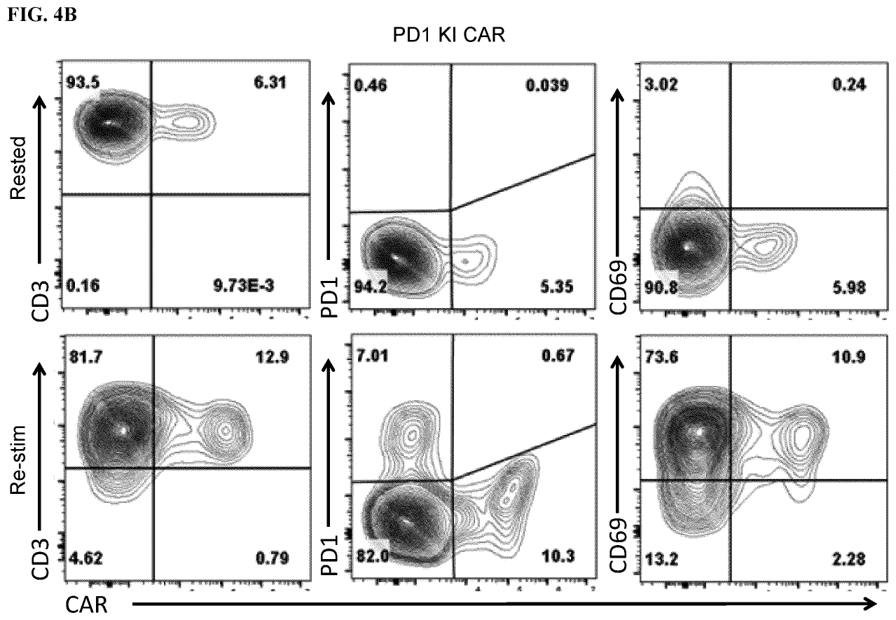

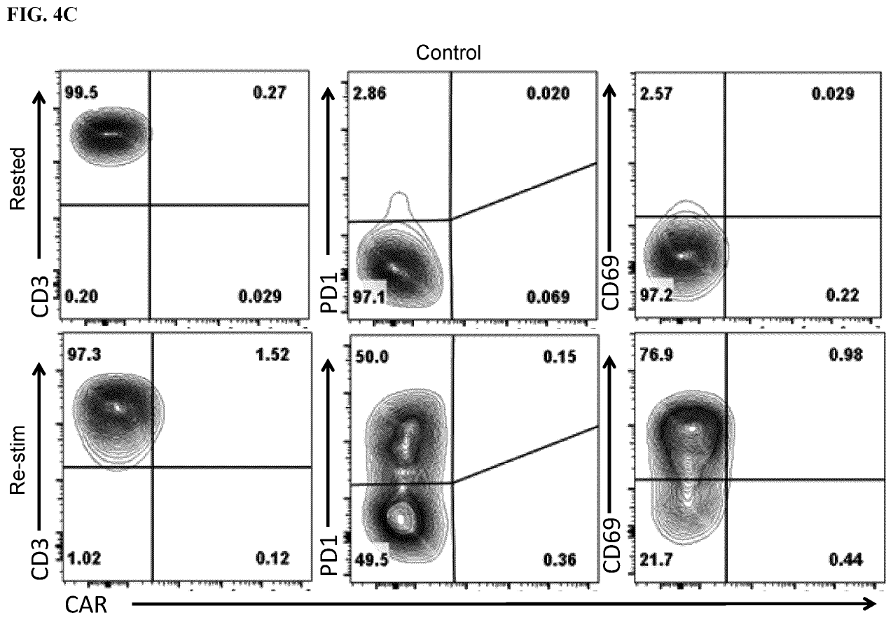

The percentage of CAR-expressing cells, engineered by integrating CAR-encoding nucleic acid sequences at the endogenous TRAC locus (TRAC KI CAR; under control of the endogenous TRAC promoter) was similar in re-stimulated cells and rested cells; and the percentage of cells expressing PD-1 and CD69 increased after re-stimulation (see FIG. 4A). In comparison, the percentage of CAR-expressing cells, engineered by integrating CAR-encoding nucleic acid sequences at the endogenous PDCD1 locus (PD1 KI CAR; under control of the endogenous PDCD1 promoter) was higher in re-stimulated cells, compared to rested cells; and the percentage of cells expressing CD69 increased after re-stimulation (see FIG. 4B). Control cells showed an increase in PD-1 and CD69 expression after re-stimulation (see FIG. 4C).

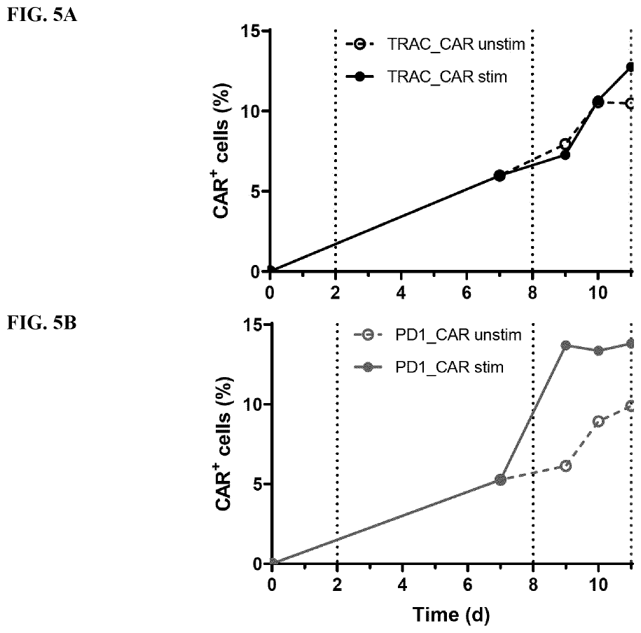

The percentage of CAR+ cells in a population of cells engineered by integration of the nucleic acid at the endogenous TRAC locus increased over time, and were similar between re-stimulated and rested cells (see FIG. 5A). In comparison, the percentage of CAR+ cells in a population of cells engineered by integration of the nucleic acid at the endogenous PDCD1 locus increased over time, and was substantially higher in cells that were re-stimulated, after the re-stimulation (see FIG. 5B).

### D. Conclusion

The expression of an exemplary CAR under the control of a promoter of an exemplary stimulation-associated endogenous locus, such as the endogenous PDCD1 locus, was observed to also be dependent on stimulatory signal of the T cells. The expression was substantially increased upon re-stimulation of the cells compared to rested cells. The results support the use of targeted integration of CAR-encoding sequences for regulating the expression of the CAR based on T cell stimulation or activation signal.

### Example 3: Assessment of Activity of Engineered T Expressing Exemplary Chimeric Antigen Receptor (CAR) Under the Control of the Promoter of Stimulation-Associated Marker Programmed Cell Death Protein 1 (PD-1)

The activity of T cells expressing an exemplary chimeric antigen receptor (CAR) from the endogenous gene loci that encodes programmed cell death protein 1 (PD-1), generated as described in Example 2 above, were assessed.

### A. Engineered T Cells

Primary human CD4+ and CD8+ T cells were engineered to express an exemplary anti-CD19 CAR, by integrating CAR-encoding nucleic acid sequences at the endogenous PDCD1 locus (under control of the endogenous PDCD1 promoter: PD1 KI CAR), generally as described in Example 2. PDCD1 KO cells (electroporated with PDCD1 targeting RNP complexes only, without template polynucleotide; PD1 KO), mock treated cells (Neg. control), and/or cells expressing the same exemplary CAR engineered using a lentiviral vector (LV control), were used as controls.

### B. Expansion

To assess antigen-specific expansion of the engineered T cells, PD1 KI CAR (expressing anti-CD19 CAR from the modified PDCD1 locus), PD1 KO, Neg. control and LV control cells described above were cultured for 7 days after electroporation. The cells were then expanded by co-culturing with irradiated CD19-expressing lymphoblastoid cell lines (LCLs), at two rounds of co-culture at 7 day intervals. Expansion of the cells after antigen-specific stimulation were assessed by flow cytometry, staining with an anti-CD8 antibody, and an anti-idiotype antibody to detect expression of the exemplary anti-CD19 CAR.

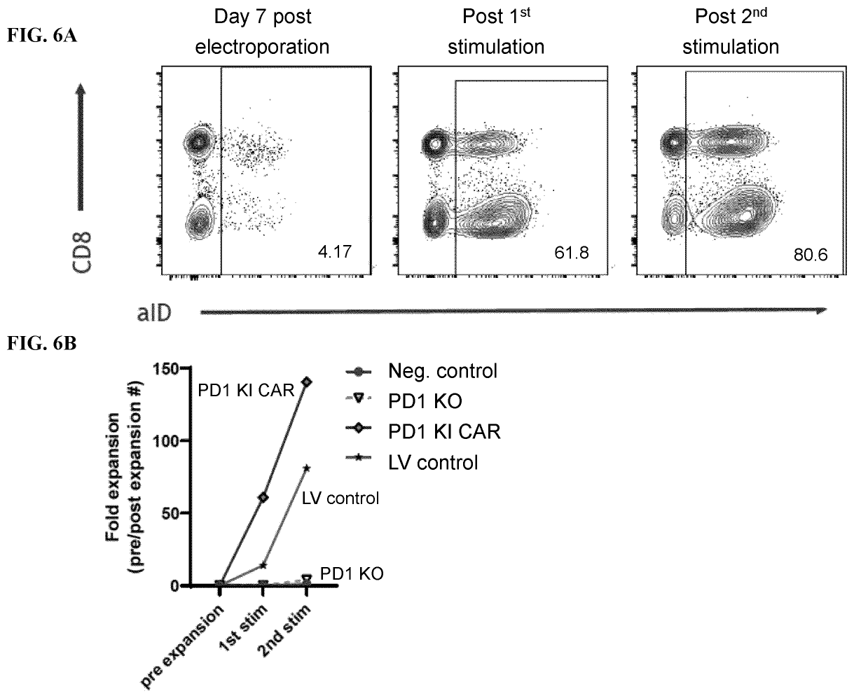

As shown in FIGS. 6A-6B, the percentage of cells expressing the CAR under the control of the PDCD1 promoter (PD1 KI CAR) substantially increased after the first and the second stimulations. Cells expressing the CAR engineered using a lentiviral vector and random integration (LV control) also showed expansion after antigen-specific stimulation, but to a lesser extent compared to the PD1 KI CAR cells. The results showed that cells expressing the CAR under the control of the stimulation-associated PDCD1 promoter expanded efficiently upon antigen-specific stimulation.

### C. Expression after Re-Stimulation

CAR expression was assessed after re-stimulation of the engineered T cells expressing the anti-CD19 CAR from the modified PDCD1 locus. PD1 KI CAR and PD1 KO cells were subject to re-stimulation after initial stimulation and expansion, by co-culturing with irradiated CD19-expressing LCLs, or rested without re-stimulation. Cells were assessed by flow cytometry, staining for stimulation-associated markers (CD25, CD69) and an anti-idiotype antibody to detect expression of the exemplary anti-CD19 CAR.

As shown in FIGS. 7A-7C, the percentage of CAR-expressing cells (FIGS. 7A and 7B) and the mean level of expression (as determined by mean fluorescence intensity, MFI; FIGS. 7B and 7C) of the anti-CD19 CAR were increased in PD1 KI CAR cells, following the re-stimulation (PD1 KI CAR restim), compared to cells that were not re-stimulated (PD1 KI CAR rested). The percentage of CD25+ CD69+ cells were also higher in cells after re-stimulation (FIG. 7B).

### D. Cytolytic Activity after Re-Stimulation

Target-specific cytolytic activity was assessed after re-stimulation of the engineered T cells expressing the anti-CD19 CAR from the modified PDCD1 locus. PD1 KI CAR and PD1 KO cells were subject to re-stimulation after initial stimulation and expansion, by co-culturing with irradiated CD19-expressing LCLs, or rested without re-stimulation. Cytolytic activity was assessed by impedance measurement of cultured the anti-CD19 CAR-expressing effector cells with target cells expressing CD19 at an effector to target (E:T) ratio of 10:1. The ability of the T cells to antigen-specifically lyse the target cells was assessed by measuring the detachment of target cells for up to 24 hours post co-culture. Mock treated cells (Neg. control), LV control and human embryonic kidney (HEK) cells were used as controls.

As shown in FIG. 7D, the re-stimulated PD1 KI CAR cells killed target cells more efficiently than PD1 KI CAR cells that were not re-stimulated or LV control.

### E. Conclusion

T cells engineered to express a CAR under the control of an exemplary stimulation-associated endogenous locus PDCD1 (PD1 KI CAR) exhibited stimulation-dependent expression of the CAR, and efficient target cell killing upon stimulation. The results support the use of targeted integration of CAR-encoding sequences for regulating the expression and function of the CAR based on T cell stimulation or activation signal.

### Example 4: Assessment of In Vivo Anti-Tumor Effects of Engineered T Expressing Exemplary CAR Under the Control of the PD1 in Mice

Anti-tumor activity of engineered T cells expressing an exemplary chimeric antigen receptor (CAR) from the endogenous gene loci that encodes programmed cell death protein 1 (PD1), generated as described in Example 2 above, was assessed by administration of the engineered cells in a tumor mouse model.

NOD.Cg.Prkdc^{scid}IL2rg^{tm1Wjl}/SzJ (NSG) mice were injected intravenously (i.v.) with 0.5×10⁶ Raji lymphoma tumor cells (an immortalized human B lymphocyte tumor cell line that expresses CD19) that were transfected with firefly luciferase (Raji-ffluc). Tumor engraftment was allowed to occur for 6 days and verified using bioluminescence imaging. 7 days after tumor cell injection, mice either received no treatment, or a single intravenous (i.v.) injection of 1×10⁶ of primary human T cells engineered to express the exemplary anti-CD19 CAR as described in Example 2 above (PD1 KI CAR), or primary human T cells engineered to express the same anti-CD19 CAR by lentiviral vector delivery and random integration (LV control). Tumor burden was assessed by bioluminescence every week, for up to 35 days after administration of the engineered T cells. For bioluminescence imaging, mice received intraperitoneal (i.p.) injections of luciferin substrate (CaliperLife Sciences, Hopkinton, MA) resuspended in PBS (15 μg/g body weight). The average radiance (p/s/cm²/sr) was determined. A schematic of the timeline of the experiment is shown in FIG. 8A.

As shown in FIGS. 8B-8D, mice administered PD1 KI CAR or LV control resulted in reduction of tumor cell growth and increased survival. These results demonstrate in vivo anti-tumor activity of T cells engineered to express a CAR under the control of an exemplary stimulation-associated endogenous locus PDCD1 (PD1 KI CAR).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ESKYGPPCPPCP | spacer |
| | | (IgG4hinge) (aa) |
| 2 | GAATCTAAGTACGGACCGCCCTGCCCCCCTTGCCCT | spacer |
| | | (IgG4hinge) (nt) |
| 3 | ESKYGPPCPPCPGQPREPQVYTLPPSQEEMTKNQVSLTCLVKGFYPSDIA | Hinge-CH3 spacer |
| | VEWESNGQPENNYKTTPPVLDSDGSFFLYSRLTVDKSRWQEGNVFSCSV | |
| | MHEALHNHYTQKSLSLSLGK | |
| 4 | ESKYGPPCPPCPAPEFLGGPSVFLFPPKPKDTLMISRTPEVTCVVVDVSQE | Hinge-CH2-CH3 |
| | DPEVQFNWYVDGVEVHNAKTKPREEQFNSTYRVVSVLTVLHQDWLNG | spacer |
| | KEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSR | |
| | WQEGNVFSCSVMHEALHNHYTQKSLSLSLGK | |
| 5 | RWPESPKAQASSVPTAQPQAEGSLAKATTAPATTRNTGRGGEEKKKEKE | IgD-hinge-Fc |
| | KEEQEERETKTPECPSHTQPLGVYLLTPAVQDLWLRDKATFTCFVVGSD | |
| | LKDAHLTWEVAGKVPTGGVEEGLLERHSNGSQSQHSRLTLPRSLWNAG | |
| | TSVTCTLNHPSLPPQRLMALREPAAQAPVKLSLNLLASSDPPEAASWLLC | |
| | EVSGFSPPNILLMWLEDQREVNTSGFAPARPPPQPGSTTFWAWSVLRVPA | |
| | PPSPQPATYTCVVSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHFKNCT | tEGFR |
| | SISGDLHILPVAFRGDSFTHTPPLDPQELDILKTVKEITGFLLIQAWPENRT | |
| | DLHAFENLEIIRGRTKQHGQFSLAVVSLNITSLGLRSLKEISDGDVIISGNK | |
| | NLCYANTINWKKLFGTSGQKTKIISNRGENSCKATGQVCHALCSPEGCW | |
| | GPEPRDCVSCRNVSRGRECVDKCNLLEGEPREFVENSECIQCHPECLPQA | |
| | MNITCTGRGPDNCIQCAHYIDGPHCVKTCPAGVMGENNTLVWKYADAG | |
| | HVCHLCHPNCTYGCTGPGLEGCPTNGPKIPSIATGMVGALLLLLVVALGI | |
| | GLFM | |
| 8 | FWVLVVVGGVLACYSLLVTVAFIIFWV | CD28 (amino |
| | | acids 153-179 of |
| | | Accession No. |
| | | P10747) |
| 9 | IEVMYPPPYLDNEKSNGTIIHVKGKHLCPSPLFPGPSKPFWVLVVVGGVL | CD28 (amino |
| | ACYSLLVTVAFIIFWV | acids 114-179 of |
| | | Accession No. |
| | | P10747) |
| 10 | RSKRSRLLHSDYMNMTPRRPGPTRKHYQPYAPPRDFAAYRS | CD28 (amino |
| | | acids 180-220 of |
| | | P10747) |
| 11 | RSKRSRGGHSDYMNMTPRRPGPTRKHYQPYAPPRDFAAYRS | CD28 (LL to GG) |
| 12 | KRGRKKLLYIFKQPFMRPVQTTQEEDGCSCRFPEEEEGGCEL | 4-1BB (amino |
| | | acids 214-255 of |
| | | Q07011.1) |
| 13 | RVKFSRSADAPAYQQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 14 | RVKFSRSAEPPAYQQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 15 | RVKFSRSADAPAYKQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 16 | RKVCNGIGIGEFKDSLSINATNIKHFKNCTSISGDLHILPVAFRGDSFTHTP | tEGFR |
| | PLDPQELDILKTVKEITGFLLIQAWPENRTDLHAFENLEIIRGRTKQHGQFS | |
| | LAVVSLNITSLGLRSLKEISDGDVIISGNKNLCYANTINWKKLFGTSGQKT | |
| | KIISNRGENSCKATGQVCHALCSPEGCWGPEPRDCVSCRNVSRGRECVD | |
| | KCNLLEGEPREFVENSECIQCHPECLPQAMNITCTGRGPDNCIQCAHYIDG | |
| | PHCVKTCPAGVMGENNTLVWKYADAGHVCHLCHPNCTYGCTGPGLEG | |
| | CPTNGPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)n-P- wherein P is proline, G is glycine and S is serine | Linker |
| 23 | GSADDAKKDAAKKDGKS | Linker |
| 24 | atgcttctcctggtgacaagccttctgctctgtgagttaccacacccagcattcctcctgatccca | GMCSFR alpha |
| | | chain signal |
| | | sequence |
| 25 | MLLLVTSLLLCELPHPAFLLIP | GMCSFR alpha |
| | | chain signal |
| | | sequence |
| 26 | MALPVTALLLPLALLLHA | CD8 alpha signal |
| | | peptide |
| 27 | EPKSCDKTHTCPPCP | Hinge |
| 28 | ERKCCVECPPCP | Hinge |
| 29 | ELKTPLGDTHTCPRCPEPKSCDTPPPCPRCPEPKSCDTPPPCPRCPEPKSCD | Hinge |
| | TPPPCPRCP | |
| 30 | ESKYGPPCPSCP | Hinge |
| 31 | X1PPX2P | Hinge |
| | X1 is glycine, cysteine or arginine | |
| | X2 is cysteine or threonine | |
| 32 | YGPPCPPCP | Hinge |
| 33 | KYGPPCPPCP | Hinge |
| 34 | EVVVKYGPPCPPCP | Hinge |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGLEWLG | VH |
| | VIWGSETTYYNSALKSRLTIIKDNSKSQVFLKMNSLQTDDTAIYYCAKHY | |
| | YYGGSYAMDYWGQGTSVTVSS | |
| 42 | DIQMTQTTSSLSASLGDRVTISCRASQDISKYLNWYQQKPDGTVKLLIYH | VL |
| | TSRLHSGVPSRFSGSGSGTDYSLTISNLEQEDIATYFCQQGNTLPYTFGGG | |
| | TKLEIT | |
| 43 | DIQMTQTTSSLSASLGDRVTISCRASQDISKYLNWYQQKPDGTVKLLIYH | scFv |
| | TSRLHSGVPSRFSGSGSGTDYSLTISNLEQEDIATYFCQQGNTLPYTFGGG | |
| | TKLEITGSTSGSGKPGSGEGSTKGEVKLQESGPGLVAPSQSLSVTCTVSGV | |
| | SLPDYGVSWIRQPPRKGLEWLGVIWGSETTYYNSALKSRLTIIKDNSKSQ | |
| | VFLKMNSLQTDDTAIYYCAKHYYYGGSYAMDYWGQGTSVTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQRPGQGLEWI | VH |
| | GQIYPGDGDTNYNGKFKGQATLTADKSSSTAYMQLSGLTSEDSAVYFCA | |
| | RKTISSVVDFYFDYWGQGTTVTVSS | |
| 51 | DIELTQSPKFMSTSVGDRVSVTCKASQNVGTNVAWYQQKPGQSPKPLIY | VL |
| | SATYRNSGVPDRFTGSGSGTDFTLTITNVQSKDLADYFCQQYNRYPYTSG | |
| | GGTKLEIKR | |
| 52 | GGGGGGGGSGGGGS | Linker |
| 53 | EVKLQQSGAELVRPGSSVKISCKASGYAFSSYWMNWVKQRPGQGLEWI | scFv |
| | GQIYPGDGDTNYNGKFKGQATLTADKSSSTAYMQLSGLTSEDSAVYFCA | |
| | RKTISSVVDFYFDYWGQGTTVTVSSGGGGSGGGGSGGGGSDIELTQSPKF | |
| | MSTSVGDRVSVTCKASQNVGTNVAWYQQKPGQSPKPLIYSATYRNSGV | |
| | PDRFTGSGSGTDFTLTITNVQSKDLADYFCQQYNRYPYTSGGGTKLEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacctccagcctgagcgccagcctgggcgaccgggtgaccatcagctgccg | Sequence |
| | ggccagccaggacatcagcaagtacctgaactggtatcagcagaagcccgacggcaccgtcaagctgctga | encoding scFv |
| | tctaccacaccagccggctgcacagcggcgtgcccagccggtttagcggcagcggctccggcaccgactac | |
| | agcctgaccatctccaacctggaacaggaagatatcgccacctacttttgccagcagggcaacacactgcc | |
| | ctacacctttggcggcggaacaaagctggaaatcaccggcagcacctccggcagcggcaagcctggcagcg | |
| | gcgagggcagcaccaagggcgaggtgaagctgcaggaaagcggccctggcctggtggcccccagccagagc | |
| | ctgagcgtgacctgcaccgtgagcggcgtgagcctgcccgactacggcgtgagctggatccggcagccccc | |
| | caggaagggcctggaatggctgggcgtgatctggggcagcgagaccacctactacaacagcgccctgaaga | |
| | gccggctgaccatcatcaaggacaacagcaagagccaggtgttcctgaagatgaacagcctgcagaccgac | |
| | gacaccgccatctactactgcgccaagcactactactacggcggcagctacgccatggactactggggcca | |
| | gggcaccagcgtgaccgtgagcagc | |
| 58 | GSTSGSGKPGSGEGSTKG | Linker |
| 59 | ASTKGPSVFPLAPCSRSTSESTAALGCLVKDYFPEPVTVSWNSGALTSGV | Human IgG2 Fc |
| | HTFPAVLQSSGLYSLSSVVTVPSSNFGTQTYTCNVDHKPSNTKVDKTVER | (Uniprot P01859) |
| | KCCVECPPCPAPPVAGPSVFLFPPKPKDTLMISRTPEVTCVVVDVSHEDPE | |
| | VQFNWYVDGVEVHNAKTKPREEQFNSTFRVVSVLTVVHQDWLNGKEY | |
| | KCKVSNKGLPAPIEKTISKTKGQPREPQVYTLPPSREEMTKNQVSLTCLV | |
| | KGFYPSDISVEWESNGQPENNYKTTPPMLDSDGSFFLYSKLTVDKSRWQ | |
| | QGNVFSCSVMHEALHNHYTQKSLSLSPGK | |
| 60 | ASTKGPSVFPLAPCSRSTSESTAALGCLVKDYFPEPVTVSWNSGALTSGV | Human IgG4 Fc |
| | HTFPAVLQSSGLYSLSSVVTVPSSSLGTKTYTCNVDHKPSNTKVDKRVES | (Uniprot P01861) |
| | KYGPPCPSCPAPEFLGGPSVFLFPPKPKDTLMISRTPEVTCVVVDVSQEDP | |
| | EVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRW | |
| | QEGNVFSCSVMHEALHNHYTQKSLSLSLGK | |
| 61 | GGAAGCGGAGAGGGCAGAGGAAGTCTTCTAACATGCGGTGACGTGG | P2A |
| | AGGAGAATCCCGGCCCA | |
| 62 | GSGEGRGSLLTCGDVEENPGP | P2A |
| 63 | GCCTCGACTGTGCCTTCTAGTTGCCAGCCATCTGTTGTTTGCCCCTCCC | Poly A signal |
| | CCGTGCCTTCCTTGACCCTGGAAGGTGCCACTCCCACTGTCCTTTCCTA | |
| | ATAAAATGAGGAAATTGCATCGCATTGTCTGAGTAGGTGTCATTCTAT | |
| | TCTGGGGGGTGGGGTGGGGCAGGACAGCAAGGGGGAGGATTGGGAA | |
| | GACAATAGCAGGCATGCTGGGGA | |
| 64 | atgctgctgctggtgaccagcctgctgctgtgcgagctgccccaccccgcctttctgctgatccccgacatccagatg | anti-CD19 CAR |
| | acccagaccacctccagcctgagcgccagcctgggcgaccgggtgaccatcagctgccgggccagccaggaca | (nt) |
| | tcagcaagtacctgaactggtatcagcagaagcccgacggcaccgtcaagctgctgatctaccacaccagccggct | |
| | gcacagcggcgtgcccagccggtttagcggcagcggctccggcaccgactacagcctgaccatctccaacctgg | |
| | aacaggaagatatcgccacctacttttgccagcagggcaacacactgccctacacctttggcggcggaacaaagct | |
| | ggaaatcaccggcagcacctccggcagcggcaagcctggcagcggcgagggcagcaccaagggcgaggtga | |
| | agctgcaggaaagcggccctggcctggtggcccccagccagagcctgagcgtgacctgcaccgtgagcggcgt | |
| | gagcctgcccgactacggcgtgagctggatcaggcagccccccaggaagggcctggaatggctgggcgtgatct | |
| | ggggcagcgagaccacctactacaacagcgccctgaagagccggctgaccatcatcaaggacaacagcaagag | |
| | ccaggtgttcctgaagatgaacagcctgcagaccgacgacaccgccatctactactgcgccaagcactactactac | |
| | ggcggcagctacgccatggactactggggccagggcaccagcgtgaccgtgagcagcgagagcaagaattgga | |
| | gccacccgcagttcgaaaaaggaggtggaggttcaggtggtggaggctcttacggaccgaattggtctcatcctca | |
| | gttcgagaaaggaggcggttctggaggtggaagcggtggctcttggagccacccacagtttgaaaagggaggcg | |
| | ggggctccggtggcggaggctcttccggatctccctgtccaccttgccctatgttctgggtgctggtagtggtaggtg | |
| | gagtgctggcctgctacagcctgctggtgacagtggccttcatcatcttttgggtgaaacggggcagaaagaaactc | |
| | ctgtatatattcaaacaaccatttatgagaccagtacaaactactcaagaggaagatggctgtagctgccgatttccag | |
| | aagaagaagaaggaggatgtgaactgcgggtgaagttcagcagaagcgccgacgcacctgcctaccagcaggg | |
| | ccagaatcagctgtacaacgagctgaacctgggacgaagggaagagtacgacgtcctggataagcggagaggcc | |
| | gggaccctgagatgggcggcaagcctcggcggaagaacccccaggaaggcctgtataacgaactgcagaaaga | |
| | caagatggccgaggcctacagcgagatcggcatgaagggcgagcggaggcggggcaagggccacgacggcc | |
| | tgtatcagggcctgtccaccgccaccaaggatacctacgacgccctgcacatgcaggccctgcccccaaggtga | |
| 65 | DIQMTQTTSSLSASLGDRVTISCRASQDISKYLNWYQQKPDGTVKLLIYH | anti-CD19 CAR |
| | TSRLHSGVPSRFSGSGSGTDYSLTISNLEQEDIATYFCQQGNTLPYTFGGG | (aa) |
| | TKLEITGSTSGSGKPGSGEGSTKGEVKLQESGPGLVAPSQSLSVTCTVSGV | |
| | SLPDYGVSWIRQPPRKGLEWLGVIWGSETTYYNSALKSRLTIIKDNSKSQ | |
| | VFLKMNSLQTDDTAIYYCAKHYYYGGSYAMDYWGQGTSVTVSSESKN | |
| | WSHPQFEKGGGGSGGGGSYGPNWSHPQFEKGGGSGGGSGGSWSHPQFE | |
| | KGGGGSGGGGSSGSPCPPCPMFWVLVVVGGVLACYSLLVTVAFIIFWVK | |
| | RGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQE | |
| | GLYNELQKDKMAEAYSEIGMKGERRRGKGHDGLYQGLSTATKDTYDA | |
| | LHMQALPPR | |
| 66 | TAATTACCTCCGCCTGAGCAGTGGAGAAGGCGGCACTCTGGTGGGGC | PDCD1 5′ |
| | TGCTCCAGGCATGCAGATCCCACAGGCGCCCTGGCCAGTCGTCTGGG | homology arm |
| | CGGTGCTA | |
| 67 | CAACTGGGCTGGCGGCCAGGATGGTTCTTAGGTAGGTGGGGTCGGCG | PDCD1 3′ |
| | GTCAGGTGTCCCAGAGCCAGGGGTCTGGAGGGACCTTCCACCCTCAG | homology arm |
| | TCCCTGGCAGGTCGGGGGGTGCTGAGGCGGGCCTGGCCCTGGCAGCC | |
| | CAGGGGTCCCGGAGCGAGGGGTCTGGAGGGACCTTTCACTCTCAGT | |
| 68 | GGGAAATGAGATCATGTCCTAACCCTGATCCTCTTGTCCCACAGATAT | TRAC 5′ |
| | CCAGAACCCTGACCCTGCCGTG | homology arm |
| 69 | TACCAGCTGAGAGACTCTAAATCCAGTGACAAGTCTGTCTGCCTATTC | TRAC 3′ |
| | ACCGATTTTG | homology arm |
| 70 | TAATTACCTCCGCCTGAGCA | PDCD1 5′ primer |
| 71 | ACTGAGAGTGAAAGGTCCCT | PDCD1 3′ primer |
| 72 | CAAAATCGGTGAATAGGCAG | TRAC 5′ primer |
| 73 | GGGAAATGAGATCATGTCCT | TRAC 3′ primer |
| 74 | CGTCTGGGCGGTGCTACAAC | PDCD1 gRNA |
| | | targeting sequence |
| 75 | CGUCUGGGCGGUGCUACAAC | PDCD1 gRNA |
| 76 | AGAGTCTCTCAGCTGGTACA | TRAC gRNA |
| | | targeting sequence |
| 77 | AGAGUCUCUCAGCUGGUACA | TRAC gRNA |
| 78 | CGTCTGGGCGGTGCTACAACGTTTTAGAGCTATGCT | PDCD1 gRNA |
| | | targeting sequence |
| 79 | MQIPQAPWPVVWAVLQLGWRPGWFLDSPDRPWNPPTFSPALLVVTEGD | PD-1 (Uniprot |
| | NATFTCSFSNTSESFVLNWYRMSPSNQTDKLAAFPEDRSQPGQDCRFRVT | Q15116-1) |
| | QLPNGRDFHMSVVRARRNDSGTYLCGAISLAPKAQIKESLRAELRVTERR | |
| | AEVPTAHPSPSPRPAGQFQTLVVGVVGGLLGSLVLLVWVLAVICSRAAR | |
| | GTIGARRTGQPLKEDPSAVPVFSVDYGELDFQWREKTPEPPVPCVPEQTE | |
| | YATIVFPSGMGTSSPARRGSADGPRSAQPLRPEDGHCSWPL | |
| 80 | gctcacctccgcctgagcagtggagaaggcggcactctggtggggctgctccaggcatgcagatcccacaggcg | PDCDI (NCBI |
| | ccctggccagtcgtctgggcggtgctacaactgggctggcggccaggatggttcttagactccccagacaggccct | Reference |
| | ggaacccccccaccttctccccagccctgctcgtggtgaccgaaggggacaacgccaccttcacctgcagcttctc | Sequence: |
| | caacacatcggagagcttcgtgctaaactggtaccgcatgagccccagcaaccagacggacaagctggccgcctt | NM_005018.3) |
| | ccccgaggaccgcagccagcccggccaggactgccgcttccgtgtcacacaactgcccaacgggcgtgacttcc | |
| | acatgagcgtggtcagggcccggcgcaatgacagcggcacctacctctgtggggccatctccctggcccccaag | |
| | gcgcagatcaaagagagcctgcgggcagagctcagggtgacagagagaagggcagaagtgcccacagcccac | |
| | cccagcccctcacccaggccagccggccagttccaaaccctggtggttggtgtcgtgggggcctgctgggcagc | |
| | ctggtgctgctagtctgggtcctggccgtcatctgctcccgggccgcacgagggacaataggagccaggcgcacc | |
| | ggccagcccctgaaggaggacccctcagccgtgcctgtgttctctgtggactatggggagctggatttccagtggc | |
| | gagagaagaccccggagccccccgtgccctgtgtccctgagcagacggagtatgccaccattgtctttcctagcgg | |
| | aatgggcacctcatcccccgcccgcaggggctcagctgacggccctcggagtgcccagccactgaggcctgagg | |
| | atggacactgctcttggcccctctgaccggcttccttggccaccagtgttctgcagaccctccaccatgagcccgggt | |
| | cagcgcatttcctcaggagaagcaggcagggtgcaggccattgcaggccgtccaggggctgagctgcctggggg | |
| | cgaccggggctccagcctgcacctgcaccaggcacagccccaccacaggactcatgtctcaatgcccacagtga | |
| | gcccaggcagcaggtgtcaccgtcccctacagggagggccagatgcagtcactgcttcaggtcctgccagcacag | |
| | agctgcctgcgtccagctccctgaatctctgctgctgctgctgctgctgctgctgctgcctgcggcccggggctgaa | |
| | ggcgccgtggccctgcctgacgccccggagcctcctgcctgaacttgggggctggttggagatggccttggagca | |
| | gccaaggtgcccctggcagtggcatcccgaaacgccctggacgcagggcccaagactgggcacaggagtggga | |
| | ggtacatggggctggggactccccaggagttatctgctccctgcaggcctagagaagtttcagggaaggtcagaag | |
| | agctcctggctgtggtgggcagggcaggaaacccctccacctttacacatgcccaggcagcacctcaggccctttg | |
| | tggggcagggaagctgaggcagtaagcgggcaggcagagctggaggcctttcaggcccagccagcactctggc | |
| | ctcctgccgccgcattccaccccagcccctcacaccactcgggagagggacatcctacggtcccaaggtcaggag | |
| | ggcagggctggggttgactcaggcccctcccagctgtggccacctgggtgttgggagggcagaagtgcaggcac | |
| | ctagggccccccatgtgcccaccctgggagctctccttggaacccattcctgaaattatttaaaggggttggccggg | |
| | ctcccaccagggcctggggggaaggtacaggcgttcccccggggcctagtacccccgccgtggcctatccactc | |
| | ctcacatccacacactgcacccccactcctggggcagggccaccagcatccaggcggccagcaggcacctgagt | |
| | ggctgggacaagggatcccccttccctgtggttctattatattataattataattaaatatgagagcatgctaa | |
| 81 | MSSENCFVAENSSLHPESGQENDATSPHFSTRHEGSFQVPVLCAVMNVV | CD69 (Uniprot |
| | FITILIIALIALSVGQYNCPGQYTFSMPSDSHVSSCSEDWVGYQRKCYFIST | Q07108-1) |
| | VKRSWTSAQNACSEHGATLAVIDSEKDMNFLKRYAGREEHWVGLKKEP | |
| | GHPWKWSNGKEFNNWFNVTGSDKCVFLKNTEVSSMECEKNLYWICNK | |
| | PYK | |
| 82 | agactcaacaagagctccagcaaagactttcactgtagcttgacttgacctgagattaactagggaatcttgagaata | CD69 (NCBI |
| | aagatgagctctgaaaattgtttcgtagcagagaacagctctttgcatccggagagtggacaagaaaatgatgccac | Reference |
| | cagtccccatttctcaacacgtcatgaagggtccttccaagttcctgtcctgtgtgctgtaatgaatgtggtcttcatcac | Sequence: |
| | cattttaatcatagctctcattgccttatcagtgggccaatacaattgtccaggccaatacacattctcaatgccatcaga | NP_001772.1) |
| | cagccatgtttcttcatgctctgaggactgggttggctaccagaggaaatgctactttatttctactgtgaagaggagct | |
| | ggacttcagcccaaaatgcttgttctgaacatggtgctactcttgctgtcattgattctgaaaaggacatgaactttctaa | |
| | aacgatacgcaggtagagaggaacactgggttggactgaaaaaggaacctggtcacccatggaagtggtcaaatg | |
| | gcaaagaatttaacaactggttcaacgttacagggtctgacaagtgtgtttttctgaaaaacacagaggtcagcagcat | |
| | ggaatgtgagaagaatttatactggatatgtaacaaaccttacaaataataaggaaacatgttcacttattgactattata | |
| | gaatggaactcaaggaaatctgtgtcagtggatgctgctctgtggtccgaagtcttccatagagactttgtgaaaaaaa | |
| | attttatagtgtcttgggaattttcttccaaacagaactatggaaaaaaaggaagaaattccaggaaaatctgcactgtg | |
| | ggcttttattgccatgagctagaagcatcacaggttgaccaataaccatgcccaagaatgagaagaatgactatgcaa | |
| | cctttggatgcactttatattattttgaatccagaaataatgaaataactaggcgtggacttactatttattgctgaatgact | |
| | accaacagtgagagcccttcatgcatttgcactattggaaggagttagatgttggtactagatactgaatgtaaacaaa | |
| | ggaattatggctggtaacataggtttttagtctaattgaatcccttaaactcagggagcatttataaatggacaaatgctt | |
| | atgaaactaagatttgtaatatttctctctttttagagaaatttgccaatttactttgttatttttccccaaaaagaatgggatg | |
| | atcatgtatttatttttttacttcctcagctgtagacaggtccttttcgatggtacatatttctttgcctttataatcttttatacag | |
| | tgtcttacagagaaaagacataagcaaagactatgaggaatatttgcaagacatagaatagtgttggaaaatgtgcaa | |
| | tatgtgatgtggcaaatctctattaggaaatattctgtaatcttcagacctagaataatactagtcttataataggtttgtga | |
| | ctttcctaaatcaattctattacgtgcaatacttcaatacttcatttaaaatatttttatgtgcaataaaatgtatttgtttgtattt | |
| | tgtgttcagtacaattataagctgtttttatatatgtgaaataaaagtagaataaacacaa | |
| 83 | MPCIQAQYGTPAPSPGPRDHLASDPLTPEFIKPTMDLASPEAAPAAPTALP | NR4A1 (Nur77) |
| | SFSTFMDGYTGEFDTFLYQLPGTVQPCSSASSSASSTSSSSATSPASASFKF | (Uniprot P22736-1) |
| | EDFQVYGCYPGPLSGPVDEALSSSGSDYYGSPCSAPSPSTPSFQPPQLSPW | |
| | DGSFGHFSPSQTYEGLRAWTEQLPKASGPPQPPAFFSFSPPTGPSPSLAQSP | |
| | LKLFPSQATHQLGEGESYSMPTAFPGLAPTSPHLEGSGILDTPVTSTKARS | |
| | GAPGGSEGRCAVCGDNASCQHYGVRTCEGCKGFFKRTVQKNAKYICLA | |
| | NKDCPVDKRRRNRCQFCRFQKCLAVGMVKEVVRTDSLKGRRGRLPSKP | |
| | KOPPDASPANLLTSLVRAHLDSGPSTAKLDYSKFQELVLPHFGKEDAGD | |
| | VQQFYDLLSGSLEVIRKWAEKIPGFAELSPADQDLLLESAFLELFILRLAY | |
| | RSKPGEGKLIFCSGLVLHRLQCARGFGDWIDSILAFSRSLHSLLVDVPAFA | |
| | CLSALVLITDRHGLQEPRRVEELQNRIASCLKEHVAAVAGEPQPASCLSR | |
| | LLGKLPELRTLCTQGLORIFYLKLEDLVPPPPIIDKIFMDTLPF | |
| 84 | gtcacggagcgcttaagaggagggtcgggctcggccggggagtcccagtggcggaggctacgaaacttggggg | NR4A1 |
| | agtgcacagaagaacttcgggagcgcacgcgggaccagggaccaggctgagactcggggcgccagtccgggc | (Nur77)(NCBI |
| | aggggcagcgggagccggccgggtagggtgcagcctgaggcttgttcagcagaacaggtgcaagccacattgtt | Reference |
| | gccaagacctgcctgaagccggattctccccactgcctccttcaaccccgcctcttcctcctcctgtgggactgctcc | Sequence: |
| | cccctcctgtgaggctagatagatgccctgtatccaagcccaatatgggacaccagcaccgagtccgggaccccgt | NP_002126.2) |
| | gaccacctggcaagcgaccccctgacccctgagttcatcaagcccaccatggacctggccagccccgaggcagc | |
| | ccccgctgcccccactgccctgcccagcttcagcaccttcatggacggctacacaggagagtttgacaccttcctct | |
| | accagctgccaggaacagtccagccatgctcctcagcctcctcctcggcctcctccacatcctcgtcctcagccacc | |
| | tcccctgcctctgcctccttcaagttcgaggacttccaggtgtacggctgctaccccggccccctgagcggcccagt | |
| | ggatgaggccctgtcctccagtggctctgactactatggcagcccctgctcggccccgtcgccctccacgcccagc | |
| | ttccagccgccccagctctctccctgggatggctccttcggccacttctcgcccagccagacttacgaaggcctgcg | |
| | ggcatggacagagcagctgcccaaagcctctgggcccccacagcctccagccttcttttccttcagtcctcccaccg | |
| | gccccagccccagcctggcccagagccccctgaagttgttcccctcacaggccacccaccagctgggggaggga | |
| | gagagctattccatgcctacggccttcccaggtttggcacccacttctccacaccttgagggctcggggatactggat | |
| | acacccgtgacctcaaccaaggcccggagcggggccccaggtggaagtgaaggccgctgtgctgtgtgtgggg | |
| | acaacgcttcatgccagcattatggtgtccgcacatgtgagggctgcaagggcttcttcaagcgcacagtgcagaaa | |
| | aacgccaagtacatctgcctggctaacaaggactgccctgtggacaagaggcggcgaaaccgctgccagttctgc | |
| | cgcttccagaagtgcctggcggtgggcatggtgaaggaagttgtccgaacagacagcctgaaggggcggcggg | |
| | gccggctaccttcaaaacccaagcagcccccagatgcctcccctgccaatctcctcacttccctggtccgtgcacac | |
| | ctggactcagggcccagcactgccaaactggactactccaagttccaggagctggtgctgccccactttgggaagg | |
| | aagatgctggggatgtacagcagttctacgacctgctctccggttctctggaggtcatccgcaagtgggcggagaa | |
| | gatccctggctttgctgagctgtcaccggctgaccaggacctgttgctggagtcggccttcctggagctcttcatcctc | |
| | cgcctggcgtacaggtctaagccaggcgagggcaagctcatcttctgctcaggcctggtgctacaccggctgcagt | |
| | gtgcccgtggcttcggggactggattgacagtatcctggccttctcaaggtccctgcacagcttgcttgtcgatgtccc | |
| | tgccttcgcctgcctctctgcccttgtcctcatcaccgaccggcatgggctgcaggagccgcggcgggtggaggag | |
| | ctgcagaaccgcatcgccagctgcctgaaggagcacgtggcagctgtggcgggcgagccccagccagccagct | |
| | gcctgtcacgtctgttgggcaaactgcccgagctgcggaccctgtgcacccagggcctgcagcgcatcttctacctc | |
| | aagctggaggacttggtgccccctccacccatcattgacaagatcttcatggacacgctgcccttctgacccctgcct | |
| | gggaacacgtgtgcacatgcgcactctcatatgccaccccatgtgcctttagtccacggacccccagagcaccccc | |
| | aagcctgggcttgagctgcagaatgactccaccttctcacctgctccaggaggtttgcagggagctcaagcccttgg | |
| | ggagggggatgccttcatgggggtgaccccacgatttgtcttatcccccccagcctggccccggcctttatgttttttg | |
| | taagataaaccgtttttaacacatagcgccgtgctgtaaataagcccagtgctgctgtaaatacaggaagaaagagct | |
| | tgaggtgggagcggggctgggaggaagggatgggccccgccttcctgggcagcctttccagcctcctgctggctc | |
| | tctcttcctaccctccttccacatgtacataaactgtcactctaggaagaagacaaatgacagattctgacatttatatttg | |
| | tgtattttcctggatttatagtatgtgacttttctgattaatatatttaatatattgaataaaaaatagacatgtagttggaact | |
| | gaaaaaaaaaaaaaa | |
| 85 | MPNPRPGKPSAPSLALGPSPGASPSWRAAPKASDLLGARGPGGTFQGRD | FoxP3 (Uniprot |
| | LRGGAHASSSSLNPMPPSQLQLPTLPLVMVAPSGARLGPLPHLQALLQDR | Q9BZS1-1) |
| | PHFMHQLSTVDAHARTPVLQVHPLESPAMISLTPPTTATGVFSLKARPGL | |
| | PPGINVASLEWVSREPALLCTFPNPSAPRKDSTLSAVPQSSYPLLANGVCK | |
| | WPGCEKVFEEPEDFLKHCQADHLLDEKGRAQCLLQREMVQSLEQQLVL | |
| | EKEKLSAMQAHLAGKMALTKASSVASSDKGSCCIVAAGSQGPVVPAWS | |
| | GPREAPDSLFAVRRHLWGSHGNSTFPEFLHNMDYFKFHNMRPPFTYATLI | |
| | RWAILEAPEKQRTLNEIYHWFTRMFAFFRNHPATWKNAIRHNLSLHKCF | |
| | VRVESEKGAVWTVDELEFRKKRSQRPSRCSNPTPGP | |
| 86 | gcacacactcatcgaaaaaaatttggattattagaagagagaggtctgcggcttccacaccgtacagcgtggtttttct | FoxP3 (NCBI |
| | tctcggtataaaagcaaagttgtttttgatacgtgacagtttcccacaagccaggctgatccttttctgtcagtccacttca | Reference |
| | ccaagcctgcccttggacaaggacccgatgcccaaccccaggcctggcaagccctcggccccttccttggcccttg | Sequence: |
| | gcccatccccaggagcctcgcccagctggagggctgcacccaaagcctcagacctgctgggggcccggggccc | NM_014009.3) |
| | agggggaaccttccagggccgagatcttcgaggcggggcccatgcctcctcttcttccttgaaccccatgccaccat | |
| | cgcagctgcagctgcccacactgcccctagtcatggtggcaccctccggggcacggctgggccccttgccccactt | |
| | acaggcactcctccaggacaggccacatttcatgcaccagctctcaacggtggatgcccacgcccggacccctgtg | |
| | ctgcaggtgcaccccctggagagcccagccatgatcagcctcacaccacccaccaccgccactggggtcttctcc | |
| | ctcaaggcccggcctggcctcccacctgggatcaacgtggccagcctggaatgggtgtccagggagccggcact | |
| | gctctgcaccttcccaaatcccagtgcacccaggaaggacagcaccctttcggctgtgccccagagctcctaccca | |
| | ctgctggcaaatggtgtctgcaagtggcccggatgtgagaaggtcttcgaagagccagaggacttcctcaagcact | |
| | gccaggcggaccatcttctggatgagaagggcagggcacaatgtctcctccagagagagatggtacagtctctgg | |
| | agcagcagctggtgctggagaaggagaagctgagtgccatgcaggcccacctggctgggaaaatggcactgacc | |
| | aaggcttcatctgtggcatcatccgacaagggctcctgctgcatcgtagctgctggcagccaaggccctgtcgtccc | |
| | agcctggtctggcccccgggaggcccctgacagcctgtttgctgtccggaggcacctgtggggtagccatggaaa | |
| | cagcacattcccagagttcctccacaacatggactacttcaagttccacaacatgcgaccccctttcacctacgccac | |
| | gctcatccgctgggccatcctggaggctccagagaagcagcggacactcaatgagatctaccactggttcacacgc | |
| | atgtttgccttcttcagaaaccatcctgccacctggaagaacgccatccgccacaacctgagtctgcacaagtgctttg | |
| | tgcgggtggagagcgagaagggggctgtgtggaccgtggatgagctggagttccgcaagaaacggagccagag | |
| | gcccagcaggtgttccaaccctacacctggcccctgacctcaagatcaaggaaaggaggatggacgaacagggg | |
| | ccaaactggtgggaggcagaggtggtgggggcagggatgataggccctggatgtgcccacagggaccaagaag | |
| | tgaggtttccactgtcttgcctgccagggcccctgttcccccgctggcagccaccccctcccccatcatatcctttgcc | |
| | ccaaggctgctcagaggggccccggtcctggccccagcccccacctccgccccagacacaccccccagtcgag | |
| | ccctgcagccaaacagagccttcacaaccagccacacagagcctgcctcagctgctcgcacagattacttcagggc | |
| | tggaaaagtcacacagacacacaaaatgtcacaatcctgtccctcactcaacacaaaccccaaaacacagagagcc | |
| | tgcctcagtacactcaaacaacctcaaagctgcatcatcacacaatcacacacaagcacagccctgacaacccaca | |
| | caccccaaggcacgcacccacagccagcctcagggcccacaggggcactgtcaacacaggggtgtgcccagag | |
| | gcctacacagaagcagcgtcagtaccctcaggatctgaggtcccaacacgtgctcgctcacacacacggcctgtta | |
| | gaattcacctgtgtatctcacgcatatgcacacgcacagccccccagtgggtctcttgagtcccgtgcagacacaca | |
| | cagccacacacactgccttgccaaaaataccccgtgtctcccctgccactcacctcactcccattccctgagccctga | |
| | tccatgcctcagcttagactgcagaggaactactcatttatttgggatccaaggcccccaacccacagtaccgtcccc | |
| | aataaactgcagccgagctccccacaaaaaaaaaaaaaaaa | |
| 87 | MAISGVPVLGFFIIAVLMSAQESWAIKEEHVIIQAEFYLNPDQSGEFMFDF | HLA-DRA |
| | DGDEIFHVDMAKKETVWRLEEFGRFASFEAQGALANIAVDKANLEIMTK | (Uniprot P01903-1) |
| | RSNYTPITNVPPEVTVLTNSPVELREPNVLICFIDKFTPPVVNVTWLRNGK | |
| | PVTTGVSETVFLPREDHLFRKFHYLPFLPSTEDVYDCRVEHWGLDEPLLK | |
| | HWEFDAPSPLPETTENVVCALGLTVGLVGIIIGTIFIIKGVRKSNAAERRGP | |
| | L | |
| 88 | ttttaatggtcagactctattacaccccacattctcttttttttattcttgtctgttctgcctcactcccgagctctactgactc | HLA-DRA (NCBI |
| | ccaacagagcgcccaagaagaaaatggccataagtggagtccctgtgctaggatttttcatcatagctgtgctgatga | Reference |
| | gcgctcaggaatcatgggctatcaaagaagaacatgtgatcatccaggccgagttctatctgaatcctgaccaatca | Sequence: |
| | ggcgagtttatgtttgactttgatggtgatgagattttccatgtggatatggcaaagaaggagacggtctggcggcttg | NM_019111.4) |
| | aagaatttggacgatttgccagctttgaggctcaaggtgcattggccaacatagctgtggacaaagccaacctggaa | |
| | atcatgacaaagcgctccaactatactccgatcaccaatgtacctccagaggtaactgtgctcacaaacagccctgtg | |
| | gaactgagagagcccaacgtcctcatctgtttcatagacaagttcaccccaccagtggtcaatgtcacgtggcttcga | |
| | aatggaaaacctgtcaccacaggagtgtcagagacagtcttcctgcccagggaagaccaccttttccgcaagttcca | |
| | ctatctccccttcctgccctcaactgaggacgtttacgactgcagggtggagcactggggcttggatgagcctcttctc | |
| | aagcactgggagtttgatgctccaagccctctcccagagactacagagaacgtggtgtgtgccctgggcctgactgt | |
| | gggtctggtgggcatcattattgggaccatcttcatcatcaagggattgcgcaaaagcaatgcagcagaacgcagg | |
| | gggcctctgtaaggcacatggaggtgatggtgtttcttagagagaagatcactgaagaaacttctgctttaatggcttt | |
| | acaaagctggcaatattacaatccttgacctcagtgaaagcagtcatcttcagcattttccagccctatagccacccca | |
| | agtgtggatatgcctcttcgattgctccgtactctaacatctagctggcttccctgtctattgccttttcctgtatctattttcc | |
| | tctatttcctatcattttattatcaccatgcaatgcctctggaataaaacatacaggagtctgtctctgctatggaatgccc | |
| | catggggcatctcttgtgtacttattgtttaaggtttcctcaaactgtgatttttctgaacacaataaactattttgatgatctt | |
| | gggtggaaaaaaaaaaaaaaaaaaaaaaaaaaaaaaaaaa | |
| 89 | MVCLKLPGGSCMTALTVTLMVLSSPLALAGDTRPRFLWQLKFECHFFNG | HLA-DRB1 |
| | TERVRLLERCIYNQEESVRFDSDVGEYRAVTELGRPDAEYWNSQKDLLE | (Uniprot P04229-1) |
| | QRRAAVDTYCRHNYGVGESFTVQRRVEPKVTVYPSKTQPLQHHNLLVC | |
| | SVSGFYPGSIEVRWFRNGQEEKAGVVSTGLIQNGDWTFQTLVMLETVPR | |
| | SGEVYTCQVEHPSVTSPLTVEWRARSESAQSKMLSGVGGFVLGLLFLGA | |
| | GLFIYFRNQKGHSGLQPTGFLS | |
| 90 | tagttctccctgagtgagacttgcctgcttctctggcccctggtcctgtcctgttctccagcatggtgtgtctgaagctcc | HLA-DRB1 |
| | ctggaggctcctgcatgacagcgctgacagtgacactgatggtgctgagctccccactggctttggctggggacac | (Genbank: |
| | ccgaccacgtttcttgtggcagcttaagtttgaatgtcatttcttcaatgggacggagcgggtgcggttgctggaaaga | X03069.1) |
| | tgcatctataaccaagaggagtccgtgcgcttcgacagcgacgtgggggagtaccgggcggtgacggagctggg | |
| | gcggcctgatgccgagtactggaacagccagaaggacctcctggagcagaggcgggccgcggtggacacctac | |
| | tgcagacacaactacggggttggtgagagcttcacagtgcagcggcgagttgagcctaaggtgactgtgtatccttc | |
| | aaagacccagcccctgcagcaccacaacctcctggtctgctctgtgagtggtttctatccaggcagcattgaagtca | |
| | ggtggttccggaacggccaggaagagaaggctggggtggtgtccacaggcctgatccagaatggagattggacc | |
| | ttccagaccctggtgatgctggaaacagttcctcggagtggagaggtttacacctgccaagtggagcacccaagtgt | |
| | gacgagccctctcacagtggaatggagagcacggtctgaatctgcacagagcaagatgctgagtggagtcgggg | |
| | gcttcgtgctgggcctgctcttccttggggccgggctgttcatctacttcaggaatcagaaaggacactctggacttca | |
| | gccaacaggattcctgagctgaaatgcagatgaccacattcaaggaagaaccttctgtcccagctttgcagaatgaa | |
| | aagctttcctgcttggcagttattcttccacaagagagggctttctcaggacctggttgctactggttcggcaactgcag | |
| | aaaatgtcctcccttgtggcttcctcagctcctgcccttggcctgaagtcccagcattgatgacagcgcctcatcttcaa | |
| | cttttgtgctcccctttgcctaaaccgtatggcctcccgtgcatctgtactcaccctgtacgacaaacacattacattatta | |
| | aatgtttctcaaagatggagtt | |
| 91 | PNIQNPDPAVYQLRDSKSSDKSVCLFTDFDSQTNVSQSKDSDVYITDKTV | Human TCR alpha |
| | LDMRSMDFKSNSAVAWSNKSDFACANAFNNSIIPEDTFFPSPESSCDVKL | constant (Uniprot |
| | VEKSFETDTNLNFQNLSVIGFRILLLKVAGFNLLMTLRLWSS | P01848) |
| 92 | NIQNPDPAVYQLRDSKSSDKSVCLFTDFDSQTNVSQSKDSDVYITDKTVL | human TCR alpha |
| | DMRSMDFKSNSAVAWSNKSDFACANAFNNSIIPEDTFFPSPESSCDVKLV | constant (Genbank |
| | EKSFETDTNLNFQNLSVIGFRILLLKVAGFNLLMTLRLWSS | Accession No. |
| | | CAA26636.1) |
| 93 | ttttgaaacccttcaaaggcagagacttgtccagcctaacctgcctgctgctcctagctcctgaggctcagggcccttg | Human TCR alpha |
| | gcttctgtccgctctgctcagggccctccagcgtggccactgctcagccatgctcctgctgctcgtcccagtgctcga | constant |
| | ggtgatttttaccctgggaggaaccagagcccagtcggtgacccagcttggcagccacgtctctgtctctgaaggag | (GenBank: X02592.1) |
| | ccctggttctgctgaggtgcaactactcatcgtctgttccaccatatctcttctggtatgtgcaataccccaaccaagga | |
| | ctccagcttctcctgaagtacacatcagcggccaccctggttaaaggcatcaacggttttgaggctgaatttaagaag | |
| | agtgaaacctccttccacctgacgaaaccctcagcccatatgagcgacgcggctgagtacttctgtgctgtgagtgat | |
| | ctcgaaccgaacagcagtgcttccaagataatctttggatcagggaccagactcagcatccggccaaatatccagaa | |
| | ccctgaccctgccgtgtaccagctgagagactctaaatccagtgacaagtctgtctgcctattcaccgattttgattctc | |
| | aaacaaatgtgtcacaaagtaaggattctgatgtgtatatcacagacaaaactgtgctagacatgaggtctatggactt | |
| | caagagcaacagtgctgtggcctggagcaacaaatctgactttgcatgtgcaaacgccttcaacaacagcattattcc | |
| | agaagacaccttcttccccagcccagaaagttcctgtgatgtcaagctggtcgagaaaagctttgaaacagatacga | |
| | acctaaactttcaaaacctgtcagtgattgggttccgaatcctcctcctgaaagtggccgggtttaatctgctcatgacg | |
| | ctgcggctgtggtccagctgagatctgcaagattgtaagacagcctgtgctccctcgctccttcctctgcattgcccct | |
| | cttctccctctccaaacagagggaactctcctacccccaaggaggtgaaagctgctaccacctctgtgcccccccgg | |
| | taatgccaccaactggatcctacccgaatttatgattaagattgctgaagagctgccaaacactgctgccaccccctct | |
| | gttcccttattgctgcttgtcactgcctgacattcacggcagaggcaaggctgctgcagcctcccctggctgtgcacat | |
| | tccctcctgctccccagagactgcctccgccatcccacagatgatggatcttcagtgggttctcttgggctctaggtcc | |
| | tggagaatgttgtgaggggtttatttttttttaatagtgttcataaagaaatacatagtattcttcttctcaagacgtggggg | |
| | gaaattatctcattatcgaggccctgctatgctgtgtgtctgggcgtgttgtatgtcctgctgccgatgccttcattaaaat | |
| | gatttggaa | |
| 94 | EDLNKVFPPEVAVFEPSEAEISHTQKATLVCLATGFFPDHVELSWWVNG | Human TCR beta |
| | KEVHSGVSTDPQPLKEQPALNDSRYCLSSRLRVSATFWQNPRNHFRCQV | constant 1 |
| | QFYGLSENDEWTQDRAKPVTQIVSAEAWGRADCGFTSVSYQQGVLSATI | (Uniprot P01850) |
| | LYEILLGKATLYAVLVSALVLMAMVKRKDF | |
| 95 | DLKNVFPPEVAVFEPSEAEISHTQKATLVCLATGFYPDHVELSWWVNGK | Human TCR beta |
| | EVHSGVSTDPQPLKEQPALNDSRYCLSSRLRVSATFWQNPRNHFRCQVQ | constant 2 |
| | FYGLSENDEWTQDRAKPVTQIVSAEAWGRADCGFTSESYQQGVLSATIL | (Uniprot A0A5B9) |
| | YEILLGKATLYAVLVSALVLMAMVKRKDSRG | |
| 96 | EDLKNVFPPEVAVFEPSEAEISHTQKATLVCLATGFYPDHVELSWWVNG | human TCR beta |
| | KEVHSGVSTDPQPLKEQPALNDSRYCLSSRLRVSATFWQNPRNHFRCQV | constant (Uniprot |
| | QFYGLSENDEWTQDRAKPVTQIVSAEAWGRADCGFTSESYQQGVLSATI | Accession No. |
| | LYEILLGKATLYAVLVSALVLMAMVKRKDSRG | A0A0G2JNG9) |
| 97 | ctggtctagaatattccacatctgctctcactctgccatggactcctggaccttctgctgtgtgtccctttgcatcctggta | human TCR beta |
| | gcgaagcatacagatgctggagttatccagtcaccccgccatgaggtgacagagatgggacaagaagtgactctg | constant |
| | agatgtaaaccaatttcaggccacaactcccttttctggtacagacagaccatgatgcggggactggagttgctcattt | (GenBank: |
| | actttaacaacaacgttccgatagatgattcagggatgcccgaggatcgattctcagctaagatgcctaatgcatcatt | X00437.1) |
| | ctccactctgaagatccagccctcagaacccagggactcagctgtgtacttctgtgccagcagtttctcgacctgttcg | |
| | gctaactatggctacaccttcggttcggggaccaggttaaccgttgtagaggacctgaacaaggtgttcccacccga | |
| | ggtcgctgtgtttgagccatcagaagcagagatctcccacacccaaaaggccacactggtgtgcctggccacaggc | |
| | ttcttccccgaccacgtggagctgagctggtgggtgaatgggaaggaggtgcacagtggggtcagcacagacccg | |
| | cagcccctcaaggagcagcccgccctcaatgactccagatactgcctgagcagccgcctgagggtctcggccacc | |
| | ttctggcagaacccccgcaaccacttccgctgtcaagtccagttctacgggctctcggagaatgacgagtggaccca | |
| | ggatagggccaaacccgtcacccagatcgtcagcgccgaggcctggggtagagcagactgtggctttacctcggt | |
| | gtcctaccagcaaggggtcctgtctgccaccatcctctatgagatcctgctagggaaggccaccctgtatgctgtgct | |
| | ggtcagcgcccttgtgttgatggccatggtcaagagaaaggatttctgaaggcagccctggaagtggagttaggag | |
| | cttctaacccgtcatggttcaatacacattcttcttttgccagcgcttctgaagagctgctctcacctctctgcatcccaat | |
| | agatatccccctatgtgcatgcacacctgcacactcacggctgaaatctccctaacccagggggac | |
| 98 | TGACGTTACCTCGTGCGGCC | PDCD1 gRNA |
| | | target sequence 1 |
| 99 | CACGAAGCTCTCCGATGTGT | PDCD1 gRNA |
| | | target sequence 2 |
| 100 | GCGTGACTTCCACATGAGCG | PDCD1 gRNA |
| | | target sequence 3 |
| 101 | TTGGAACTGGCCGGCTGGCC | PDCD1 gRNA |
| | | target sequence 4 |
| 102 | GTGGCATACTCCGTCTGCTC | PDCD1 gRNA |
| | | target sequence 5 |
| 103 | GATGAGGTGCCCATTCCGCT | PDCD1 gRNA |
| | | target sequence 6 |
| 104 | UGACGUUACCUCGUGCGGCC | PDCD1 gRNA |
| | | sequence 1 |
| 105 | CACGAAGCUCUCCGAUGUGU | PDCD1 gRNA |
| | | sequence 2 |
| 106 | GCGUGACUUCCACAUGAGCG | PDCD1 gRNA |
| | | sequence 3 |
| 107 | UUGGAACUGGCCGGCUGGCC | PDCD1 gRNA |
| | | sequence 4 |
| 108 | GUGGCAUACUCCGUCUGCUC | PDCD1 gRNA |
| | | sequence 5 |
| 109 | GAUGAGGUGCCCAUUCCGCU | PDCD1 gRNA |
| | | sequence 6 |
| 110 | AACTTTCTAAAACGATACGC | CD69 gRNA |
| | | target sequence 1 |
| 111 | TTTGACAGGTTCAACGTTAC | CD69 gRNA |
| | | target sequence 2 |
| 112 | CATTTCTCAACACGTCATGA | CD69 gRNA |
| | | target sequence 3 |
| 113 | CTTCATGACGTGTTGAGAAA | CD69 gRNA |
| | | target sequence 4 |
| 114 | GTGGGCCAATACAATTGTCC | CD69 gRNA |
| | | target sequence 5 |
| 115 | ATGGCATTGAGAATGTGTAT | CD69 gRNA |
| | | target sequence 6 |
| 116 | AACUUUCUAAAACGAUACGC | CD69 gRNA |
| | | sequence 1 |
| 117 | UUUGACAGGUUCAACGUUAC | CD69 gRNA |
| | | sequence 2 |
| 118 | CAUUUCUCAACACGUCAUGA | CD69 gRNA |
| | | sequence 3 |
| 119 | CUUCAUGACGUGUUGAGAAA | CD69 gRNA |
| | | sequence 4 |
| 120 | GUGGGCCAAUACAAUUGUCC | CD69 gRNA |
| | | sequence 5 |
| 121 | AUGGCAUUGAGAAUGUGUAU | CD69 gRNA |
| | | sequence 6 |
| 122 | CGGGGTAGCAGCCGTACACC | Nur77 (NR4A1) |
| | | gRNA target |
| | | sequence 1 |
| 123 | CCCTGTATCCAAGCCCAATA | Nur77 (NR4A1) |
| | | gRNA target |
| | | sequence 2 |
| 124 | TGTCCGAACAGACAGCCTGA | Nur77 (NR4A1) |
| | | gRNA target |
| | | sequence 3 |
| 125 | AAACGCCAAGTACATCTGCC | Nur77 (NR4A1) |
| | | gRNA target |
| | | sequence 4 |
| 126 | CACCTTGGAGTAGTCCAGTT | Nur77 (NR4A1) |
| | | gRNA target |
| | | sequence 5 |
| 127 | TTTCGCCGCCTCTTGTCCAC | Nur77 (NR4A1) |
| | | gRNA target |
| | | sequence 6 |
| 128 | CGGGGUAGCAGCCGUACACC | Nur77 (NR4A1) |
| | | gRNA sequence 1 |
| 129 | CCCUGUAUCCAAGCCCAAUA | Nur77 (NR4A1) |
| | | gRNA sequence 2 |
| 130 | UGUCCGAACAGACAGCCUGA | Nur77 (NR4A1) |
| | | gRNA sequence 3 |
| 131 | AAACGCCAAGUACAUCUGCC | Nur77 (NR4A1) |
| | | gRNA sequence 4 |
| 132 | CACCUUGGAGUAGUCCAGUU | Nur77 (NR4A1) |
| | | gRNA sequence 5 |
| 133 | UUUCGCCGCCUCUUGUCCAC | Nur77 (NR4A1) |
| | | gRNA sequence 6 |
| 134 | TCATTGACAAGATCTTCATG | Nur77 (NR4A1) |
| | | gRNA target |
| | | sequence 7 |
| 135 | GCCTGGGAACACGTGTGCA | Nur77 (NR4A1) |
| | | gRNA target |
| | | sequence 8 |
| 136 | CCATATTGGGCTTGGATACA | Nur77 (NR4A1) |
| | | gRNA target |
| | | sequence 9 |
| 137 | CAUGAAGAUCUUGUCAAUGA | Nur77 (NR4A1) |
| | | gRNA sequence 7 |
| 138 | UGCACACGUGUUCCCAGGC | Nur77 (NR4A1) |
| | | gRNA sequence 8 |
| 139 | CCAUAUUGGGCUUGGAUACA | Nur77 (NR4A1) |
| | | gRNA sequence 9 |
| 140 | CCCACCCACAGGGATCAACG | FoxP3 gRNA |
| | | target sequence1 |
| 141 | TTCGAAGACCTTCTCACATC | FoxP3 gRNA |
| | | target sequence2 |
| 142 | AGCTCTGGGGCACAGCCGAA | FoxP3 gRNA |
| | | target sequence3 |
| 143 | CCACTTACAGGCACTCCTCC | FoxP3 gRNA |
| | | target sequence4 |
| 144 | CCTGGACACCCATTCCAGGC | FoxP3 gRNA |
| | | target sequence5 |
| 145 | TGCCCCCCAGCTCTCAACGG | FoxP3 gRNA |
| | | target sequence6 |
| 146 | TCCAGCTGGGCGAGGCTCCT | FoxP3 gRNA |
| | | target sequence7 |
| 147 | TTTGGGTGCAGCCCTCCAGC | FoxP3 gRNA |
| | | target sequence8 |
| 148 | CCCACCCACAGGGAUCAACG | FoxP3 gRNA |
| | | sequence1 |
| 149 | UUCGAAGACCUUCUCACAUC | FoxP3 gRNA |
| | | sequence2 |
| 150 | AGCUCUGGGGCACAGCCGAA | FoxP3 gRNA |
| | | sequence3 |
| 151 | CCACUUACAGGCACUCCUCC | FoxP3 gRNA |
| | | sequence4 |
| 152 | CCUGGACACCCAUUCCAGGC | FoxP3 gRNA |
| | | sequence5 |
| 153 | UGCCCCCCAGCUCUCAACGG | FoxP3 gRNA |
| | | sequence6 |
| 154 | UCCAGCUGGGCGAGGCUCCU | FoxP3 gRNA |
| | | sequence7 |
| 155 | UUUGGGUGCAGCCCUCCAGC | FoxP3 gRNA |
| | | sequence8 |
| 156 | GTCGTAAACGTCCTCAGTTG | HLA-DRA gRNA |
| | | target sequence1 |
| 157 | CAAACATAAACTCGCCTGAT | HLA-DRA gRNA |
| | | target sequence2 |
| 158 | GATTCAGATAGAACTCGGCC | HLA-DRA gRNA |
| | | target sequence3 |
| 159 | AGACAAGTTCACCCCACCAG | HLA-DRA gRNA |
| | | target sequence4 |
| 160 | AGCTGTGCTGATGAGCGCTC | HLA-DRA gRNA |
| | | target sequence5 |
| 161 | AAAGCAATGCAGCAGAACGC | HLA-DRA gRNA |
| | | target sequence6 |
| 162 | GUCGUAAACGUCCUCAGUUG | HLA-DRA gRNA |
| | | sequence1 |
| 163 | CAAACAUAAACUCGCCUGAU | HLA-DRA gRNA |
| | | sequence2 |
| 164 | GAUUCAGAUAGAACUCGGCC | HLA-DRA gRNA |
| | | sequence3 |
| 165 | AGACAAGUUCACCCCACCAG | HLA-DRA gRNA |
| | | sequence4 |
| 166 | AGCUGUGCUGAUGAGCGCUC | HLA-DRA gRNA |
| | | sequence5 |
| 167 | AAAGCAAUGCAGCAGAACGC | HLA-DRA gRNA |
| | | sequence6 |
| 168 | GACGGAGCGGGTGCGGTTCC | HLA-DRB1 |
| | | gRNA target |
| | | sequence 1 |
| 169 | CACGTCGCTGTCGAAGCGCA | HLA-DRB1 |
| | | gRNA target |
| | | sequence2 |
| 170 | CACTGTCAGCGCTGTCATGC | HLA-DRB1 |
| | | gRNA target |
| | | sequence3 |
| 171 | CCTGAAGTAGATGAACAGCC | HLA-DRB1 |
| | | gRNA target |
| | | sequence4 |
| 172 | GAAGGATATACAGTCACCTT | HLA-DRB1 |
| | | gRNA target |
| | | sequence5 |
| 173 | AGCTCCCCACTGGCTTTGTC | HLA-DRB1 |
| | | gRNA target |
| | | sequence6 |
| 174 | ATCCAGGCAGCATTGAAGTCAGG | HLA-DRB1 |
| | | gRNA target |
| | | sequence7 |
| 175 | CCAGGCAGCATTGAAGTCAGGTG | HLA-DRB1 |
| | | gRNA target |
| | | sequence8 |
| 176 | CCTTCCAGACCCTGGTGATGCTG | HLA-DRB1 |
| | | gRNA target |
| | | sequence9 |
| 177 | CCAGACCCTGGTGATGCTGGAAA | HLA-DRB1 |
| | | gRNA target |
| | | sequence 10 |
| 178 | GACGGAGCGGGUGCGGUUCC | HLA-DRB1 |
| | | gRNA sequence1 |
| 179 | CACGUCGCUGUCGAAGCGCA | HLA-DRB1 |
| | | gRNA sequence2 |
| 180 | CACUGUCAGCGCUGUCAUGC | HLA-DRB1 |
| | | gRNA sequence3 |
| 181 | CCUGAAGUAGAUGAACAGCC | HLA-DRB1 |
| | | gRNA sequence4 |
| 182 | GAAGGAUAUACAGUCACCUU | HLA-DRB1 |
| | | gRNA sequence5 |
| 183 | AGCUCCCCACUGGCUUUGUC | HLA-DRB1 |
| | | gRNA sequence6 |
| 184 | AUCCAGGCAGCAUUGAAGUCAGG | HLA-DRB1 |
| | | gRNA sequence7 |
| 185 | CCAGGCAGCAUUGAAGUCAGGUG | HLA-DRB1 |
| | | gRNA sequence8 |
| 186 | CCUUCCAGACCCUGGUGAUGCUG | HLA-DRB1 |
| | | gRNA sequence9 |
| 187 | CCAGACCCUGGUGAUGCUGGAAA | HLA-DRB1 |
| | | gRNA sequence10 |
| 188 | UCUCUCAGCUGGUACACGGC | TRAC-10 |
| 189 | UGGAUUUAGAGUCUCUCAGC | TRAC-110 |
| 190 | ACACGGCAGGGUCAGGGUUC | TRAC-116 |
| 191 | GAGAAUCAAAAUCGGUGAAU | TRAC-16 |
| 192 | GCUGGUACACGGCAGGGUCA | TRAC-4 |
| 193 | CUCAGCUGGUACACGGC | TRAC-49 |
| 194 | UGGUACACGGCAGGGUC | TRAC-2 |
| 195 | GCUAGACAUGAGGUCUA | TRAC-30 |
| 196 | GUCAGAUUUGUUGCUCC | TRAC-43 |
| 197 | UCAGCUGGUACACGGCA | TRAC-23 |
| 198 | GCAGACAGACUUGUCAC | TRAC-34 |
| 199 | GGUACACGGCAGGGUCA | TRAC-25 |
| 200 | CUUCAAGAGCAACAGUGCUG | TRAC-128 |
| 201 | AGAGCAACAGUGCUGUGGCC | TRAC-105 |
| 202 | AAAGUCAGAUUUGUUGCUCC | TRAC-106 |
| 203 | ACAAAACUGUGCUAGACAUG | TRAC-123 |
| 204 | AAACUGUGCUAGACAUG | TRAC-64 |
| 205 | UGUGCUAGACAUGAGGUCUA | TRAC-97 |
| 206 | GGCUGGGGAAGAAGGUGUCUUC | TRAC-148 |
| 207 | GCUGGGGAAGAAGGUGUCUUC | TRAC-147 |
| 208 | GGGGAAGAAGGUGUCUUC | TRAC-234 |
| 209 | GUUUUGUCUGUGAUAUACACAU | TRAC-167 |
| 210 | GGCAGACAGACUUGUCACUGGAUU | TRAC-177 |
| 211 | GCAGACAGACUUGUCACUGGAUU | TRAC-176 |
| 212 | GACAGACUUGUCACUGGAUU | TRAC-257 |
| 213 | GUGAAUAGGCAGACAGACUUGUCA | TRAC-233 |
| 214 | GAAUAGGCAGACAGACUUGUCA | TRAC-231 |
| 215 | GAGUCUCUCAGCUGGUACACGG | TRAC-163 |
| 216 | GUCUCUCAGCUGGUACACGG | TRAC-241 |
| 217 | GGUACACGGCAGGGUCAGGGUU | TRAC-179 |
| 218 | GUACACGGCAGGGUCAGGGUU | TRAC-178 |
| 219 | CACCCAGAUCGUCAGCGCCG | TRBC-40 |
| 220 | CAAACACAGCGACCUCGGGU | TRBC-52 |
| 221 | UGACGAGUGGACCCAGGAUA | TRBC-25 |
| 222 | GGCUCUCGGAGAAUGACGAG | TRBC-35 |
| 223 | GGCCUCGGCGCUGACGAUCU | TRBC-50 |
| 224 | GAAAAACGUGUUCCCACCCG | TRBC-39 |
| 225 | AUGACGAGUGGACCCAGGAU | TRBC-49 |
| 226 | AGUCCAGUUCUACGGGCUCU | TRBC-51 |
| 227 | CGCUGUCAAGUCCAGUUCUA | TRBC-26 |
| 228 | AUCGUCAGCGCCGAGGCCUG | TRBC-47 |
| 229 | UCAAACACAGCGACCUCGGG | TRBC-45 |
| 230 | CGUAGAACUGGACUUGACAG | TRBC-34 |
| 231 | AGGCCUCGGCGCUGACGAUC | TRBC-227 |
| 232 | UGACAGCGGAAGUGGUUGCG | TRBC-41 |
| 233 | UUGACAGCGGAAGUGGUUGC | TRBC-30 |
| 234 | UCUCCGAGAGCCCGUAGAAC | TRBC-206 |
| 235 | CGGGUGGGAACACGUUUUUC | TRBC-32 |
| 236 | GACAGGUUUGGCCCUAUCCU | TRBC-276 |
| 237 | GAUCGUCAGCGCCGAGGCCU | TRBC-274 |
| 238 | GGCUCAAACACAGCGACCUC | TRBC-230 |
| 239 | UGAGGGUCUCGGCCACCUUC | TRBC-235 |
| 240 | AGGCUUCUACCCCGACCACG | TRBC-38 |
| 241 | CCGACCACGUGGAGCUGAGC | TRBC-223 |
| 242 | UGACAGGUUUGGCCCUAUCC | TRBC-221 |
| 243 | CUUGACAGCGGAAGUGGUUG | TRBC-48 |
| 244 | AGAUCGUCAGCGCCGAGGCC | TRBC-216 |
| 245 | GCGCUGACGAUCUGGGUGAC | TRBC-210 |
| 246 | UGAGGGCGGGCUGCUCCUUG | TRBC-268 |
| 247 | GUUGCGGGGGUUCUGCCAGA | TRBC-193 |
| 248 | AGCUCAGCUCCACGUGGUCG | TRBC-246 |
| 249 | GCGGCUGCUCAGGCAGUAUC | TRBC-228 |
| 250 | GCGGGGGUUCUGCCAGAAGG | TRBC-43 |
| 251 | UGGCUCAAACACAGCGACCU | TRBC-272 |
| 252 | ACUGGACUUGACAGCGGAAG | TRBC-33 |
| 253 | GACAGCGGAAGUGGUUGCGG | TRBC-44 |
| 254 | GCUGUCAAGUCCAGUUCUAC | TRBC-211 |
| 255 | GUAUCUGGAGUCAUUGAGGG | TRBC-253 |
| 256 | CUCGGCGCUGACGAUCU | TRBC-18 |
| 257 | CCUCGGCGCUGACGAUC | TRBC-6 |
| 258 | CCGAGAGCCCGUAGAAC | TRBC-85 |
| 259 | CCAGAUCGUCAGCGCCG | TRBC-129 |
| 260 | GAAUGACGAGUGGACCC | TRBC-93 |
| 261 | GGGUGACAGGUUUGGCCCUAUC | TRBC-415 |
| 262 | GGUGACAGGUUUGGCCCUAUC | TRBC-414 |
| 263 | GUGACAGGUUUGGCCCUAUC | TRBC-310 |
| 264 | GACAGGUUUGGCCCUAUC | TRBC-308 |
| 265 | GAUACUGCCUGAGCAGCCGCCU | TRBC-401 |
| 266 | GACCACGUGGAGCUGAGCUGGUGG | TRBC-468 |
| 267 | GUGGAGCUGAGCUGGUGG | TRBC-462 |
| 268 | GGGCGGGCUGCUCCUUGAGGGGCU | TRBC-424 |
| 269 | GGCGGGCUGCUCCUUGAGGGGCU | TRBC-423 |
| 270 | GCGGGCUGCUCCUUGAGGGGCU | TRBC-422 |
| 271 | GGGCUGCUCCUUGAGGGGCU | TRBC-420 |
| 272 | GGCUGCUCCUUGAGGGGCU | TRBC-419 |
| 273 | GCUGCUCCUUGAGGGGCU | TRBC-418 |
| 274 | GGUGAAUGGGAAGGAGGUGCACAG | TRBC-445 |
| 275 | GUGAAUGGGAAGGAGGUGCACAG | TRBC-444 |
| 276 | GAAUGGGAAGGAGGUGCACAG | TRBC-442 |
| 277 | AGCGCTCTCGTACAGAGTTGGCATTATAATACGACTCACTATAGGGG | TRAC gRNA |
| | AGAATCAAAATCGGTGAATGTTTTAGAGCTAGAAATAGCAAGTTAAA | |
| | ATAAGGCTAGTCCGTTATCAACTTGAAAAAGTGGCACCGAGTCGGTG | |
| | CTTTTTTT | |
| 278 | GAGAAUCAAAAUCGGUGAAUGUUUUAGAGCUAGAAAUAGCAAGUU | TRAC gRNA |
| | AAAAUAAGGCUAGUCCGUUAUCAACUUGAAAAAGUGGCACCGAGUC | |
| | GGUGCUUUU | |
| 279 | KKPYSIGLDIGTNSVGWAVVTDDYKVPAKKMKVLGNTDKSHIEKNLLG | Streptococcus |
| | ALLFDSGNTAEDRRLKRTARRRYTRRRNRILYLQEIFSEEMGKVDDSFFH | mutans Cas9 |
| | RLEDSFLVTEDKRGERHPIFGNLEEEVKYHENFPTIYHLRQYLADNPEKV | |
| | DLRLVYLALAHIIKFRGHFLIEGKFDTRNNDVQRLFQEFLAVYDNTFENS | |
| | SLQEQNVQVEEILTDKISKSAKKDRVLKLFPNEKSNGRFAEFLKLIVGNQ | |
| | ADFKKHFELEEKAPLQFSKDTYEEELEVLLAQIGDNYAELFLSAKKLYDS | |
| | ILLSGILTVTDVGTKAPLSASMIQRYNEHQMDLAQLKQFIRQKLSDKYNE | |
| | VFSDVSKDGYAGYIDGKTNQEAFYKYLKGLLNKIEGSGYFLDKIEREDFL | |
| | RKQRTFDNGSIPHQIHLQEMRAIIRRQAEFYPFLADNQDRIEKLLTFRIPYY | |
| | VGPLARGKSDFAWLSRKSADKITPWNFDEIVDKESSAEAFINRMTNYDL | |
| | YLPNQKVLPKHSLLYEKFTVYNELTKVKYKTEQGKTAFFDANMKQEIFD | |
| | GVFKVYRKVTKDKLMDFLEKEFDEFRIVDLTGLDKENKVFNASYGTYH | |
| | DLCKILDKDFLDNSKNEKILEDIVLTLTLFEDREMIRKRLENYSDLLTKEQ | |
| | VKKLERRHYTGWGRLSAELIHGIRNKESRKTILDYLIDDGNSNRNFMQLI | |
| | NDDALSFKEEIAKAQVIGETDNLNQVVSDIAGSPAIKKGILQSLKIVDELV | |
| | KIMGHQPENIVVEMARENQFTNQGRRNSQQRLKGLTDSIKEFGSQILKEH | |
| | PVENSQLQNDRLFLYYLQNGRDMYTGEELDIDYLSQYDIDHIIPQAFIKD | |
| | NSIDNRVLTSSKENRGKSDDVPSKDVVRKMKSYWSKLLSAKLITQRKFD | |
| | NLTKAERGGLTDDDKAGFIKRQLVETRQITKHVARILDERFNTETDENNK | |
| | KIRQVKIVTLKSNLVSNFRKEFELYKVREINDYHHAHDAYLNAVIGKALL | |
| | GVYPQLEPEFVYGDYPHFHGHKENKATAKKFFYSNIMNFFKKDDVRTD | |
| | KNGEIIWKKDEHISNIKKVLSYPQVNIVKKVEEQTGGFSKESILPKGNSDK | |
| | LIPRKTKKFYWDTKKYGGFDSPIVAYSILVIADIEKGKSKKLKTVKALVG | |
| | VTIMEKMTFERDPVAFLERKGYRNVQEENIIKLPKYSLFKLENGRKRLLA | |
| | SARELQKGNEIVLPNHLGTLLYHAKNIHKVDEPKHLDYVDKHKDEFKEL | |
| | LDVVSNFSKKYTLAEGNLEKIKELYAQNNGEDLKELASSFINLLTFTAIGA | |
| | PATFKFFDKNIDRKRYTSTTEILNATLIHQSITGLYETRIDLNKLGGD | |
| 280 | DKKYSIGLDIGTNSVGWAVITDEYKVPSKKFKVLGNTDRHSIKKNLIGAL | Streptococcus |
| | LFDSGETAEATRLKRTARRRYTRRKNRICYLQEIFSNEMAKVDDSFFHRL | pyogenes Cas9 |
| | EESFLVEEDKKHERHPIFGNIVDEVAYHEKYPTIYHLRKKLVDSTDKADL | |
| | RLIYLALAHMIKFRGHFLIEGDLNPDNSDVDKLFIQLVQTYNQLFEENPIN | |
| | ASGVDAKAILSARLSKSRRLENLIAQLPGEKKNGLFGNLIALSLGLTPNFK | |
| | SNFDLAEDAKLQLSKDTYDDDLDNLLAQIGDQYADLFLAAKNLSDAILL | |
| | SDILRVNTEITKAPLSASMIKRYDEHHQDLTLLKALVRQQLPEKYKEIFFD | |
| | QSKNGYAGYIDGGASQEEFYKFIKPILEKMDGTEELLVKLNREDLLRKQR | |
| | TFDNGSIPHQIHLGELHAILRRQEDFYPFLKDNREKIEKILTFRIPYYVGPL | |
| | ARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLF | |
| | KTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLT | |
| | FKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVE | |
| | NTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDN | |
| | LTKAERGGLSELDKAGFIKRQLVETRQITKHVAQILDSRMNTKYDENDK | |
| | LIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFK | |
| | TEITLANGEIRKRPLIETNGETGEIVWDKGRDFATVRKVLSMPQVNIVKK | |
| | TEVQTGGFSKESILPKRNSDKLIARKKDWDPKKYGGFDSPTVAYSVLVV | |
| | AKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNK | |
| | HRDKPIREQAENIIHLFTLTNLGAPAAFKYFDTTIDRKRYTSTKEVLDATLI | |
| | HQSITGLYETRIDLSQLGGD | |
| 281 | TKPYSIGLDIGTNSVGWAVTTDNYKVPSKKMKVLGNTSKKYIKKNLLGV | Streptococcus |
| | LLFDSGITAEGRRLKRTARRRYTRRRNRILYLQEIFSTEMATLDDAFFQRL | thermophilus Cas9 |
| | DDSFLVPDDKRDSKYPIFGNLVEEKAYHDEFPTIYHLRKYLADSTKKADL | |
| | RLVYLALAHMIKYRGHFLIEGEFNSKNNDIQKNFQDFLDTYNAIFESDLS | |
| | LENSKQLEEIVKDKISKLEKKDRILKLFPGEKNSGIFSEFLKLIVGNQADFR | |
| | KCFNLDEKASLHFSKESYDEDLETLLGYIGDDYSDVFLKAKKLYDAILLS | |
| | GFLTVTDNETEAPLSSAMIKRYNEHKEDLALLKEYIRNISLKTYNEVFKD | |
| | DTKNGYAGYIDGKTNQEDFYVYLKKLLAEFEGADYFLEKIDREDFLRKQ | |
| | RTFDNGSIPYQIHLQEMRAILDKQAKFYPFLAKNKERIEKILTFRIPYYVGP | |
| | LARGNSDFAWSIRKRNEKITPWNFEDVIDKESSAEAFINRMTSFDLYLPEE | |
| | KVLPKHSLLYETFNVYNELTKVRFIAESMRDYQFLDSKQKKDIVRLYFK | |
| | DKRKVTDKDIIEYLHAIYGYDGIELKGIEKQFNSSLSTYHDLLNIINDKEFL | |
| | DDSSNEAIIEEIIHTLTIFEDREMIKQRLSKFENIFDKSVLKKLSRRHYTGW | |
| | GKLSAKLINGIRDEKSGNTILDYLIDDGISNRNFMQLIHDDALSFKKKIQK | |
| | AQIIGDEDKGNIKEVVKSLPGSPAIKKGILQSIKIVDELVKVMGGRKPESIV | |
| | VEMARENQYTNQGKSNSQQRLKRLEKSLKELGSKILKENIPAKLSKIDNN | |
| | ALQNDRLYLYYLQNGKDMYTGDDLDIDRLSNYDIDHIIPQAFLKDNSIDN | |
| | KVLVSSASNRGKSDDVPSLEVVKKRKTFWYQLLKSKLISQRKFDNLTKA | |
| | ERGGLSPEDKAGFIQRQLVETRQITKHVARLLDEKFNNKKDENNRAVRT | |
| | VKIITLKSTLVSQFRKDFELYKVREINDFHHAHDAYLNAVVASALLKKYP | |
| | KLEPEFVYGDYPKYNSFRERKSATEKVYFYSNIMNIFKKSISLADGRVIER | |
| | PLIEVNEETGESVWNKESDLATVRRVLSYPQVNVVKKVEEQNHGLDRG | |
| | KPKGLFNANLSSKPKPNSNENLVGAKEYLDPKKYGGYAGISNSFTVLVK | |
| | GTIEKGAKKKITNVLEFQGISILDRINYRKDKLNFLLEKGYKDIELIIELPK | |
| | YSLFELSDGSRRMLASILSTNNKRGEIHKGNQIFLSQKFVKLLYHAKRISN | |
| | TINENHRKYVENHKKEFEELFYYILEFNENYVGAKKNGKLLNSAFQSWQ | |
| | NHSIDELCSSFIGPTGSERKGLFELTSRGSAADFEFLGVKIPRYRDYTPSSL | |
| | LKDATLIHQSVTGLYETRIDLAKLGEG | |
| 282 | KKPYTIGLDIGTNSVGWAVLTDQYDLVKRKMKIAGDSEKKQIKKNFWG | Listeria innocua |
| | VRLFDEGQTAADRRMARTARRRIERRRNRISYLQGIFAEEMSKTDANFFC | Cas9 |
| | RLSDSFYVDNEKRNSRHPFFATIEEEVEYHKNYPTIYHLREELVNSSEKAD | |
| | LRLVYLALAHIIKYRGNFLIEGALDTQNTSVDGIYKQFIQTYNQVFASGIE | |
| | DGSLKKLEDNKDVAKILVEKVTRKEKLERILKLYPGEKSAGMFAQFISLI | |
| | VGSKGNFQKPFDLIEKSDIECAKDSYEEDLESLLALIGDEYAELFVAAKN | |
| | AYSAVVLSSIITVAETETNAKLSASMIERFDTHEEDLGELKAFIKLHLPKH | |
| | YEEIFSNTEKHGYAGYIDGKTKQADFYKYMKMTLENIEGADYFIAKIEKE | |
| | NFLRKQRTFDNGAIPHQLHLEELEAILHQQAKYYPFLKENYDKIKSLVTF | |
| | RIPYFVGPLANGQSEFAWLTRKADGEIRPWNIEEKVDFGKSAVDFIEKMT | |
| | NKDTYLPKENVLPKHSLCYQKYLVYNELTKVRYINDQGKTSYFSGQEKE | |
| | QIFNDLFKQKRKVKKKDLELFLRNMSHVESPTIEGLEDSFNSSYSTYHDL | |
| | LKVGIKQEILDNPVNTEMLENIVKILTVFEDKRMIKEQLQQFSDVLDGVV | |
| | LKKLERRHYTGWGRLSAKLLMGIRDKQSHLTILDYLMNDDGLNRNLMQ | |
| | LINDSNLSFKSIIEKEQVTTADKDIQSIVADLAGSPAIKKGILQSLKIVDELV | |
| | SVMGYPPQTIVVEMARENQTTGKGKNNSRPRYKSLEKAIKEFGSQILKEH | |
| | PTDNQELRNNRLYLYYLQNGKDMYTGQDLDIHNLSNYDIDHIVPQSFITD | |
| | NSIDNLVLTSSAGNREKGDDVPPLEIVRKRKVFWEKLYQGNLMSKRKFD | |
| | YLTKAERGGLTEADKARFIHRQLVETRQITKNVANILHQRFNYEKDDHG | |
| | NTMKQVRIVTLKSALVSQFRKQFQLYKVRDVNDYHHAHDAYLNGVVA | |
| | NTLLKVYPQLEPEFVYGDYHQFDWFKANKATAKKQFYTNIMLFFAQKD | |
| | RIIDENGEILWDKKYLDTVKKVMSYRQMNIVKKTEIQKGEFSKATIKPKG | |
| | NSSKLIPRKTNWDPMKYGGLDSPNMAYAVVIEYAKGKNKLVFEKKIIRV | |
| | TIMERKAFEKDEKAFLEEQGYRQPKVLAKLPKYTLYECEEGRRRMLASA | |
| | NEAQKGNQQVLPNHLVTLLHHAANCEVSDGKSLDYIESNREMFAELLA | |
| | HVSEFAKRYTLAEANLNKINQLFEQNKEGDIKAIAQSFVDLMAFNAMGA | |
| | PASFKFFETTIERKRYNNLKELLNSTIIYQSITGLYESRKRLDD | |
| 283 | MAAFKPNSINYILGLDIGIASVGWAMVEIDEEENPIRLIDLGVRVFERAEV | Neisseria |
| | PKTGDSLAMARRLARSVRRLTRRRAHRLLRTRRLLKREGVLQAANFDEN | meningitidis Cas9 |
| | GLIKSLPNTPWQLRAAALDRKLTPLEWSAVLLHLIKHRGYLSQRKNEGE | |
| | TADKELGALLKGVAGNAHALQTGDFRTPAELALNKFEKESGHIRNQRSD | |
| | YSHTFSRKDLQAELILLFEKQKEFGNPHVSGGLKEGIETLLMTQRPALSG | |
| | DAVQKMLGHCTFEPAEPKAAKNTYTAERFIWLTKLNNLRILEQGSERPLT | |
| | DTERATLMDEPYRKSKLTYAQARKLLGLEDTAFFKGLRYGKDNAEASTL | |
| | MEMKAYHAISRALEKEGLKDKKSPLNLSPELQDEIGTAFSLFKTDEDITG | |
| | RLKDRIQPEILEALLKHISFDKFVQISLKALRRIVPLMEQGKRYDEACAEIY | |
| | GDHYGKKNTEEKIYLPPIPADEIRNPVVLRALSQARKVINGVVRRYGSPA | |
| | RIHIETAREVGKSFKDRKEIEKRQEENRKDREKAAAKFREYFPNFVGEPK | |
| | SKDILKLRLYEQQHGKCLYSGKEINLGRLNEKGYVEIDHALPFSRTWDDS | |
| | FNNKVLVLGSENQNKGNQTPYEYFNGKDNSREWQEFKARVETSRFPRSK | |
| | KQRILLQKFDEDGFKERNLNDTRYVNRFLCQFVADRMRLTGKGKKRVF | |
| | ASNGQITNLLRGFWGLRKVRAENDRHHALDAVVVACSTVAMQQKITRF | |
| | VRYKEMNAFDGKTIDKETGEVLHQKTHFPQPWEFFAQEVMIRVFGKPDG | |
| | KPEFEEADTLEKLRTLLAEKLSSRPEAVHEYVTPLFVSRAPNRKMSGQGH | |
| | METVKSAKRLDEGVSVLRVPLTQLKLKDLEKMVNREREPKLYEALKAR | |
| | LEAHKDDPAKAFAEPFYKYDKAGNRTQQVKAVRVEQVQKTGVWVRNH | |
| | NGIADNATMVRVDVFEKGDKYYLVPIYSWQVAKGILPDRAVVQGKDEE | |
| | DWQLIDDSFNFKFSLHPNDLVEVITKKARMFGYFASCHRGTGNINIRIHD | |
| | LDHKIGKNGILEGIGVKTALSFQKYQIDELGKEIRPCRLKKRPPVR | |
| 284 | MDKKYSIGLDIGTNSVGWAVITDEYKVPSKKFKVLGNTDRHSIKKNLIGA | Streptococcus |
| | LLFDSGETAEATRLKRTARRRYTRRKNRICYLQEIFSNEMAKVDDSFFHR | pyogenes Cas9 |
| | LEESFLVEEDKKHERHPIFGNIVDEVAYHEKYPTIYHLRKKLVDSTDKAD | |
| | LRLIYLALAHMIKFRGHFLIEGDLNPDNSDVDKLFIQLVQTYNQLFEENPI | |
| | NASGVDAKAILSARLSKSRRLENLIAQLPGEKKNGLFGNLIALSLGLTPNF | |
| | KSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLL | |
| | FKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLT | |
| | FKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVE | |
| | NTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDN | |
| | LTKAERGGLSELDKAGFIKRQLVETRQITKHVAQILDSRMNTKYDENDK | |
| | LIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFK | |
| | TEITLANGEIRKRPLIETNGETGEIVWDKGRDFATVRKVLSMPQVNIVKK | |
| | TEVQTGGFSKESILPKRNSDKLIARKKDWDPKKYGGFDSPTVAYSVLVV | |
| | AKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNK | |
| | HRDKPIREQAENIIHLFTLTNLGAPAAFKYFDTTIDRKRYTSTKEVLDATLI | |
| | HQSITGLYETRIDLSQLGGD | |
| 285 | MDKKYSIGLDIGTNSVGWAVITDEYKVPSKKFKVLGNTDRHSIKKNLIGA | S. pyogenes Cas9 |
| | LLFDSGETAEATRLKRTARRRYTRRKNRICYLQEIFSNEMAKVDDSFFHR | |
| | LEESFLVEEDKKHERHPIFGNIVDEVAYHEKYPTIYHLRKKLVDSTDKAD | |
| | LRLIYLALAHMIKFRGHFLIEGDLNPDNSDVDKLFIQLVQTYNQLFEENPI | |
| | NASGVDAKAILSARLSKSRRLENLIAQLPGEKKNGLFGNLIALSLGLTPNF | |
| | KSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLL | |
| | FKTNRKVTVKQLKEDYFKKIECFDSVEISGVEDRFNASLGTYHDLLKIIKD | |
| | KDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLT | |
| | FKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVE | |
| | NTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDN | |
| | LTKAERGGLSELDKAGFIKRQLVETRQITKHVAQILDSRMNTKYDENDK | |
| | LIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFK | |
| | TEITLANGEIRKRPLIETNGETGEIVWDKGRDFATVRKVLSMPQVNIVKK | |
| | TEVQTGGFSKESILPKRNSDKLIARKKDWDPKKYGGFDSPTVAYSVLVV | |
| | AKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNK | |
| | HRDKPIREQAENIIHLFTLTNLGAPAAFKYFDTTIDRKRYTSTKEVLDATLI | |
| | HQSITGLYETRIDLSQLGGD | |
| 286 | MAAFKPNPINYILGLDIGIASVGWAMVEIDEDENPICLIDLGVRVFERAEV | N. meningitidis |
| | PKTGDSLAMARRLARSVRRLTRRRAHRLLRARRLLKREGVLQAADFDE | Cas9 |
| | NGLIKSLPNTPWQLRAAALDRKLTPLEWSAVLLHLIKHRGYLSQRKNEG | |
| | ETADKELGALLKGVADNAHALQTGDFRTPAELALNKFEKESGHIRNQRG | |
| | DYSHTFSRKDLQAELILLFEKQKEFGNPHVSGGLKEGIETLLMTQRPALS | |
| | GDAVQKMLGHCTFEPAEPKAAKNTYTAERFIWLTKLNNLRILEQGSERP | |
| | LTDTERATLMDEPYRKSKLTYAQARKLLGLEDTAFFKGLRYGKDNAEAS | |
| | TLMEMKAYHAISRALEKEGLKDKKSPLNLSPELQDEIGTAFSLFKTDEDIT | |
| | GRLKDRIQPEILEALLKHISFDKFVQISLKALRRIVPLMEQGKRYDEACAEI | |
| | YGDHYGKKNTEEKIYLPPIPADEIRNPVVLRALSQARKVINGVVRRYGSP | |
| | ARIHIETAREVGKSFKDRKEIEKRQEENRKDREKAAAKFREYFPNFVGEP | |
| | KSKDILKLRLYEQQHGKCLYSGKEINLGRLNEKGYVEIDHALPFSRTWD | |
| | DSFNNKVLVLGSENQNKGNQTPYEYFNGKDNSREWQEFKARVETSRFPR | |
| | SKKQRILLQKFDEDGFKERNLNDTRYVNRFLCQFVADRMRLTGKGKKR | |
| | VFASNGQITNLLRGFWGLRKVRAENDRHHALDAVVVACSTVAMQQKIT | |
| | RFVRYKEMNAFDGKTIDKETGEVLHQKTHFPQPWEFFAQEVMIRVFGKP | |
| | DGKPEFEEADTPEKLRTLLAEKLSSRPEAVHEYVTPLFVSRAPNRKMSGQ | |
| | GHMETVKSAKRLDEGVSVLRVPLTQLKLKDLEKMVNREREPKLYEALK | |
| | ARLEAHKDDPAKAFAEPFYKYDKAGNRTQQVKAVRVEQVQKTGVWVR | |
| | NHNGIADNATMVRVDVFEKGDKYYLVPIYSWQVAKGILPDRAVVQGKD | |
| | EEDWQLIDDSFNFKFSLHPNDLVEVITKKARMFGYFASCHRGTGNINIRIH | |
| | DLDHKIGKNGILEGIGVKTALSFQKYQIDELGKEIRPCRLKKRPPVR | |
| 287 | MKRNYILGLDIGITSVGYGIIDYETRDVIDAGVRLFKEANVENNEGRRSK | S. aureus Cas9 |
| | RGARRLKRRRRHRIQRVKKLLFDYNLLTDHSELSGINPYEARVKGLSQKL | |
| | SEEEFSAALLHLAKRRGVHNVNEVEEDTGNELSTKEQISRNSKALEEKYV | |
| | AELQLERLKKDGEVRGSINRFKTSDYVKEAKQLLKVQKAYHQLDQSFID | |
| | TYIDLLETRRTYYEGPGEGSPFGWKDIKEWYEMLMGHCTYFPEELRSVK | |
| | YAYNADLYNALNDLNNLVITRDENEKLEYYEKFQIIENVFKQKKKPTLK | |
| | QIAKEILVNEEDIKGYRVTSTGKPEFTNLKVYHDIKDITARKEIIENAELLD | |
| | QIAKILTIYQSSEDIQEELTNLNSELTQEEIEQISNLKGYTGTHNLSLKAINL | |
| | ILDELWHTNDNQIAIFNRLKLVPKKVDLSQQKEIPTTLVDDFILSPVVKRS | |
| | FIQSIKVINAIIKKYGLPNDIIIELAREKNSKDAQKMINEMQKRNRQTNERI | |
| | EEIIRTTGKENAKYLIEKIKLHDMQEGKCLYSLEAIPLEDLLNNPFNYEVD | |
| | HIIPRSVSFDNSFNNKVLVKQEENSKKGNRTPFQYLSSSDSKISYETFKKHI | |
| | LNLAKGKGRISKTKKEYLLEERDINRFSVQKDFINRNLVDTRYATRGLM | |
| | NLLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKEIFIT | |
| | PHQIKHIKDFKDYKYSHRVDKKPNRELINDTLYSTRKDDKGNTLIVNNLN | |
| | GLYDKDNDKLKKLINKSPEKLLMYHHDPQTYQKLKLIMEQYGDEKNPL | |
| | YKYYEETGNYLTKYSKKDNGPVIKKIKYYGNKLNAHLDITDDYPNSRNK | |
| | VVKLSLKPYRFDVYLDNGVYKFVTVKNLDVIKKENYYEVNSKCYEEAK | |
| | KLKKISNQAEFIASFYNNDLIKINGELYRVIGVNNDLLNRIEVNMIDITYRE | |
| | YLENMNDKRPPRIIKTIASKTQSIKKYSTDILGNLYEVKSKKHPQIIKKG | |
| 288 | ATGCTGCTGCTGGTGACCAGCCTGCTGCTGTGCGAGCTGCCCCACCCC | GMCSFR alpha |
| | GCCTTTCTGCTGATCCCCGA | chain signal |
| | | sequence |
| 289 | CTGACCTCTTCTCTTCCTCCCACAG | human HBB gene |
| | | splice acceptor |
| 290 | TTTCTCTCCACAG | human IgG gene |
| | | splice acceptor |
| 291 | ESKYGPPCPPCPAPPVAGPSVFLFPPKPKDTLMISRTPEVTCVVVDVSQED | Spacer |
| | PEVQFNWYVDGVEVHNAKTKPREEQFQSTYRVVSVLTVLHQDWLNGK | |
| | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSR | |
| | WQEGNVFSCSVMHEALHNHYTQKSLSLSLGK | |
| 292 | MKWKALFTAAILQAQLPITEAQSFGLLDPKLCYLLDGILFIYGVILTALFL | CD3 zeta |
| | RVKFSRSADAPAYQQGQNQLYNELNLGRREEYDVLDKRRGRDPEMGGK | precursor |
| | PQRRKNPQEGLYNELQKDKMAEAYSEIGMKGERRRGKGHDGLYQGLST | |
| | ATKDTYDALHMQALPPR | |
| 293 | GVQVETISPGDGRTFPKRGQTCVVHYTGMLEDGKKMDSSRDRNKPFKF | FKBP |
| | MLGKQEVIRGWEEGVAQMSVGQRAKLTISPDYAYGATGHPGIIPPHATL | |
| | VFDVELLKLE | |
| 294 | GVQVETISPGDGRTFPKRGQTCVVHYTGMLEDGKKVDSSRDRNKPFKF | FKBP12v36 |
| | MLGKQEVIRGWEEGVAQMSVGQRAKLTISPDYAYGATGHPGIIPPHATL | |
| | VFDVELLKLE | |
| 295 | MGSNKSKPKDASQRRR | human C-Src |
| | | acylation motif |
| 296 | MGCXC | dual acylation |
| | | motif |
| 297 | CAAX | CAAX motif |

1. An engineered T cell comprising a modified T cell stimulation-associated locus comprising a transgene encoding a recombinant receptor integrated into an endogenous T cell stimulation-associated locus of the T cell, wherein the transgene is operably linked to an endogenous transcriptional regulatory element of the endogenous T cell stimulation-associated locus, wherein the endogenous transcriptional regulatory element induces or upregulates the expression of the operably linked transgene following a stimulation or activation signal in the T cell.

2. The engineered T cell of claim 1, wherein:
the endogenous transcriptional regulatory element is a promoter of the endogenous T cell stimulation-associated locus, and the transgene encoding the recombinant receptor or a portion thereof is present downstream of the promoter.

3. The engineered T cell of claim 1, wherein the endogenous transcriptional regulatory element comprises a response element or elements recognized by a transcription factor that is activated following the stimulation or activation signal.

4-7. (canceled)

8. The engineered T cell of claim 1, wherein the endogenous T cell stimulation-associated locus is selected from PDCD1, CD69, Nur77, FoxP3 and a HLA-DR locus.

9. The engineered T cell of claim 1, wherein the recombinant receptor comprises an extracellular binding domain.

10. The engineered T cell of claim 1, wherein
the recombinant receptor comprises an intracellular region comprising an intracellular signaling domain of a component of the T cell receptor (TCR) complex.

11. The engineered T cell of claim 10, wherein the intracellular region comprises an intracellular signaling domain of a CD3 chain or a portion thereof.

12-14. (canceled)

15. The engineered T cell of claim 1, wherein the recombinant receptor is a chimeric antigen receptor (CAR).

16-17. (canceled)

18. The engineered T cell of claim 1, wherein the recombinant receptor is a recombinant T cell receptor (TCR).

19-20. (canceled)

21. The engineered T cell of claim 1, wherein the modified T cell stimulation-associated locus is produced by integration of the transgene encoding the recombinant receptor into the endogenous T cell stimulation-associated locus by:
a) inducing a genetic disruption within the endogenous T cell stimulation-associated locus, wherein the genetic disruption is effected by a zinc finger nuclease (ZFN), a TAL-effector nuclease (TALEN), or a CRISPR-Cas9 combination that specifically binds to, recognizes, or hybridizes to the target site; and
b) introducing a polynucleotide for homology directed repair (HDR).

22. (canceled)

23. The engineered T cell of claim 21, wherein the T cell stimulation-associated locus is PDCD1, wherein the genetic disruption is effected by a CRISPR-Cas9 combination comprising a gRNA and wherein the gRNA has a targeting domain that is complementary to a target site in a PDCD1 gene.

24. The engineered T cell of claim 21, wherein the T cell stimulation-associated locus is CD69, wherein the genetic disruption is effected by a CRISPR-Cas9 combination comprising a gRNA and wherein the gRNA has a targeting domain that is complementary to a target site in a CD69 gene.

25. The engineered T cell of claim 21, wherein the T cell stimulation-associated locus is Nur77, wherein the genetic disruption is effected by a CRISPR-Cas9 combination comprising a gRNA and wherein the gRNA has a targeting domain that is complementary to a target site in a Nur77 gene.

26-27. (canceled)

28. The engineered T cell of claim 1, wherein the T cell further comprises a genetic disruption at an endogenous T cell receptor alpha constant region (TRAC) gene or an endogenous T cell receptor beta constant region (TRBC) gene.

29-31. (canceled)

32. The engineered T cell of claim 1, wherein the T cell is a T cell derived from a human subject.

33. A polynucleotide comprising:
(a) a transgene encoding a recombinant receptor or a portion thereof, and
(b) a 5′ homology arm and/or a 3′ homology arm linked to the transgene, wherein the 5′ homology arm and/or 3′ homology arm comprises a nucleic acid sequence homologous to a nucleic acid sequence of a target site within an endogenous T cell stimulation-associated locus in a T cell.

34-35. (canceled)

36. The polynucleotide of claim 33, wherein the target site is downstream of an endogenous transcriptional regulatory element of the T cell stimulation-associated locus.

37. (canceled)

38. The polynucleotide of claim 33, wherein the 5′ homology arm and 3′ homology arm are between 50 and 750 nucleotides.

39. The polynucleotide of claim 33, wherein the endogenous T cell stimulation-associated locus is selected from PDCD1, CD69, Nur77, FoxP3 and a HLA-DR locus.

40. The polynucleotide of claim 39, wherein the T cell stimulation-associated locus is PDCD1, and wherein the 5′ homology arm and 3′ homology arm comprise a nucleic acid sequence homologous to one or more region(s) of PDCD1.

41. The polynucleotide of claim 40, wherein:
the 5′ homology arm comprises:
a) a nucleic acid sequence comprising at least 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% to the nucleic acid sequence set forth in SEQ ID NO: 66;
b) a nucleic acid sequence comprising at least 150, 200, 250, 300, 350, 400, 450, 500, 550, or 600 contiguous nucleotides of the nucleic acid sequence set forth in SEQ ID NO:66; or
c) the nucleic acid sequence set forth in SEQ ID NO: 66.

42-45. (canceled)

46. The polynucleotide of claim 33, wherein the recombinant receptor comprises an extracellular binding domain.

47. The polynucleotide of claim 33, wherein:
the recombinant receptor comprises an intracellular region comprising an intracellular signaling domain of a component of the T cell receptor (TCR) complex.

48. The polynucleotide of claim 47, wherein the intracellular signaling region comprises an intracellular signaling domain of a CD3 chain, or a portion thereof.

49-61. (canceled)

62. A method of producing a genetically engineered T cell, the method comprising:
(a) introducing, into a T cell, one or more agent(s) that induces a genetic disruption at a target site within an endogenous T cell stimulation-associated locus of the T cell; and
(b) introducing the polynucleotide of claim 33 into the T cell,
wherein the method produces a modified T cell stimulation-associated locus, wherein the modified T cell stimulation-associated locus comprises a transgene encoding the recombinant receptor or a portion thereof, and wherein the transgene encoding a recombinant receptor or a portion thereof is integrated within the endogenous T cell stimulation-associated locus via homology directed repair (HDR).

63-65. (canceled)

66. The method of claim 62, wherein the target site is downstream of an endogenous transcriptional regulatory element of the endogenous T cell stimulation-associated locus.

67. The method of claim 62, wherein the endogenous T cell stimulation-associated locus is selected from PDCD1, CD69, Nur77, FoxP3 and a HLA-DR locus.

68. The method of claim 62, wherein the genetic disruption is effected by a zinc finger nuclease (ZFN), a TAL-effector nuclease (TALEN), or a CRISPR-Cas9 combination that specifically binds to, recognizes, or hybridizes to the target site.

69. (canceled)

70. The method of claim 62, wherein the endogenous T cell stimulation-associated locus is PDCD1, wherein the genetic disruption is effected by a CRISPR-Cas9 combination comprising a gRNA, and wherein gRNA comprises a targeting domain that is complementary to a target site in a PDCD1 gene.

71. The method of claim 62, wherein the endogenous T cell stimulation-associated locus is CD69, wherein the genetic disruption is effected by a CRISPR-Cas9 combination comprising a gRNA, and wherein the gRNA comprises a targeting domain that is complementary to a target site in a CD69 gene.

72. The method of claim 62, wherein the endogenous T cell stimulation-associated locus is Nur77, wherein the genetic disruption is effected by a CRISPR-Cas9 combination comprising a gRNA, and wherein the gRNA comprises a targeting domain that is complementary to a target site in a Nur77 gene.

73-74. (canceled)

75. The method of claim 62, wherein the T cell further comprises a genetic disruption at an endogenous T cell receptor alpha constant region (TRAC) gene or an endogenous T cell receptor beta constant region (TRBC) gene.

76-79. (canceled)

80. The method of claim 62, wherein the T cell is a human T cell.

81-86. (canceled)

87. An engineered T cell generated using the method of claim 62.

88. A composition comprising the engineered T cell of claim 1 or a plurality of the engineered T cells of claim 1.

89. A composition comprising the engineered T cell of claim 87 or a plurality of the engineered T cells of claim 87.

90-92. (canceled)

93. The composition of claim 88, wherein the composition comprises CD4+ T cells and/or CD8+ T cells.

94. The composition of claim 88, wherein the composition comprises CD4+ and CD8+ T cells and the ratio of CD4+ to CD8+ T cells is from 1:3 to 3:1 or 1:1.

95. A method of treatment comprising administering the engineered T cell of claim 1 to a subject having a disease or disorder.

96. The method of claim 95, wherein the disease or disorder is a cancer or a tumor.

97. The method of claim 96, wherein the cancer or the tumor is a hematologic malignancy, and wherein the hematologic malignancy is a lymphoma, a leukemia, or a plasma cell malignancy.

98. The method of claim 97, wherein the leukemia is chronic lymphocytic leukemia (CLL), plasma cell leukemia or acute lymphocytic leukemia (ALL).

99. The method of claim 97, wherein the plasma cell malignancy is multiple myeloma (MM).

100. The method of claim 96, wherein the tumor is a solid tumor, and wherein the solid tumor is a non-small cell lung cancer (NSCLC) or a head and neck squamous cell carcinoma (HNSCC).

101. (canceled)

102. A kit comprising:
one or more agent(s) capable of inducing a genetic disruption at a target site within a T cell stimulation-associated locus; and
the polynucleotide of claim 33.

103. (canceled)

104. The engineered T cell of claim 9, wherein binding of an agent to the extracellular binding domain of the recombinant receptor results in the inducing or transmitting of the stimulation or activation signal in the cell.

105. The engineered T cell of claim 1, wherein the recombinant receptor comprises an intracellular region comprising an intracellular signaling domain comprising an immunoreceptor tyrosine-based activation motif (ITAM).

106. The polynucleotide of claim 40, wherein:
the 3′ homology arm comprises:
a) a nucleic acid sequence comprising at least 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% to the nucleic acid sequence set forth in SEQ ID NO: 67;
b) a nucleic acid sequence comprising at least 150, 200, 250, 300, 350, 400, 450, 500, 550, or 600 contiguous nucleotides of the nucleic acid sequence set forth in SEQ ID NO:67; or
c) the nucleic acid sequence set forth in SEQ ID NO: 67.

107. The method of claim 97, wherein the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL).


SEQUENCE LISTING (297 sequences)

SEQ ID NO: 1 - protein, 12 aa, Artificial Sequence
  spacer (IgG4hinge)
ESKYGPPCPPCP

SEQ ID NO: 2 - DNA, 36 bp, Artificial Sequence
  spacer (IgG4hinge)
gaatctaagtacggaccgccctgccccccttgccct

SEQ ID NO: 3 - protein, 119 aa, Artificial Sequence
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Artificial Sequence
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Artificial Sequence
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Artificial Sequence
  CD28 (amino acids 153-179)
FWVLVVVGGVLACYSLLVTVAFIIFWV

SEQ ID NO: 9 - protein, 66 aa, Artificial Sequence
  CD28 (amino acids 114-179)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Artificial Sequence
  CD28 (amino acids 180-220)
RSKRSRLLHSDYMNMTPRRPGPTRKHYQPYAPPRDFAAYRS

SEQ ID NO: 11 - protein, 41 aa, Artificial Sequence
  CD28 (LL to GG)
RSKRSRGGHSDYMNMTPRRPGPTRKHYQPYAPPRDFAAYRS

SEQ ID NO: 12 - protein, 42 aa, Artificial Sequence
  4-1BB (amino acids 214-255)
KRGRKKLLYIFKQPFMRPVQTTQEEDGCSCRFPEEEEGGCEL

SEQ ID NO: 13 - protein, 112 aa, Artificial Sequence
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Artificial Sequence
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Artificial Sequence
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 10 aa, Artificial Sequence
  Linker
  REPEAT (5)...(9): Repeats 5 or 6 times
PGGGSGGGGP

SEQ ID NO: 23 - protein, 17 aa, Artificial Sequence
  Linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 5 aa, Artificial Sequence
  Hinge
  VARIANT (1)...(1): Xaa is gly, cys or arg
  VARIANT (4)...(4): Xaa is cys or thr
XPPXP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  HC-CDR3
HYYYGGSYAMDY

SEQ ID NO: 55 - protein, 7 aa, Artificial Sequence
  LC-CDR2
HTSRLHS

SEQ ID NO: 56 - protein, 9 aa, Artificial Sequence
  LC-CDR3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 18 aa, Artificial Sequence
  Linker
GSTSGSGKPGSGEGSTKG

SEQ ID NO: 59 - protein, 326 aa, Homo sapiens
  Human IgG2 Fc
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 60 - protein, 327 aa, Homo sapiens
  Human IgG4 Fc
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - DNA, 63 bp, Artificial Sequence
  P2A
ggaagcggagagggcagaggaagtcttctaacatgcggtgacgtggaggagaatcccggccca

SEQ ID NO: 62 - protein, 21 aa, Artificial Sequence
  P2A
GSGEGRGSLLTCGDVEENPGP

SEQ ID NO: 63 - DNA, 215 bp, Artificial Sequence
  Poly A signal
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 64 - DNA, 1566 bp, Artificial Sequence
  anti-CD19 CAR
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgagagcaagaattggagccacccgcagttcgaaaaaggaggtggaggttcaggtggtggaggctcttacggaccgaattggtctcatcctcagttcgagaaaggaggcggttctggaggtggaagcggtggctcttggagccacccacagtttgaaaagggaggcgggggctccggtggcggaggctcttccggatctccctgtccaccttgccctatgttctgggtgctggtagtggtaggtggagtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcacctgcctaccagcagggccagaatcagctgtacaacgagctgaacctgggacgaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 65 - protein, 499 aa, Artificial Sequence
  anti-CD19 CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKNWSHPQFEKGGGGSGGGGSYGPNWSHPQFEKGGGSGGGSGGSWSHPQFEKGGGGSGGGGSSGSPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 102 bp, Artificial Sequence
  PDCD1 5′ homology arm
taattacctccgcctgagcagtggagaaggcggcactctggtggggctgctccaggcatgcagatcccacaggcgccctggccagtcgtctgggcggtgcta

SEQ ID NO: 67 - DNA, 187 bp, Artificial Sequence
  PDCD1 3′ homology arm
caactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagt

SEQ ID NO: 68 - DNA, 70 bp, Artificial Sequence
  TRAC 5′ homology arm
gggaaatgagatcatgtcctaaccctgatcctcttgtcccacagatatccagaaccctgaccctgccgtg

SEQ ID NO: 69 - DNA, 58 bp, Artificial Sequence
  TRAC 3′ homology arm
taccagctgagagactctaaatccagtgacaagtctgtctgcctattcaccgattttg

SEQ ID NO: 70 - DNA, 20 bp, Artificial Sequence
  PDCD1 5′ primer
taattacctccgcctgagca

SEQ ID NO: 71 - DNA, 20 bp, Artificial Sequence
  PDCD1 3′ primer
actgagagtgaaaggtccct

SEQ ID NO: 72 - DNA, 20 bp, Artificial Sequence
  TRAC 5′ primer
caaaatcggtgaataggcag

SEQ ID NO: 73 - DNA, 20 bp, Artificial Sequence
  TRAC 3′ primer
gggaaatgagatcatgtcct

SEQ ID NO: 74 - DNA, 20 bp, Artificial Sequence
  PDCD1 gRNA targeting sequence
cgtctgggcggtgctacaac

SEQ ID NO: 75 - RNA, 20 nt, Artificial Sequence
  PDCD1 gRNA
cgucugggcggugcuacaac

SEQ ID NO: 76 - DNA, 20 bp, Artificial Sequence
  TRAC gRNA targeting sequence
agagtctctcagctggtaca

SEQ ID NO: 77 - RNA, 20 nt, Artificial Sequence
  TRAC gRNA
agagucucucagcugguaca

SEQ ID NO: 78 - DNA, 36 bp, Artificial Sequence
  PDCD1 gRNA targeting sequence
cgtctgggcggtgctacaacgttttagagctatgct

SEQ ID NO: 79 - protein, 288 aa, Artificial Sequence
  PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 80 - DNA, 2097 bp, Artificial Sequence
  PDCD1
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 81 - protein, 199 aa, Artificial Sequence
  CD69
MSSENCFVAENSSLHPESGQENDATSPHFSTRHEGSFQVPVLCAVMNVVFITILIIALIALSVGQYNCPGQYTFSMPSDSHVSSCSEDWVGYQRKCYFISTVKRSWTSAQNACSEHGATLAVIDSEKDMNFLKRYAGREEHWVGLKKEPGHPWKWSNGKEFNNWFNVTGSDKCVFLKNTEVSSMECEKNLYWICNKPYK

SEQ ID NO: 82 - DNA, 1676 bp, Artificial Sequence
  CD69
agactcaacaagagctccagcaaagactttcactgtagcttgacttgacctgagattaactagggaatcttgagaataaagatgagctctgaaaattgtttcgtagcagagaacagctctttgcatccggagagtggacaagaaaatgatgccaccagtccccatttctcaacacgtcatgaagggtccttccaagttcctgtcctgtgtgctgtaatgaatgtggtcttcatcaccattttaatcatagctctcattgccttatcagtgggccaatacaattgtccaggccaatacacattctcaatgccatcagacagccatgtttcttcatgctctgaggactgggttggctaccagaggaaatgctactttatttctactgtgaagaggagctggacttcagcccaaaatgcttgttctgaacatggtgctactcttgctgtcattgattctgaaaaggacatgaactttctaaaacgatacgcaggtagagaggaacactgggttggactgaaaaaggaacctggtcacccatggaagtggtcaaatggcaaagaatttaacaactggttcaacgttacagggtctgacaagtgtgtttttctgaaaaacacagaggtcagcagcatggaatgtgagaagaatttatactggatatgtaacaaaccttacaaataataaggaaacatgttcacttattgactattatagaatggaactcaaggaaatctgtgtcagtggatgctgctctgtggtccgaagtcttccatagagactttgtgaaaaaaaattttatagtgtcttgggaattttcttccaaacagaactatggaaaaaaaggaagaaattccaggaaaatctgcactgtgggcttttattgccatgagctagaagcatcacaggttgaccaataaccatgcccaagaatgagaagaatgactatgcaacctttggatgcactttatattattttgaatccagaaataatgaaataactaggcgtggacttactatttattgctgaatgactaccaacagtgagagcccttcatgcatttgcactattggaaggagttagatgttggtactagatactgaatgtaaacaaaggaattatggctggtaacataggtttttagtctaattgaatcccttaaactcagggagcatttataaatggacaaatgcttatgaaactaagatttgtaatatttctctctttttagagaaatttgccaatttactttgttatttttccccaaaaagaatgggatgatcatgtatttatttttttacttcctcagctgtagacaggtccttttcgatggtacatatttctttgcctttataatcttttatacagtgtcttacagagaaaagacataagcaaagactatgaggaatatttgcaagacatagaatagtgttggaaaatgtgcaatatgtgatgtggcaaatctctattaggaaatattctgtaatcttcagacctagaataatactagtcttataataggtttgtgactttcctaaatcaattctattacgtgcaatacttcaatacttcatttaaaatatttttatgtgcaataaaatgtatttgtttgtattttgtgttcagtacaattataagctgtttttatatatgtgaaataaaagtagaataaacacaa

SEQ ID NO: 83 - protein, 598 aa, Artificial Sequence
  NR4A1 (Nur77)
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 84 - DNA, 2692 bp, Artificial Sequence
  NR4A1 (Nur77)
gtcacggagcgcttaagaggagggtcgggctcggccggggagtcccagtggcggaggctacgaaacttgggggagtgcacagaagaacttcgggagcgcacgcgggaccagggaccaggctgagactcggggcgccagtccgggcaggggcagcgggagccggccgggtagggtgcagcctgaggcttgttcagcagaacaggtgcaagccacattgttgccaagacctgcctgaagccggattctccccactgcctccttcaaccccgcctcttcctcctcctgtgggactgctcccccctcctgtgaggctagatagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaactgaaaaaaaaaaaaaa

SEQ ID NO: 85 - protein, 431 aa, Artificial Sequence
  FoxP3
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 86 - DNA, 2397 bp, Artificial Sequence
  FoxP3
gcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctccccacaaaaaaaaaaaaaaaa

SEQ ID NO: 87 - protein, 254 aa, Artificial Sequence
  HLA-DRA
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 88 - DNA, 1312 bp, Artificial Sequence
  HLA-DRA
ttttaatggtcagactctattacaccccacattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaaaaaaaaaaaaaaaaaaa

SEQ ID NO: 89 - protein, 266 aa, Artificial Sequence
  HLA-DRB1
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 90 - DNA, 1182 bp, Artificial Sequence
  HLA-DRB1
tagttctccctgagtgagacttgcctgcttctctggcccctggtcctgtcctgttctccagcatggtgtgtctgaagctccctggaggctcctgcatgacagcgctgacagtgacactgatggtgctgagctccccactggctttggctggggacacccgaccacgtttcttgtggcagcttaagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggattcctgagctgaaatgcagatgaccacattcaaggaagaaccttctgtcccagctttgcagaatgaaaagctttcctgcttggcagttattcttccacaagagagggctttctcaggacctggttgctactggttcggcaactgcagaaaatgtcctcccttgtggcttcctcagctcctgcccttggcctgaagtcccagcattgatgacagcgcctcatcttcaacttttgtgctcccctttgcctaaaccgtatggcctcccgtgcatctgtactcaccctgtacgacaaacacattacattattaaatgtttctcaaagatggagtt

SEQ ID NO: 91 - protein, 142 aa, Artificial Sequence
  Human TCR alpha constant
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 141 aa, Artificial Sequence
  Human TCR alpha constant
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 93 - DNA, 1508 bp, Artificial Sequence
  Human TCR alpha constant
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaa

SEQ ID NO: 94 - protein, 177 aa, Artificial Sequence
  Human TCR beta constant 1
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 95 - protein, 178 aa, Artificial Sequence
  Human TCR beta constant 2
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 96 - protein, 179 aa, Artificial Sequence
  Human TCR beta constant
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 97 - DNA, 1151 bp, Artificial Sequence
  Human TCR beta constant
ctggtctagaatattccacatctgctctcactctgccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttctcgacctgttcggctaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaaggcagccctggaagtggagttaggagcttctaacccgtcatggttcaatacacattcttcttttgccagcgcttctgaagagctgctctcacctctctgcatcccaatagatatccccctatgtgcatgcacacctgcacactcacggctgaaatctccctaacccagggggac

SEQ ID NO: 98 - DNA, 20 bp, Artificial Sequence
  PDCD1 gRNA target sequence 1
tgacgttacctcgtgcggcc

SEQ ID NO: 99 - DNA, 20 bp, Artificial Sequence
  PDCD1 gRNA target sequence 2
cacgaagctctccgatgtgt

SEQ ID NO: 100 - DNA, 20 bp, Artificial Sequence
  PDCD1 gRNA target sequence 3
gcgtgacttccacatgagcg

SEQ ID NO: 101 - DNA, 20 bp, Artificial Sequence
  PDCD1 gRNA target sequence 4
ttggaactggccggctggcc

SEQ ID NO: 102 - DNA, 20 bp, Artificial Sequence
  PDCD1 gRNA target sequence 5
gtggcatactccgtctgctc

SEQ ID NO: 103 - DNA, 20 bp, Artificial Sequence
  PDCD1 gRNA target sequence 6
gatgaggtgcccattccgct

SEQ ID NO: 104 - RNA, 20 nt, Artificial Sequence
  PDCD1 gRNA sequence 1
ugacguuaccucgugcggcc

SEQ ID NO: 105 - RNA, 20 nt, Artificial Sequence
  PDCD1 gRNA sequence 2
cacgaagcucuccgaugugu

SEQ ID NO: 106 - RNA, 20 nt, Artificial Sequence
  PDCD1 gRNA sequence 3
gcgugacuuccacaugagcg

SEQ ID NO: 107 - RNA, 20 nt, Artificial Sequence
  PDCD1 gRNA sequence 4
uuggaacuggccggcuggcc

SEQ ID NO: 108 - RNA, 20 nt, Artificial Sequence
  PDCD1 gRNA sequence 5
guggcauacuccgucugcuc

SEQ ID NO: 109 - RNA, 20 nt, Artificial Sequence
  PDCD1 gRNA sequence 6
gaugaggugcccauuccgcu

SEQ ID NO: 110 - DNA, 20 bp, Artificial Sequence
  CD69 gRNA target sequence 1
aactttctaaaacgatacgc

SEQ ID NO: 111 - DNA, 20 bp, Artificial Sequence
  CD69 gRNA target sequence 2
tttgacaggttcaacgttac

SEQ ID NO: 112 - DNA, 20 bp, Artificial Sequence
  CD69 gRNA target sequence 3
catttctcaacacgtcatga

SEQ ID NO: 113 - DNA, 20 bp, Artificial Sequence
  CD69 gRNA target sequence 4
cttcatgacgtgttgagaaa

SEQ ID NO: 114 - DNA, 20 bp, Artificial Sequence
  CD69 gRNA target sequence 5
gtgggccaatacaattgtcc

SEQ ID NO: 115 - DNA, 20 bp, Artificial Sequence
  CD69 gRNA target sequence 6
atggcattgagaatgtgtat

SEQ ID NO: 116 - RNA, 20 nt, Artificial Sequence
  CD69 gRNA sequence 1
aacuuucuaaaacgauacgc

SEQ ID NO: 117 - RNA, 20 nt, Artificial Sequence
  CD69 gRNA sequence 2
uuugacagguucaacguuac

SEQ ID NO: 118 - RNA, 20 nt, Artificial Sequence
  CD69 gRNA sequence 3
cauuucucaacacgucauga

SEQ ID NO: 119 - RNA, 20 nt, Artificial Sequence
  CD69 gRNA sequence 4
cuucaugacguguugagaaa

SEQ ID NO: 120 - RNA, 20 nt, Artificial Sequence
  CD69 gRNA sequence 5
gugggccaauacaauugucc

SEQ ID NO: 121 - RNA, 20 nt, Artificial Sequence
  CD69 gRNA sequence 6
auggcauugagaauguguau

SEQ ID NO: 122 - DNA, 20 bp, Artificial Sequence
  Nur77 (NR4A1) gRNA target sequence 1
cggggtagcagccgtacacc

SEQ ID NO: 123 - DNA, 20 bp, Artificial Sequence
  Nur77 (NR4A1) gRNA target sequence 2
ccctgtatccaagcccaata

SEQ ID NO: 124 - DNA, 20 bp, Artificial Sequence
  Nur77 (NR4A1) gRNA target sequence 3
tgtccgaacagacagcctga

SEQ ID NO: 125 - DNA, 20 bp, Artificial Sequence
  Nur77 (NR4A1) gRNA target sequence 4
aaacgccaagtacatctgcc

SEQ ID NO: 126 - DNA, 20 bp, Artificial Sequence
  Nur77 (NR4A1) gRNA target sequence 5
caccttggagtagtccagtt

SEQ ID NO: 127 - DNA, 20 bp, Artificial Sequence
  Nur77 (NR4A1) gRNA target sequence 6
tttcgccgcctcttgtccac

SEQ ID NO: 128 - RNA, 20 nt, Artificial Sequence
  Nur77 (NR4A1) gRNA sequence 1
cgggguagcagccguacacc

SEQ ID NO: 129 - RNA, 20 nt, Artificial Sequence
  Nur77 (NR4A1) gRNA sequence 2
cccuguauccaagcccaaua

SEQ ID NO: 130 - RNA, 20 nt, Artificial Sequence
  Nur77 (NR4A1) gRNA sequence 3
uguccgaacagacagccuga

SEQ ID NO: 131 - RNA, 20 nt, Artificial Sequence
  Nur77 (NR4A1) gRNA sequence 4
aaacgccaaguacaucugcc

SEQ ID NO: 132 - RNA, 20 nt, Artificial Sequence
  Nur77 (NR4A1) gRNA sequence 5
caccuuggaguaguccaguu

SEQ ID NO: 133 - RNA, 20 nt, Artificial Sequence
  Nur77 (NR4A1) gRNA sequence 6
uuucgccgccucuuguccac

SEQ ID NO: 134 - DNA, 20 bp, Artificial Sequence
  Nur77 (NR4A1) gRNA target sequence 7
tcattgacaagatcttcatg

SEQ ID NO: 135 - DNA, 19 bp, Artificial Sequence
  Nur77 (NR4A1) gRNA target sequence 8
gcctgggaacacgtgtgca

SEQ ID NO: 136 - DNA, 20 bp, Artificial Sequence
  Nur77 (NR4A1) gRNA target sequence 9
ccatattgggcttggataca

SEQ ID NO: 137 - RNA, 20 nt, Artificial Sequence
  Nur77 (NR4A1) gRNA sequence 7
caugaagaucuugucaauga

SEQ ID NO: 138 - RNA, 19 nt, Artificial Sequence
  Nur77 (NR4A1) gRNA sequence 8
ugcacacguguucccaggc

SEQ ID NO: 139 - RNA, 20 nt, Artificial Sequence
  Nur77 (NR4A1) gRNA sequence 9
ccauauugggcuuggauaca

SEQ ID NO: 140 - DNA, 20 bp, Artificial Sequence
  FoxP3 gRNA target sequence1
cccacccacagggatcaacg

SEQ ID NO: 141 - DNA, 20 bp, Artificial Sequence
  FoxP3 gRNA target sequence 2
ttcgaagaccttctcacatc

SEQ ID NO: 142 - DNA, 20 bp, Artificial Sequence
  FoxP3 gRNA target sequence 3
agctctggggcacagccgaa

SEQ ID NO: 143 - DNA, 20 bp, Artificial Sequence
  FoxP3 gRNA target sequence 4
ccacttacaggcactcctcc

SEQ ID NO: 144 - DNA, 20 bp, Artificial Sequence
  FoxP3 gRNA target sequence 5
cctggacacccattccaggc

SEQ ID NO: 145 - DNA, 20 bp, Artificial Sequence
  FoxP3 gRNA target sequence 6
tgccccccagctctcaacgg

SEQ ID NO: 146 - DNA, 20 bp, Artificial Sequence
  FoxP3 gRNA target sequence 7
tccagctgggcgaggctcct

SEQ ID NO: 147 - DNA, 20 bp, Artificial Sequence
  FoxP3 gRNA target sequence 8
tttgggtgcagccctccagc

SEQ ID NO: 148 - RNA, 20 nt, Artificial Sequence
  FoxP3 gRNA sequence 1
cccacccacagggaucaacg

SEQ ID NO: 149 - RNA, 20 nt, Artificial Sequence
  FoxP3 gRNA sequence 2
uucgaagaccuucucacauc

SEQ ID NO: 150 - RNA, 20 nt, Artificial Sequence
  FoxP3 gRNA sequence 3
agcucuggggcacagccgaa

SEQ ID NO: 151 - RNA, 20 nt, Artificial Sequence
  FoxP3 gRNA sequence 4
ccacuuacaggcacuccucc

SEQ ID NO: 152 - RNA, 20 nt, Artificial Sequence
  FoxP3 gRNA sequence 5
ccuggacacccauuccaggc

SEQ ID NO: 153 - RNA, 20 nt, Artificial Sequence
  FoxP3 gRNA sequence 6
ugccccccagcucucaacgg

SEQ ID NO: 154 - RNA, 20 nt, Artificial Sequence
  FoxP3 gRNA sequence 7
uccagcugggcgaggcuccu

SEQ ID NO: 155 - RNA, 20 nt, Artificial Sequence
  FoxP3 gRNA sequence 8
uuugggugcagcccuccagc

SEQ ID NO: 156 - DNA, 20 bp, Artificial Sequence
  HLA-DRA gRNA target sequence 1
gtcgtaaacgtcctcagttg

SEQ ID NO: 157 - DNA, 20 bp, Artificial Sequence
  HLA-DRA gRNA target sequence 2
caaacataaactcgcctgat

SEQ ID NO: 158 - DNA, 20 bp, Artificial Sequence
  HLA-DRA gRNA target sequence 3
gattcagatagaactcggcc

SEQ ID NO: 159 - DNA, 20 bp, Artificial Sequence
  HLA-DRA gRNA target sequence 4
agacaagttcaccccaccag

SEQ ID NO: 160 - DNA, 20 bp, Artificial Sequence
  HLA-DRA gRNA target sequence 5
agctgtgctgatgagcgctc

SEQ ID NO: 161 - DNA, 20 bp, Artificial Sequence
  HLA-DRA gRNA target sequence 6
aaagcaatgcagcagaacgc

SEQ ID NO: 162 - RNA, 20 nt, Artificial Sequence
  HLA-DRA gRNA sequence 1
gucguaaacguccucaguug

SEQ ID NO: 163 - RNA, 20 nt, Artificial Sequence
  HLA-DRA gRNA sequence 2
caaacauaaacucgccugau

SEQ ID NO: 164 - RNA, 20 nt, Artificial Sequence
  HLA-DRA gRNA sequence 3
gauucagauagaacucggcc

SEQ ID NO: 165 - RNA, 20 nt, Artificial Sequence
  HLA-DRA gRNA sequence 4
agacaaguucaccccaccag

SEQ ID NO: 166 - RNA, 20 nt, Artificial Sequence
  HLA-DRA gRNA sequence 5
agcugugcugaugagcgcuc

SEQ ID NO: 167 - RNA, 20 nt, Artificial Sequence
  HLA-DRA gRNA sequence 6
aaagcaaugcagcagaacgc

SEQ ID NO: 168 - DNA, 20 bp, Artificial Sequence
  HLA-DRB1 gRNA target sequence 1
gacggagcgggtgcggttcc

SEQ ID NO: 169 - DNA, 20 bp, Artificial Sequence
  HLA-DRB1 gRNA target sequence 2
cacgtcgctgtcgaagcgca

SEQ ID NO: 170 - DNA, 20 bp, Artificial Sequence
  HLA-DRB1 gRNA target sequence 3
cactgtcagcgctgtcatgc

SEQ ID NO: 171 - DNA, 20 bp, Artificial Sequence
  HLA-DRB1 gRNA target sequence 4
cctgaagtagatgaacagcc

SEQ ID NO: 172 - DNA, 20 bp, Artificial Sequence
  HLA-DRB1 gRNA target sequence 5
gaaggatatacagtcacctt

SEQ ID NO: 173 - DNA, 20 bp, Artificial Sequence
  HLA-DRB1 gRNA target sequence 6
agctccccactggctttgtc

SEQ ID NO: 174 - DNA, 23 bp, Artificial Sequence
  HLA-DRB1 gRNA target sequence 7
atccaggcagcattgaagtcagg

SEQ ID NO: 175 - DNA, 23 bp, Artificial Sequence
  HLA-DRB1 gRNA target sequence 8
ccaggcagcattgaagtcaggtg

SEQ ID NO: 176 - DNA, 23 bp, Artificial Sequence
  HLA-DRB1 gRNA target sequence 9
ccttccagaccctggtgatgctg

SEQ ID NO: 177 - DNA, 23 bp, Artificial Sequence
  HLA-DRB1 gRNA target sequence 10
ccagaccctggtgatgctggaaa

SEQ ID NO: 178 - RNA, 20 nt, Artificial Sequence
  HLA-DRB1 gRNA sequence 1
gacggagcgggugcgguucc

SEQ ID NO: 179 - RNA, 20 nt, Artificial Sequence
  HLA-DRB1 gRNA sequence 2
cacgucgcugucgaagcgca

SEQ ID NO: 180 - RNA, 20 nt, Artificial Sequence
  HLA-DRB1 gRNA sequence 3
cacugucagcgcugucaugc

SEQ ID NO: 181 - RNA, 20 nt, Artificial Sequence
  HLA-DRB1 gRNA sequence 4
ccugaaguagaugaacagcc

SEQ ID NO: 182 - RNA, 20 nt, Artificial Sequence
  HLA-DRB1 gRNA sequence 5
gaaggauauacagucaccuu

SEQ ID NO: 183 - RNA, 20 nt, Artificial Sequence
  HLA-DRB1 gRNA sequence 6
agcuccccacuggcuuuguc

SEQ ID NO: 184 - RNA, 23 nt, Artificial Sequence
  HLA-DRB1 gRNA sequence 7
auccaggcagcauugaagucagg

SEQ ID NO: 185 - RNA, 23 nt, Artificial Sequence
  HLA-DRB1 gRNA sequence 8
ccaggcagcauugaagucaggug

SEQ ID NO: 186 - RNA, 23 nt, Artificial Sequence
  HLA-DRB1 gRNA sequence 9
ccuuccagacccuggugaugcug

SEQ ID NO: 187 - RNA, 23 nt, Artificial Sequence
  HLA-DRB1 gRNA sequence 10
ccagacccuggugaugcuggaaa

SEQ ID NO: 188 - RNA, 20 nt, Artificial Sequence
  TRAC-10
ucucucagcugguacacggc

SEQ ID NO: 189 - RNA, 20 nt, Artificial Sequence
  TRAC-110
uggauuuagagucucucagc

SEQ ID NO: 190 - RNA, 20 nt, Artificial Sequence
  TRAC-116
acacggcagggucaggguuc

SEQ ID NO: 191 - RNA, 20 nt, Artificial Sequence
  TRAC-16
gagaaucaaaaucggugaau

SEQ ID NO: 192 - RNA, 20 nt, Artificial Sequence
  TRAC-4
gcugguacacggcaggguca

SEQ ID NO: 193 - RNA, 17 nt, Artificial Sequence
  TRAC-49
cucagcugguacacggc

SEQ ID NO: 194 - RNA, 17 nt, Artificial Sequence
  TRAC-2
ugguacacggcaggguc

SEQ ID NO: 195 - RNA, 17 nt, Artificial Sequence
  TRAC-30
gcuagacaugaggucua

SEQ ID NO: 196 - RNA, 17 nt, Artificial Sequence
  TRAC-43
gucagauuuguugcucc

SEQ ID NO: 197 - RNA, 17 nt, Artificial Sequence
  TRAC-23
ucagcugguacacggca

SEQ ID NO: 198 - RNA, 17 nt, Artificial Sequence
  TRAC-34
gcagacagacuugucac

SEQ ID NO: 199 - RNA, 17 nt, Artificial Sequence
  TRAC-25
gguacacggcaggguca

SEQ ID NO: 200 - RNA, 20 nt, Artificial Sequence
  TRAC-128
cuucaagagcaacagugcug

SEQ ID NO: 201 - RNA, 20 nt, Artificial Sequence
  TRAC-105
agagcaacagugcuguggcc

SEQ ID NO: 202 - RNA, 20 nt, Artificial Sequence
  TRAC-106
aaagucagauuuguugcucc

SEQ ID NO: 203 - RNA, 20 nt, Artificial Sequence
  TRAC-123
acaaaacugugcuagacaug

SEQ ID NO: 204 - RNA, 17 nt, Artificial Sequence
  TRAC-64
aaacugugcuagacaug

SEQ ID NO: 205 - RNA, 20 nt, Artificial Sequence
  TRAC-97
ugugcuagacaugaggucua

SEQ ID NO: 206 - RNA, 22 nt, Artificial Sequence
  TRAC-148
ggcuggggaagaaggugucuuc

SEQ ID NO: 207 - RNA, 21 nt, Artificial Sequence
  TRAC-147
gcuggggaagaaggugucuuc

SEQ ID NO: 208 - RNA, 18 nt, Artificial Sequence
  TRAC-234
ggggaagaaggugucuuc

SEQ ID NO: 209 - RNA, 22 nt, Artificial Sequence
  TRAC-167
guuuugucugugauauacacau

SEQ ID NO: 210 - RNA, 24 nt, Artificial Sequence
  TRAC-177
ggcagacagacuugucacuggauu

SEQ ID NO: 211 - RNA, 23 nt, Artificial Sequence
  TRAC-176
gcagacagacuugucacuggauu

SEQ ID NO: 212 - RNA, 20 nt, Artificial Sequence
  TRAC-257
gacagacuugucacuggauu

SEQ ID NO: 213 - RNA, 24 nt, Artificial Sequence
  TRAC-233
gugaauaggcagacagacuuguca

SEQ ID NO: 214 - RNA, 22 nt, Artificial Sequence
  TRAC-231
gaauaggcagacagacuuguca

SEQ ID NO: 215 - RNA, 22 nt, Artificial Sequence
  TRAC-163
gagucucucagcugguacacgg

SEQ ID NO: 216 - RNA, 20 nt, Artificial Sequence
  TRAC-241
gucucucagcugguacacgg

SEQ ID NO: 217 - RNA, 22 nt, Artificial Sequence
  TRAC-179
gguacacggcagggucaggguu

SEQ ID NO: 218 - RNA, 21 nt, Artificial Sequence
  TRAC-178
guacacggcagggucaggguu

SEQ ID NO: 219 - RNA, 20 nt, Artificial Sequence
  TRBC-40
cacccagaucgucagcgccg

SEQ ID NO: 220 - RNA, 20 nt, Artificial Sequence
  TRBC-52
caaacacagcgaccucgggu

SEQ ID NO: 221 - RNA, 20 nt, Artificial Sequence
  TRBC-25
ugacgaguggacccaggaua

SEQ ID NO: 222 - RNA, 20 nt, Artificial Sequence
  TRBC-35
ggcucucggagaaugacgag

SEQ ID NO: 223 - RNA, 20 nt, Artificial Sequence
  TRBC-50
ggccucggcgcugacgaucu

SEQ ID NO: 224 - RNA, 20 nt, Artificial Sequence
  TRBC-39
gaaaaacguguucccacccg

SEQ ID NO: 225 - RNA, 20 nt, Artificial Sequence
  TRBC-49
augacgaguggacccaggau

SEQ ID NO: 226 - RNA, 20 nt, Artificial Sequence
  TRBC-51
aguccaguucuacgggcucu

SEQ ID NO: 227 - RNA, 20 nt, Artificial Sequence
  TRBC-26
cgcugucaaguccaguucua

SEQ ID NO: 228 - RNA, 20 nt, Artificial Sequence
  TRBC-47
aucgucagcgccgaggccug

SEQ ID NO: 229 - RNA, 20 nt, Artificial Sequence
  TRBC-45
ucaaacacagcgaccucggg

SEQ ID NO: 230 - RNA, 20 nt, Artificial Sequence
  TRBC-34
cguagaacuggacuugacag

SEQ ID NO: 231 - RNA, 20 nt, Artificial Sequence
  TRBC-227
aggccucggcgcugacgauc

SEQ ID NO: 232 - RNA, 20 nt, Artificial Sequence
  TRBC-41
ugacagcggaagugguugcg

SEQ ID NO: 233 - RNA, 20 nt, Artificial Sequence
  TRBC-30
uugacagcggaagugguugc

SEQ ID NO: 234 - RNA, 20 nt, Artificial Sequence
  TRBC-206
ucuccgagagcccguagaac

SEQ ID NO: 235 - RNA, 20 nt, Artificial Sequence
  TRBC-32
cgggugggaacacguuuuuc

SEQ ID NO: 236 - RNA, 20 nt, Artificial Sequence
  TRBC-276
gacagguuuggcccuauccu

SEQ ID NO: 237 - RNA, 20 nt, Artificial Sequence
  TRBC-274
gaucgucagcgccgaggccu

SEQ ID NO: 238 - RNA, 20 nt, Artificial Sequence
  TRBC-230
ggcucaaacacagcgaccuc

SEQ ID NO: 239 - RNA, 20 nt, Artificial Sequence
  TRBC-235
ugagggucucggccaccuuc

SEQ ID NO: 240 - RNA, 20 nt, Artificial Sequence
  TRBC-38
aggcuucuaccccgaccacg

SEQ ID NO: 241 - RNA, 20 nt, Artificial Sequence
  TRBC-223
ccgaccacguggagcugagc

SEQ ID NO: 242 - RNA, 20 nt, Artificial Sequence
  TRBC-221
ugacagguuuggcccuaucc

SEQ ID NO: 243 - RNA, 20 nt, Artificial Sequence
  TRBC-48
cuugacagcggaagugguug

SEQ ID NO: 244 - RNA, 20 nt, Artificial Sequence
  TRBC-216
agaucgucagcgccgaggcc

SEQ ID NO: 245 - RNA, 20 nt, Artificial Sequence
  TRBC-210
gcgcugacgaucugggugac

SEQ ID NO: 246 - RNA, 20 nt, Artificial Sequence
  TRBC-268
ugagggcgggcugcuccuug

SEQ ID NO: 247 - RNA, 20 nt, Artificial Sequence
  TRBC-193
guugcggggguucugccaga

SEQ ID NO: 248 - RNA, 20 nt, Artificial Sequence
  TRBC-246
agcucagcuccacguggucg

SEQ ID NO: 249 - RNA, 20 nt, Artificial Sequence
  TRBC-228
gcggcugcucaggcaguauc

SEQ ID NO: 250 - RNA, 20 nt, Artificial Sequence
  TRBC-43
gcggggguucugccagaagg

SEQ ID NO: 251 - RNA, 20 nt, Artificial Sequence
  TRBC-272
uggcucaaacacagcgaccu

SEQ ID NO: 252 - RNA, 20 nt, Artificial Sequence
  TRBC-33
acuggacuugacagcggaag

SEQ ID NO: 253 - RNA, 20 nt, Artificial Sequence
  TRBC-44
gacagcggaagugguugcgg

SEQ ID NO: 254 - RNA, 20 nt, Artificial Sequence
  TRBC-211
gcugucaaguccaguucuac

SEQ ID NO: 255 - RNA, 20 nt, Artificial Sequence
  TRBC-253
guaucuggagucauugaggg

SEQ ID NO: 256 - RNA, 17 nt, Artificial Sequence
  TRBC-18
cucggcgcugacgaucu

SEQ ID NO: 257 - RNA, 17 nt, Artificial Sequence
  TRBC-6
ccucggcgcugacgauc

SEQ ID NO: 258 - RNA, 17 nt, Artificial Sequence
  TRBC-85
ccgagagcccguagaac

SEQ ID NO: 259 - RNA, 17 nt, Artificial Sequence
  TRBC-129
ccagaucgucagcgccg

SEQ ID NO: 260 - RNA, 17 nt, Artificial Sequence
  TRBC-93
gaaugacgaguggaccc

SEQ ID NO: 261 - RNA, 22 nt, Artificial Sequence
  TRBC-415
gggugacagguuuggcccuauc

SEQ ID NO: 262 - RNA, 21 nt, Artificial Sequence
  TRBC-414
ggugacagguuuggcccuauc

SEQ ID NO: 263 - RNA, 20 nt, Artificial Sequence
  TRBC-310
gugacagguuuggcccuauc

SEQ ID NO: 264 - RNA, 18 nt, Artificial Sequence
  TRBC-308
gacagguuuggcccuauc

SEQ ID NO: 265 - RNA, 22 nt, Artificial Sequence
  TRBC-401
gauacugccugagcagccgccu

SEQ ID NO: 266 - RNA, 24 nt, Artificial Sequence
  TRBC-468
gaccacguggagcugagcuggugg

SEQ ID NO: 267 - RNA, 18 nt, Artificial Sequence
  TRBC-462
guggagcugagcuggugg

SEQ ID NO: 268 - RNA, 24 nt, Artificial Sequence
  TRBC-424
gggcgggcugcuccuugaggggcu

SEQ ID NO: 269 - RNA, 23 nt, Artificial Sequence
  TRBC-423
ggcgggcugcuccuugaggggcu

SEQ ID NO: 270 - RNA, 22 nt, Artificial Sequence
  TRBC-422
gcgggcugcuccuugaggggcu

SEQ ID NO: 271 - RNA, 20 nt, Artificial Sequence
  TRBC-420
gggcugcuccuugaggggcu

SEQ ID NO: 272 - RNA, 19 nt, Artificial Sequence
  TRBC-419
ggcugcuccuugaggggcu

SEQ ID NO: 273 - RNA, 18 nt, Artificial Sequence
  TRBC-418
gcugcuccuugaggggcu

SEQ ID NO: 274 - RNA, 24 nt, Artificial Sequence
  TRBC-445
ggugaaugggaaggaggugcacag

SEQ ID NO: 275 - RNA, 23 nt, Artificial Sequence
  TRBC-444
gugaaugggaaggaggugcacag

SEQ ID NO: 276 - RNA, 21 nt, Artificial Sequence
  TRBC-442
gaaugggaaggaggugcacag

SEQ ID NO: 277 - DNA, 149 bp, Artificial Sequence
  TRAC gRNA
agcgctctcgtacagagttggcattataatacgactcactataggggagaatcaaaatcggtgaatgttttagagctagaaatagcaagttaaaataaggctagtccgttatcaacttgaaaaagtggcaccgagtcggtgcttttttt

SEQ ID NO: 278 - RNA, 100 nt, Artificial Sequence
  TRAC gRNA
gagaaucaaaaucggugaauguuuuagagcuagaaauagcaaguuaaaauaaggcuaguccguuaucaacuugaaaaaguggcaccgagucggugcuuuu

SEQ ID NO: 279 - protein, 1344 aa, Streptococcus mutans
  Cas9
KKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 280 - protein, 1367 aa, Streptococcus pyogenes
  Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 281 - protein, 1387 aa, Streptococcus thermophilus
  Cas9
TKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 282 - protein, 1333 aa, Listeria innocua
  Cas9
KKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 283 - protein, 1082 aa, Neisseria meningitidis
  Cas9
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 284 - protein, 1368 aa, Streptococcus pyogenes [sequence omitted]

SEQ ID NO: 285 - protein, 1368 aa, Streptococcus pyogenes [sequence omitted]

SEQ ID NO: 286 - protein, 1082 aa, Neisseria meningitidis
  Cas9
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 287 - protein, 1053 aa, Staphylococcus aureus
  Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 288 - DNA, 68 bp, Artificial Sequence
  GMCSFR alpha chain signal sequence
atgctgctgctggtgaccagcctgctgctgtgcgagctgccccaccccgcctttctgctgatccccga

SEQ ID NO: 289 - DNA, 25 bp, Artificial Sequence
  human HBB gene splice acceptor
ctgacctcttctcttcctcccacag

SEQ ID NO: 290 - DNA, 13 bp, Artificial Sequence
  human IgG gene splice acceptor
tttctctccacag

SEQ ID NO: 291 - protein, 228 aa, Artificial Sequence
  Spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 292 - protein, 164 aa, Artificial Sequence
  CD3 zeta precursor
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 293 - protein, 107 aa, Artificial Sequence
  FKBP
GVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLE

SEQ ID NO: 294 - protein, 107 aa, Artificial Sequence
  FKBP12v36
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 295 - protein, 16 aa, Artificial Sequence
  human C-Src acylation motif
MGSNKSKPKDASQRRR

SEQ ID NO: 296 - protein, 5 aa, Artificial Sequence
  dual acylation motif
  VARIANT (4)...(4): Xaa is any amino acid
MGCXC

SEQ ID NO: 297 - protein, 4 aa, Artificial Sequence
  CAAX motif
  VARIANT (4)...(4): Xaa is any amino acid
CAAX